SEQUENCE LISTING (131 sequences)

SEQ ID NO: 1 - protein, 130 aa, synthetic construct
  source 1..130: note = VHH R3_C4
QVQLQESGGGLVQAGDSLRLSCAVSGRPFSTYTMGWFRQAPGKEREFVAAMRWSGGTIYYADSVKGRFTISRDNDKNTVNLEMNSLKPEDTAVYYCAAAYVSKANYGSLWYRASGLYDYWGQGTQVTVSS

SEQ ID NO: 2 - protein, 130 aa, synthetic construct
  source 1..130: note = VHH R4_DC16
QVQLQESGGGLVQAGDSLRLSCAVSGRPFSTYTMGWFRQAPGKEREFVAAMRWSGGTIYYADSVKGRFTISRDNAQNTVNLQMNSLKPEDTAVYYCAAAYVSKANYGSSWYRNSGLYDYWGQGTQVTVSS

SEQ ID NO: 3 - protein, 130 aa, synthetic construct
  source 1..130: note = VHH R3_DC20
QVQLQESGGGLVQAGDSLRLSCAVSGRPFSTYTMGWFRQAPGKEREFVAAMRWSGGTIYYADSVKGRFTISRDNAQNTVNLQMNSLEPEDTAVYYCAAAYVSKANYGSLWYRNSGLYDYWGQGTQVTVSS

SEQ ID NO: 4 - protein, 130 aa, synthetic construct
  source 1..130: note = VHH R3_DC2
QVQLQESGGGLVQTGDSLRLSCAVSGRPFSTYTMGWFRQAPGKEREFVASMRWSGGTKYYSDSVKGRFSISRDNDQNTVNLQMNSLKPEDTAVYYCAAAYVSKANYGSLWYRNSGLYDYWGQGTQVTVSS

SEQ ID NO: 5 - protein, 130 aa, synthetic construct
  source 1..130: note = VHH R4_DC20
QVQLQESGGGLVQTGDSLRLSCAVSGRPFSTYTMGWFRQAPGKEREFVASMRWSGGTMNYADSVKGRFTISRDNDQNTVNLQMNSLKPEDTAVYYCAAAYVSKANYGSLWYRNSGLYDYWGQGTQVTVSS

SEQ ID NO: 6 - protein, 130 aa, synthetic construct
  source 1..130: note = VHH R4_DC9
QVQLQESGGGLVQAGDSLRLSCAASGRIFSTYTMGWFRQAPGMEREFVAAIRWSGSTVYYADSVKGRFTISRDNAKNTVYLQMNSLKPEDTAVYYCAAARVSKANYGTLWYRASGLYDYWGQGTQVTVSS

SEQ ID NO: 7 - protein, 130 aa, synthetic construct
  source 1..130: note = VHH R4_DC6
QVQLQESGGGLVQAGDSLRLSCAASGRILSTFTMGWFRQAPGKEREFVAAMRWNGGTKNYADSVKGRFTISRDNANSVVYLQMNSTKPEDTAVYYCAAAYVSKANYGSLWYRTPSLYDYWGQGTQVTVSS

SEQ ID NO: 8 - protein, 130 aa, synthetic construct
  source 1..130: note = VHH R3_DC23
QVQLQESGGGLVQAGDSLTLSCAVSGRIFSTYTMGWFRQAPGKEREFVAAVRWGAGTIYYADSMKGRFTISRDSAKNTVDLQMNSTKPEDTAVYYCGAAYVSKANYGSLWYQDSRRYDYWGQGTQVTVSS

SEQ ID NO: 9 - protein, 130 aa, synthetic construct
  source 1..130: note = VHH R3_DC9
QVQLQESGGGLVQTGDSLRLSCAVSGRPFSTYTMGWFRQAPGKEREFVASMRWSGGTMYYSDSVKGRFTISRDNDKNTVNLQMNSLKPEDTAVYYCAAAYVSKANYGSLWYRNSGLYDYWGQGTQVTVSS

SEQ ID NO: 10 - protein, 130 aa, synthetic construct
  source 1..130: note = VHH R4_DC13
QVQLQESGGGLVQTGDSLRLSCAVSGRPFSTYTMGWFRQAPGKEREFVASMRWSGGTVYYSDSVKGRFTISRDNDKNTVNLQMNNLKPEDTAVYYCAAAYVSKANYGSFWYRNSDLYDYWGQGTQVTVSS

SEQ ID NO: 11 - protein, 5 aa, synthetic construct
  source 1..5: note = CDR1 of VHH R3_C4, VHH R4_DC16, VHH R3_DC20, VHH R3_DC2, VHHR4_DC20, VHH R4_DC9, VHH R3_DC23, VHH R3_DC9, VHH R4_DC13 Kabat
TYTMG

SEQ ID NO: 12 - protein, 5 aa, synthetic construct
  source 1..5: note = CDR1 of VHH R4_DC6 Kabat
TFTMG

SEQ ID NO: 13 - protein, 17 aa, synthetic construct
  source 1..17: note = CDR2 of VHH R3_C4, VHH R4_DC16, VHH R3_DC20 Kabat
AMRWSGGTIYYADSVKG

SEQ ID NO: 14 - protein, 17 aa, synthetic construct
  source 1..17: note = CDR2 of VHH R3_DC9 Kabat
SMRWSGGTMYYSDSVKG

SEQ ID NO: 15 - protein, 17 aa, synthetic construct
  source 1..17: note = CDR2 of VHH R3_DC2 Kabat
SMRWSGGTKYYSDSVKG

SEQ ID NO: 16 - protein, 17 aa, synthetic construct
  source 1..17: note = CDR2 of VHH R4_DC20 Kabat
SMRWSGGTMNYADSVKG

SEQ ID NO: 17 - protein, 17 aa, synthetic construct
  source 1..17: note = CDR2 of VHH R4_DC13 Kabat
SMRWSGGTVYYSDSVKG

SEQ ID NO: 18 - protein, 17 aa, synthetic construct
  source 1..17: note = CDR2 of VHH R4_DC9 Kabat
AIRWSGSTVYYADSVKG

SEQ ID NO: 19 - protein, 17 aa, synthetic construct
  source 1..17: note = CDR2 of VHH R4_DC6 Kabat
AMRWNGGTKNYADSVKG

SEQ ID NO: 20 - protein, 17 aa, synthetic construct
  source 1..17: note = CDR2 of VHH R3_DC23 Kabat
AVRWGAGTIYYADSMKG

SEQ ID NO: 21 - protein, 21 aa, synthetic construct
  source 1..21: note = CDR3 of VHH R3_C4 Kabat
AYVSKANYGSLWYRASGLYDY

SEQ ID NO: 22 - protein, 21 aa, synthetic construct
  source 1..21: note = CDR3 of VHH R4_DC16 Kabat
AYVSKANYGSSWYRNSGLYDY

SEQ ID NO: 23 - protein, 21 aa, synthetic construct
  source 1..21: note = CDR3 of VHH R3_DC20, VHH R3_DC9, VHH R3_DC2, VHH R4_DC20 Kabat
AYVSKANYGSLWYRNSGLYDY

SEQ ID NO: 24 - protein, 21 aa, synthetic construct
  source 1..21: note = CDR3 of VHH R4_DC13 Kabat
AYVSKANYGSFWYRNSDLYDY

SEQ ID NO: 25 - protein, 21 aa, synthetic construct
  source 1..21: note = CDR3 of VHH R4_DC9 Kabat
ARVSKANYGTLWYRASGLYDY

SEQ ID NO: 26 - protein, 21 aa, synthetic construct
  source 1..21: note = CDR3 of VHH R4_DC6 Kabat
AYVSKANYGSLWYRTPSLYDY

SEQ ID NO: 27 - protein, 21 aa, synthetic construct
  source 1..21: note = CDR3 of VHH R3_DC23 Kabat
AYVSKANYGSLWYQDSRRYDY

SEQ ID NO: 28 - protein, 30 aa, synthetic construct
  source 1..30: note = FR1 of VHH R3_C4, VHH R4_DC16, VHH R3_DC20 Kabat
QVQLQESGGGLVQAGDSLRLSCAVSGRPFS

SEQ ID NO: 29 - protein, 30 aa, synthetic construct
  source 1..30: note = FR1 of VHH R3_DC9, VHH R3_DC2, VHH R4_DC20, VHH R4_DC13 Kabat
QVQLQESGGGLVQTGDSLRLSCAVSGRPFS

SEQ ID NO: 30 - protein, 30 aa, synthetic construct
  source 1..30: note = FR1 of VHH R4_DC9 Kabat
QVQLQESGGGLVQAGDSLRLSCAASGRIFS

SEQ ID NO: 31 - protein, 30 aa, synthetic construct
  source 1..30: note = FR1 of VHH R4_DC6 Kabat
QVQLQESGGGLVQAGDSLRLSCAASGRILS

SEQ ID NO: 32 - protein, 30 aa, synthetic construct
  source 1..30: note = FR1 of VHH R3_DC23 Kabat
QVQLQESGGGLVQAGDSLTLSCAVSGRIFS

SEQ ID NO: 33 - protein, 14 aa, synthetic construct
  source 1..14: note = FR2 of VHH R3_C4, VHH R4_DC16, VHH R3_DC20, VHH R3_DC2, VHHR4_DC20, VHH R4_DC6, HH R3_DC23, VHH R3_DC9, VHH R4_DC13 Kabat, Martin
WFRQAPGKEREFVA

SEQ ID NO: 34 - protein, 14 aa, synthetic construct
  source 1..14: note = FR2 of VHH R4_DC9 Kabat, Martin
WFRQAPGMEREFVA

SEQ ID NO: 35 - protein, 32 aa, synthetic construct
  source 1..32: note = FR3 of VHH R3_C4 Kabat
RFTISRDNDKNTVNLEMNSLKPEDTAVYYCAA

SEQ ID NO: 36 - protein, 32 aa, synthetic construct
  source 1..32: note = FR3 of VHH R4_DC16 Kabat
RFTISRDNAQNTVNLQMNSLKPEDTAVYYCAA

SEQ ID NO: 37 - protein, 32 aa, synthetic construct
  source 1..32: note = FR3 of VHH R3_DC20 Kabat
RFTISRDNAQNTVNLQMNSLEPEDTAVYYCAA

SEQ ID NO: 38 - protein, 32 aa, synthetic construct
  source 1..32: note = FR3 of VHH R3_DC9 Kabat
RFTISRDNDKNTVNLQMNSLKPEDTAVYYCAA

SEQ ID NO: 39 - protein, 32 aa, synthetic construct
  source 1..32: note = FR3 of VHH R3_DC2 Kabat
RFSISRDNDQNTVNLQMNSLKPEDTAVYYCAA

SEQ ID NO: 40 - protein, 32 aa, synthetic construct
  source 1..32: note = FR3 of VHH R4_DC20 Kabat
RFTISRDNDQNTVNLQMNSLKPEDTAVYYCAA

SEQ ID NO: 41 - protein, 32 aa, synthetic construct
  source 1..32: note = FR3 of VHH R4_DC13 Kabat
RFTISRDNDKNTVNLQMNNLKPEDTAVYYCAA

SEQ ID NO: 42 - protein, 32 aa, synthetic construct
  source 1..32: note = FR3 of VHH R4_DC9 Kabat
RFTISRDNAKNTVYLQMNSLKPEDTAVYYCAA

SEQ ID NO: 43 - protein, 32 aa, synthetic construct
  source 1..32: note = FR3 of VHH R4_DC6 Kabat
RFTISRDNANSVVYLQMNSTKPEDTAVYYCAA

SEQ ID NO: 44 - protein, 32 aa, synthetic construct
  source 1..32: note = FR3 of VHH R3_DC23 Kabat
RFTISRDSAKNTVDLQMNSTKPEDTAVYYCGA

SEQ ID NO: 45 - protein, 11 aa, synthetic construct
  source 1..11: note = FR4 of VHH R3_C4, VHH R4_DC16, VHH R3_DC20, VHH R3_DC9, VHHR3_DC2, VHH R4_DC20, VHH R4_DC13, VHH R4_DC9, VHH R4_DC6, VHHR3_DC23 Kabat, Martin
WGQGTQVTVSS

SEQ ID NO: 46 - protein, 5 aa, synthetic construct
  SITE 2: note = MISC_FEATURE - Xaa is Y (Tyr, Tyrosine), F (Phe, Phenylalanine) or W (Trp, Tryptophan)
  source 1..5: note = CDR1 Kabat
TXTMG

SEQ ID NO: 47 - protein, 17 aa, synthetic construct
  SITE 1: note = MISC_FEATURE - Xaa is A (Ala, Alanine), G (Gly, Glycine), S (Ser, Serine) or T(Thr, Threonine)
  SITE 2: note = MISC_FEATURE - Xaa is M (Met, Methionine), I (Ile, Isoleucine), L (Leu, Leucine)or V (Val, Valine)
  SITE 5: note = MISC_FEATURE - Xaa is S (Ser, Serine), N (Asn, Asparagine), T (Thr, Threonine),Q (Gln, Glutamine) or G (Gly, Glycine)
  SITE 6: note = MISC_FEATURE - Xaa is G (Gly, Glycine), A (Ala, Alanine) or P (Pro, Proline)
  SITE 7: note = MISC_FEATURE - Xaa is G (Gly, Glycine) or S (Ser, Serine)
  SITE 9: note = MISC_FEATURE - Xaa is I (Ile, Isoleucine), L (Leu, Leucine), M (Met,Methionine), K (Lys, Lysine), V (Val, Valine) or R (Arg,Arginine)
  SITE 10: note = MISC_FEATURE - Xaa is Y (Tyr, Tyrosine), W (Trp, Tryptophan), N (Asn,Asparagine), S (Ser, Serine), F (Phe, Phenylalanine), Q (Gln,Glutamine) or T (Thr, Threonine)
  SITE 12: note = MISC_FEATURE - Xaa is A (Ala, Alanine), G (Gly, Glycine), S (Ser, Serine), N(Asn, Asparagine), T (Thr, Threonine) or V (Val, Valine)
  SITE 15: note = MISC_FEATURE - Xaa is V (Val, Valine) or M (Met, Methionine)
  source 1..17: note = CDR2 Kabat
XXRWXXXTXXYXDSXKG

SEQ ID NO: 48 - protein, 21 aa, synthetic construct
  SITE 2: note = MISC_FEATURE - Xaa is Y (Tyr, Tyrosine), W (Trp, Tryptophan), R (Arg, Arginine)or K (Lys, Lysine)
  SITE 10: note = MISC_FEATURE - Xaa is S (Ser, Serine), N (Asn, Asparagine) or T (Thr, Threonine)
  SITE 11: note = MISC_FEATURE - Xaa is L (Leu, Leucine), I (Ile, Isoleucine), S (Ser, Serine), N(Asn, Asparagine), F (Phe, Phenylalanine), M (Met, Methionine), V(Val, Valine) or T (Thr, Threonine)
  SITE 14: note = MISC_FEATURE - Xaa is R (Arg, Arginine), S (Ser, Serine), K (Lys, Lysine) or N(Asn, Asparagine)
  SITE 15: note = MISC_FEATURE - Xaa is A (Ala, Alanine), G (Gly, Glycine), N (Asn, Asparagine), S(Ser, Serine), T (Thr, Threonine), D (Asp, Aspartic Acid) or E(Glu, Glutamic Acid)
  SITE 16: note = MISC_FEATURE - Xaa is S (Ser, Serine), P (Pro, Proline), A (Ala, Alanine), G(Gly, Glycine) or T (Thr, Threonine)
  SITE 17: note = MISC_FEATURE - Xaa is G (Gly, Glycine), A (Ala, Alanine), D (Asp, AsparticAcid), S (Ser, Serine), R (Arg, Arginine), T (Thr, Threonine), K(Lys, Lysine), E (Glu, Glutamic Acid)
  SITE 18: note = MISC_FEATURE - Xaa is L (Leu, Leucine), I (Ile, Isoleucine), R (Arg, Arginine),V (Val, Valine), M (Met, Methionine) or K (Lys, Lysine)
  source 1..21: note = CDR3 Kabat, Martin
AXVSKANYGXXWYXXXXXYDY

SEQ ID NO: 49 - protein, 5 aa, synthetic construct
  SITE 2: note = MISC_FEATURE - Xaa is Y (Tyr, Tyrosine) or F (Phe, Phenylalanine)
  source 1..5: note = variation CDR1 Kabat
TXTMG

SEQ ID NO: 50 - protein, 17 aa, synthetic construct
  SITE 1: note = MISC_FEATURE - Xaa is A (Ala, Alanine) or S (Ser, Serine)
  SITE 2: note = MISC_FEATURE - Xaa is M (Met, Methionine), I (Ile, Isoleucine) or V (Val,Valine)
  SITE 5: note = MISC_FEATURE - Xaa is S (Ser, Serine) or N (Asn, Asparagine)
  SITE 6: note = MISC_FEATURE - Xaa is G (Gly, Glycine) or A (Ala, Alanine)
  SITE 9: note = MISC_FEATURE - Xaa is I (Ile, Isoleucine), M (Met, Methionine), K (Lys, Lysine)or V (Val, Valine)
  SITE 10: note = MISC_FEATURE - Xaa is Y (Tyr, Tyrosine) or N (Asn, Asparagine)
  SITE 12: note = MISC_FEATURE - Xaa is A (Ala, Alanine) or S (Ser, Serine)
  SITE 15: note = MISC_FEATURE - Xaa is V (Val, Valine) or M (Met, Methionine)
  source 1..17: note = variation CDR2 Kabat
XXRWXXGTXXYXDSXKG

SEQ ID NO: 51 - protein, 21 aa, synthetic construct
  SITE 2: note = MISC_FEATURE - Xaa is Y (Tyr, Tyrosine) or R (Arg, Arginine)
  SITE 10: note = MISC_FEATURE - Xaa is S (Ser, Serine) or T (Thr, Threonine)
  SITE 11: note = MISC_FEATURE - Xaa is L (Leu, Leucine), S (Ser, Serine) or F (Phe,Phenylalanine)
  SITE 14: note = MISC_FEATURE - Xaa is R (Arg, Arginine) or Q (Gln, Glutamine)
  SITE 15: note = MISC_FEATURE - Xaa is A (Ala, Alanine), N (Asn, Asparagine), T (Thr, Threonine)or D (Asp, Aspartic Acid)
  SITE 16: note = MISC_FEATURE - Xaa is S (Ser, Serine) or P (Pro, Proline)
  SITE 17: note = MISC_FEATURE - Xaa is G (Gly, Glycine), D (Asp, Aspartic Acid), S (Ser, Serine)or R (Arg, Arginine)
  SITE 18: note = MISC_FEATURE - Xaa is L (Leu, Leucine) or R (Arg, Arginine)
  source 1..21: note = variation CDR3 Kabat, Martin
AXVSKANYGXXWYXXXXXYDY

SEQ ID NO: 52 - protein, 10 aa, synthetic construct
  source 1..10: note = CDR1 R3_C4, R4_DC16, R3_DC20, R3_DC9, R3_DC2, R4_DC20, R4_DC13 Martin
GRPFSTYTMG

SEQ ID NO: 53 - protein, 10 aa, synthetic construct
  source 1..10: note = CDR1 R3_DC23, R4_DC9 Martin
GRIFSTYTMG

SEQ ID NO: 54 - protein, 11 aa, synthetic construct
  source 1..11: note = CDR1 R4_DC6 Martin
GRILSTFTMGW

SEQ ID NO: 55 - protein, 10 aa, synthetic construct
  source 1..10: note = CDR2 of VHH R3_C4, VHH R4_DC16, VHH R3_DC20 Martin
AMRWSGGTIY

SEQ ID NO: 56 - protein, 10 aa, synthetic construct
  source 1..10: note = CDR2 of VHH R3_DC9 Martin
SMRWSGGTMY

SEQ ID NO: 57 - protein, 10 aa, synthetic construct
  source 1..10: note = CDR2 of VHH R3_DC2 Martin
SMRWSGGTKY

SEQ ID NO: 58 - protein, 10 aa, synthetic construct
  source 1..10: note = CDR2 of VHH R4_DC20 Martin
SMRWSGGTMN

SEQ ID NO: 59 - protein, 10 aa, synthetic construct
  source 1..10: note = CDR2 of VHH R4_DC13 Martin
SMRWSGGTVY

SEQ ID NO: 60 - protein, 10 aa, synthetic construct
  source 1..10: note = CDR2 of VHH R4_DC9 Martin
AIRWSGSTVY

SEQ ID NO: 61 - protein, 10 aa, synthetic construct
  source 1..10: note = CDR2 of VHH R4_DC6 Martin
AMRWNGGTKN

SEQ ID NO: 62 - protein, 10 aa, synthetic construct
  source 1..10: note = CDR2 of VHH R3_DC23 Martin
AVRWGAGTIY

SEQ ID NO: 63 - protein, 10 aa, synthetic construct
  SITE 3: note = MISC_FEATURE - Xaa is P (Pro, Proline), I (Ile, Isoleucine), A (Ala, Alanine), L(Leu, Leucine) or V (Val, Valine)
  SITE 4: note = MISC_FEATURE - Xaa is F (Phe, Phenylalanine), L (Leu, Leucine), I (Ile,Isoleucine) or M (Met, Methionine)
  SITE 7: note = MISC_FEATURE - Xaa is Y (Tyr, Tyrosine), F (Phe, Phenylalanine) or W (Trp,Tryptophan)
  source 1..10: note = CDR1 Martin, AbM, IMGT
GRXXSTXTMG

SEQ ID NO: 64 - skipped

SEQ ID NO: 65 - protein, 10 aa, synthetic construct
  SITE 3: note = MISC_FEATURE - Xaa is P (Pro, Proline) or I (Ile, Isoleucine)
  SITE 4: note = MISC_FEATURE - Xaa is F (Phe, Phenylalanine) or L (Leu, Leucine)
  SITE 7: note = MISC_FEATURE - Xaa is Y (Tyr, Tyrosine) or F (Phe, Phenylalanine)
  source 1..10: note = variation CDR1 Martin, AbM, IMGT
GRXXSTXTMG

SEQ ID NO: 66 - protein, 10 aa, synthetic construct
  SITE 1: note = MISC_FEATURE - Xaa is A (Ala, Alanine) or S (Ser, Serine)
  SITE 2: note = MISC_FEATURE - Xaa is M (Met, Methionine), I (Ile, Isoleucine) or V (Val,Valine)
  SITE 5: note = MISC_FEATURE - Xaa is S (Ser, Serine) or N (Asn, Asparagine)
  SITE 6: note = MISC_FEATURE - Xaa is G (Gly, Glycine) or A (Ala, Alanine)
  SITE 9: note = MISC_FEATURE - Xaa is I (Ile, Isoleucine), M (Met, Methionine), K (Lys, Lysine) or V (Val, Valine)
  SITE 10: note = MISC_FEATURE - Xaa is Y (Tyr, Tyrosine) or N (Asn, Asparagine)
  source 1..10: note = variation CDR2 Martin, AbM
XXRWXXGTXX

SEQ ID NO: 67 - protein, 20 aa, synthetic construct
  SITE 2: note = MISC_FEATURE - Xaa is Y (Tyr, Tyrosine), W (Trp, Tryptophan), R (Arg, Arginine)or K (Lys, Lysine)
  SITE 10: note = MISC_FEATURE - Xaa is S (Ser, Serine), N (Asn, Asparagine) or T (Thr, Threonine)
  SITE 11: note = MISC_FEATURE - Xaa is L (Leu, Leucine), I (Ile, Isoleucine), S (Ser, Serine), N(Asn, Asparagine), F (Phe, Phenylalanine), M (Met, Methionine), V(Val, Valine) or T (Thr, Threonine)
  SITE 14: note = MISC_FEATURE - Xaa is R (Arg, Arginine), Q (Gln, Glutamine), K, (Lys, Lysine) orN (Asn, Asparagine)
  SITE 15: note = MISC_FEATURE - Xaa is A (Ala, Alanine), G (Gly, Glycine), N (Asn, Asparagine), S(Ser, Serine), T (Thr, Threonine), D (Asp, Aspartic Acid) or E(Glu, Glutamic Acid)
  SITE 16: note = MISC_FEATURE - Xaa is S (Ser, Serine), P (Pro, Proline), A (Ala, Alanine), G(Gly, Glycine) or T (Thr, Threonine)
  SITE 17: note = MISC_FEATURE - Xaa is G (Gly, Glycine), A (Ala, Alanine), D (Asp, AsparticAcid), S (Ser, Serine), R (Arg, Arginine), T (Thr, Threonine), K(Lys, Lysine) or E (Glu, Glutamic Acid)
  SITE 18: note = misc_feature - Xaa can be any naturally occurring amino acid
  source 1..20: note = CDR3 AbM, Chothia
AXVSKANYGXXWYXXXXXYD

SEQ ID NO: 68 - protein, 20 aa, synthetic construct
  SITE 2: note = MISC_FEATURE - Xaa is Y (Tyr, Tyrosine) or R (Arg, Arginine)
  SITE 10: note = MISC_FEATURE - Xaa is S (Ser, Serine) or T (Thr, Threonine)
  SITE 11: note = MISC_FEATURE - Xaa is L (Leu, Leucine), S (Ser, Serine) or F (Phe,Phenylalanine)
  SITE 14: note = MISC_FEATURE - Xaa is R (Arg, Arginine) or Q (Gln, Glutamine)
  SITE 15: note = MISC_FEATURE - Xaa is A (Ala, Alanine), N (Asn, Asparagine), T (Thr, Threonine)or D (Asp, Aspartic Acid)
  SITE 16: note = MISC_FEATURE - Xaa is S (Ser, Serine) or P (Pro, Proline)
  SITE 17: note = MISC_FEATURE - Xaa is G (Gly, Glycine), D (Asp, Aspartic Acid), S (Ser, Serine)or R (Arg, Arginine)
  SITE 18: note = MISC_FEATURE - Xaa is L (Leu, Leucine) or R (Arg, Arginine)
  source 1..20: note = variation CDR3 AbM, Chothia
AXVSKANYGXXWYXXXXXYD

SEQ ID NO: 69 - protein, 9 aa, synthetic construct
  SITE 3: note = MISC_FEATURE - Xaa is P (Pro, Proline), I (Ile, Isoleucine), A (Ala, Alanine), L(Leu, Leucine) or V (Val, Valine)
  SITE 4: note = MISC_FEATURE - Xaa is F (Phe, Phenylalanine), L (Leu, Leucine), I (Ile,Isoleucine) or M (Met, Methionine)
  SITE 7: note = MISC_FEATURE - Xaa is Y (Tyr, Tyrosine), F (Phe, Phenylalanine) or W (Trp,Tryptophan)
  source 1..9: note = CDR1 Chothia
GRXXSTXTM

SEQ ID NO: 70 - skipped

SEQ ID NO: 71 - protein, 9 aa, synthetic construct
  SITE 3: note = MISC_FEATURE - Xaa is P (Pro, Proline) or I (Ile, Isoleucine)
  SITE 4: note = MISC_FEATURE - Xaa is F (Phe, Phenylalanine) or L (Leu, Leucine)
  SITE 7: note = MISC_FEATURE - Xaa is Y (Tyr, Tyrosine) or F (Phe, Phenylalanine)
  source 1..9: note = variation CDR1 Chothia
GRXXSTXTM

SEQ ID NO: 72-73 - skipped (2 entries)

SEQ ID NO: 74 - protein, 23 aa, synthetic construct
  SITE 1: note = MISC_FEATURE - Xaa is A (Ala, Alanine), G (Gly, Glycine), S (Ser, Serine), T(Thr, Threonine), V (Val, Valine) or N (Asn, Asparagine)
  SITE 4: note = MISC_FEATURE - Xaa is Y (Tyr, Tyrosine), W (Trp, Tryptophan), R (Arg, Arginine)or K (Lys, Lysine)
  SITE 12: note = MISC_FEATURE - Xaa is S (Ser, Serine), N (Asn, Asparagine) or T (Thr, Threonine)
  SITE 13: note = MISC_FEATURE - Xaa is L (Leu, Leucine), I (Ile, Isoleucine), S (Ser, Serine), N(Asn, Asparagine), F (Phe, Phenylalanine), M (Met, Methionine), V(Val, Valine) or T (Thr, Threonine)
  SITE 16: note = MISC_FEATURE - Xaa is R (Arg, Arginine), Q (Gln, Glutamine), K (Lys, Lysine) orN (Asn, Asparagine)
  SITE 17: note = MISC_FEATURE - Xaa is A (Ala, Alanine), G (Gly, Glycine), N (Asn, Asparagine), S(Ser, Serine), T (Thr, Threonine), D (Asp, Aspartic Acid) or E(Glu, Glutamic Acid)
  SITE 18: note = MISC_FEATURE - Xaa is S (Ser, Serine), P (Pro, Proline), A (Ala, Alanine), G(Gly, Glycine) or T (Thr, Threonine)
  SITE 19: note = MISC_FEATURE - Xaa is G (Gly, Glycine), A (Ala, Alanine), D (Asp, AsparticAcid), S (Ser, Serine), R (Arg, Arginine, T (Thr, Threonine), K(Lys, Lysine) or E (Glu, Glutamic Acid)
  SITE 20: note = MISC_FEATURE - Xaa is L (Leu, Leucine), I (Ile, Isoleucine), R (Arg, Arginine),V (Val, Valine), M (Met, Methionine) or K (Lys, Lysine)
  source 1..23: note = CDR3 IMGT
XAAXVSKANYGXXWYXXXXXYDY

SEQ ID NO: 75 - protein, 8 aa, synthetic construct
  SITE 1: note = MISC_FEATURE - Xaa is M (Met, Methionine), I (Ile, Isoleucine) or V (Val,Valine)
  SITE 4: note = MISC_FEATURE - Xaa is S (Ser, Serine) or N (Asn, Asparagine)
  SITE 5: note = MISC_FEATURE - Xaa is G (Gly, Glycine) or A (Ala, Alanine)
  SITE 8: note = MISC_FEATURE - Xaa is I (Ile, Isoleucine), M (Met, Methionine), K (Lys, Lysine)or V (Val, Valine)
  source 1..8: note = variation CDR2 IMGT
XRWXXGTX

SEQ ID NO: 76 - protein, 23 aa, synthetic construct
  SITE 1: note = MISC_FEATURE - Xaa is A (Ala, Alanine) or G (Gly, Glycine)
  SITE 4: note = MISC_FEATURE - Xaa is Y (Tyr, Tyrosine) or R (Arg, Arginine)
  SITE 12: note = MISC_FEATURE - Xaa is S (Ser, Serine) or T (Thr, Threonine)
  SITE 13: note = MISC_FEATURE - Xaa is L (Leu, Leucine), S (Ser, Serine) or F (Phe,Phenylalanine)
  SITE 16: note = MISC_FEATURE - Xaa is R (Arg, Arginine) or Q (Gln, Glutamine)
  SITE 17: note = MISC_FEATURE - Xaa is A (Ala, Alanine), N (Asn, Asparagine), T (Thr, Threonine)or D (Asp, Aspartic Acid)
  SITE 18: note = MISC_FEATURE - Xaa is S (Ser, Serine) or P (Pro, Proline)
  SITE 19: note = MISC_FEATURE - Xaa is G (Gly, Glycine), D (Asp, Aspartic Acid), S (Ser, Serine)or R (Arg, Arginine)
  SITE 20: note = MISC_FEATURE - Xaa is L (Leu, Leucine) or R (Arg, Arginine)
  source 1..23: note = variation CDR3 IMGT
XAAXVSKANYGXXWYXXXXXYDY

SEQ ID NO: 77 - protein, 6 aa, synthetic construct
  SITE 3: note = MISC_FEATURE - Xaa is Y (Tyr, Tyrosine), F (Phe, Phenylalanine) or W (Trp,Tryptophan)
  source 1..6: note = CDR1 MacCallum
STXTMG

SEQ ID NO: 78 - protein, 13 aa, synthetic construct
  SITE 4: note = MISC_FEATURE - Xaa is A (Ala, Alanine), G (Gly, Glycine), S (Ser, Serine) or T(Thr, Threonine)
  SITE 5: note = MISC_FEATURE - Xaa is M (Met, Methionine), I (Ile, Isoleucine), L (Leu, Leucine)or V (Val, Valine)
  SITE 8: note = MISC_FEATURE - Xaa is S (Ser, Serine), N (Asn, Asparagine), T (Thr, Threonine),Q (Gln, Glutamine) or G (Gly, Glycine)
  SITE 9: note = MISC_FEATURE - Xaa is G (Gly, Glycine), A (Ala, Alanine) or P (Pro, Proline)
  SITE 10: note = MISC_FEATURE - Xaa is G (Gly, Glycine) or S (Ser, Serine)
  SITE 12: note = MISC_FEATURE - Xaa is I (Ile, Isoleucine), L (Leu, Leucine), M (Met,Methionine), K (Lys, Lysine), V (Val, Valine) or R (Arg,Arginine)
  SITE 13: note = MISC_FEATURE - Xaa is Y (Tyr, Tyrosine), W (Trp, Tryptophan), N (Asn,Asparagine), S (Ser, Serine), F (Phe, Phenylalanine), Q (Gln,Glutamine) or T (Thr, Threonine)
  source 1..13: note = CDR2 MacCallum
FVAXXRWXXXTXX

SEQ ID NO: 79 - protein, 21 aa, synthetic construct
  SITE 1: note = MISC_FEATURE - Xaa is A (Ala, Alanine), G (Gly, Glycine), S (Ser, Serine), T(Thr, Threonine), V (Val, Valine) or N (Asn, Asparagine)
  SITE 4: note = MISC_FEATURE - Xaa is Y (Tyr, Tyrosine), W (Trp, Tryptophan), R (Arg, Arginine)or K (Lys, Lysine)
  SITE 12: note = MISC_FEATURE - Xaa is S (Ser, Serine), N (Asn, Asparagine) or T (Thr, Threonine)
  SITE 13: note = MISC_FEATURE - Xaa is L (Leu, Leucine), I (Ile, Isoleucine), S (Ser, Serine), F(Phe, Phenylalanine), M (Met, Methionine), V (Val, Valine) or T(Thr, Threonine)
  SITE 16: note = MISC_FEATURE - Xaa is R (Arg, Arginine), Q (Gln, Glutamine), K (Lys, Lysine) orN (Asn, Asparagine)
  SITE 17: note = MISC_FEATURE - Xaa is A (Ala, Alanine), G (Gly, Glycine), N (Asn, Asparagine), S(Ser, Serine), T (Thr, Threonine), D (Asp, Aspartic Acid) or E(Glu, Glutamic Acid)
  SITE 18: note = MISC_FEATURE - Xaa is S (Ser, Serine), P (Pro, Proline), A (Ala, Alanine), G(Gly, Glycine) or T (Thr, Threonine)
  SITE 19: note = MISC_FEATURE - Xaa is G (Gly, Glycine), A (Ala, Alanine), D (Asp, AsparticAcid), S (Ser, Serine), R (Arg, Arginine), T (Thr, Threonine), K(Lys, Lysine) or E (Glu, Glutamic Acid)
  SITE 20: note = MISC_FEATURE - Xaa is L (Leu, Leucine), I (Ile, Isoleucine), R (Arg, Arginine),V (Val, Valine), M (Met, Methionine)or K (Lys, Lysine)
  source 1..21: note = CDR3 MacCallum
XAAXVSKANYGXXWYXXXXXY

SEQ ID NO: 80 - protein, 6 aa, synthetic construct
  SITE 3: note = MISC_FEATURE - Xaa is Y (Tyr, Tyrosine) or F (Phe, Phenylalanine)
  source 1..6: note = variation CDR1 MacCallum
STXTMG

SEQ ID NO: 81 - protein, 13 aa, synthetic construct
  SITE 4: note = MISC_FEATURE - Xaa is A (Ala, Alanine), G (Gly, Glycine), S (Ser, Serine) or T(Thr, Threonine)
  SITE 5: note = MISC_FEATURE - Xaa is M (Met, Methionine), I (Ile, Isoleucine), L (Leu, Leucine)or V (Val, Valine)
  SITE 8: note = MISC_FEATURE - Xaa is S (Ser, Serine), N (Asn, Asparagine), T (Thr, Threonine),Q (Gln, Glutamine) or G (Gly, Glycine)
  SITE 9: note = MISC_FEATURE - Xaa is G (Gly, Glycine), A (Ala, Alanine) or P (Pro, Proline)
  SITE 10: note = MISC_FEATURE - Xaa is G (Gly, Glycine) or S (Ser, Serine)
  SITE 12: note = MISC_FEATURE - Xaa is I (Ile, Isoleucine), L (Leu, Leucine), M (Met,Methionine), K (Lys, Lysine), V (Val, Valine) or R (Arg,Arginine)
  SITE 13: note = MISC_FEATURE - Xaa is Y (Tyr, Tyrosine), W (Trp, Tryptophan), N (Asn,Asparagine), S (Ser, Serine), F (Phe, Phenylalanine), Q (Gln,Glutamine) or T (Thr, Threonine)
  source 1..13: note = variation CDR2 MacCallum
FVAXXRWXXXTXX

SEQ ID NO: 82 - protein, 21 aa, synthetic construct
  SITE 1: note = MISC_FEATURE - Xaa is A (Ala, Alanine) or G (Gly, Glycine)
  SITE 4: note = MISC_FEATURE - Xaa is Y (Tyr, Tyrosine) or R (Arg, Arginine)
  SITE 12: note = MISC_FEATURE - Xaa is S (Ser, Serine) or T (Thr, Threonine)
  SITE 13: note = MISC_FEATURE - Xaa is L (Leu, Leucine), S (Ser, Serine) or F (Phe,Phenylalanine)
  SITE 16: note = MISC_FEATURE - Xaa is R (Arg, Arginine) or Q (Gln, Glutamine)
  SITE 17: note = MISC_FEATURE - Xaa is A (Ala, Alanine), N (Asn, Asparagine), T (Thr, Threonine)or D (Asp, Aspartic Acid)
  SITE 18: note = MISC_FEATURE - Xaa is S (Ser, Serine) or P (Pro, Proline)
  SITE 19: note = MISC_FEATURE - Xaa is G (Gly, Glycine), D (Asp, Aspartic Acid), S (Ser, Serine)or R (Arg, Arginine)
  SITE 20: note = MISC_FEATURE - Xaa is L (Leu, Leucine) or R (Arg, Arginine)
  source 1..21: note = variation CDR3 MacCallum
XAAXVSKANYGXXWYXXXXXY

SEQ ID NO: 83 - protein, 130 aa, synthetic construct
  source 1..130: note = VHH R3_DC19
QVQLQESGGGLVQPGDSLRLSCAVSGRPFSTYTMGWFRQAPGKEREFVAAMRWSGGTIYYADSVKGRFTISRDNDKNTVNLEMNSLKPEDTAVYYCAAAYVSKANYGSLWYRASGLYDYWGQGTQVTVSS

SEQ ID NO: 84 - protein, 130 aa, synthetic construct
  source 1..130: note = VHH R3_DC21, VHH R3_DC22
QVQLQESGGGLVQAGDSLRLSCAVSGRPFSTYTMGWFRQAPGKEREFVAAMRWSGGTIYYADSVRGRFTISRDNDKNTVNLEMNSLKPEDTAVYYCAAAYVSKANYGSLWYRASGLYDYWGQGTQVTVSS

SEQ ID NO: 85 - protein, 130 aa, synthetic construct
  source 1..130: note = VHH R3_DC5
QVQLQESGGGLVQAGDSLRLSCAASGRIFSTYTMGWFRQAPGMEREFVAAIRWSGSTVYYADSVKGRFTISRDNAKNTVYLQMNSLKPEDTAVYYCAAARVSKANYGTLWYRATGLYDYWGQGTQVTVSS

SEQ ID NO: 86 - protein, 1273 aa, SARS-CoV-2
  source 1..1273: note = Sarbecovirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 87 - protein, 34 aa, SARS-CoV-2
  source 1..34: note = Sarbecovirus
ISGINASVVNIQKEIDRLNEVAKNLNESLIDLQE

SEQ ID NO: 88 - protein, 72 aa, SARS-CoV-2
  source 1..72: note = Sarbecovirus
PLQPELDSFKEELDKYFKNHTSPDVDLGDISGINASVVNIQKEIDRLNEVAKNLNESLIDLQELGKYEQYIK

SEQ ID NO: 89 - protein, 72 aa, Lyra11 Sarbecovirus
  source 1..72: note = Sarbecovirus
PLQPELDSFKEELDKYFKNHTSPDVDLGDISRINASVVNIQKEIDRLNEVAKNLNESLIDLQELGKYEQYIK

SEQ ID NO: 90 - protein, 72 aa, 273-2005 Sarbecovirus
  source 1..72: note = Sarbecovirus
PLQPELDSFKQELDKYFKNHTSPDVDLGDISGINASVVDIQKEIDRLNEVAKNLNESLIDLQELGKYEQYIK

SEQ ID NO: 91 - protein, 72 aa, GX2013 Sarbecovirus
  source 1..72: note = Sarbecovirus
PLQPELDSFKDELDKYFKNHTSPDVDLGDISGINASVVNIQKEIDRLNEVAKNLNESLIDLQELGKYEQYIK

SEQ ID NO: 92 - protein, 72 aa, ZC45 Sarbecovirus
  source 1..72: note = Sarbecovirus
PLQPELDSFKEELDKYFKNHTSPDIDLGDISGINASVVNIQKEIDRLNEVARNLNESLIDLQELGKYEQYIK

SEQ ID NO: 93 - protein, 72 aa, ZXC21 Sarbecovirus
  source 1..72: note = Sarbecovirus
PLQPELDSFKEELDKYFKNHTSPDIDLGDISGINASVVNIQKEIDRLNEVARNLNESLIDLQELGKYEHYIK

SEQ ID NO: 94 - protein, 72 aa, BM48-31 Sarbecovirus
  source 1..72: note = Sarbecovirus
PLQPELDSFKEELDKYFKNHTSQNVSLDGLNNINASVVDIKKEIEHLNEIAKSLNESLIDLQELGKYEQYIK

SEQ ID NO: 95 - protein, 72 aa, BtKY72 Sarbecovirus
  source 1..72: note = Sarbecovirus
PLQPELESFKQELDKYFKNHTSPDVDFGDISGINASVVDIKKEIAHLNEIAKNLNESLIDLQELGKYEQYVK

SEQ ID NO: 96 - protein, 366 aa, synthetic construct
  source 1..366: note = R3_DC23(hum)-Fc(YTE)
DVQLVESGGGLVQPGGSLRLSCAVSGRIFSTYTMGWFRQAPGKEREFVAAVRWGAGTIYYADSVKGRFTISRDNAKNTVYLQMNSLRPEDTAVYYCGAAYVSKANYGSLWYQDSRRYDYWGQGTLVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 25 aa, synthetic construct
  source 1..25: note = FR1 of VHH R3_C4, VHH R4_DC16, VHH R3_DC20 Martin
QVQLQESGGGLVQAGDSLRLSCAVS

SEQ ID NO: 98 - protein, 25 aa, synthetic construct
  source 1..25: note = FR1 of VHH R3_DC9, VHH R3_DC2, VHH R4_DC20, VHH R4_DC13 Martin
QVQLQESGGGLVQTGDSLRLSCAVS

SEQ ID NO: 99 - protein, 25 aa, synthetic construct
  source 1..25: note = FR1 of VHH R4_DC9, R4_DC6 Martin
QVQLQESGGGLVQAGDSLRLSCAAS

SEQ ID NO: 100 - protein, 25 aa, synthetic construct
  source 1..25: note = FR1 of VHH R3_DC23 Martin
QVQLQESGGGLVQAGDSLTLSCAVS

SEQ ID NO: 101 - protein, 39 aa, synthetic construct
  source 1..39: note = FR3 of VHH R3_C4 Martin
YADSVKGRFTISRDNDKNTVNLEMNSLKPEDTAVYYCAA

SEQ ID NO: 102 - protein, 39 aa, synthetic construct
  source 1..39: note = FR3 of VHH R4_DC16 Martin
YADSVKGRFTISRDNAQNTVNLQMNSLKPEDTAVYYCAA

SEQ ID NO: 103 - protein, 39 aa, synthetic construct
  source 1..39: note = FR3 of VHH R3_DC20 Martin
YADSVKGRFTISRDNAQNTVNLQMNSLEPEDTAVYYCAA

SEQ ID NO: 104 - protein, 39 aa, synthetic construct
  source 1..39: note = FR3 of VHH R3_DC9 Martin
YSDSVKGRFTISRDNDKNTVNLQMNSLKPEDTAVYYCAA

SEQ ID NO: 105 - protein, 39 aa, synthetic construct
  source 1..39: note = FR3 of VHH R3_DC2 Martin
YSDSVKGRFSISRDNDQNTVNLQMNSLKPEDTAVYYCAA

SEQ ID NO: 106 - protein, 39 aa, synthetic construct
  source 1..39: note = FR3 of VHH R4_DC20 Martin
YADSVKGRFTISRDNDQNTVNLQMNSLKPEDTAVYYCAA

SEQ ID NO: 107 - protein, 39 aa, synthetic construct
  source 1..39: note = FR3 of VHH R4_DC13 Martin
YSDSVKGRFTISRDNDKNTVNLQMNNLKPEDTAVYYCAA

SEQ ID NO: 108 - protein, 39 aa, synthetic construct
  source 1..39: note = FR3 of VHH R4_DC9 Martin
YADSVKGRFTISRDNAKNTVYLQMNSLKPEDTAVYYCAA

SEQ ID NO: 109 - protein, 39 aa, synthetic construct
  source 1..39: note = FR3 of VHH R4_DC6 Martin
YADSVKGRFTISRDNANSVVYLQMNSTKPEDTAVYYCAA

SEQ ID NO: 110 - protein, 39 aa, synthetic construct
  source 1..39: note = FR3 of VHH R3_DC23 Martin
YADSMKGRFTISRDSAKNTVDLQMNSTKPEDTAVYYCGA

SEQ ID NO: 111 - protein, 1255 aa, SARS-CoV-1
  source 1..1255: note = Sarbecovirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 112 - protein, 499 aa, synthetic construct
  source 1..499: note = TD R3DC23-117(10)-Fc
DVQLVESGGGLVQPGGSLRLSCAVSGRIFSTYTMGWFRQAPGKEREFVAAVRWGAGTIYYADSVKGRFTISRDNAKNTVYLQMNSLRPEDTAVYYCGAAYVSKANYGSLWYQDSRRYDYWGQGTLVTVSSGGGGSGGGGSDVQLVESGGGLVQPGGSLRLSCAASGKAVSISDMGWYRQPPGKQRELVATITKTGSTNYADSVKGRFTISRDNTKNTVYLEMNSLRPEDTAVYYCNAWLPYGLGPDYYGLELWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 113 - protein, 509 aa, synthetic construct
  source 1..509: note = TD R3DC23-117(20)-Fc
DVQLVESGGGLVQPGGSLRLSCAVSGRIFSTYTMGWFRQAPGKEREFVAAVRWGAGTIYYADSVKGRFTISRDNAKNTVYLQMNSLRPEDTAVYYCGAAYVSKANYGSLWYQDSRRYDYWGQGTLVTVSSGGGGSGGGGSGGGGSGGGGSDVQLVESGGGLVQPGGSLRLSCAASGKAVSISDMGWYRQPPGKQRELVATITKTGSTNYADSVKGRFTISRDNTKNTVYLEMNSLRPEDTAVYYCNAWLPYGLGPDYYGLELWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 114 - protein, 519 aa, synthetic construct
  source 1..519: note = TD R3DC23-117(30)-Fc
DVQLVESGGGLVQPGGSLRLSCAVSGRIFSTYTMGWFRQAPGKEREFVAAVRWGAGTIYYADSVKGRFTISRDNAKNTVYLQMNSLRPEDTAVYYCGAAYVSKANYGSLWYQDSRRYDYWGQGTLVTVSSGGGGSGGGGSGGGGSGGGGSGGGGSGGGGSDVQLVESGGGLVQPGGSLRLSCAASGKAVSISDMGWYRQPPGKQRELVATITKTGSTNYADSVKGRFTISRDNTKNTVYLEMNSLRPEDTAVYYCNAWLPYGLGPDYYGLELWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 115 - protein, 504 aa, synthetic construct
  source 1..504: note = R3DC23-Fc-117
DVQLVESGGGLVQPGGSLRLSCAVSGRIFSTYTMGWFRQAPGKEREFVAAVRWGAGTIYYADSVKGRFTISRDNAKNTVYLQMNSLRPEDTAVYYCGAAYVSKANYGSLWYQDSRRYDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSGGGGSGGGGSDVQLVESGGGLVQPGGSLRLSCAASGKAVSISDMGWYRQPPGKQRELVATITKTGSTNYADSVKGRFTISRDNTKNTVYLEMNSLRPEDTAVYYCNAWLPYGLGPDYYGLELWGQGTLVTVSS

SEQ ID NO: 116 - protein, 506 aa, synthetic construct
  source 1..506: note = TD 83-117 (20)- Fc
DVQLVESGGGLVQPGDSLRLSCVLSGGVFTSYAMGWFRQAPGKEREFLAAITFNSDATYYADSVKGRFTISRDNAKNTAYLQMNSLRPEDTAVYSCAAGGNHYNPQYYHDYDKYDHWGQGTLVTVSSGGGGSGGGGSGGGGSGGGGSDVQLVESGGGLVQPGGSLRLSCAASGKAVSISDMGWYRQPPGKQRELVATITKTGSTNYADSVKGRFTISRDNTKNTVYLEMNSLRPEDTAVYYCNAWLPYGLGPDYYGLELWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 117 - protein, 666 aa, synthetic construct
  source 1..666: note = TD R3DC23-117-83 (20)- Fc
DVQLVESGGGLVQPGGSLRLSCAVSGRIFSTYTMGWFRQAPGKEREFVAAVRWGAGTIYYADSVKGRFTISRDNAKNTVYLQMNSLRPEDTAVYYCGAAYVSKANYGSLWYQDSRRYDYWGQGTLVTVSSGGGGSGGGGSGGGGSGGGGSDVQLVESGGGLVQPGGSLRLSCAASGKAVSISDMGWYRQPPGKQRELVATITKTGSTNYADSVKGRFTISRDNTKNTVYLEMNSLRPEDTAVYYCNAWLPYGLGPDYYGLELWGQGTLVTVSSGGGGSGGGGSGGGGSGGGGSDVQLVESGGGLVQPGDSLRLSCVLSGGVFTSYAMGWFRQAPGKEREFLAAITFNSDATYYADSVKGRFTISRDNAKNTAYLQMNSLRPEDTAVYSCAAGGNHYNPQYYHDYDKYDHWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 118 - protein, 366 aa, synthetic construct
  source 1..366: note = XVR013
DVQLVESGGGLVQPGGSLRLSCAVSGRIFSTYTMGWFRQAPGKEREFVAAVRWGAGTIYYADSVKGRFTISRDNAKNTVYLQMNSLRPEDTAVYYCGAAYVSKANYGSLWYQDSRRYDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 119 - protein, 496 aa, synthetic construct
  source 1..496: note = XVR014
DVQLVESGGGLVQPGGSLRLSCAASGKAVSISDMGWYRQPPGKQRELVATITKTGSTNYADSVKGRFTISRDNTKNTVYLEMNSLRPEDTAVYYCNAWLPYGLGPDYYGLELWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGGGSGGGGSDVQLVESGGGLVQPGDSLRLSCVLSGGVFTSYAMGWFRQAPGKEREFLAAITFNSDATYYADSVKGRFTISRDNAKNTAYLQMNSLRPEDTAVYSCAAGGNHYNPQYYHDYDKYDHWGQGTLVTVSS

SEQ ID NO: 120 - protein, 30 aa, synthetic construct
  source 1..30: note = Gly-Ser linker (G4S)6
GGGGSGGGGSGGGGSGGGGSGGGGSGGGGS

SEQ ID NO: 121 - protein, 10 aa, synthetic construct
  source 1..10: note = Gly-Ser linker (G4S)2
GGGGSGGGGS

SEQ ID NO: 122 - protein, 15 aa, synthetic construct
  source 1..15: note = Gly-Ser linker (G4S)3
GGGGSGGGGSGGGGS

SEQ ID NO: 123 - protein, 20 aa, synthetic construct
  source 1..20: note = Gly-Ser linker (G4S)4
GGGGSGGGGSGGGGSGGGGS

SEQ ID NO: 124 - protein, 125 aa, synthetic construct
  source 1..125: note = VHH72
QVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSS

SEQ ID NO: 125 - protein, 127 aa, synthetic construct
  source 1..127: note = VHH83
QVQLQESGGGLVQPGDSLRLSCVLSGGVFTSYAMGWFRQAPGKEREFLAAITFNSDATYYADSVKGRFTISRDNAKNTAYLQMNSLKPNDTAVYSCAAGGNHYNPQYYHDYDKYDHWGQGTQVTVSS

SEQ ID NO: 126 - protein, 123 aa, synthetic construct
  source 1..123: note = VHH3.117
QVQLQESGGGLVQPGGSLRLSCAASGKAVSISDMGWYRQPPGKQRELVATITKTGSTNYADSAQGRFTISRDNTKSAVYLEMKSLKPEDTAVYYCNAWLPYGMGPDYYGMELWGKGTQVTVSS

SEQ ID NO: 127 - protein, 130 aa, synthetic construct
  source 1..130: note = humanized R3_DC23
DVQLVESGGGLVQPGGSLRLSCAVSGRIFSTYTMGWFRQAPGKEREFVAAVRWGAGTIYYADSVKGRFTISRDNAKNTVYLQMNSLRPEDTAVYYCGAAYVSKANYGSLWYQDSRRYDYWGQGTLVTVSS

SEQ ID NO: 128 - protein, 130 aa, synthetic construct
  source 1..130: note = humanized R3_C4
DVQLVESGGGLVQPGGSLRLSCAVSGRPFSTYTMGWFRQAPGKEREFVAAIRWSGGTIYYADSVKGRFTISRDNAKNTVYLQMNSLRPEDTAVYYCAAAYVSKANYGSLWYRASGLYDYWGQGTLVTVSS

SEQ ID NO: 129 - protein, 130 aa, synthetic construct
  source 1..130: note = humanized R4_DC20
DVQLVESGGGLVQPGGSLRLSCAVSGRPFSTYTMGWFRQAPGKEREFVASIRWSGGTTNYADSVKGRFTISRDNAKNTVYLQMNSLRPEDTAVYYCAAAYVSKANYGSLWYRNSGLYDYWGQGTLVTVSS

SEQ ID NO: 130 - protein, 366 aa, synthetic construct
  source 1..366: note = huR3C4-Fc
DVQLVESGGGLVQPGGSLRLSCAVSGRPFSTYTMGWFRQAPGKEREFVAAIRWSGGTIYYADSVKGRFTISRDNAKNTVYLQMNSLRPEDTAVYYCAAAYVSKANYGSLWYRASGLYDYWGQGTLVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 366 aa, synthetic construct
  source 1..366: note = huR4DC20-Fc
DVQLVESGGGLVQPGGSLRLSCAVSGRPFSTYTMGWFRQAPGKEREFVASIRWSGGTTNYADSVKGRFTISRDNAKNTVYLQMNSLRPEDTAVYYCAAAYVSKANYGSLWYRNSGLYDYWGQGTLVTVSSGGGGSGGGGSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0313611 A1
(43) Pub. Date: Oct. 9, 2025

(54) SARBECOVIRUS SPIKE S2 SUBUNIT BINDERS

(71) Applicants: VIB vzw, Gent (BE); UNIVERSITEIT GENT, Gent (BE); VRIJE UNIVERSITEIT BRUSSEL, Brussel (BE); ExeVir Bio BV, Zwijnaarde (BE)

(72) Inventors: Sieglinde DE CAE, Lennik (BE); Bert SCHEPENS, Gent (BE); Xavier SAELENS, leper (BE); Loes VAN SCHIE, Gent (BE); Wim NERINCKX, Astene (BE); Kenny ROOSE, Gent (BE); Nico CALLEWAERT, Nevele (BE); Gholamreza HASSENZADEH GHASSABEH, Sint-Genesius-Rode (BE); Han REMAUT, Roosbeek (BE); Inge VAN MOLLE, Sint-Gillis (BE); Viki BOCKSTAL, Melsele (BE); Manuela RINALDI, Hamme (BE); Christophe BLANCHETOT, Destelbergen (BE)

(21) Appl. No.: 18/866,449

(22) PCT Filed: May 17, 2023
(86) PCT No.: PCT/EP2023/063375

§ 371 Date: Nov. 15, 2024

(30) Foreign Application Priority Data
May 18, 2022 (EP) 22174102.8
Aug. 3, 2023 (EP) 23160838.1

(51) Int. Cl. C07K 16/10; A61K 39/00; A61P 31/14; G01N 33/569

(52) U.S. Cl. CPC C07K 16/1003; A61P 31/14; G01N 33/56983; A61K 2039/505; C07K 2317/22; C07K 2317/24; C07K 2317/31; C07K 2317/33; C07K 2317/34; C07K 2317/569; C07K 2317/732; C07K 2317/76; C07K 2317/92; C07K 2317/94; G01N 2333/165

(57) ABSTRACT

Sarbecovirus binding agents, in particular antibodies and antigen-binding fragments thereof, which are capable of potently neutralizing a Sarbecovirus, in particular capable of neutralizing any one or both of SARS-COV-2, including SARS-COV-2 variants, and SARS-COV. 1. The binding agents, in particular the antibodies and antibody fragments, bind to heptad repeat 2 (HR2) domain of spike protein of the Sarbecovirus, more particularly to a quaternary epitope located within 2 adjacent HR2 domains. Also disclosed are methods using these binding agents and uses thereof.

### FIELD OF THE INVENTION

The invention is broadly in the field of binding agents, in particular antibodies. More particularly, the invention pertains to binding agents, in particular antibodies and antigen-binding fragments thereof, binding to the spike protein of a Sarbocovirus, which are capable of potently neutralizing a Sarbecovirus such as SARS-COV-2, including SARS-COV-2 variants, and SARS-COV-1. The invention also relates to methods using these binding agents and uses thereof.

### BACKGROUND OF THE INVENTION

Severe acute respiratory syndrome coronavirus 2 (SARS-COV-2) is the causative agent of COVID-19 (Zhu et al. 2020, N Engl J Med 382:727-733). SARS-COV-2 infections can be asymptomatic or present with mild to moderately severe symptoms. However, in approximately 10% of patients, COVID-19 progresses to a more severe stage that is characterized by dyspnoca and hypoxemia, which may progress further to acute respiratory distress requiring often long-term intensive care and causing death in a proportion of patients. “Long-COVID” furthermore refers to long-term effects of COVID-19 infection, even when no SARS-COV-2 virus can be detected anymore.

A particular type of therapeutic approach potentially relies on neutralizing antibodies, i.e. on passive antibody therapy/immunotherapy. The spike of SARS coronaviruses is a major target for neutralizing antibodies. This spike protein is a class I fusion protein and is comprised of a membrane distal S1 subunit and a membrane proximal S2 subunit. The S1 subunit comprises the receptor-binding domain (RBD) and antibodies directed against this domain can have very strong neutralizing activity (Wheatley et al. 2021. Cell Rep 37:109822). The S1 subunit, in particular the N-terminal domain and the RBD, can tolerate mutations that result in antigenic variation and immune escape. The RBD is also immunodominant (Piccoli et al. 2020. Cell 183:1024-1042).

The S2 subunit is responsible for the membrane fusion, a process during which S2 undergoes major conformational changes (Dodero-Rojas et al. 2021. eLife 10:e70362). The S2 subunit is more conserved and therefore, at least in theory, appears to be an attractive target for the development of neutralizing antibodies with broad anti-Sarbecovirus protective potential. Several monoclonal antibodies that recognize conserved epitopes in the S2 subunit of SARS coronaviruses have been described. In general, however, these monoclonal antibodies display poor virus neutralizing activity. For example, S2 subunit-specific monoclonal antibody L19 neutralizes authentic SARS-COV-2 virus with an IC₁₀₀ of 9.9-19.8 μg/ml (Andreano et al. 2021. Cell 184:1821-1835). Wu et al. (2022. JCI Insight 7:ee157597) identified monoclonal antibodies, Mab5 and Mab3-2, that target the HR2 domain at an epitope located at the N-terminal end of the HR2 domain. The 2 mAbs possessed neutralizing ability against SARS-COV-2, with an IC₅₀ value of 12.3 μg/mL for Mab5 and an IC₅₀ of 87.4 μg/mL for Mab3-2. Single domain antibodies, also known as nanobodies or VHHs, directed against the SARS-COV-2 S2 subunit have also been reported (Mast et al. 2021. eLife 110:e73027; Rossotti et al. 2021. DOI: 10.1101/2021.12.20.473401). Again, the reported S2 subunit-binding VHHs displayed very low SARS-COV-2-neutralizing potency. S2 subunit-specific VHH S2A3 fused to an IgG1-Fc as described in Rossotti et al. (2021) could neutralize the Wuhan strain of SARS-COV-2 with an IC₅₀ of 12.2 nM but was non-neutralizing without formatting.

Hence, there remains a need in the art for potent neutralizing antibodies that target the spike protein of a Sarbecovirus.

### SUMMARY OF THE INVENTION

As demonstrated in the experimental section, which illustrates certain embodiments of the present invention, the inventors identified Sarbecovirus-specific Variable Domains of Heavy-chain Antibodies (VHHs) that potently neutralized SARS-COV-2, including SARS-COV-2 variants such as SARS-COV-2 D614G variant, SARS-COV-2 Alpha variant, SARS-COV-2 Omicron BA.1 variant SARS-COV-2 Omicron BA.2 variant, SARS-COV-2 Omicron BA.5 variant, SARS-COV-2 Omicron BA.2.75.2 variant, SARS-COV-2 Omicron BA.4.6 variant, SARS-COV-2 Omicron BF.7 variant, SARS-COV-2 Omicron BQ.1.1 variant, SARS-COV-2 Omicron XBB variant, and SARS-COV-2 Omicron XBB.1.5 variant, and SARS-COV-1. By further analysis, it was found that these VHHs interact with amino acids within the S2 subunit of the spike protein, in particular within the heptad repeat 2 (HR2) domain of the S2 subunit, more particularly within a C-terminal region of the HR2 domain proximal to the viral membrane, which amino acids are very conserved in the spike protein of Sarbecoviruses of multiple clades. This region is therefore expected to be more stable and less amenable to frequent mutational changes.

Accordingly, in an aspect the invention relates to a binding agent capable of neutralizing a Sarbecovirus, characterized in that said binding agent specifically binds to a region of heptad repeat 2 (HR2) domain of spike protein of the Sarbecovirus proximal to the viral membrane.

An aspect provides a binding agent capable of neutralizing a Sarbecovirus, characterized in that said binding agent specifically binds to or within a region of spike protein of the Sarbecovirus corresponding to the region from amino acid E1188 to amino acid Y1206 of the SARS-COV-2 spike protein as defined in SEQ ID NO: 86.

In certain preferred embodiments, the binding agent specifically binds to or within a region of spike protein corresponding to the region from amino acid E1188 to amino acid L1203 of the SARS-COV-2 spike protein as defined in SEQ ID NO: 86.

In certain preferred embodiments, the binding agent specifically binds to or within a region of spike protein corresponding to the region from amino acid E1188 to amino acid L1202 of the SARS-COV-2 spike protein as defined in SEQ ID NO: 86.

Such Sarbecovirus-neutralizing binding agents binding the more conserved S2 subunit of the spike protein are valuable tools to be added to the overall still limited number of SARS-COV-2 treatment options currently available, particularly in view of the multiple emerging SARS-COV-2 variants, some of these being more infectious and/or causing more severe disease symptoms (including in younger people) and/or escaping some of the existing vaccines and/or diagnostic tests.

In a further aspect, the invention relates to a nucleic acid molecule comprising a polynucleotide sequence encoding the binding agent according to the invention, as well as to a vector comprising such nucleic acid molecule; and a cell comprising such nucleic acid molecule or such vector or a cell expressing the binding agent according to the invention.

The invention further relates to a pharmaceutical composition comprising the binding agent according to the invention, or the nucleic acid molecule or the vector as described hereinabove; and a pharmaceutically acceptable carrier; as well as to a kit such as a diagnostic kit comprising the binding agent according to the invention.

A further aspect is directed to the binding agent according to the invention, the nucleic acid molecule or the vector as described hereinabove, the pharmaceutical composition or the kit as described hereinabove for use in medicine such as use in the prevention or treatment of a Sarbecovirus infection in a subject or for use in the diagnosis of a Sarbecovirus infection in a subject.

The invention further relates to an in vitro or ex vivo method for detecting a Sarbecovirus in a sample, said method comprising:
contacting the sample with a binding agent according to the invention, and
determining binding of the binding agent with a Sarbecovirus or a part thereof.

Those skilled in the art will recognize the many other effects and advantages of the present methods, uses or products, and the numerous possibilities for end uses of the present invention from the detailed description and examples provided below.

### DESCRIPTION OF THE DRAWINGS

FIG. 1 VHHs present in E. coli periplasmic extracts (PE) of isolated clones bind to recombinant SARS-COV-2 spike protein (SC2 S (6P)), SARS-COV-2 RBD (SC2 RBD), SARS-COV-2 S2 subunit (SC2 S2) and SARS-COV-1 spike protein (SC1 S) in ELISA. (A) ELISA of clones (R3_Cxx) isolated after 3 rounds of biopanning on SARS-COV-2 spike proteins captured by anti-His antibodies coated on ELISA plates (R1-R3). (B) ELISA of clones (R3_DCxx) isolated after 3 rounds of biopanning on SARS-COV-2 spike proteins captured by anti-His antibodies (R1-2) followed by biopanning on directly coated SARS-COV-2 spike proteins (R3). (C) ELISA of clones (R4_Cxx) isolated after 4 rounds of biopanning on SARS-COV-2 spike proteins captured by anti-His antibodies coated on ELISA plates (R1-R4). (D) ELISA of clones (R4_DCxx) isolated after 4 rounds of biopanning on SARS-COV-2 spike proteins captured by anti-His antibodies (R1-2) followed by biopanning on directly coated SARS-COV-2 spike proteins (R3-R4). The graphs show for each PE sample, the ratio of the ELISA OD 450 signal for the indicated antigen over the ELISA OD 450 signal of the corresponding PE sample for the control antigen (BSA). Periplasmic extracts prepared from E. coli cells that express the RBD binding VHH. VHH3.83 (3.83) were used as control. Buffer used to prepare the periplasmic extracts was used as negative control (TES).

FIG. 2 Sequence analysis of the VHHs able to bind the S2 subunit of SARS-COV spikes. (A) Alignment of Family 1 VHHs that bind the S2 subunit of SARS-COV spikes. Based on alignment of the full VHH sequence, two families (Family 1 and Family 2) of S2 binding VHHs could be identified. Sequences of Family 1 VHHs are shown: R3_C4 and R3_DC13 (SEQ ID NO:1). R3_DC19 (SEQ ID NO:83). R3_DC21 and R3_DC22 (SEQ ID NO:84), R3_C22 and R4_DC16 (SEQ ID NO:2). R3_DC20 (SEQ ID NO:3), R3_DC1. R3_DC9. R3_DC14 and R3_DC15 (SEQ ID NO: 9). R3_DC2 (SEQ ID NO:4). R4_DC20 (SEQ ID NO:5). R3_DC12 and R4_DC13 (SEQ ID NO: 10). R3_DC5 (SEQ ID NO:85). R4_DC24. R4_DC21. R3_DC11 and R4_DC9 (SEQ ID NO:6). R3_DC8. R4_DC3 and R4_DC6 (SEQ ID NO:7). R3_DC23 (SEQ ID NO:8). Amino acid residue 20) numbering was done according to Kabat numbering. CDR1, 2 and 3 annotated according to Kabat are indicated by respectively the left, middle and right box. (B) Phylogenetic analysis of Family 1 VHHs based on their CDR3 amino acid sequences. The VHHs marked in grey were selected for medium scale production and Ni-NTA purification. The VHHs indicated with a “*” contain an N-glycosylation site motive.

FIG. 3 The selected S2 binding VHHs recognize the Spike proteins of SARS-COV-1, the SARS-CoV-2 Wuhan variant, the SARS-COV-2 Omicron BA.1 variant and the SARS-COV-2 S2 subunit but not the SARS-COV-2 RBD. The graphs display the OD 450 ELISA signal of dilution series of the indicated VHHs, including the control GFP-binding VHH (GBP), or of the S309 control monoclonal antibody to the spike protein S-6P (A), the RBD (E) and the S2 subunit (D) of Wuhan SARS-COV-2, the spike protein of Omicron BA.1 SARS-COV-2 (B) and the spike protein of SARS-CoV-1 (C) coated to the substrate, or to BSA coated substrate (F).

FIG. 4 S2 targeting VHHs efficiently bind to cell surface expressed spike proteins. (A) Flow cytometric analysis of the binding of R3_DC23. R4_DC6 and the GFP binding VHH (GBP) control to cells expressing SARS-COV-2 spike proteins. The graph shows the mean fluorescence intensity (MFI) of the AF647-conjugated anti-mouse IgG to detect binding of VHHs to GFP expressing cells that were transfected with a GFP expression vector in combination with a SARS-COV-2 spike (614G-del18) expression vector (spike D614G). (B) Flow cytometric analysis of the binding of R3_DC23. R4 DC6 and the GBP control VHH to cells that do not express SARS-COV-2 spike proteins. The graph shows the MFI of the AF647-conjugated anti-mouse IgG to detect binding of VHHs to GFP expressing cells that were transfected with a GFP expression vector in combination with a control expression vector.

FIG. 5 S2 targeting VHHs neutralize VSV-GFP reporter viruses pseudotyped with SARS-COV-2 614G spike protein. Vero E6 cells were transduced with VSV-GFP reporter viruses pseudotyped with SARS-COV-2 614G spike protein that had been pre-incubated with different concentrations of the indicated VHHs. Fifteen hours later, the GFP fluorescence was measured with a fluorimeter. (A) The graphs show the mean GFP fluorescence intensity of the VHH dilution series (N=3±SD) each normalized to the GFP fluorescence intensity value of non-infected control cells and of infected cells that were not treated, which were both included in each dilution series. (B) For each VHH dilution series the IC50 was calculated using a linear regression curve fitting (log (inhibitor) vs. normalized response with variable slope). The graph shows the calculated IC50 values (N=3±SD) for each tested neutralizing VHH.

FIG. 6 S2 targeting VHHs can prevent TMPRSS2-mediated infection of VSV-GFP reporter viruses pseudotyped with SARS-COV-2 614G spike protein. Vero E6-TMPRSS2 cells were transduced with VSV-GFP reporter viruses pseudotyped with SARS-COV-2 614G spike protein that had been pre-incubated with different concentrations of the indicated VHHs. Fifteen hours later, the GFP fluorescence was measured with a fluorimeter. (A) The graphs show the mean GFP fluorescence intensity of the VHH dilution series (N=3±SD) each normalized to the GFP fluorescence intensity value of non-infected control cells and of infected cells that were not treated, which were both included on each plate. (B) For each VHH dilution series the IC50 was calculated using a linear regression curve fitting (log (inhibitor) vs. normalized response with variable slope). The graph shows the calculated IC50 values (N=3±SD) for each tested neutralizing VHH.

FIG. 7 S2 targeting VHHs neutralize replication-competent VSV-GFP reporter viruses pseudotyped with SARS-COV-2 Wuhan spike protein. One hundred PFU of the replication-competent VSV-GFP reporter virus (VSV-AG SC2S EGFP S10a) as described by Koenig et al. (2021) was pre-incubated with the indicated VHHs and used to infect Vero E6 cells. Two days after infection, the GFP fluorescence was measured with a fluorimeter. (A) The graphs show the mean GFP fluorescence intensity of the VHH dilution series (N=2±SD) each normalized to the GFP fluorescence intensity value of non-infected control cells and of infected cells that were not treated, which were both included in each dilution series. (B) For each VHH dilution series the IC50 was calculated using a linear regression curve fitting (log (inhibitor) vs. normalized response with variable slope). The graph shows the calculated IC50 values (N=2±SD) for each tested neutralizing VHH.

FIG. 8 S2 targeting VHHs neutralize VSV-GFP reporter viruses pseudotyped with SARS-COV-2 Omicron BA.1 spike protein. Vero E6 cells were transduced with VSV-GFP reporter viruses pseudotyped with SARS-COV-2 Omicron BA.1 spike protein that had been pre-incubated with different concentrations of the indicated VHHs. The S309 monoclonal antibody known to neutralize the SARS-COV-2 Omicron BA.2 variant was used as positive control. Fifteen hours later, the GFP fluorescence was measured with a fluorimeter. (A) The graphs show the mean GFP fluorescence intensity of the VHH dilution series (N=1) each normalized to the GFP fluorescence intensity value of non-infected control cells and of infected cells that were treated with the lowest VHH concentration included in each dilution series. (B) For each VHH dilution series the IC50 was calculated using a linear regression curve fitting (log (inhibitor) vs. normalized response with variable slope). The graph shows the calculated IC50 values (N=1) for each tested neutralizing VHH.

FIG. 9 S2 targeting VHHs neutralize VSV-GFP reporter viruses pseudotyped with SARS-COV-1 spike protein. Vero E6 cells (A) or Vero E6-TMPRSS2 cells (B) were transduced with VSV-GFP reporter viruses pseudotyped with SARS-COV-1 spike protein that had been pre-incubated with different concentrations of the indicated VHHs. The VHH72-S56A nanobody know to neutralize SARS-COV-1 was used as positive control. Fifteen hours later, the GFP fluorescence was measured with a fluorimeter. The graphs show the mean GFP fluorescence intensity of the VHH dilution series (N=2±SD) each normalized to the GFP fluorescence intensity value of non-infected control cells and of infected cells that were not treated, which were both included in each dilution series. (C) For each VHH dilution series the IC50 was calculated using a linear regression curve fitting (log (inhibitor) vs. normalized response with variable slope). The graph shows the calculated IC50 values (N=2±SD) for each tested neutralizing VHH on both Vero E6 and Vero E6-TMPRSS2 cells.

FIG. 10 S2 targeting VHHs neutralize VSV-GFP reporter viruses pseudotyped with SARS-CoV-2 Omicron BA.2 spike protein. Vero E6 cells were transduced with VSV-GFP reporter viruses pseudotyped with either 614G spike proteins, or spike proteins of Omicron BA.1 or Omicron BA.2 variant that had been pre-incubated with different concentrations of the indicated VHHs. Fifteen hours later, the GFP fluorescence was measured with a fluorimeter. (A) The graphs show the mean GFP fluorescence intensity of the VHH dilution series (N=2±SD) each normalized to the GFP fluorescence intensity value of non-infected control cells and of infected cells that were not treated, which were both included in each dilution series. (B) For each VHH dilution series the IC50 was calculated using a linear regression curve fitting (log (inhibitor) vs. normalized response with variable slope). The graph shows the calculated IC50 values (N=2±SD) for each tested neutralizing VHH on Vero E6 cells transduced with VSV-GFP reporter viruses pseudotyped with SARS-COV-2 614G spike proteins (614G), or with spike proteins of Omicron BA.1 (BA.1) or Omicron BA.2 (BA.2) variant.

FIG. 11 S2 targeting VHHs can neutralize authentic 614G and Omicron SARS-COV-2 viruses. Dilution series of R3_DC23 or R4_DC6 were pre-incubated with about 40 PFU of 614G variant SARS-COV-2 or Omicron BA.1 SARS-COV-2 virus for 1 hour at 37° C. and subsequently used to infect Vero E6-TMPRSS2 cells. Antibody S309, known to neutralize Alpha and Omicron BA.1 SARS-COV-2 variants was used as positive control. Two days post-infection the cells were fixed and stained with crystal violet to visualize the viral plaques. The graphs show the average (N=2±SD for R3_DC23 and R4° DC6 and N=1 for S309) number of 614G (A) or Omicron BA.1 (B) viral plaques at the indicated VHH or antibody concentrations. Curves were fitted using non-linear regression (log (inhibitor) vs. response with variable slope (four parameters).

FIG. 12 S2 targeting VHHs do not evoke shedding of the S1 subunit. (A) Panel showing anti-S1 Western blot analysis of the growth medium (SN) and cell lysates (LYS) of Raji cells expressing the SARS-COV-2 spike protein (Raji spike) or not (Raji) incubated for 30 minutes with the indicated VHH constructs. The CB6 and S309 antibodies, know to respectively evoke and not evoke S1 shedding were used as controls. The lower and upper triangles at the right side of the blots indicate respectively, the S1 spike subunit generated after furin-mediated cleavage of the spike protein and cellular uncleaved spike proteins. (B) Quantification of S1 shedding. The graph shows the calculated ratio of the S1 Western blot signal detected in the growth medium (shedded) over the S1 Western blot signal detected in the cell lysate (non-shedded+intracellular).

FIG. 13 S2 targeting VHHs potently inhibit fusion. VHH R3_DC23 prevents syncytium formation of confluent monolayers of Vero E6-TMPRSS2 cells infected with SARS-COV-2 spike pseudotyped replication-competent VSV virus expressing GFP. Vero E6-TMPRSS2 cells were infected with 40 PFU of SARS-COV-2 spike pseudotyped replication-competent VSV virus and two hours later the indicated monoclonal antibodies (palivizumab. S309 or CB6) or VHHs (GBP or R3_DC23) were added to a final concentration of 10 μg/ml. Non-infected cells were used as negative controls. Cells were incubated overnight and imaged with a fluorescence microscope. GFP fluorescence was measured with a fluorimeter. (A) Representative images of GFP expressed by infected cells treated with the indicated VHHs or monoclonal antibodies. (B) Graph showing the GFP fluorescence intensity (mean±SEM. N=4). The GFP fluorescence measured in the S309 treated samples was significantly lower than in the samples treated with the palivizumab control antibody (p<0.05. Mann-Whitney). The GFP fluorescence measured in the R3_DC23 treated samples was significantly lower than in the samples treated with the GBP control VHH (p<0.05. Mann-Whitney). The GFP fluorescence measured in the R3_DC23 treated samples was significantly lower than in the samples treated with the S309 (p<0.05. Mann-Whitney).

FIG. 14 S2 targeting VHHs potently inhibit fusion. Vero E6-TMPRSS2 cells were infected with 40 PFU of SARS-COV-2 spike pseudotyped replication-competent VSV virus and two hours later dilution series of the indicated monoclonal antibodies (S309 or CB6) or VHHs (GBP. R3_DC23. R3_C4 or R3_DC20) were added. Non-infected cells were used as negative controls. Cells were incubated overnight and imaged with a fluorescence microscope. GFP fluorescence was measured with a fluorimeter. (A) Representative images of GFP expressed by infected cells treated with the indicated VHHs or monoclonal antibodies at 10, 0.4 or 0.0032 μg/ml. The arrows indicate single GFP-positive infected cells. (B) Graph showing the GFP fluorescence intensity (mean±SEM. N=2).

FIG. 15 S2 targeting VHHs potently inhibit fusion of spike expressing Vero E6 cells. Vero E6 cells were transfected with an GFP expression vector in combination with either a control expression vector (No Spike) or an SARS-COV-2 spike expression vector. Two hours after transfection PBS or the indicated monoclonal antibodies (S309 or palivizumab) or VHHs (R3_DC23 or R3_C4) were added to a final concentration of 10 μg/ml. Twenty-two hours after transfection the cells were fixed and imaged using a fluorescence microscope. The panels show a representative image of the indicated samples. No Spike=PBS treated cells that were transfected with an GFP expression vector in combination with a control expression vector; PBS, palivizumab. R3_DC23. R3_C4 and S309=cells transfected with an GFP expression vector in combination with a SARS-COV-2 spike expression vector and treated with the respective constructs.

FIG. 16 VHH.R3_DC23 binds the spike protein at a membrane proximal site in the HR2 region. Viral escape selection was performed on Vero E6-TMPSS2 cells using a replication-competent GFP expressing VSV virus pseudotyped with the Wuhan SARS-COV-2 spike protein with a fully intact furin cleavage site. From the wells that displayed syncytia formation in the presence of 10 μg/ml VHH.R3_DC23 single plaques were isolated using limiting dilution. The spike protein coding sequence of the obtained escape variants was sequenced and aligned to the sequence of WT virus. Each of the selected viruses contained a single amino acid substitution. (A) Sequence of the R3_DC23 binding region. Viral escape selection from VHH.R3_DC23 is associated with 5 different AA substitutions a 4 positions within a confined membrane proximal region within the HR2. The shown sequence (SEQ ID NO:88) corresponds to the spike stem region (amino acids 1140-1211) composed of the stem-helix and the Heptad Repeat 2 (HR2) domain. The amino acids in bold and underlined indicate the positions that are mutated in the 9 escape variants that were isolated. The Asn (N) between the Asp (N) and Leu (L) in bold and underlined is a N-glycosylation site (B) Structural details of the R3_DC23 binding region. The left image represents a model of the full length SARS-COV-2 spike protein on which the transmembrane region TM and HR2 are indicated (Casalino et al. (2020) ACS Cent Sci. 6:1722-1734). The middle and right images are a zoom of the HR2 domain respectively shown in surface and cartoon representation (2FXP, Hakansson-McReynolds et al. (2006) J Biol Chem. 281:11965-71). In the surface display images, the sticks represent modeled sugar moieties, the TM is colored in grey and the positions at which the selected mutations localize in 3 protomers are indicated in black. In the middle image the arrow respectively pinpoints the N1192 and Q1201 mutated positions within protomer 1. The other 2 visible escape mutations (indicated in black) respectively localize in protomers 3 and 2. On one of the protomers shown in the cartoon representation the positions at which mutations were observed are indicated in black as sticks.

FIG. 17 VHH.R3_DC23 binds the spike protein at a site in the HR2 domain that is highly conserved among Sarbecoviruses. The amino acid sequences of the spike stem region of a set of clade 1, 2 and 3 Sarbecoviruses studied by Letko et. al (2020. Nature Microbiology 5:5 62-569) and supplemented with that of the BtKY72 clade 3 Sarbecovirus were aligned to visualize its conservation: Wuhan SARS-COV-2, WIV1, Rs4084, SHC014, Rs7327, Rs4231, SARS-CoV_Urbani, Longquan-140, HKU3-8, HKU3-13, Rs4237, Rs4247, As5626, Rp3, 279, Rs4081, Yunnan2011, Hub2013, Shaanxi2011, YN2013 and Rf4098 (SEQ ID NO:88); LYRa11 (SEQ ID NO: 89); 273-2005, Rfl, HcB2013 and JL2012 (SEQ ID NO:90); GX2013 (SEQ ID NO:91); ZC45 (SEQ ID NO:92); ZXC21 (SEQ ID NO:93); BM48-31 (SEQ ID NO:94); and BtKY72 (SEQ ID NO: 95). The linear sequence marked in grey (corresponding to amino acids 1192-1201 of the SARS-COV2 Wuhan strain spike protein as depicted in SEQ ID NO:86) comprises the positions that were mutated in the isolated R3_DC23 escape variants. The amino acids in bold and underlined are those that were mutated in the isolated R3_DC23 escape variants. The sequences are grouped according to the clade (indicated on the right) of the respective Sarbecoviruses.

FIG. 18 Fc fusions of the VHH.R3_DC23 efficiently bind to cell surface expressed spike proteins. Flow cytometric analysis of the binding of R3_DC23-Fc(YTE) and the control antibody palivizumab to cells expressing SARS-COV-2 spike proteins. The graphs show the mean fluorescence intensity (MFI) of the Alexa Fluor (AF) 633-conjugated anti-human IgG used to detect binding of R3_DC23-Fc(YTE) or palivizumab to GFP expressing cells that were transfected with a GFP expression vector in combination with an SARS-COV-2 spike (614G-del18) expression vector (spike D614G) (A) or in combination with a control expression vector (empty vector) (B).

FIG. 19 R3_DC23-Fc(YTE) potently neutralizes VSV-GFP reporter viruses pseudotyped with SARS-COV-2 614G spike protein or with Omicron BA.1 and BA.2 spike proteins. Vero E6 cells were transduced with VSV-GFP reporter viruses pseudotyped with SARS-COV-2 spike proteins 614G or with the spike protein of the SARS-COV-2 Omicron BA.1 or Omicron BA.2 variants, which reporter viruses had been pre-incubated with different concentrations of R3 DC23-Fc(YTE) (N=3), palivizumab (N=1), CB6 (N=1) or monovalent VHH.R3_DC23 (N=1). Fifteen hours later, the GFP fluorescence was measured with a fluorimeter. (A) The graphs show the mean GFP fluorescence intensity of the VHH dilution series (N=3±SD for R3_DC23-Fc(YTE)) each normalized to the GFP fluorescence intensity value of non-infected control cells and of infected cells that were not treated, which were both included in each dilution series. (B) For each VHH dilution series the IC50 was calculated using an linear regression curve fitting (log (inhibitor) vs. normalized response with variable slope). The graph shows the calculated IC50 values (N=3±SD for R3_DC23-Fc(YTE)) for each tested antibody formats.

FIG. 20 VHH R3_DC23 amino acid sequence and illustration of the different CDR annotations as used herein. CDR annotations according to AbM. Chothia. Martin. Kabat. IMGT and MacCallum in grey labeled boxes corresponding to the sequences of VHH R3_DC23 (SEQ ID NO: 8).

FIG. 21 Prophylactic treatment with R3_DC23-Fc protects K18-hACE2 mice from lethal SC2 infection. K18-hACE2 mice were intraperitoneally injected with 100 μg R3_DC23-Fc or Isotype control antibody (palivizumab) or were left untreated twenty hours prior to intratracheal infection with 3*10² PFU of SARS-COV-2 614G variant virus. Animals were monitored on a daily base by measuring weight change and scoring for humane endpoints. (A) The graph shows the mean relative bodyweight change of mice treated with R3_DC23-Fc (n=3±SEM), palivizumab (n=2±SEM) or untreated mice (n=6±SEM). Mice treated with R3_DC23-Fc displayed significantly lower bodyweight loss as compared to mice treated with palivizumab (p<0.05) or untreated mice (p<0.01) (mixed-effect analysis with Sidak's multiple comparisons test). (B) The graph shows the Kaplan-Meier curve of animal survival portion of the indicated groups. Euthanasia was performed when mice lost more than 25% of their bodyweight as defined on day 0 or when a high score for humane endpoints was reached.

FIG. 22 Identification of SARS-COV-1 and -2 S2 subunit-specific VHHs (A) Screen of E. coli periplasmic extracts (PE) of VHH clones isolated after 3 (R3) or 4 (R4) of bio-panning on SARS-CoV-2 spike protein that was either directly coated (DC) or captured via coated anti-HIS IgG (C) for binding to the indicated recombinant spike proteins or fragments thereof. The heat map shows for each PE sample (10-fold diluted), the ratio of the ELISA OD 450 signal for the indicated antigen over the ELISA OD 450 signal of the corresponding PE sample for the control antigen (BSA). Periplasmic extracts prepared from E. coli cells that express the RBD binding VHH, were used as control. Buffer used to prepare the periplasmic extracts (TES) was used as negative control. (B) Screen of E. coli periplasmic extracts (PE) of VHH clones isolated after 3 (R3) or 4 (R4) of bio-panning on SARS-COV-2 spike protein that was either directly coated (DC) or captured via coated anti-HIS IgG (C) for neutralization of VSV particles pseudotyped with SARS-COV-2 spikes. The heat map shows for each PE sample for each PE sample (100-fold diluted), the level of neutralization of SARS-COV-2 spike protein pseudotyped VSV particles.

FIG. 23 Binding of S2 targeting VHHs to cells expressing the spike protein of SARS-COV-2 614G, BA.1, BA.2, BA.5, BQ1.1 and MERS. The SARS-COV-2 RBD binding VHH72-S56A and S309 were used as positive controls and the GFP binding VHH GBP was used as negative control. The expression of the MERS spike was confirmed by binding of the MERS specific VHH55 (data not shown). The graph shows the ratio of the MFI of transfected (GFP+) cells and the MFI of non-transfected (GFP−) cells.

FIG. 24 Neutralization of VSV particles pseudotyped with the spike protein of SARS-COV-2 614G, BA2, BA.5, XBB, BQ1.1 and SARS-COV-1 by S2 binding VHHs. The graph shows the mean (line) and individual (dots) IC50 values calculated from at least 2 independent neutralization assays (E).

FIG. 25 S2 targeting VHHs potently neutralize replication competent SARS-COV-2 pseudotyped virus. Vero E6 (A) or Vero E6-TMPRSS2 (B) cells were infected with replication competent GFP reporter virus pseudotyped with SARS-COV-2 VSV-S that had been pre-incubated with a dilution series of the indicated VHH. VHH72-S56A was included as a positive control. GFP− binding protein (GBP) was included as a negative control. The mean GFP intensity of two technical replicates for each dilution is shown, error bars represent the standard deviation.

FIG. 26 S2 targeting VHHs do inhibit spike mediated membrane fusion. (A) S2 targeting VHHs do not interfere with the binding of the spike protein with ACE2. The graph shows the OD 450 signal of an ELISA in which the binding of human ACE2-muFc to coated recombinant spike proteins containing an inactivated furin cleavage site was tested in the presence of a dilution series of R3DC23. The GFP binding VHH (GBP) was used as negative control and VHH72-S56A and CB6, both competing with ACE2 for the binding of RBD, were used as positive control. (B) S2 targeting VHHs do not induce shedding of S1. The panel shows anti-S1 Western blot analysis of the growth medium (SN) and cell lysates (LYS) of Raji cells expressing the SARS-COV-2 spike protein (Raji Spike) or not (Raji) incubated for 30 minutes with the indicated VHH constructs. The CB6 and S309 antibodies, know to respectively evoke and not to evoke S1 shedding were used as controls. The lower and upper triangle at the right side of the blots indicate respectively the S1 spike subunit generated after furin mediated cleavage of the spike protein and cellular uncleaved spike proteins. (C) S2 targeting VHHs do not interfere with the binding of human ACE2-muFc to cells expressing the spike protein with intact furin cleavage site. The graph shows the ratio of MFI (detection of cell bound ACE2-muFc) of GFP+ cells over that of GFP− cells in the presence of R3DC23. GBP was used as negative control and VHH72-S56A that induces S1 shedding and competes with human ACE2 for the binding to the RBD was used as positive control. The dashed line represents the binding of ACE2-muFc to cells not expressing spike protein. The dotted line represents the binding of ACE2-muFc to spike expressing cells in the absence of antibody (D) S2 targeting VHHs potently prevent syncytia formation by infected cells. The graph shows the mean±SD (N=2) GFP fluorescence of wells of Vero E6 cells treated with dilutions series of R3DC23. GBP or S309 four hours after infection with replicating VSV pseudotyped with the Wuhan spike protein and containing a GFP expression cassette. The images on the left show the GFP expression of the indicated samples at 40 hours post-infection. (E) Quantification of syncytia formation by spike expressing cells in the presence of R3DC23. GBP or PBS during live cell imaging. The graph shows the mean±SD (N=3) GFP+ area of wells treated with the indicated VHHs four hours after co-transfection of an GFP and a spike expression vector or of only a GFP expression vector (no Spike).

FIG. 27 VHH.R3_DC23 binds the spike protein at a membrane proximal site in the HR2 region. (A and B) Replication of viral escape variants N1192D, L1197P, L1200P, Q1201R and Q1201K on Vero E6-TMPRSS2 and Vero E6 cells in the presence of R3DC23. The graphs show the mean±SEM (N=4) level of GFP normalized by the GFP fluorescence of mock infected cells and infected cells in the absence of R3DC23. (C) Binding of R3DC23 to cells expressing the Wuhan and N1192D, L1197P, L1200P, Q1201R or Q1201K spike variants. The graph shows the ratio of the MFI of transfected (GFP+) cells and the MFI of non-transfected (GFP−) cells stained with the indicated concentrations of R3DC23, with 10 μg/ml VHH55 or 1 μg/ml S309. (D) Kinetics of viral replication of replication competent VSV pseudotyped with SARS-COV-2 Wuhan (parental) or the selected escape variants as measured by life cell imaging of infected Vero E6 cells. The graph shows the mean±SEM (N=5) GFP+ area per well of infected cells at the indicated time points post-infection. (E) Representation of the HR2 coiled coil structure (PDB:2FXP) on which the positions at which a substitution was observed in the escape variants are indicated (N1192, L1197, L1200 and Q1201). The glycans conjugated at N1194 as modelled in 6XVV_1_1_1 are indicated in stick representation. The dashed line represents the viral membrane. The central long coiled coil of alpha helices in the left panel corresponds to the sequence that is underlined in (A). The right panel represents a top view of the HR2 coiled coil with the N1192. L1197. L1200 and Q1201. The N1194 glycosylation site is indicated and the first GlcNac of the N-glycans as modelled in 6XVV_1_1_1 indicated in stick representation (ref same as 6vSB_1_1_2). (F) Binding of R3DC23 to cells expressing the indicated SARS-COV-2 spike proteins variants. The graph shows the ratio of the MFI of transfected (GFP+) cells and the MFI of non-transfected (GFP−) cells stained with the indicated concentrations of R3DC23, with 10 μg/ml GBP or 1 μg/ml S309. (G and H) Identification of the R3DC23 epitope on recombinant spike protein by HDX-MS. The panels show the HDX-MS uptake plots of the two peptides: peptide (1187-1199) (left panel) and peptide (1200-1205) (right panel) with high degrees of protection from deuteration upon the binding of R3DC23. The residue at position 1194 of peptide (1187-1199) is glycosylated, the glycosylated peptide has taken up more deuterium than the number of backbone exchangeable sites (11 sites) because the glycan can uptake and retain deuterium at amide sites similarly to the backbone as noted by Guttman. Scian and Lee (2011. ACS Analytical Chemistry). (H) The Woods plot shows for each indicated peptide (indicated by the residues numbering in the x-axis) for the indicated time points the difference in the number of deuterons acquired between apo spikes and R3DC23 bound S-2P spikes.

FIG. 28 X-ray structure of the R3_DC23-HR2 complex. (A) left: model of the full length prefusion S protein (6VSB_1_1_2; Woo et al. (2020 J. Phys. Chem B 124:7128-7137) in superimposition with the R3_DC23-HR2 complex, all shown in molecular surface representation, with N-glycans in stick representation. Labelled S protein regions are: cytoplasmic domain (CP), transmembrane domain (TM), heptad repeat 2 (HR2), S2 stem helix (SH), heptad repeat 1 (HR1), central helic and connector domain (CH-CD). The S1 regions encompassing the N-terminal domain (NTD) and receptor binding domain (RBD) are proteolytically removed prior to postfusion transition. Right: model of the proteolytically processed postfusion S protein (7E9T; Tai et al. (2021 PNAS 118:e2112703118). (B) Side and axial view (inset) of the R3_DC23-HR2 complex superimposed with prefusion HR2 coiled coil. The HR2 binding epitope spanning N1192-Y1206 is shown in stick representation. (C) Close-up of boxed region of B, encompassing a single VHH and two HR2 copies (i and ii) forming the adjoined binding epitope. Escape mutant positions are indicated (N1192, L1197, Q1201). (D) Axial view of HR1-HR2 region of postfusion S protein, superimposed with R3-DC23-HR2 complex. (E) Structural view of paratope-epitope contacts in the R3_DC23-HR2 complex. Close-up view of a single R3_DC23 copy bound to two HR2 helices (i and ii), with key interacting residues in the paratope and epitope shown in stick representation. Candidate H-bonds and salt bridges are shown as dashed lines. Escape mutant positions are shown (N1192. L1197. L1200 and Q1201).

FIG. 29 R3DC23 binds to a quaternary epitope within the HR2 coiled coil. The left and right panels show the binding (OD 450 nm) of R3DC23 to respectively trimeric full length spikes (S-2P) and monomeric SUMO-HR3 coated to half-well 96 well ELISA plates at different amounts as indicated on the x-axis. The GFP binding VHH (GBP) was used as negative control.

FIG. 30 Fc-fusion of R3DC23 potently neutralize a broad range of SARS-COV-2 variants. (A) Neutralization of SARS-COV-2 614G and BA.5 spike VSV pseudotypes by monovalent R3DC23. R3C4 and R4DC20 and their corresponding humanized counterparts fused to human IgG1 Fc containing the half-life extending YTE mutation (huR3DC23-Fc, huR3C4-Fc and huR4DC20-Fc). The symbols represent the mean±SD (N=3) relative infection as measured by GFP fluorescence by infected cells. (B) Neutralization of SARS-COV-2 614G spike VSV pseudotypes by Fc fusions of humanized (huR3DC23-Fc) and non-humanized (R3DC23-Fc) R3DC23. (C) Graph shows the median (line) and individual (diamond) IC50 values calculated from at least 2 independent neutralization assays using VSV pseudotyped with the spike protein of the indicated SARS-COV-2 variants. (D) Neutralization of authentic SARS-COV-2 D614G and BA.1 virus by S2 binding huR3DC23-Fc and/or R3DC23 VHH. The graphs show the mean±SEM (N=4 for R3DC23 and huR3DC23-Fc and N=2 for S309) number of counted plaques for each VHH dilution. (E) Analytical hydrophobic interaction chromatography of R3DC23-Fc, huR3DC23-Fc, huR3C4-Fc and huR4DC20-Fc as compared to that of clinically validated VHH-Fc XVR011. Apparent hydrophobicity was assessed on ProPac HIC-10 HPLC over an (NH₄)₂SO₄ elution gradient, short retention times indicate low apparent hydrophobicity. The panel shows duplicate curves for each indicated VHH-Fc construct.

FIG. 31 LS mutants of humanized R3DC23 Fc fusions potently neutralize a broad range of SARS-COV-2 variants and control viral replication in hamsters. (A) Analytical hydrophobic interaction chromatography of LS and YTE variants of huR3DC23-Fc overlap. Apparent hydrophobicity was assessed on ProPac HIC-10 HPLC over an (NH₄)₂SO₄ elution gradient, short retention times indicate low apparent hydrophobicity. Duplicate curves are shown for each VHH-Fc. (B) Both LS and YTE variants of huR3DC23-Fc elute as a single (overlapping) peak from analytical SEC. Molar weight markers of a gel filtration standard (Bio-Rad) are indicated in grey. Curves and shading indicate mean and SD of triplicate runs. (C) FcRn binding of huR3DC23-Fc_LS at pH 6.0 as determined by SPR. After low density immobilization of huR3DC23-Fc_LS to a sensor chip, a 250 to 0.97 nM two-fold dilution series of human FcRn was injected in solution (grey curves). A 1:1 binding model was fit (black curves). Supporting data in Table 9. (D-E) Treatment of SARS-COV-2 Wuhan infection by huR3DC23-Fc_LS in Syrian Golden hamsters. Male Syrian Golden hamsters were intranasally infected with SARS-COV-2 (Wuhan strain) on day 0 and received intraperitoneal treatment with either 10 or 2 mg/kg huR3DC23-Fc_LS, 10 mg/kg bebtelovimab (positive control) or 10 mg/kg Palivizumab (negative control) 4 hours post-infection. Animals were euthanised at day 4 and infectious virus (D) and viral RNA (E) were measured in lung tissue on day 4. Horizontal bars indicate the median TCID50/gram (left panel) and RNA copies/gram (right panel) lung tissue. Dotted horizontal lines indicate the LLOD. *2 animals in high dose (10 mg/kg) group were experimentally confirmed to not have been exposed to R3DC23hum-Fc_LS treatment. Data were analyzed with the one-way ANOVA and Dunn's multiple comparison test (*** P<0.0001). (#) Data points corresponding to hamsters for which no or very low levels of huR3DC23-Fc_LS was detected in the scrum were omitted from statistical analysis.

FIG. 32: Specificity of binding of R3_DC23-Fc(LS) to SARS-COV-2 spike protein. R3_DC23hum-Fc(LS) at 2.5 μg/mL was assessed for binding against 6101 full-length human plasma membrane proteins and cell surface tethered secreted proteins plus 396 human heterodimers expressed on transfected HEK293 cells in a human plasma membrane protein cell array. The fixed cell confirmation screen for the initial hits of the library screen is shown. (A) Binding of R3_DC23hum-Fc(LS) at 2.5 μg/mL. (B) Binding of Rituximab at 1 μg/mL. (C) Binding of IgG1 isotype control. (D) binding of the secondary antibody (PBS instead of primary antibody). Rep: replicate.

FIG. 33 Multispecific constructs comprising S1 and S2 targeting VHHs. (A, B, C) Schematic representation of bi-specific tandem VHHx-VHHy-Fc constructs with an S2 targeting VHH (C23) (a humanized form of VHH R3_DC23) and an S1 targeting VHH (117) (a humanized form of VHH3.117) interspaced with a 10 (A), 20 (B) or 30 (C) GS linker, fused to an Fc domain (human Fc (LS)) via a 10 GS linker. (D) Schematic representation of a VHHx-Fc-VHHy construct with an S2 targeting VHH (C23) (a humanized form of VHH R3_DC23) fused to the N-terminus of an Fc domain (human Fc(LS)) via a 10 GS linker and an S1 targeting VHH (117) (a humanized form of VHH3.117) fused to the C-terminus via a 15 GS linker. (E) Schematic representation of a tandem VHHx-VHHy-Fc construct with an S1 targeting VHH binding to or competing for the VHH72 epitopic (83) (a humanized version of VHH3.83) and an S1 targeting VHH binding to or competing for the VHH3.117 epitope (117) (a humanized form of VHH3.117) interspaced with a 20 GS linker, fused to an Fc domain (human Fc(LS)) via a 10 GS linker. (F) Schematic representation of a tri-specific tandem VHHx-VHHy-VHHz-Fc construct with an S2 targeting VHH (C23) (a humanized form of VHH R3_DC23), an S1 targeting VHH binding to or competing for the VHH3.117 epitopic (117) (a humanized form of VHH3.117) and an S1 targeting VHH binding to or competing for the VHH72 epitope (83) (a humanized version of VHH3.83) interspaced via a 20 GS linker, fused to an Fc domain (human Fc(LS)) via a 10 GS linker.

FIG. 34 Composition comprising S1 and S2 targeting VHHs. Schematic representation of a composition (XVR012) comprising S1 and S2 targeting binding agents. The composition comprises an S2 targeting VHH-Fc construct (XVR013) (a humanized form of R3_DC23 fused to a human Fc domain), and an S1 targeting VHHx-Fc-VHHy construct (XVR014), which comprises a VHH capable of binding to or competing for the VHH3.117 epitope (117) (a humanized form of VHH3.117) and a VHH capable of binding to or competing for the VHH72 epitope (83) (a humanized version of VHH3.83) fused to a human Fc domain.

FIG. 35 In vivo efficacy of XVR012, XVR013 and XVR014 in Syrian Golden hamster SARS-CoV-2 challenge model. Syrian golden hamsters were infected intranasally with SARS-COV-2 (Wuhan strain). The molecules XVR012 (4 and 20 mg/kg). XVR013 and XVR014 (2 and 10 mg/kg). Palivizumab (10 mg/kg, negative control) and bebtelovimab (10 mg/kg, positive control) were administered by intraperitoneal injection 4 hours after SARS-COV2 challenge. Viral replication in the lungs (A) and viral RNA load in lung tissue (B) are shown in the graphs. For each group, the median values with 95% confidence interval are reported. Dotted horizontal lines indicate the lower limit of detection (LLOD).

FIG. 36 XVR012, XVR013 and XVR014 mediated ADCC responses. A FcγRIIIa reporter assay was performed to assess the antibody-dependent cellular cytotoxicity (ADCC) of XVR012. XVR013 and XVR014. CHO-K1 expressing SARS COV-2 Spike Protein target cell line were used as target cells and Jurkat FcγRIIIa (CD16) V176-NFAT-RE Luc as reporter cells. Three independent assay runs were performed. The assay employed an effector to target cell ratio of 40:1, with the samples assessed in an 8-point dilution series starting at 30 μg/mL for XVR013 and XVR014 or at 60 μg/mL for XVR012 as three independent replicates (3 assay plates per run). An isotype control was assessed at a single concentration of 30 μg/ml. The assay plates were incubated overnight (21 hours #1 hour) prior to the addition of SteadyGlo (luminescence endpoint). Raw luminescence values are presented as the mean value for the test sample control wells (the response) and for the negative control wells (wells comprising of the target cell line in the absence of test sample). The mean values (N=3±SD) are reported for XVR012. XVR013 and XVR014.

FIG. 37 In vivo prophylactic efficacy of XVR012, XVR013 and XVR014 in Syrian Golden hamster SARS-COV-2 challenge model. Syrian golden hamsters were administered a cocktail of 10 mg/kg of XVR014 and 1 mg/kg of XVR013 (XVR012), 1 mg/kg XVR013 or 10 mg/kg XVR014 by intraperitoneal injection approximately 24 hours prior to SARS-COV2 challenge. Viral loads in the lungs are shown in the graphs. For each group, 6 animals were included and median values with 95% confidence intervals are shown. Dotted horizontal lines in the graph indicate the lower limit of detection (LLOD).

FIG. 38 In vivo therapeutic efficacy of XVR012, XVR013 and XVR014 in Syrian Golden hamster SARS-COV-2 challenge model. Syrian golden hamsters were infected with SARS-COV-2 (Wuhan strain). Cocktails of 5 mg/kg of XVR014 and 0.5 mg/kg of XVR013, of 10 mg/kg of XVR014 and 1 mg/kg of XVR013, or of 20 mg/kg of XVR014 and 2 mg/kg of XVR013 (XVR012); 0.5, 1 or 2 mg/kg XVR013; or 5, 10 or 20 mg/kg XVR014 were administered by intraperitoneal injection 4 hours after the SARS-COV2 challenge. Control animals received 10 mg/kg of palivizumab (negative control), and one group of animals received 10 mg/kg of bebtelovimab (used as positive control). Viral loads in the lungs are shown in the graphs. For each group, 6 animals were included and median values with 95% confidence intervals are shown. Dotted horizontal lines in the graph indicate the lower limit of detection (LLOD).

FIG. 39. Prophylactic treatment with R3_DC23-Fc protects K18-hACE2 mice against lethal SARS-COV-2 infection. Twenty hours prior to intratracheal infection with 3*10² PFU of SARS-CoV-2 614G variant virus, 100 μg R3_DC23-Fc was administered to K18-hACE2 mice and 100 μg of isotype control antibody (palivizumab) was administered to a second group of K18-hACE2 mice and non-permissive wild-type (WT) mice. Animals were monitored on a daily base by measuring weight change and scoring for humane endpoints. (A) The graph shows the Kaplan-Meier curve of animal survival portion of the indicated groups. Euthanasia was performed when mice lost more than 25% of their bodyweight as defined on day 0 or when a high score for 25 humane endpoints was reached. K18-hACE2 mice treated with R3_DC23-Fc (n=5) were significantly protected from lethality as compared to K18-hACE2 mice (n=5) treated with palivizumab (p=0.0016. Log-rank. Mantel-cox test). (B) The graph shows the mean relative bodyweight of K18-hACE2 mice treated with R3_DC23-Fc (n=5±SEM) or palivizumab (n=5±SEM) and of WT mice treated with palivizumab (n=5±SEM). From day 4 on mice treated with R3_DC23-Fc displayed significantly lower bodyweight loss as compared to mice treated with palivizumab (p<0.001) (2-way ANOVA. Tukey's multiple comparisons test).

FIG. 40. Prophylactic treatment with R3_DC23-Fc reduces viral replication of SARS-COV-2 in the lungs of infected K18-hACE2 mice. Twenty hours prior to intratracheal infection with 3*10² PFU of SARS-COV-2 614G variant virus, 100 μg R3_DC23-Fc was administered to K18-hACE2 mice and 100 μg of isotype control antibody (palivizumab) was administered to a second group of K18-hACE2 mice and non-permissive wild-type (WT) mice. Animals were monitored on a daily base by measuring weight change and scoring for humane endpoints. (A) The graph shows the Kaplan-Meier curve of animal survival portion of K18-mice treated with R3_DC23-Fc (n=5) as compared to K18-hACE2 mice (n=4) treated with palivizumab and WT mice treated with palivizumab (n=5). (B) The graph shows the mean relative bodyweight of K18-hACE2 mice treated with R3_DC23-Fc (n=5±SEM) or palivizumab (n=4±SEM) and of WT mice treated with palivizumab (n=5±SEM). From day 5 on K18-hACE2 mice treated with R3_DC23-Fc displayed significantly lower bodyweight loss as compared to K18-hACE2 mice treated with palivizumab (p<0.01) (2-way ANOVA. Tukey's multiple comparisons test). (C) The graph shows the median and individual viral titer in the lungs of mice from the indicated groups sacrificed at 5 days post infection. K18-hACE2 mice treated with R3_DC23-Fc and WT mice treated with palivizumab had significantly less replicating SARS-COV-2 virus in their lungs as compared to K18-hACE2 mice treated with palivizumab (p<0.0005. Kruskal-Wallis test. Dunn's multiple comparisons test). (D) The graph shows the median and individual levels of viral RNA in the lungs of mice from the indicated groups sacrificed at 5 days post infection. K18-hACE2 mice treated with R3_DC23-Fc and WT mice treated with palivizumab had significantly less replicating SARS-COV-2 virus in their lungs as compared to K18-hACE2 mice treated with palivizumab (p<0.005, Kruskal-Wallis test, Dunn's multiple comparisons test).

FIG. 41. S-2P:R3_DC23 interaction via BL1. Immobilization of S-2P trimer on a biolayer interferometry biosensor followed by association with 20 nM R3_DC23 monomer in solution. Raw data (no reference subtraction) of triplicate experiments.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of ±10% or less, preferably ±5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation is meant to apply throughout this specification, i.e., also in the context of other aspects or embodiments of the invention, unless otherwise defined.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

As corroborated by the experimental section, which illustrates certain representative embodiments of the invention, the inventors identified VHHs that specifically bind to Sarbecovirus spike protein, in particular to Sarbecovirus spike protein S2 subunit such as to SARS-COV-2 and SARS-COV-1 spike protein S2 subunit. The VHHs were found to potently neutralize SARS-COV-2, including SARS-COV-2 variants such as SARS-COV-2 D614G variant, SARS-COV-2 Alpha variant, SARS-CoV-2 Omicron BA.1 variant, SARS-COV-2 Omicron BA.2 variant, SARS-COV-2 Omicron BA.5 variant, SARS-COV-2 Omicron BA.2.75.2 variant, SARS-COV-2 Omicron BA.4.6 variant, SARS-CoV-2 Omicron BF.7 variant, SARS-COV-2 Omicron BQ.1.1 variant, SARS-COV-2 Omicron XBB variant, and SARS-COV-2 Omicron XBB.1.5 variant, and SARS-COV-1. It was found that these VHHs interact with S2 amino acids in the heptad repeat 2 (HR2) domain, more particularly with amino acids within a C-terminal region of the HR2 domain proximal to the viral membrane, which amino acids are very conserved within the spike protein of Sarbecoviruses of multiple clades.

Accordingly, an aspect relates to binding agents, in particular antibodies and antigen-binding fragments thereof, capable of neutralizing a Sarbecovirus, characterized in that said binding agents, in particular antibodies and antibody fragments, specifically bind to heptad repeat 2 (HR2) domain of spike protein of the Sarbecovirus.

An aspect provides a binding agent capable of neutralizing a Sarbecovirus, characterized in that said binding agent specifically binds to or within a region of spike protein of the Sarbecovirus corresponding to the region from amino acid E1188 to amino acid Y1206 of the SARS-COV-2 spike protein as defined in SEQ ID NO: 86.

A “binding agent” generally relates to a molecule that is capable of binding to at least one other molecule, wherein said binding is preferably a specific binding, such as on a defined binding site, pocket or epitope. The binding agent may be of any nature or type and is not dependent on its origin. The binding agent may be chemically synthesized, naturally occurring, recombinantly produced (and optionally purified), as well as designed and synthetically produced (and optionally purified). Said binding agent may hence be, e.g., a small molecule, a chemical, a peptide, a polypeptide, an antibody, or any derivative of any thereof, such as a peptidomimetic, an antibody mimetic, an active fragment, a chemical derivative, among others. A functional fragment of a binding agent or a functional part of a binding agent refers to a fragment or part of that binding agent that is functionally equivalent to that binding agent. In particular, such functional fragment or part of a binding agent as described herein ideally retains one or more of the functional features (1) to (21) of that binding agent as outlined extensively elsewhere herein.

The term “antibody” refers to an immunoglobulin (Ig) molecule or a molecule comprising an immunoglobulin (Ig) domain, which specifically binds with an antigen, as well as multimers thereof. “Antibodies” can be intact immunoglobulinsor immunoreactive portions of intact immunoglobulins. The term encompasses naturally, recombinantly, semi-synthetically or synthetically produced antibodies. Hence, for example, an antibody can be present in or isolated from nature, e.g., produced or expressed natively or endogenously by a cell or tissue and optionally isolated therefrom; or an antibody can be recombinant, i.e., produced by recombinant DNA technology, and/or can be, partly or entirely, chemically or biochemically synthesised.

By “isolated” or “purified” is meant material that is substantially or essentially free from components that normally accompany it in its native state. For example, an “isolated polypeptide” or “purified polypeptide” refers to a polypeptide which has been isolated or purified by any suitable means from a mixture of molecules comprising the to be isolated or to be purified polypeptide of interest. An isolated or purified polypeptide of interest can for instance be an immunoglobulin, antibody or nanobody, and the mixture can be a mixture or molecules as present in a cell producing the immunoglobulin, antibody or nanobody, and/or the culture medium into which the immunoglobulin, antibody or nanobody is secreted into (likely together with other molecules secreted by the cell). The terms “antibody fragment”, “antigen-binding fragment”, “functional antibody fragment” and “active antibody fragment” refer to a portion of any antibody that by itself has high affinity for an antigenic determinant, or epitope, and contains one or more complementarity determining regions (CDRs) accounting for such specificity. The terms “antibody fragment” and “antigen-binding fragment” and “active antibody fragment” and “functional antibody fragment” as used herein refer to a protein or peptide comprising an immunoglobulin domain or an antigen-binding domain capable of specifically binding to a Sarbecovirus spike protein such as SARS-COV-2 spike protein, in particular to the S2 subunit of the Sarbecovirus spike protein, more particularly to the HR2 domain of (the S2 subunit of) the Sarbecovirus spike protein. Non-limiting examples include immunoglobulin domains, Fab, F(ab)′2, scFv, heavy-light chain dimers, immunoglobulin single variable domains. Nanobodies (or VHH antibodies), domain antibodies, and single chain structures, such as a complete light chain or complete heavy chain.

The term “immunoglobulin (Ig) domain”, or more specifically “immunoglobulin variable domain” (abbreviated as “IVD”, also referred to herein as “variable domain”), means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs), and in particular the CDRs therein, even more particularly CDR3 therein, that confer specificity to an antibody for the antigen by carrying the antigen- or epitope-binding site. Typically, in conventional immunoglobulins, a heavy chain variable domain (VH) and a light chain variable domain (VL) interact to form an antigen-binding site. In this case, the complementarity determining regions (CDRs) of both VH and VL contribute (although not necessarily evenly) to the antigen-binding site, i.e. a total of 6 CDRs will be involved in antigen-binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, will bind to the respective epitope of an antigen by a pair of (associated) immunoglobulin domains such as light and heavy chain variable domains. i.e., by a VH-VL pair of immunoglobulin domains, which jointly bind to an epitope of the respective antigen.

An “immunoglobulin single variable domain” (abbreviated as “ISVD”), equivalent to the term “single variable domain”, defines molecules wherein the antigen-binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen-binding site. An “immunoglobulin single variable domain” (or “ISVD”) as used herein, refers to a protein or peptide with an amino acid sequence comprising 4 framework regions (FR) and 3 complementary determining regions (CDR) according to the format of FR1-CDR1-FR2-CDR2-FR3-CDR3-FR4. The antigen-binding site of an immunoglobulin single variable domain is formed by a single VH/VHH or VL domain. Hence, the antigen-binding site of an immunoglobulin single variable domain is formed by no more than three CDRs. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen-binding unit (i.e., a functional antigen-binding unit that essentially consists of the single variable domain, such that the single antigen-binding domain does not need to interact with another variable domain to form a functional antigen-binding unit). In certain embodiments, the immunoglobulin single variable domains are heavy chain variable domain sequences (e.g., a VH-sequence or a VHH-sequence); more specifically, the immunoglobulin single variable domains can be heavy chain variable domain sequences that are derived from a conventional four-chain antibody or heavy chain variable domain sequences that are derived from a heavy chain antibody. For example, the immunoglobulin single variable domain may be a (single) domain antibody (or an amino acid sequence that is suitable for use as a (single) domain antibody), a variable domain of a heavy (VH) or light (VL) chain of a conventional antibody (also referred to as a “dAb”) (or an amino acid sequence that is suitable for use as a dAb) or a Nanobody (as defined herein, and including but not limited to a VHH); or any suitable fragment of any one thereof.

In embodiments, the immunoglobulin single variable domain may be a Nanobody (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs. VHH domains. VHH antibody fragments, and VHH antibodies, have originally been described as the antigen-binding immunoglobulin (Ig) (variable) domain of “heavy chain antibodies” (i.e., of “antibodies devoid of light chains”; Hamers-Casterman et al. 1993, Nature 363:446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (2001. Rev Mol Biotechnol 74:277-302), as well as to the following patent applications, which are mentioned as general background art: WO 94/04678, WO 95/04079, WO 96/34103, WO 94/25591, WO 99/37681, WO 00/40968, WO 00/43507, WO 00/65057, WO 01/40310, WO 01/44301, EP 1134231, WO 02/48193, WO 97/49805, WO 01/21817, WO 03/035694, WO 03/054016, WO 03/055527, WO 03/050531, WO 01/90190, WO 03/025020 (=EP 1433793), WO 04/041867, WO 04/041862, WO 04/041865, WO 04/041863, WO 04/062551, WO 05/044858, WO 06/40153, WO 06/079372, WO 06/122786, WO 06/122787 and WO 06/122825. As described in these references. Nanobody (in particular VHH sequences and partially humanized Nanobody) can in particular be characterized by the presence of one or more “hallmark residues” in one or more of the framework sequences.

The binding agents or Sarbecovirus binding agents (can be used interchangeably) according to the current invention can in one aspect be described functionally by any individual function/embodiment or by any combination of any number of the individual functions/embodiments described hereafter and given an arbitrary number “n” between brackets “(n)”. The numerical order of these individual functions is random and not imposing any preference on an individual function; similarly, this random numerical order is not imposing any preference on any combination of two or more of the individual functions. Any such combination is furthermore not to be considered as arbitrary as the binding agents or Sarbecovirus binding agents herein exert each of these individual functions.

The present invention thus provides binding agents, in particular antibodies or antigen-binding fragments thereof, that (1) specifically bind to a Sarbecovirus such as SARS-COV-2 and SARS-COV-1 and may also be referred to herein as Sarbecovirus binding agents or Sarbecovirus antibodies and antibody fragments. In certain embodiments, the binding agents (2) do not bind Middle East respiratory syndrome coronavirus (MERS-COV).

“Binding” means any interaction, be it direct or indirect. A direct interaction implies a contact (e.g. physical or chemical) between two binding partners. An indirect interaction means any interaction whereby the interaction partners interact in a complex of more than two molecules. An interaction can be completely indirect (e.g. two molecules are part of the same complex with the help of one or more bridging molecules but don't bind in the absence of the bridging molecule(s)). An interaction may be partly direct or partly indirect: there is still a direct contact between two interaction partners, but such contact is e.g. not stable, and is stabilized by the interaction with one or more additional molecules.

“Specificity of binding” or “binding specificity” or “specifically binding” refers to the situation in which a molecule A is, at a certain concentration (e.g. sufficient to inhibit or neutralize a protein or process of interest) binding to a target of interest (e.g. protein) with higher affinity (e.g. at least 2-fold, 5-fold, or at least 10-fold higher affinity, e.g. at least 20-, 50- or 100-fold or more higher affinity) than the affinity with which it is possibly (if at all) binding to other targets (targets not of interest). Specific binding does not mean exclusive binding. However, specific binding does mean that a binder has a certain increased affinity or preference for one or a few of its targets. Exclusivity of binding refers to the situation in which a binder is binding only to the target of interest. The term “affinity”, as used herein, generally refers to the degree to which one molecule (e.g. ligand, chemical, protein or peptide, antibody or antibody fragment) binds to another molecule (e.g. (target) protein or peptide) so as to shift the equilibrium of single molecule monomers towards a complex formed by (specific) (non-covalent) binding of the two molecules. Non-covalent interaction or binding between 2 or more binding partners may involve interactions such as van der Waals interaction, hydrogen bonding, and salt bridges. The “dissociation constant” or “binding constant” (K_{D})) is commonly used to describe the affinity between the two molecules and it is often calculated by the ratio of the rate constant for the complex formation (referred to as the “kₒₙ” value) to the rate constant for dissociation of said complex (the “k_{off}” or “kais” value). The measurement of binding affinity of a molecule to another molecule, such as an antibody or antibody-fragment to an antigen, or a ligand to a receptor, is known to the skilled person and includes, e.g., real-time, label free bio-layer interferometry assay, e.g., an Octet® RED96 system (ForteBio), or surface plasmon resonance (SPR), e.g., BIACORE™, or solution-affinity ELISA.

The terms “Coronaviridae” and the more common name “coronavirus” refer to a family of viruses, which has its name from the large spike protein molecules that are present on the virus surface and give the virions a crown-like shape. The Coronoviridae family comprises four genera: Alphacoronavirus. Betacoronavirus. Gammacoronavirus, and Deltacoronavirus. Coronaviruses represent a diverse family of large enveloped positive-stranded RNA viruses that infect a wide range of animals, a wide variety of vertebrate species, and humans. The spike(S) proteins of coronaviruses are essential for host receptor-binding and subsequent fusion of the viral and host cell membrane, effectively resulting in the release of the viral nucleocapsids in the host cell cytoplasm (Letko et al. (2020) Nat Microbiol 5:562-569).

Four coronaviruses, presumably from a zoonotic origin, are endemic in humans: HCoV-NL63 and HCoV-229E (α-coronaviruses) and HCoV-OC43 and HCoV-HKU1 (β-coronaviruses). In addition, 3 episodes of severe respiratory disease caused by β-coronaviruses have occurred since 2000: severe acute respiratory syndrome virus (SARS), caused by SARS-COV-1, emerged from a zoonotic origin (bats via civet cats as an intermediate species) and disappeared in 2004 (Drosten et al. 2003. N Engl J Med 348:1967-1976). Over 8000 SARS cases were reported with a mortality rate of approximately 10%. In 2012, Middle East respiratory syndrome (MERS) emerged in the Arabian Peninsula. MERS is caused by MERS-COV, has been confirmed in over 2500 cases and has a case fatality rate of 34% (de Groot et al. 2013. N Engl J Virol 87:7790-7792). Starting at the end of 2019, the third zoonotic human coronavirus emerged with cases of severe acquired pneumonia reported in the city of Wuhan (China) being caused by a new β-coronavirus, now known as severe acute respiratory syndrome coronavirus 2 (SARS-COV-2), given its genetic relationship with SARS-COV-1 (Chen et al. (2020) Lancet 395:507-513). Similar to severe acute respiratory syndrome coronavirus (SARS-COV) and Middle East respiratory syndrome coronavirus (MERS-COV) infections, patients exhibited symptoms of viral pneumonia including fever, difficult breathing, and bilateral lung infiltration in the most severe cases (Gralinski et al. (2020) Viruses 12:135).

The term “Sarbecovirus” as used herein refers to a subgenus within the genus Betacoronavirus and includes the species Severe acute respiratory syndrome-related coronavirus (SARS-COV or SARS-CoV, also known as SARS coronavirus, SARS-related coronavirus, and Severe acute respiratory syndrome coronavirus, which are used as synonyms herein). Non-limiting examples of strains belonging to the SARS-COV species include SARS-COV-1 and SARS-COV-2.

The first available genome sequence placed the novel human pathogen SARS-COV-2 in the Sarbecovirus subgenus of Coronaviridae, the same subgenus as the SARS virus. Although SARS-CoV-2 belongs to the same genus Betacoronavirus as SARS-COV (lineage B) and MERS-COV (lineage C), genomic analysis revealed greater similarity between SARS-COV-2 and SARS-COV, supporting its classification as a member of lineage B (from the International Committee on Taxonomy of Viruses).

Among other Betacoronaviruses, this virus is characterized by a unique combination of polybasic cleavage sites, a distinctive feature known to increase pathogenicity and transmissibility. A bat Sarbecovirus. Bat CoV RaTG13, sampled from a Rhinolophus affinis horseshoe bat was reported to cluster with SARS-COV-2 in almost all genomic regions with approximately 96% genome sequence identity (and over 93% similarity in the receptor binding domain (RBD) of the spike protein); another mammalian species may have acted as intermediate host. One of the suspected intermediate hosts, the Malayan pangolin, harbours coronaviruses showing high similarity to SARS-COV-2 in the receptor-binding domain, which contains mutations believed to promote binding to the angiotensin-converting enzyme 2 (ACE2) receptor and demonstrates a 97% amino acid sequence similarity. SARS-COV-1 and -2 both use angiotensin converting enzyme 2 (ACE2) as a receptor on human cells. SARS-COV-2 binds ACE2 with a higher affinity than SARS-COV-1 (Wrapp et al. (2020) Science 367:1260-1263). SARS-COV-2 differentiates from SARS-COV-1 and several SARS-related coronaviruses (SARSr-CoVs) as outlined in e.g. Abdelrahman et al. (2020. Front Immunol 11:552909).

SARS-COV-2 refers to the newly-emerged Sarbecovirus which was identified as the cause of a serious and worldwide outbreak of severe acquired pneumonia starting in the city of Wuhan (China). The long-term global spread of SARS-COV-2, together with selective pressure for immune escape, led to adaptation of the virus to the host and generation of new SARS-COV-2 variants. Specifically, multiple mutations in the spike glycoprotein evolved and are evolving, including mutations that are located in the spike S1 subunit. For example, a SARS-COV-2 variant may comprise a mutation at one or more positions selected from N439, K417, S477, L452, T478, E484, P384, N501 and D614 (relative to the SARS-COV-2 spike amino acid sequence as defined in SEQ ID NO:86). Further non-limiting examples of SARS-COV-2 variants include a SARS-COV-2 variant comprising a mutation at position N501 such as a N501Y variant (e.g. SARS-COV-2 Alpha variant); a SARS-COV-2 variant comprising a mutation at positions N501 and E484 such as a N501Y and E484K variant (e.g. SARS-CoV-2 Alpha+E484K variant); a SARS-COV-2 variant comprising a mutation at positions K417. E484 and N501 such as a K417N. E484K and N501Y variant (e.g. SARS-COV-2 beta variant); a SARS-COV-2 variant comprising a mutation at positions P384, K417, E484 and N501 such as a P384L, K417N, E484K and N501Y variant (e.g. SARS-COV-2 beta+P384L variant); a SARS-COV-2 variant comprising a mutation at positions L452 and E484 such as a L452R and E484Q variant (e.g. SARS-COV-2 kappa variant); a SARS-COV-2 variant comprising a mutation at positions L452 and T478 such as a L452R and T478K variant (e.g. SARS-COV-2 delta variant); a SARS-COV-2 variant comprising a mutation at position L452 such as a L452R variant (e.g. SARS-COV-2 epsilon variant); a SARS-COV-2 variant comprising a mutation at position K417 such as a K417T variant (e.g. SARS-COV-2 gamma variant); a SARS-COV-2 variant comprising a mutation at position D614 such as a D614G variant (e.g. SARS-COV-2 D614G variant, SARS-COV-2 Omicron BA.1 variant or SARS-COV-2 Omicron BA.2 variant); a SARS-COV-2 variant comprising a mutation at positions K147, W152R, F157, 1210, G257, D339, G446 and N460 such as a K147E, W152R, F157L, 1210V, G257S, D339H, G446S and N460K variant (e.g. SARS-COV-2 Omicron BA.2.75 variant, SARS-CoV-2 Omicron BA.2.75.2 variant); a SARS-COV-2 variant comprising a mutation at positions R346, F486 and D1199 such as a R346T, F486S and D1199N variant (e.g. SARS-COV-2 Omicron BA.2.75.2 variant); a SARS-COV-2 variant comprising a mutation at positions H69, V70, L452 and F486 such as a H69-, V70-, L452R and F486V variant (e.g. SARS-COV-2 Omicron BA.4/BA.5 variant); a SARS-COV-2 variant comprising a mutation at positions R346 and N658 such as a R346T and N658S variant (e.g. SARS-COV-2 Omicron BA.4.6 variant); a SARS-COV-2 variant comprising a mutation at positions R346 such as a R346T variant (e.g. SARS-COV-2 Omicron BF.7 variant); a SARS-COV-2 variant comprising a mutation at positions R346, K444 and N460 such as a R346T, K444T and N460K variant (e.g. SARS-COV-2 Omicron BQ.1.1 variant); a SARS-COV-2 variant comprising a mutation at positions V83, Y144, H146, Q183, V213, R346, L368, V445, G446, N460, F486 and F490 such as a V83A, Y144-, H146Q, Q183E, V213E, R346T, L368I, V445P, G446S, N460K, F486S and F490S variant (e.g. SARS-COV-2 Omicron XBB variant) or a V83A, Y144-, H146Q, Q183E, V213E, R346T, L368I, V445P, G446S, N460K, F486P and F490S variant (e.g. SARS-COV-2 Omicron XBB.1.5 variant). The Alpha variant (also known as B.1.1.1.7 lineage) of SARS-COV-2 was first detected in the UK late 2020 and was one of the first reported variants of concern of SARS-COV-2. It contained several mutations in the spike protein, including N501Y mutation and D614G mutation. The Omicron variant of SARS-COV-2 was first identified in South Africa and Botswana and was reported to the World Health Organization (WHO) on Nov. 24, 2021, as a novel variant (Fan et al. 2022. Signal Transduct Target Ther. 7:141). The Omicron variant is not a single strain, but evolved into at least three lineages, including BA.1. BA.2, and BA.3. Up to 60 mutations have been identified in the BA.1 lineage, with as many as 38 of these occurring in the spike(S) protein, one in the envelope (E) protein, two in the membrane (M) protein, and six in the nucleocapsid (N) protein. BA.2 lineage possesses 57 mutations, with 31 in the S protein, of which the N-terminus is significantly different from that of BA.1. The term “SARS-COV-2” as used herein covers both the original strain identified in Wuhan as well as variants thereof.

The binding agents, in particular the antibodies and antibody fragments (3) specifically bind or bind to spike protein of a Sarbecovirus such as SARS-COV-2 spike protein or SARS-COV-1 spike protein, in particular the binding agents, in particular the antibodies and antibody fragments, (4) specifically bind or bind to S2 subunit, or to a part of the S2 subunit, of the Sarbecovirus spike protein, more particularly, the binding agents, in particular the antibodies and antibody fragments, (22) specifically bind or bind to or within a region of the S2 subunit located from amino acid E1188 to amino acid Y 1206, preferably a region located from amino acid N1192 to amino acid Y1206 or a region located from amino acid E1188 to amino acid L1203, more preferably a region located from amino acid N1192 to amino acid L1203, even more preferably a region located from amino acid N1194 to amino acid L1203, most preferably a region located from amino acid N1194 to amino acid Q1201 of the SARS-COV-2 spike protein as defined in SEQ ID NO:86. In certain embodiments, the binding agents, in particular the antibodies and antibody fragments, (23) specifically bind or bind to or within a region of spike protein of a Sarbecovirus or S2 subunit of the Sarbecovirus spike protein corresponding to the region from amino acid E1188 to amino acid Y1206, preferably amino acid N1192 to amino acid Y1206 or amino acid E1188 to amino acid L1203, more preferably amino acid N1192 to amino acid L1203, even more preferably amino acid N1194 to amino acid L1203, most preferably amino acid N1194 to amino acid Q1201 of the SARS-COV-2 spike protein as defined in SEQ ID NO:86. More particularly, the binding agents, in particular the antibodies and antibody fragments, (5) specifically bind or bind to heptad repeat 2 (HR2) domain, or to a part of the HR2 domain, of (the S2 subunit of) the Sarbecovirus spike protein. In certain embodiments, the binding agents, in particular the antibodies and antibody fragments, (6) specifically bind or bind to or within a region of the HR2 domain proximal to the viral membrane, preferably a region located from amino acid A1174 to amino acid E1202, more preferably a region located from amino acid I1179 to amino acid E1202, even more preferably a region located from amino acid D1184 to amino acid E1202, still more preferably a region located from amino acid E1188 to amino acid E1202 or a region located from amino acid V1189 to amino acid E1202, yet more preferably a region located from amino acid N1194 to amino acid E1202, most preferably a region located from amino acid N1194 to amino acid Q1201 of the SARS-COV-2 spike protein as defined in SEQ ID NO:86, or (7) specifically bind or bind to a region of the HR2 domain (or of the S2 subunit) corresponding to the region from amino acid E1188 to amino acid Y1206 of the SARS-COV-2 spike protein as defined in SEQ ID NO:86, preferably a region of the HR2 domain (or of the S2 subunit) corresponding to the region from amino acid E1188 to amino acid Y1203 of the SARS-COV-2 spike protein as defined in SEQ ID NO:86, more preferably a region of the HR2 domain (or of the S2 subunit) corresponding to the region from amino acid A1190 to amino acid L1203 of the SARS-COV-2 spike protein as defined in SEQ ID NO: 86, such as a region of the HR2 domain (or of the S2 subunit) corresponding to the region from amino acid K1191 to amino acid E1202 of the SARS-COV-2 spike protein as defined in SEQ ID NO: 86, or a region of the HR2 domain (or of the S2 subunit) corresponding to the region from amino acid N1192 to amino acid Q1201 of the SARS-COV-2 spike protein as defined in SEQ ID NO:86 (such as the region from amino acid N1192 to amino acid Q1201 of the SARS-COV-2 spike protein as defined in SEQ ID NO:86), even more preferably a region of the HR2 domain (or of the S2 subunit) corresponding to the region from amino acid N1194 to amino acid L1203 of the SARS-COV-2 spike protein as defined in SEQ ID NO:86, most preferably a region of the HR2 domain (or of the S2 subunit) corresponding to the region from amino acid N1194 to amino acid Q1201 of the SARS-CoV-2 spike protein as defined in SEQ ID NO:86 such as a region of the HR2 domain (or of the S2 subunit) corresponding to the region from amino acid S1196 to amino acid Q1201 of the SARS-CoV-2 spike protein as defined in SEQ ID NO:86. In particular embodiments, the binding agents, in particular the antibodies and antibody fragments, (8) specifically bind or bind to at least one, at least two, at least three, at least four, at least five, at least six, at least seven or all, of the amino acid residues N1192, N1194, S1196, L1197, D1199, L1200, Q1201 and E1202, of the SARS-COV-2 spike protein as defined in SEQ ID NO:86, preferably to at least one, at least two, at least three, at least four or all of the amino acid residues N1194, S1196, D1199, Q1201 and E1202 of the SARS-COV-2 spike protein as defined in SEQ ID NO:86, more preferably to at least one, at least two, at least three or all of the amino acid residues N1194, S1196, D1199 and Q1201 of the SARS-COV-2 spike protein as defined in SEQ ID NO:86, most preferably to at least one or both of the amino acid residues S1196 and Q1201 of the SARS-COV-2 spike protein as defined in SEQ ID NO:86. In embodiments, the binding agents, in particular the antibodies and antibody fragments, (24) specifically bind or bind to at least one, at least two, at least three, at least four, at least five, at least six, at least seven or all, amino acid residue(s) of spike protein of a Sarbecovirus or S2 subunit or HR2 domain of the Sarbecovirus spike protein corresponding to the amino acid residues N1192, N1194, S1196, L1197, D1199, L1200, Q1201 and E1202 of the SARS-COV-2 spike protein as defined in SEQ ID NO:86, preferably to at least one, at least two, at least three, at least four or all amino acid residue(s) corresponding to the amino acid residues N1194, S1196, D1199, Q1201 and E1202 of the SARS-CoV-2 spike protein as defined in SEQ ID NO:86, more preferably to at least one, at least two, at least three or all amino acid residues(s) corresponding to the amino acid residues N1194. S1196. D1199 and Q1201 of the SARS-COV-2 spike protein as defined in SEQ ID NO:86, most preferably to at least one or both amino acid residue(s) corresponding to the amino acid residues S1196 and Q1201 of the SARS-COV-2 spike protein as defined in SEQ ID NO:86. In particular embodiments, the binding agents, in particular the antibodies and antibody fragments, (25) specifically bind or bind to the amino acid residues S1196 and Q1201 of the SARS-COV-2 spike protein as defined in SEQ ID NO: 86 or to the amino acid residues of spike protein corresponding to said amino acid residues of S1196 and Q1201 of the SARS-COV-2 spike protein as defined in SEQ ID NO:86, optionally to the amino acid residues N1194, S1196, D1199 and Q1201 of the SARS-COV-2 spike protein as defined in SEQ ID NO:86 or to the amino acid residues of spike protein corresponding to said amino acid residues N1194, S1196, D1199 and Q1201 of the SARS-COV-2 spike protein as defined in SEQ ID NO: 86.

In particular embodiments, (26) at least one, at least two, at least three, at least four, at least five, at least six, at least seven or all, of the amino acid residues N1192, N1194, S1196, L1197, D1199, L1200, and Q1201 and E1202, of the SARS-COV-2 spike protein as defined in SEQ ID NO:86, preferably at least one, at least two, at least three, at least four or all of the amino acid residues N1194, S1196, D1199, Q1201 and E1202 of the SARS-COV-2 spike protein as defined in SEQ ID NO:86, more preferably at least one, at least two, at least three or all of the amino acid residues N1194, S1196, D1199 and Q1201 of the SARS-COV-2 spike protein as defined in SEQ ID NO:86, most preferably at least one or both of the amino acid residues S1196 and Q1201 of the SARS-COV-2 spike protein as defined in SEQ ID NO:86, are indispensable for binding of the binding agents, in particular the antibodies and antibody fragments, to spike protein. In embodiments, (27) at least one, at least two, at least three, at least four, at least five, at least six, at least seven or all, amino acid residue(s) of spike protein of a Sarbecovirus or S2 subunit or HR2 domain of the Sarbecovirus spike protein corresponding to the amino acid residues N1192, N1194, S1196, L1197, D1199, L1200, Q1201 and E1202 of the SARS-COV-2 spike protein as defined in SEQ ID NO:86, preferably at least one, at least two, at least three, at least four or all amino acid residue(s) corresponding to the amino acid residues N1194, S1196, D1199, Q1201 and E1202 of the SARS-COV-2 spike protein as defined in SEQ ID NO:86, more preferably at least one, at least two, at least three or all amino acid residues(s) corresponding to the amino acid residues N1194, S1196, D1199 and Q1201 of the SARS-COV-2 spike protein as defined in SEQ ID NO:86, most preferably at least one or both amino acid residue(s) corresponding to the amino acid residues S1196 and Q1201 of the SARS-COV-2 spike protein as defined in SEQ ID NO:86, are indispensable for binding of the binding agents, in particular the antibodies and antibody fragments, to spike protein. In particular embodiments, (28) the amino acid residues S1196 and Q1201 of the SARS-COV-2 spike protein as defined in SEQ ID NO:86 or the amino acid residues of spike protein corresponding to said amino acid residues of S1196 and Q1201 of the SARS-COV-2 spike protein as defined in SEQ ID NO:86, optionally the amino acid residues N1194, S1196, D1199 and Q1201 of the SARS-COV-2 spike protein as defined in SEQ ID NO:86 or the amino acid residues of spike protein corresponding to said amino acid residues N1194, S1196, D1199 and Q1201 of the SARS-COV-2 spike protein as defined in SEQ ID NO:86, are indispensable for binding of the binding agents, in particular the antibodies and antibody fragments, to spike protein.

Assessment of the binding site may be evaluated by determining the crystal structure of a complex of the binding agent, in particular the antibody or antibody fragment, and a spike protein, or an S2 subunit or a peptide comprising a HR2 domain, for example by applying the crystal structure determination method as shown in the examples, and/or by selection and analysis of viral escape variants/mutants, for example by applying the viral escape selection method as shown in the examples, and/or by analysing hydrogen-deuterium exchange on recombinant spike protein (or S2 subunit or HR2 containing peptides) in the presence and absence of the binding agent, for example by applying the hydrogen-deuterium exchange method monitored by mass spectrometry (HDX-MS method) as shown in the examples.

Advantageously, these amino acid residues are conserved between different clades of Sarbecoviruses, in particular between clade 1, clade 2, and clade 3 Sarbecoviruses. In preferred embodiments, the binding agents, in particular the antibodies or antibody fragments, (9) do not bind to the RBD of the Sarbecovirus spike protein.

The binding agents, in particular the antibodies and antibody fragments (29) specifically bind or bind to a quaternary epitope of the spike protein. In particular, the binding agents, in particular the antibodies and antibody fragments (30) specifically bind or bind to a trimeric HR2 domain (or a trimeric S2 subunit or a trimeric spike protein). In particular, the binding agents, in particular the antibodies and antibody fragments (31) specifically bind or bind to a quaternary epitope within a trimeric HR2 domain (or a trimeric S2 subunit or a trimeric spike protein). More particularly, the binding agents, in particular the antibodies and antibody fragments, (32) specifically bind or bind to a quaternary epitope located within two adjacent HR2 domains or helices. In particular embodiments, the binding agents, in particular the antibodies and antibody fragments, (33) specifically bind or bind to a quaternary epitope comprising or consisting of one or more interacting amino acid residues as described herein in one HR2 domain or helix as well as one or more interacting amino acid residues as described herein in an adjacent HR2 domain or helix. In particular embodiments, the binding agents, in particular the antibodies and antibody fragments, (34) specifically bind or bind to a quaternary epitope within a trimeric spike protein, wherein amino acid residues, particularly one or more interacting amino acid residues as described herein, from at least two such as two monomers of the trimeric spike protein contribute to said quaternary epitope.

As used herein, the term “quaternary epitope” refers to a conformational epitope whose structure depends upon or is enhanced by the arrangement of multiple protomers or monomers into a multimeric complex. A quaternary epitope may be located in a single protein (or monomer) of a multimeric complex; or it may span multiple protomers, being formed de novo by their interaction. Specific binding or binding to a quaternary epitope or a multimeric protein can be assessed by evaluating binding to monomeric and/or (stabilized) multimeric protein by means of an Enzyme Linked Immunosorbent Assay (ELISA) assay, for example by applying the ELISA assay as shown in the examples. Stabilization of trimeric spike protein may be achieved by fusing the spike protein to the foldon domain of the trimeric protein fibritin from bacteriophage T4. Correlation between binding to the monomeric protein and density of the monomeric protein at elevated densities of the monomeric protein only, such as at a density of 1.0 ng/mm² or more, preferably 1.2 ng/mm² or more, or 1.5 ng/mm² or more, may be indicative for specific binding or binding to a multimeric conformation of the protein. For a given density of monomeric and multimeric protein, enhanced binding to multimeric protein compared to the monomeric protein may indicate specific binding or binding to the multimeric protein.

Without wishing to be bound by any theory, upon binding to the trimeric spike protein, in particular to a quaternary epitope within the trimeric spike protein, the binding agents, in particular the antibodies and antibody fragments, described herein may stabilize the prefusion conformation of the spike protein. More particularly, the binding agent may stabilise or lock the HR2 coiled-coil. As such, the binding agents may prevent the unravelling of the HR2 coiled-coil, which is considered a critical early step in the spike-controlled membrane fusion process; or the binding agents may interfere with or block migration of the HR2 alpha helices towards the extended HR1 alpha helices, which is considered a critical step in the refolding of the spike protein from a prehairpin intermediate to a postfusion conformation; and/or the binding agents may prevent the completion of the 6 helix bundle formation, which is considered crucial for the fusion process. In embodiments, the binding agents, in particular the antibodies and antibody fragments (35) are capable of stabilizing the prefusion conformation of spike protein of a Sarbecovirus. In embodiments, the binding agents, in particular the antibodies and antibody fragments are (36) capable of stabilizing the HR2 coiled-coil. SARS-COV-2 contains as structural proteins the spike(S) protein, the envelope (E) protein, the membrane (M) protein, and the nucleocapsid (N) protein. Furthermore, sixteen nonstructural proteins (nsp1-16) have been discerned, which are involved in replication and modifying the host defense. The Nsp12 protein corresponds to a RNA-dependent RNA polymerase (RdRp).

Of specific interest in the current invention is the spike or S protein, which is a transmembrane glycoprotein forming homotrimers protruding from the viral surface and giving the virus a crown-like look. The spike protein has two subunits: S1 and S2.

The S1 subunit comprises an N-terminal domain (NTD), a receptor binding domain (RBD), and subdomains 1 and 2 (SD1, SD2). The S1 subunit is involved in host receptor binding. The spike protein binds to human host cell receptor angiotensin-converting enzyme 2 (ACE2) via the receptor binding domain (RBD) present in the S1 subunit.

The S2 subunit is involved in fusing the membranes of viruses and host cells and viral entry, and comprises multiple domains: an S2′ protease cleavage site (cleavage by a host protease required for fusion), a fusion peptide (FP), a heptad repeat 1 (HR1) domain, a central helix (CH) domain, a connector domain (CD), a heptad repeat 2 (HR2) domain, a transmembrane (TM) domain, and a cytoplasmic tail (CT) domain (Wang et al. (2020). Front Cell Infect Microbiol 10:587269).

The S protein normally exists in a prefusion conformation. In said prefusion conformation. S1 and S2, cleaved at the S1-S2 furin cleavage site during biosynthesis, remain non-covalently bound to each other—this is different from SARS-COV in which S1 and S2 remain uncleaved. In the closed state of the S protein (PDB:6VXX), the 3 RBD domains in the trimer do not protrude from the trimer whereas in the open state (PDB:6VYB), or “up” conformation, one of the RBD does protrude from the trimer. The S-trimer ectodomain with triangular cross-section has a length of approximately 160-Angstrom wherein the S1 domain adopts a V-shaped form. Sixteen of the 22 N-linked glycosylation sites per protomer appear glycosylated (Walls et al. (2020) Cell 180:281-292).

The S1 subunit of the S protein binds with ACE2 through its RBD region to promote the formation of endosomes, which triggers viral fusion activity. After S1-ACE2 binding. S is cleaved by cellular proteases, such as transmembrane protease serine subtype 2 (TMPRSS2) or endosomal cathepsins, which exposes the fusion peptide (FP) that is located in the S2 subunit. The FP inserts into the host cell membrane, thereby shortening the distance between the viral membrane and host cell membrane, the HR1 domain of the S protein is in close proximity to the host cell membrane, whereas the HR2 domain is closer to the viral membrane side. Then, HR2 folds back to HR1, whereby the two HR domains form a six-helix structure in an antiparallel format of the fusion core. The viral membrane is so pulled toward the host cell membrane and tightly binds to it, and the two membranes fuse, resulting in the release of the viral genome into the host cell (Huang et al. (2020) Acta Pharmalogica Sinica 41:1141-1149).

The terms “spike protein”, “S” or “S protein” as used herein as synonyms refer to the spike protein of a Sarbecovirus, and can refer to specific S proteins such as SARS-COV-2 S protein and SARS-CoV-1 S protein. The terms “spike protein” and “SARS-COV-2 spike protein” include protein variants of Sarbecovirus or SARS-COV-2 spike protein isolated from different Sarbecovirus or SARS-COV-2 isolates, as well as recombinant Sarbecovirus or SARS-COV-2 spike protein, or a fragment thereof. The terms also encompass Sarbecovirus spike protein or SARS-COV-2 spike protein coupled to, for example, a histidine tag, mouse or human Fc, or a signal sequence. The SARS-COV-2 spike protein sequence can be found under/corresponds with or to Genbank Accession: QHQ82464, version QHQ82464.1; and is also defined herein as SEQ ID NO:86:

MFVFLVLLPLVSSQCVNLTTRTQLPPAYTNSFTRGVYYPDKVFRSSVLHSTQDLFLPFFSNVTWFH
AIHVSGTNGTKRFDNPVLPFNDGVYFASTEKSNIIRGWIFGTTLDSKTQSLLIVNNATNVVIKVCE
FQFCNDPFLGVYYHKNNKSWMESEFRVYSSANNCTFEYVSQPFLMDLEGKQGNFKNLREFVFKNID
GYFKIYSKHTPINLVRDLPQGFSALEPLVDLPIGINITRFQTLLALHRSYLTPGDSSSGWTAGAAA
YYVGYLQPRTFLLKYNENGTITDAVDCALDPLSETKCTLKSFTVEKGIYQTSNFRVQPTESIVRFP
NITNLCPFGEVFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYLYRLFRKSNLK
PFERDISTEIYQAGSTPCNGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAVRDPQTLEILDITPCSFGG
VSVITPGTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTISVTTEI
LPVSMTKTSVDCTMYICGDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCLGDIAARDLICAQKFNGL
TVLPPLLTDEMIAQYTSALLAGTITSGWTFGAGAALQIPFAMQMAYRFNGIGVTQNVLYENQKLIA
NQFNSAIGKIQDSLSSTASALGKLQDVVNQNAQALNTLVKQLSSNFGAISSVLNDILSRLDKVEAE
VQIDRLITGRLQSLQTYVTQQLIRAAEIRASANLAATKMSECVLGQSKRVDFCGKGYHLMSFPQSA
PHGVVFLHVTYVPAQEKNFTTAPAICHDGKAHFPREGVFVSNGTHWFVTQRNFYEPQIITTDNTFV
SGNCDVVIGIVNNTVYDPLQPELDSFKEELDKYFKNHTSPDVDLGDISGINASVVNIQKEIDRLNE
VAKNLNESLIDLQELGKYEQYIKWPWYIWLGFIAGLIAIVMVTIMLCCMTSCCSCLKGCCSCGSCC
KFDEDDSEPVLKGVKLHYT

Herein, the SARS-COV-2 spike protein HR2 domain corresponds with/to amino acids 1169-1202 of SEQ ID NO:86 and as depicted hereafter (SEQ ID NO:87):

(SEQ ID NO: 87)
ISGINASVVNIQKEIDRLNEVAKNLNESLIDLQE

Herein, the SARS-COV-2 spike protein TM domain corresponds with/to amino acids 1214-1237 of SEQ ID NO:86.

As used herein a region of the HR2 domain “proximal to the viral membrane” refers to a region within the HR2 domain that is within 40 amino acids from the viral membrane.

The Sars-Cov-1 spike protein sequence can be found under/corresponds with or to GenBank accession NP_828851.1; and is also defined herein as SEQ ID NO: 111. Herein, the SARS-COV-1 spike protein HR2 domain corresponds with/to amino acids 1151-1184 of SEQ ID NO: 111 and as depicted in SEQ ID NO:87. The amino acids and amino acid numbering referred to herein is relative to/corresponding to the SARS-COV-2 spike protein as defined in SEQ ID NO:86; corresponding amino acids in spike proteins or spike protein fragments, domains or regions of other Sarbecoviruses can be easily determined by aligning multiple amino acid sequences.

“Angiotensin converting enzyme 2”, “ACE2”, or “ACE-2” as used herein interchangeably refers to mammalian protein belonging to the family of dipeptidyl carboxydipeptidases, and sometimes classified as EC:3.4.17.23. The genomic location of the human ACE2 gene is on chrX:15,561,033-15,602,158 (GRCh38/hg38; minus alternatively strand), or on chrX:15,579,156-15,620,271 (GRCh37/hg19; minus strand). ACE2 acts as a receptor for at least human coronaviruses SARS-COV and SARS-COV-2, and NL63/HCoV-NL63 (also known as New Haven coronavirus). UniProtKB identifier of human ACE2 protein: Q9BYF1. Isoform 1 (identifier: Q9BYF1-1) has been chosen as the canonicali sequence. Reference DNA sequence of the human ACE2 gene in GenBank: NC 000023.11. Reference mRNA sequences of human ACE2 in GenBank NM_001371415.1 and NM 021804.3.

A further functional characteristic of the binding agents, in particular the antibodies and antibody fragments, described herein is that they are (10) capable of neutralizing a Sarbecovirus, in particular (11) capable of neutralizing any one or both, preferably both, of SARS-COV-2 and SARS-COV-1.

As used herein, a “neutralizing binding agent” or a “neutralizing antibody” (or “binding agent or antibody that is capable of neutralizing a Sarbecovirus, in particular SARS-COV-2 and/or SARS-CoV-1”) refers to a binding agent or antibody that binds to a Sarbecovirus, in particular SARS-COV-2 and/or SARS-COV-1, to inhibit or suppress the ability of the Sarbecovirus, or SARS-COV-2 or SARS-COV-1, to initiate and/or perpetuate an infection in a host. Neutralizing binding agents or antibodies may, for example, interfere with binding of a Sarbecovirus such as SARS-COV-2 or SARS-COV-1 to a host receptor, in particular ACE2; and/or with viral entry, e.g. by inducing S1 shedding and/or by interfering with viral fusion. At present it is not fully clear how the binding agents and antibodies according to the current invention are neutralizing, inhibiting, blocking or suppressing a Sarbecovirus infection. In certain embodiments, the binding agents, in particular the antibodies and antibody fragments, described herein (44) do not modulate or interfere with S1 shedding. In certain embodiments, the binding agents, in particular the antibodies and antibody fragments, described herein (12) do not induce S1 shedding. In certain embodiments, the binding agents, in particular the antibodies and antibody fragments, described herein (45) do not prevent S1 shedding. In certain embodiments, the binding agents, in particular the antibodies and antibody fragments, described herein are (13) capable of inhibiting spike-mediated syncytia formation. Consequently, the binding agents, in particular the antibodies and antibody fragments, may be (14) capable of inhibiting viral fusion and, without wishing to be bound by any theory, may as such not allow the Sarbecovirus to complete the infection process into a host cell. In certain embodiments, the binding agents, in particular the antibodies and antibody fragments, described herein (46) do not prevent HR1 unfolding. In certain embodiments, the binding agents, in particular the antibodies and antibody fragments, described herein (47) do not prevent folding of HR1 onto HR2 (e.g. as during formation of a S2 6 helix bundle). Independent of their mechanism of action, the binding agents, in particular the antibodies and antibody fragments, according to the invention are capable of neutralizing a Sarbecovirus infection potently.

Neutralizing activity can be measured using a standard neutralization assay as known to one of skill in the art, including, without limitation, a pseudovirus neutralization assay and a plaque reduction test. Exemplary methods for performing such neutralization assays are described herein in the examples. Neutralizing activity can also be evaluated by measuring one or more indicators of a Sarbecovirus, or SARS-COV-2 or SARS-COV-1, infection, such as syncytia formation between cells expressing a Sarbecovirus spike protein and cells expressing the Sarbecovirus receptor ACE2.

In particular embodiments, the binding agents, in particular the antibodies and antibody fragments, are (15) capable of neutralizing a Sarbecovirus, in particular SARS-COV-2 and/or SARS-COV-1, with a half maximum inhibitory concentration or 50% inhibitory concentration (IC₅₀) of 100 ng/ml or less, preferably 50 ng/ml or less or 20 ng/ml or less, more preferably 10 ng/ml or less, even more preferably 1 ng/ml or less, preferably as determined in a Sarbecovirus spike protein pseudovirus neutralization assay such as a vesicular stomatitis virus (VSV)-Sarbecovirus spike protein pseudovirus neutralization assay, more preferably as determined in a SARS-COV-2 spike protein and/or SARS-COV-1 spike protein pseudovirus neutralization assay such as a VSV-SARS-COV-2 spike protein pseudovirus neutralization assay or a VSV-SARS-COV-1 spike protein pseudovirus neutralization assay. In particular, the pseudovirus neutralization assay may be based on pseudotyped VSV-delG virus containing the spike protein of a Sarbecovirus such as SARS-COV-2 spike protein. SARS-COV-2 variant spike protein or SARS-COV-1 spike protein. As used herein in connection with the neutralizing activity of a binding agent or antibody, “half maximum inhibitory concentration” or “IC₅₀” refers to a quantity such as a concentration of a binding agent or antibody required for 50% neutralization of the Sarbecovirus.

In particular embodiments, the binding agents, in particular the antibodies and antibody fragments, are (16) capable of neutralizing at least one SARS-COV-2 variant such as a SARS-COV-2 variant comprising a mutation at position D614 (relative to the SARS-COV-2 spike amino acid sequence as defined in SEQ ID NO:86) such as a D614G variant, in particular at least any one or more, preferably all, of SARS-COV-2 Alpha variant, SARS-COV-2 Omicron BA.1 variant SARS-COV-2 Omicron BA.2 variant, SARS-COV-2 Omicron BA.5 variant, SARS-COV-2 Omicron BA.2.75.2 variant, SARS-COV-2 Omicron BA.4.6 variant, SARS-COV-2 Omicron BF.7 variant, SARS-COV-2 Omicron BQ.1.1 variant, SARS-COV-2 Omicron XBB variant and SARS-COV-2 Omicron XBB.1.5 variant.

In particular embodiments, the binding agents, in particular the antibodies and antibody fragments described herein are characterized in that they are (17) capable of neutralizing SARS-COV-2 Alpha variant, (18) capable of neutralizing SARS-COV-2 Omicron BA.1 variant, (19) capable of neutralizing SARS-COV-2 Omicron BA.2 variant, (37) capable of neutralizing SARS-COV-2 Omicron BA.5 variant, (38) capable of neutralizing SARS-COV-2 Omicron BA.2.75.2 variant, (39) capable of neutralizing SARS-COV-2 Omicron BA.4.6 variant, (40) capable of neutralizing SARS-CoV-2 Omicron BF.7 variant, (41) capable of neutralizing SARS-COV-2 Omicron BQ.1.1 variant, (42) capable of neutralizing SARS-COV-2 Omicron XBB variant, and/or (43) capable of neutralizing SARS-COV-2 Omicron XBB.1.5 variant with an IC₅₀ of 100 ng/ml or less, preferably 50 ng/ml or less or 20 ng/ml or less, more preferably 10 ng/ml or less, even more preferably 1 ng/ml or less, preferably as determined in a SARS-COV-2 variant spike pseudovirus neutralization assay such as a VSV-SARS-COV-2 variant spike pseudovirus neutralization assay.

The binding agents, in particular the antibodies and antibody fragments, described herein are further characterized in that they are (14) capable of inhibiting viral fusion. In particular embodiments, the binding agents, in particular the antibodies and antibody fragments, described herein are (13) capable of inhibiting spike-mediated syncytia formation, more particularly they are (20) capable of inhibiting the formation of syncytia between cells expressing a Sarbecovirus spike protein, such as SARS-COV-2 and/or SARS-COV-1 spike protein, and cells expressing the Sarbocovirus host receptor, in particular ACE2 receptor.

As used herein “viral fusion” refers to fusion of a viral membrane and a host cell membrane. Viral fusion assays are well-known to the skilled person and exemplary methods for performing such methods are described herein in the examples. As will be clear to a skilled person, complete inhibition is not required and a skilled person is able to identify binding agents, antibodies and antibody fragments that significantly inhibit viral fusion or spike-mediated syncytia formation. Preferably, binding agents, in particular antibodies and antibody fragments, as described herein (21) may induce at least 50% inhibition, preferably at least 60%, at least 70%, at least 80% or at least 90% inhibition. In particular embodiments, some of the functional characteristics of a Sarbecovirus binding agent, in particular a Sarbecovirus antibody or antibody fragment, as described hereinabove are combined such as to characterize such binding agent, antibody or antibody fragment. e.g. to bind or specifically bind to the Sarbecovirus spike protein HR2 domain and to be capable of neutralizing a Sarbecovirus, in particular at least one or both of SARS-COV-2 (such as any one or more of SARS-COV-2 Wuhan strain, SARS-COV-2 D614G variant, SARS-COV-2 Alpha variant, SARS-COV-2 Omicron BA.1 variant, SARS-COV-2 Omicron BA.2 variant, SARS-COV-2 Omicron BA.5 variant, SARS-COV-2 Omicron BA.2.75.2 variant, SARS-COV-2 Omicron BA.4.6 variant, SARS-COV-2 Omicron BF.7 variant, SARS-COV-2 Omicron BQ.1.1 variant, SARS-COV-2 Omicron XBB variant, and SARS-CoV-2 Omicron XBB.1.5 variant) and SARS-COV-1, preferably to be capable of neutralizing the Sarbecovirus with a 50% inhibitory concentration (IC₅₀) of 100 ng/ml or less, preferably 10 ng/ml or less, more preferably 1 ng/ml or less, as determined in a vesicular stomatitis virus (VSV)-Sarbecovirus spike protein pseudovirus neutralization assay. Such binding agent, antibody or antibody fragment may further be characterized to be capable of inhibiting spike-mediated syncytia formation between cells expressing the Sarbecovirus spike protein and cells expressing the angiotensin-converting enzyme 2 (ACE2) receptor and/or to be capable of inhibiting viral fusion; and/or by not binding a Middle East respiratory syndrome coronavirus (MERS-COV).

The binding agents described herein can also be structurally defined as polypeptidic binding agents (i.e. binding agents comprising a peptidic, polypeptidic or proteic moiety, or binding agents comprising a peptide, polypeptide, protein or protein domain) or polypeptide binding agents (i.e. binding agents being peptides, polypeptides or proteins).

The terms “protein”, “polypeptide”, and “peptide” are interchangeably used herein to refer to a polymer of amino acid residues and to variants and synthetic analogues of the same; the sequential linear arrangement of the amino acids together resulting in/forming the “amino acid sequence” or “protein sequence”. A “peptide” may also be referred to as a partial amino acid sequence derived from its original protein, for instance after enzymatic (e.g. tryptic) digestion. These terms apply to naturally-occurring amino acid polymers as well as to amino acid polymers in which one or more amino acid residues is a synthetic non-naturally occurring amino acid, such as a chemical analogue of a corresponding naturally occurring amino acid. Also included are proteins comprising one or more posttranslational modifications such as covalent addition of functional groups or proteins (such as glycosylation, phosphorylation, acetylation, ubiquitination, methylation, lipidation and nitrosylation) or such as proteolytic processing. Based on the amino acid sequence and the modifications, the atomic or molecular mass or weight of a polypeptide is expressed in (kilo) dalton (kDa). A further modification of proteins includes addition of a tag, such as a His-tag or shortage. By sortagging (sortase-mediated transpeptidation; Popp et al. 2007, Nat Chem Biol 3:707-708) for instance, a multi-arm PEG nanobody neutralizing SARS-COV2 was constructed (Moliner-Morro et al. 2020, Biomolecules 10:1661).

A “protein domain” is a distinct functional and/or structural unit in or part of a protein. Usually, a protein domain is responsible for a particular function or interaction, contributing to the overall (biological) role of a protein. Domains may exist in a variety of biological contexts, where similar domains can be found in different proteins with similar or different functions. Protein domains can have a rigid 3D-structure if confined by e.g. a number of intramolecular cysteines (e.g. cysteine-knot proteins) or can, depending on e.g. presence or absence of a bound ligand or e.g. presence or absence of a posttranslational modification, assume different 3D-conformations, or can have a less defined, more fluid 3D-structure.

Amino acids are presented herein by their 3- or 1-lettercode nomenclature as defined and provided also in the IUPAC-IUB Joint Commission on Biochemical Nomenclature (Nomenclature and Symbolism for Amino Acids and Peptides. Eur. J. Biochem. 138:9-37 (1984)); as follows: Alanine (A or Ala), Cysteine (C or Cys), Aspartic acid (D or Asp), Glutamic acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr).

More in particular, the binding agents described herein can be structurally defined as polypeptidic or polypeptide binding agents comprising a complementarity determining region (CDR) as comprised in any of the immunoglobulin single variable domains (ISVDs) defined herein. In preferred embodiments, the polypeptidic or polypeptide binding agents are (isolated) antibodies or antibody fragments.

In certain embodiments, the binding agents, in particular the antibodies and antibody fragments, according to the current invention can be structurally defined as polypeptidic or polypeptide binding agents, in particular antibodies an antibody-fragments, comprising at least CDR3 as comprised in an immunoglobulin single variable domain (ISVD) as defined herein. In other embodiments, the binding agents, in particular the antibodies and antibody fragments, according to the current invention can be structurally defined as polypeptidic or polypeptide binding agents, in particular antibodies and antibody fragments, comprising at least two of CDR1, CDR2 and CDR3 (e.g. CDR1 and CDR3, CDR2 and CDR3, CDR1 and CDR2), or all three of CDR1, CDR2 and CDR3, as comprised in an immunoglobulin single variable domains (ISVDs) as defined herein. Such CDRs may be comprised in any of VHH R3_C4 (defined by/set forth in SEQ ID NO:1), VHH R4_DC16 (defined by/set forth in SEQ ID NO:2), VHH R3_DC20 (defined by/set forth in SEQ ID NO:3), VHH R3_DC2 (defined by/set forth in SEQ ID NO:4), VHH R4_DC20 (defined by/set forth in SEQ ID NO:5), VHH R4_DC9 (defined by/set forth in SEQ ID NO:6), VHH R4_DC6 (defined by/set forth in SEQ ID NO: 7), VHH R3_DC23 (also referred to herein as VHH R3DC23 or R3DC23; defined by/set forth in SEQ ID NO:8), VHH R3_DC9 (defined by/set forth in SEQ ID NO:9), or VHH R4_DC13 (defined by/set forth in SEQ ID NO:10), as depicted hereafter:

VHH R3_C4:
(SEQ ID NO: 1)
QVQLQESGGGLVQAGDSLRLSCAVSGRPFSTYTMGWFRQAPGKEREFVA
AMRWSGGTIYYADSVKGRFTISRDNDKNTVNLEMNSLKPEDTAVYYCAA
AYVSKANYGSLWYRASGLYDYWGQGTQVTVSS
VHH R4_DC16:
(SEQ ID NO: 2)
QVQLQESGGGLVQAGDSLRLSCAVSGRPFSTYTMGWFRQAPGKEREFVA
AMRWSGGTIYYADSVKGRFTISRDNAQNTVNLQMNSLKPEDTAVYYCAA
AYVSKANYGSSWYRNSGLYDYWGQGTQVTVSS
VHH R3_DC20:
(SEQ ID NO: 3)
QVQLQESGGGLVQAGDSLRLSCAVSGRPFSTYTMGWFRQAPGKEREFVA
AMRWSGGTIYYADSVKGRFTISRDNAQNTVNLQMNSLEPEDTAVYYCAA
AYVSKANYGSLWYRNSGLYDYWGQGTQVTVSS
VHH R3_DC2:
(SEQ ID NO: 4)
QVQLQESGGGLVQTGDSLRLSCAVSGRPFSTYTMGWFRQAPGKEREFVA
SMRWSGGTKYYSDSVKGRFSISRDNDQNTVNLQMNSLKPEDTAVYYCAA
AYVSKANYGSLWYRNSGLYDYWGQGTQVTVSS
VHH R4_DC20:
(SEQ ID NO: 5)
QVQLQESGGGLVQTGDSLRLSCAVSGRPFSTYTMGWFRQAPGKEREFVA
SMRWSGGTMNYADSVKGRFTISRDNDQNTVNLQMNSLKPEDTAVYYCAA
AYVSKANYGSLWYRNSGLYDYWGQGTQVTVSS
VHH R4_DC9:
(SEQ ID NO: 6)
QVQLQESGGGLVQAGDSLRLSCAASGRIFSTYTMGWERQAPGMEREFVA
AIRWSGSTVYYADSVKGRFTISRDNAKNTVYLQMNSLKPEDTAVYYCAA
ARVSKANYGTLWYRASGLYDYWGQGTQVTVSS
VHH R4_DC6:
(SEQ ID NO: 7)
QVQLQESGGGLVQAGDSLRLSCAASGRILSTFTMGWFRQAPGKEREFVA
AMRWNGGTKNYADSVKGRFTISRDNANSVVYLQMNSTKPEDTAVYYCAA
AYVSKANYGSLWYRTPSLYDYWGQGTQVTVSS
VHH R3_DC23:
(SEQ ID NO: 8)
QVQLQESGGGLVQAGDSLTLSCAVSGRIFSTYTMGWFRQAPGKEREFVA
AVRWGAGTIYYADSMKGRFTISRDSAKNTVDLQMNSTKPEDTAVYYCGA
AYVSKANYGSLWYQDSRRYDYWGQGTQVTVSS
VHH R3_DC9:
(SEQ ID NO: 9)
QVQLQESGGGLVQTGDSLRLSCAVSGRPFSTYTMGWFRQAPGKEREFVA
SMRWSGGTMYYSDSVKGRFTISRDNDKNTVNLQMNSLKPEDTAVYYCAA
AYVSKANYGSLWYRNSGLYDYWGQGTQVTVSS
VHH R4_DC13:
(SEQ ID NO: 10)
QVQLQESGGGLVQTGDSLRLSCAVSGRPFSTYTMGWFRQAPGKEREFVA
SMRWSGGTVYYSDSVKGRFTISRDNDKNTVNLQMNNLKPEDTAVYYCAA
AYVSKANYGSFWYRNSDLYDYWGQGTQVTVSS

For numbering of the amino acid residues of any IVD or ISVD different numbering schemes can be applied. For example, numbering can be performed according to the AHo numbering scheme for all heavy (VH) and light chain variable domains (VL) given by Honegger & Plückthun (2001. J Mol Biol 309:657-70), as applied to VHH domains from camelids. Alternative methods for numbering the amino acid residues of VH domains, which can also be applied in an analogous manner to VHH domains, are known in the art. For example, the delineation of the FR and CDR sequences can be done by using the Kabat numbering system as applied to VHH domains from camelids by Riechmann & Muyldermans (1999. J Immunol Methods 231:25-38). It should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein.

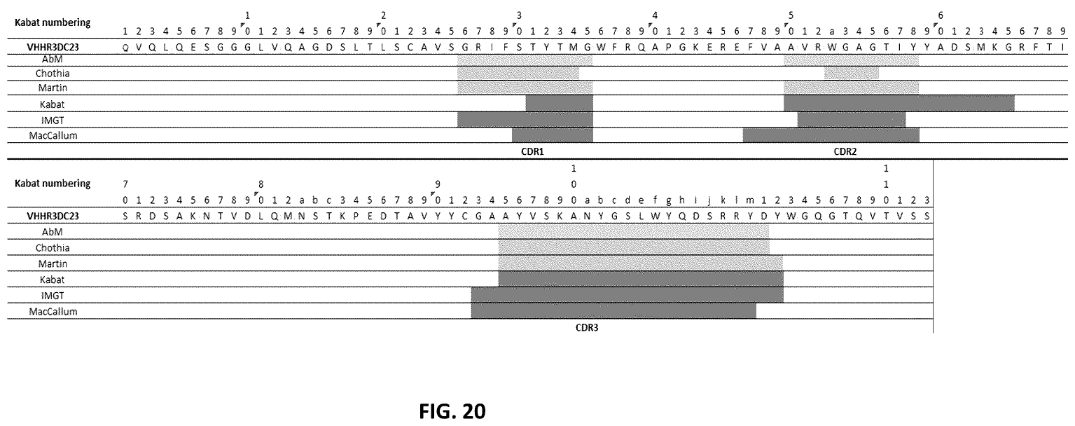

The determination of the CDR regions in an antibody/immunoglobulin sequence generally depends on the algorithm/methodology applied. For example, determination of CDR regions may be done according to the designation based on contact analysis and binding site topography as described in MacCallum et al. (J. Mol. Biol. (1996) 262, 732-745), AbM (AbM is Oxford Molecular Ltd.'s antibody modelling package as described on http://www.bioinf.org.uk/abs/index.html), Chothia (Chothia and Lesk, 1987; Mol Biol. 196:901-17), Martin (Abhinandan, and Martin. Molecular Immunology 45 (2008) 3832-3839; as shown in http://bioinf.org.uk/abs/info.html), Kabat (Kabat et al., 1991; 5th edition. NIH publication 91-3242), or IMGT (LeFranc, 2014; Frontiers in Immunology. 5 (22): 1-22). Said annotations further include delineation of CDRs and framework regions (FRs) in immunoglobulin-domain-containing proteins, and are known methods and systems to a skilled artisan who thus can apply these annotations onto any antibody/immunoglobulin protein sequences without undue burden. As an example, FIG. 20 illustrates the different annotation-schemes or -methods as applied to the amino acid sequence of VHH R3_DC23 (SEQ ID NO:8).

Applying different methods to the same antibody/immunoglobulin sequence may give rise to different CDR amino acid sequences wherein the differences may reside in CDR sequence length and/or -delineation within the antibody/immunoglobulin/IVD sequence (as illustrated in FIG. 20 for VHH R3_DC23). The CDRs of the ISVD binding agents, in particular antibodies and antibody fragments, as described herein can therefore be described as the CDR sequences as present in the ISVDs characterized herein. Alternatively, these CDRs can be described as the CDR sequences present in the ISVDs (as described herein) as determined or delineated according to a well-known methodology such as according to any one of the Kabat-, Martin-, Chothia-, aHo, MacCallum et al. 1996, AbM-, or IMGT, numbering scheme or method, such as preferably the Martin numbering scheme or method.

VHHs or Nbs are often classified in different families according to amino acid sequences, or even in superfamilies, as to cluster the clonally related sequences derived from the same progenitor during B cell maturation (Deschaght et al. 2017, Front Immunol 8:420). This classification is often based on the CDR sequence of the VHHs or Nbs, and wherein for instance each VHH or Nb family is defined as a cluster of (clonally) related sequences with a sequence identity threshold of the CDR3 region. Within a single VHH family defined herein, the CDR3 sequence is thus identical or very similar in amino acid composition, preferably with at least 80% identity, or at least 85% identity, or at least 90% identity in the CDR3 sequence, resulting in VHHs or Nbs of the same family binding to the same binding site, and having the same effect such as functional effect.

As outlined above, many systems or methods (Kabat. MacCallum, IMGT. AbM. Chothia. Martin) exist for numbering amino acids in immunoglobulin protein sequences, including for delineation of CDRs and framework regions (FRs) in these protein sequences. These systems or methods are known to a skilled artisan who thus can apply these systems or methods on any immunoglobulin protein sequences without undue burden (as illustrated in FIG. 20 for VHH R3_DC23).

In certain embodiments, a binding agent, in particular an antibody or antibody fragment, more particularly an ISVD, as described herein may be characterized in that it comprises a CDR1 defined by/set forth in any one of SEQ ID NOs: 63, 46, 69 or 77. In certain embodiments, a binding agent, in particular an antibody or antibody fragment, more particularly an ISVD, as described herein may be characterized in that it comprises a CDR2 defined by/set forth in any one of SEQ ID NOs: 64, 47, 70, 73 or 78. In certain embodiments, a binding agent, in particular an antibody or antibody fragment, more particularly an ISVD, as described herein may be characterized in that it comprises a CDR3 defined by/set forth in any one of SEQ ID NOs: 48, 67, 74 or 79. In certain embodiments, a binding agent, in particular an antibody or antibody fragment, more particularly an ISVD, as described herein may be characterized in that it comprises a CDR1 defined by/set forth in any one of SEQ ID NOs: 63, 46, 69 or 77, a CDR2 defined by/set forth in any one of SEQ ID NOs: 64, 47, 70, 73 or 78, and a CDR3 defined by/set forth in any one of SEQ ID NOs: 48, 67, 74 or 79.

In certain embodiments, the binding agent, in particular the antibody or antibody fragment, more particularly the ISVD, as described herein may comprise:
a CDR1 defined by/set forth in SEQ ID NO:63; a CDR2 defined by/set forth in SEQ ID NO:64; and a CDR3 defined by/set forth in SEQ ID NO:67; or
a CDR1 defined by/set forth in SEQ ID NO:69; a CDR2 defined by/set forth in SEQ ID NO: 70; and a CDR3 defined by/set forth in SEQ ID NO:67; or
a CDR1 defined by/set forth in SEQ ID NO:63; a CDR2 defined by/set forth in SEQ ID NO:64; and a CDR3 defined by/set forth in SEQ ID NO:48; or
a CDR1 defined by/set forth in SEQ ID NO:46; a CDR2 defined by/set forth in SEQ ID NO:47; and a CDR3 defined by/set forth in SEQ ID NO:48; or
a CDR1 defined by/set forth in SEQ ID NO:63; a CDR2 defined by/set forth in SEQ ID NO:73; and a CDR3 defined by/set forth in SEQ ID NO: 74; or
a CDR1 defined by/set forth in SEQ ID NO:77; a CDR2 defined by/set forth in SEQ ID NO: 78; and a CDR3 defined by/set forth in SEQ ID NO:79.

TABLE 1
Sequences of CDRs in the VHHs according to certain embodiments:

| Annotation | | | |
|---|---|---|---|
| method | CDR1 | CDR2 | CDR3 |
| AbM | GRXXSTXTM | XXRWXXXTXX | AXVSKANYGXXWYXXXXXYD |
| | G | wherein X at position 1 is A, | wherein X at position 2 is Y, W, R or K; |
| | wherein X at | G , S or T; X at position 2 is | X at position 10 is S, N or T; X at |
| | position 3 is P, I, | M, I, L or V; X at position 5 | position 11 is L, I, S, N, F, M, V or T,; |
| | A, L or V; X at | is S, N, T, Q or G; X at | X at position 14 is R, Q, K or N; X at |
| | position 4 is F, | position 6 is G, A or P; X at | position 15 is A, G, N, S, T, D or E; X at |
| | L, I or M; X at | position 7 is G or S; X at | position 16 is S, P, A, G or T,; X at |
| | position 7 is Y, F | position 9 is I , L, M, K, V or | position 17 is G, A, D, S, R, T, K, E; X |
| | or W (SEQ ID | R; X at position 10 is Y, W, | at position 18 is L, I, R, V, Mor K (SEQ |
| | NO: 63) | N, S, F, Q or T (SEQ ID | ID NO: 67) |
| | | NO: 64) | |
| Chothia | GRXXSTXTM | WXXX | AXVSKANYGXXWYXXXXXYD |
| | wherein X at | wherein X at position 2 is S, | wherein X at position 2 is Y, W, R or K; |
| | position 3 is P, I, | N, T, Q or G; X at position 3 | X at position 10 is S, N or T; X at |
| | A, L or V; X at | is G, A or P; X at position 4 is | position 11 is L, I, S, N, F, M, V or T,; |
| | position 4 is F, | G or S (SEQ ID NO: 70) | X at position 14 is R, Q, K or N; X at |
| | L, I or M; X at | | position 15 is A, G, N, S, T, D or E; X at |
| | position 7 is Y, F | | position 16 is S, P, A, G or T,; X at |
| | or W (SEQ ID | | position 17 is G, A, D, S, R, T, K, E; X |
| | NO: 69) | | at position 18 is L, I, R, V, M or K (SEQ |
| | | | ID NO: 67) |
| Martin | GRXXSTXTM | XXRWXXXTXX | AXVSKANYGXXWYXXXXXYDY |
| | G | wherein X at position 1 is A , | wherein X at position 2 is Y, W, R or K; |
| | wherein X at | G , S or T; X at position 2 is | X at position 10 is S, N or T; X at |
| | position 3 is P, I, | M, I, L or V; X at position 5 | position 11 is L, I, S, N, F, M, V or T,; |
| | A, L or V; X at | is S, N, T, Q or G; X at | X at position 14 is R, Q, K or N; X at |
| | position 4 is F, | position 6 is G, A or P; X at | position 15 is A, G, N, S, T, D or E; X at |
| | L, I or M; X at | position 7 is G or S; X at | position 16 is S, P, A, G or T,; X at |
| | position 7 is Y, F | position 9 is I , L, M, K, V or | position 17 is G, A, D, S, R, T, K, E; X |
| | or W (SEQ ID | R; X at position 10 is Y, W, | at position 18 is L, I, R, V, Mor K (SEQ |
| | NO: 63) | N, S, F, Q or T (SEQ ID | ID NO: 48) |
| | | NO: 64) | |
| Kabat | TXTMG | XXRWXXXTXXYXDSXK | AXVSKANYGXXWYXXXXXYDY |
| | wherein X at | G | wherein X at position 2 is Y, W, R or K; |
| | position 2 is Y or | wherein X at position 1 is A, | X at position 10 is S, N or T; X at |
| | For W (SEQ ID | G, S or T; X at position 2 is | position 11 is L, I, S, N, F, M, V or T,; |
| | NO: 46) | M, I, L, or V; X at position 5 | X at position 14 is R, Q, K or N; X at |
| | | is S, N, T, Q or G; X at | position 15 is A, G, N, S, T, D or E; X at |
| | | position 6 is G, A or P; X at | position 16 is S, P, A, G or T,; X at |
| | | position 7 is G or S; X at | position 17 is G, A, D, S, R, T, K, E; X |
| | | position 9 is I, L, M, K, V or | at position 18 is L, I, R, V, M or K (SEQ |
| | | R; X at position 10 is Y, W, | ID NO: 48) |
| | | N, S, F, Q or T; X at position | |
| | | 12 is A, G, S, N, T or V; X at | |
| | | position 15 is V or M (SEQ | |
| | | ID NO: 47) | |
| IMGT | GRXXSTXTM | XRWXXXTX | XAAXVSKANYGXXWYXXXXXYD |
| | G | wherein X at position 1 is M, | Y |
| | wherein X at | I, L or V; X at position 4 is S, | wherein X at position 1 is A, G, S, T, V |
| | position 3 is P, I, | N, T, Q or G; X at position 5 | or N; X at position 4 is Y, W, R or K; X |
| | A, L or V; X at | is G, A or P; X at position 6 is | at position 12 is S, N or T; X at position |
| | position 4 is F, | Gor S; X at position 8 is I , L, | 13 is L, I, S, N, F, M, V or T,; X at |
| | L, I or M; X at | M, K, V or R (SEQ ID | position 16 is R, Q, K or N; X at position |
| | position 7 is Y, F | NO: 73) | 17 is A, G, N, S, T, D or E; X at position |
| | or W (SEQ ID | | 18 is S, P, A, G or T,; X at position 19 is |
| | NO: 63) | | G, A, D, S, R, T, K, E; X at position 20 |
| | | | is L, I, R, V, M or K (SEQ ID NO: 74) |
| MacCallum | STXTMG | FVAXXRWXXXTXX | XAAXVSKANYGXXWYXXXXXY |
| | wherein X at | wherein X at position 4 is A , | wherein X at position 1 is A, G, S, T, V |
| | position 3 is Y | G, Sor T; X at position 5 is | or N; X at position 4 is Y, W, R or K; X |
| | or F or W (SEQ | M, I, L or V; X at position 8 | at position 12 is S, N or T; X at position |
| | ID NO: 77) | is S, N, T, Q or G; X at | 13 is L, I, S, N, F, M, V or T,; X at |
| | | position 9 is G, A or P; X at | position 16 is R, Q, K or N; X at |
| | | position 10 is G or S; X at | position 17 is A, G, N, S, T, D or E; X |
| | | position 12 is I , L, M, K, V | at position 18 is S, P, A, G or T,; X at |
| | | or R; X at position 13 is Y, | position 19 is G, A, D, S, R, T, K, E; X |
| | | W, N, S, F, Q or T (SEQ ID | at position 20 is L, I, R, V, M or K |
| | | NO: 78) | (SEQ ID NO: 79) |

In certain embodiments, a binding agent, in particular an antibody or antibody fragment, more particularly an ISVD, as described herein may be characterized in that it comprises a CDR1 defined by/set forth in any one of SEQ ID NOs: 65, 71, 49, or 80. In certain embodiments, a binding agent, in particular an antibody or antibody fragment, more particularly an ISVD, as described herein may be characterized in that it comprises a CDR2 defined by/set forth in any one of SEQ ID NOs: 66, 72, 50, 75, or 81. In certain embodiments, a binding agent, in particular an antibody or antibody fragment, more particularly an ISVD, as described herein may be characterized in that it comprises; and a CDR3 defined by/set forth in any one of SEQ ID NOs: 51, 68, 76, or 82. In certain embodiments, a binding agent, in particular an antibody or antibody fragment, more particularly an ISVD, as described herein may be characterized in that it comprises a CDR1 defined by/set forth in any one of SEQ ID NOs: 65, 71, 49, or 80, a CDR2 defined by/set forth in any one of SEQ ID NOs: 66, 72, 50, 75, or 81, and a CDR3 defined by/set forth in any one of SEQ ID NOs: 51, 68, 76, or 82.

In certain embodiments, the binding agent, in particular the antibody or antibody fragment, more particularly the ISVD, as described herein may comprise:
a CDR1 defined by/set forth in SEQ ID NO:65; a CDR2 defined by/set forth in SEQ ID NO:66; and a CDR3 defined by/set forth in SEQ ID NO:68; or
a CDR1 defined by/set forth in SEQ ID NO:71; a CDR2 defined by/set forth in SEQ ID NO:72; and a CDR3 defined by/set forth in SEQ ID NO:68; or
a CDR1 defined by/set forth in SEQ ID NO:65; a CDR2 defined by/set forth in SEQ ID NO:66; and a CDR3 defined by/set forth in SEQ ID NO:51; or
a CDR1 defined by/set forth in SEQ ID NO:49; a CDR2 defined by/set forth in SEQ ID NO:50; and a CDR3 defined by/set forth in SEQ ID NO:51; or
a CDR1 defined by/set forth in SEQ ID NO:65; a CDR2 defined by/set forth in SEQ ID NO: 75; and a CDR3 defined by/set forth in SEQ ID NO: 76; or
a CDR1 defined by/set forth in SEQ ID NO:80; a CDR2 defined by/set forth in SEQ ID NO:81; and a CDR3 defined by/set forth in SEQ ID NO:82.

TABLE 2
Example definitions/sequences of the CDRs in the VHHs of certain embodiments as
described herein by employing different annotation methodologies as indicated.

| Annotation | | | |
|---|---|---|---|
| method | CDR1 | CDR2 | CDR3 |
| AbM | GRXXSTXTM | XXRWXXXTXX | AXVSKANYGXXWYXXXXXYD |
| | G | wherein X at position 1 is A , | wherein X at position 2 is Y or R; X at |
| | wherein X at | G , S or T; X at position 2 is | position 10 S or T; X at position 11 is L, |
| | position 3 is P or | M, I, L or V; X at position 5 | S or F; X at position 14 is R or Q; X at |
| | I; X at position 4 | is S, N, T, Q or G; X at | position 15 is A, N, T or D; X at position |
| | is F or L; X at | position 6 is G, A or P; X at | 16 is S or P; X at position 17 is G, D, S |
| | position 7 is Y or | position 7 is G or S; X at | or R; X at position 18 is L or R (SEQ ID |
| | F (SEQ ID | position 9 is I , L, M, K, V or | NO: 68) |
| | NO: 65) | R; X at position 10 is Y, W, | |
| | | N, S, F, Q or T (SEQ ID | |
| | | NO: 66) | |
| Chothia | GRXXSTXTM | WXXX | AXVSKANYGXXWYXXXXXYD |
| | wherein X at | wherein X at position 2 is S, | wherein X at position 2 is Y or R; X at |
| | position 3 is Por | N, T, Q or G; X at position 3 | position 10 S or T; X at position 11 is L, |
| | I; X at position 4 | is G, A or P; X at position 4 is | S or F; X at position 14 is R or Q; X at |
| | is F or L; X at | G or S (SEQ ID NO: 72) | position 15 is A, N, T or D; X at position |
| | position 7 is Y or | | 16 is S or P; X at position 17 is G, D, S |
| | F (SEQ ID | | or R; X at position 18 is L or R (SEQ ID |
| | NO: 71) | | NO: 68) |
| Martin | GRXXSTXTM | XXRWXXGTXX | AXVSKANYGXXWYXXXXXYDY |
| | G | wherein X at position 1 is A | wherein X at position 2 is Y or R; X at |
| | wherein X at | or S; X at position 2 is M, I or | position 10 S or T; X at position 11 is L, |
| | position 3 is P or | V; X at position 5 is S or N; X | S or F; X at position 14 is R or Q; X at |
| | I; X at position 4 | at position 6 is G or A; X at | position 15 is A, N, T or D; X at position |
| | is F or L; X at | position 9 is I, M, K or V; X | 16 is S or P; X at position 17 is G, D, S |
| | position 7 is Y or | at position 10 is Y or N (SEQ | or R; X at position 18 is L or R (SEQ ID |
| | F (SEQ ID | ID NO: 66) | NO: 51) |
| | NO: 65) | | |
| Kabat | TXTMG | XXRWXXGTXXYXDSXK | AXVSKANYGXXWYXXXXXYDY |
| | wherein X at | G | wherein X at position 2 is Y or R; X at |
| | position 2 is Y or | wherein X at position 1 is A | position 10 S or T; X at position 11 is L, |
| | F (SEQ ID | or S; X at position 2 is M, I or | S or F; X at position 14 is R or Q; X at |
| | NO: 49) | V; X at position 5 is S or N; X | position 15 is A, N, T or D; X at position |
| | | at position 6 is G or A; X at | 16 is S or P; X at position 17 is G, D, S |
| | | position 9 is I, M, K or V; X | or R; X at position 18 is L or R (SEQ ID |
| | | at position 10 is Y or N; X at | NO: 51) |
| | | position 12 is A or S; X at | |
| | | position 15 is V or M (SEQ | |
| | | ID NO: 50) | |
| IMGT | GRXXSTXTM | XRWXXGTX | XAAXVSKANYGXXWYXXXXXYD |
| | G | wherein X at position 1 is M, | Y |
| | wherein X at | I or V; X at position 4 is S or | wherein X at position 1 is A or G; X at |
| | position 3 is Por | N; X at position 5 is G or A; | position 4 is Y or R; X at position 12 S |
| | I; X at position 4 | X at position 8 is I, M, K or V | or T; X at position 13 is L, S or F; X at |
| | is F or L; X at | (SEQ ID NO: 75) | position 16 is R or Q; X at position 17 is |
| | position 7 is Y or | | A, N, T or D; X at position 18 is S or P; |
| | F (SEQ ID | | X at position 19 is G, D, S or R; X at |
| | NO: 65) | | position 20 is L or R (SEQ ID NO: 76) |
| MacCallum | STXTMG | FVAXXRWXXXTXX | XAAXVSKANYGXXWYXXXXXY |
| | wherein X at | wherein X at position 4 is A , | wherein X at position 1 is A or G; X at |
| | position 3 is Y | G , S or T; X at position 5 is | position 4 is Y or R; X at position 12 S |
| | or F (SEQ ID | M, I, L or V; X at position 8 | or T; X at position 13 is L, S or F; X at |
| | NO: 80) | is S, N, T, Q or G; X at | position 16 is R or Q; X at position 17 is |
| | | position 9 is G, A or P; X at | A, N, T or D; X at position 18 is S or P; |
| | | position 10 is G or S; X at | X at position 19 is G, D, S or R; X at |
| | | position 12 is I , L, M, K, V | position 20 is L or R (SEQ ID NO: 82) |
| | | or R; X at position 13 is Y, | |
| | | W, N, S, F, Q or T (SEQ ID | |
| | | NO: 81) | |

In certain preferred embodiments, a binding agent or Sarbecovirus binding agent, in particular an antibody or antibody fragment or Sarbecovirus antibody or antibody-fragments, more particularly an ISVD, as described herein, may be characterized in that it comprises a CDR1 as present in any of SEQ ID NOs: 1 to 10, wherein the CDR1 is annotated according to any one of Kabat, MacCallum, IMGT, AbM, Martin or Chothia. In certain preferred embodiments, a binding agent or Sarbecovirus binding agent, in particular an antibody or antibody fragment or Sarbecovirus antibody or antibody-fragments, more particularly an ISVD, as described herein, may be characterized in that it comprises a CDR2 as present in any of SEQ ID NOs: 1 to 10, wherein the CDR2 is annotated according to any one of Kabat, MacCallum, IMGT, AbM, Martin or Chothia. In certain preferred embodiments, a binding agent or Sarbecovirus binding agent, in particular an antibody or antibody fragment or Sarbecovirus antibody or antibody-fragments, more particularly an ISVD, as described herein, may be characterized in that it comprises a CDR3 as present in any of SEQ ID NOs: 1 to 10, wherein the CDR3 is annotated according to any one of Kabat. MacCallum, IMGT. AbM, Martin or Chothia. In certain preferred embodiments, a binding agent or Sarbecovirus binding agent, in particular an antibody or antibody fragment or Sarbecovirus antibody or antibody-fragments, more particularly an ISVD, as described herein, may be characterized in that it comprises a CDR1, CDR2 and CDR3, each independently as present in any of SEQ ID NOs: 1 to 10, wherein the CDR1, CDR2 and CDR3 are annotated according to any one of Kabat, MacCallum, IMGT, AbM, Martin or Chothia.

In certain preferred embodiments, a binding agent or Sarbecovirus binding agent, in particular an antibody or antibody fragment or Sarbecovirus antibody or antibody-fragments, more particularly an ISVD, as described herein, may be characterized in that it comprises a combination of CDR1, CDR2 and CDR3, wherein the CDR1, CDR2 and CDR3 are as present in a particular one of the sequences set forth in SEQ ID NOs: 1 to 10, wherein the CDR1, CDR2 and CDR3 are annotated according to any one of Kabat, MacCallum, IMGT, AbM, Martin or Chothia.

In certain embodiments, a binding agent, in particular an antibody or antibody fragment, more particularly an ISVD, as described herein may be characterized in that it comprises a CDR1 defined by/set forth in any one of SEQ ID NOs: 52, 53, 54, 11, or 12. In certain embodiments, a binding agent, in particular an antibody or antibody fragment, more particularly an ISVD, as described herein may be characterized in that it comprises a CDR2 defined by/set forth in any one of SEQ ID NOs: 55-62 or 13-20. In certain embodiments, a binding agent, in particular an antibody or antibody fragment, more particularly an ISVD, as described herein may be characterized in that it comprises: and a CDR3 defined by/set forth in any one of SEQ ID NOs: 21-27. In certain embodiments, a binding agent, in particular an antibody or antibody fragment, more particularly an ISVD, as described herein may be characterized in that it comprises a CDR1 defined by/set forth in any one of SEQ ID NOs: 52, 53, 54, 11, or 12, a CDR2 defined by/set forth in any one of SEQ ID NOs: 55-62 or 13-20, and a CDR3 defined by/set forth in any one of SEQ ID NOs: 21-27.

In certain embodiments, the binding agent, in particular the antibody or antibody fragment, more particularly the ISVD, as described herein may comprise:
a CDR1 defined by/set forth in any one of SEQ ID NO:52-54; a CDR2 defined by/set forth in any one of SEQ ID NO:55-62; and a CDR3 defined by/set forth in any one of SEQ ID NO:21-27; or
a CDR1 defined by/set forth in any one of SEQ ID NO:11 or 12; a CDR2 defined by/set forth in any one of SEQ ID NO: 13-20; and a CDR3 defined by/set forth in any one of SEQ ID NO:21-27.

TABLE 3
Example definitions/sequences of the CDRs in the VHHs of certain embodiments as
described herein by employing different annotation methodologies as indicated, in
particular CDRs comprised in any of VHH R3_C4, VHH R4_DC16, VHH R3_DC20, VHH R3_DC2, VHH
R4_DC20, VHH R4_DC9, VHH R4_DC6, VHH R3_DC23, VHH R3_DC9, and VHH R4_DC13,
determined according to Kabat or Martin system or method.

| Annotation | | | |
|---|---|---|---|
| method | CDR1 | CDR2 | CDR3 |
| Martin | GRPFSTYTMG | AMRWSGGTIY (SEQ ID | AYVSKANYGSLWYRASGLYDY |
| | (SEQ ID NO: 52), | NO: 55), | (SEQ ID NO: 21), |
| | GRIFSTYTMG | SMRWSGGTMY (SEQ ID | AYVSKANYGSSWYRNSGLYDY |
| | (SEQ ID NO: 53) | NO: 56), | (SEQ ID NO: 22), |
| | or | SMRWSGGTKY (SEQ ID | AYVSKANYGSLWYRNSGLYDY |
| | GRILSTFTMGW | NO: 57), | (SEQ ID NO: 23), |
| | (SEQ ID NO: 54) | SMRWSGGTMN (SEQ ID | AYVSKANYGSFWYRNSDLYDY |
| | | NO: 58), | (SEQ ID NO: 24), |
| | | SMRWSGGTVY (SEQ ID | ARVSKANYGTLWYRASGLYDY |
| | | NO: 59), | (SEQ ID NO: 25), |
| | | AIRWSGSTVY (SEQ ID | AYVSKANYGSLWYRTPSLYDY |
| | | NO: 60), | (SEQ ID NO: 26) or |
| | | AMRWNGGTKN (SEQ ID | AYVSKANYGSLWYQDSRRYDY |
| | | NO: 61) or | (SEQ ID NO: 27) |
| | | AVRWGAGTIY (SEQ ID | |
| | | NO: 62) | |
| Kabat | TYTMG (SEQ | AMRWSGGTIYYADSVKG | AYVSKANYGSLWYRASGLYDY |
| | ID NO: 11) or | (SEQ ID NO: 13), | (SEQ ID NO: 21), |
| | TFTMG (SEQ ID | SMRWSGGTMYYSDSVKG | AYVSKANYGSSWYRNSGLYDY |
| | NO: 12) | (SEQ ID NO: 14), | (SEQ ID NO: 22), |
| | | SMRWSGGTKYYSDSVKG | AYVSKANYGSLWYRNSGLYDY |
| | | (SEQ ID NO: 15), | (SEQ ID NO: 23), |
| | | SMRWSGGTMNYADSVKG | AYVSKANYGSFWYRNSDLYDY |
| | | (SEQ ID NO: 16), | (SEQ ID NO: 24), |
| | | SMRWSGGTVYYSDSVKG | ARVSKANYGTLWYRASGLYDY |
| | | (SEQ ID NO: 17), | (SEQ ID NO: 25), |
| | | AIRWSGSTVYYADSVKG | AYVSKANYGSLWYRTPSLYDY |
| | | (SEQ ID NO: 18), | (SEQ ID NO: 26) or |
| | | AMRWNGGTKNYADSVKG | AYVSKANYGSLWYQDSRRYDY |
| | | (SEQ ID NO: 19) or | (SEQ ID NO: 27) |
| | | AVRWGAGTIYYADSMKG | |
| | | (SEQ ID NO: 20) | |

For example, polypeptidic or polypeptide binding agents, in particular antibodies or antibody fragments, more particularly ISVDs, as described herein can be defined as comprising the complementarity determining regions (CDRs) present in any one of SEQ ID NOs: 1-10, wherein the CDRs are defined according to Kabat. In certain embodiments, the binding agents, in particular antibodies or antibody fragments, more particularly ISVDs, comprise one of following sets of three complementarity determining regions (CDRs):
CDR1 defined by/set forth in SEQ ID NO:11, CDR2 defined by/set forth in SEQ ID NO:13, and CDR3 defined by/set forth in SEQ ID NO:21; or
CDR1 defined by/set forth in SEQ ID NO:11, CDR2 defined by/set forth in SEQ ID NO: 13, and CDR3 defined by/set forth in SEQ ID NO:22; or
CDR1 defined by/set forth in SEQ ID NO:11, CDR2 defined by/set forth in SEQ ID NO: 13, and CDR3 defined by/set forth in SEQ ID NO:23; or
CDR1 defined by/set forth in SEQ ID NO:11, CDR2 defined by/set forth in SEQ ID NO: 14, and CDR3 defined by/set forth in SEQ ID NO:23; or
CDR1 defined by/set forth in SEQ ID NO:11, CDR2 defined by/set forth in SEQ ID NO: 12, and CDR3 defined by/set forth in SEQ ID NO:23; or
CDR1 defined by/set forth in SEQ ID NO:11, CDR2 defined by/set forth in SEQ ID NO:16, and CDR3 defined by/set forth in SEQ ID NO:23;
CDR1 defined by/set forth in SEQ ID NO:11, CDR2 defined by/set forth in SEQ ID NO: 17, and CDR3 defined by/set forth in SEQ ID NO:24;
CDR1 defined by/set forth in SEQ ID NO:11, CDR2 defined by/set forth in SEQ ID NO: 18, and CDR3 defined by/set forth in SEQ ID NO:25;
CDR1 defined by/set forth in SEQ ID NO:12, CDR2 defined by/set forth in SEQ ID NO: 19, and CDR3 defined by/set forth in SEQ ID NO:26;
CDR1 defined by/set forth in SEQ ID NO:11, CDR2 defined by/set forth in SEQ ID NO:20, and CDR3 defined by/set forth in SEQ ID NO:27.

In particular embodiments, polypeptidic or polypeptide binding agents, in particular antibodies or antibody fragments, more particularly ISVDs, as described herein can be defined as comprising the complementarity determining regions (CDRs) present in any one of SEQ ID NOs: 1-10, wherein the CDRs are defined according to Martin. In certain embodiments, the binding agents, in particular antibodies or antibody fragments, more particularly ISVDs, comprise one of following sets of three complementarity determining regions (CDRs):
CDR1 defined by/set forth in SEQ ID NO:52, CDR2 defined by/set forth in SEQ ID NO:55, and CDR3 defined by/set forth in SEQ ID NO:21; or
CDR1 defined by/set forth in SEQ ID NO:52, CDR2 defined by/set forth in SEQ ID NO:55, and CDR3 defined by/set forth in SEQ ID NO:22; or
CDR1 defined by/set forth in SEQ ID NO:52, CDR2 defined by/set forth in SEQ ID NO:55, and CDR3 defined by/set forth in SEQ ID NO:23; or
CDR1 defined by/set forth in SEQ ID NO:52, CDR2 defined by/set forth in SEQ ID NO:57, and CDR3 defined by/set forth in SEQ ID NO:23; or
CDR1 defined by/set forth in SEQ ID NO:52, CDR2 defined by/set forth in SEQ ID NO:58, and CDR3 defined by/set forth in SEQ ID NO:23; or
CDR1 defined by/set forth in SEQ ID NO:53, CDR2 defined by/set forth in SEQ ID NO:60, and CDR3 defined by/set forth in SEQ ID NO:25; or
CDR1 defined by/set forth in SEQ ID NO:54, CDR2 defined by/set forth in SEQ ID NO:61, and CDR3 defined by/set forth in SEQ ID NO:26;
CDR1 defined by/set forth in SEQ ID NO:53, CDR2 defined by/set forth in SEQ ID NO:62, and CDR3 defined by/set forth in SEQ ID NO:27;
CDR1 defined by/set forth in SEQ ID NO:52, CDR2 defined by/set forth in SEQ ID NO:56, and CDR3 defined by/set forth in SEQ ID NO:23;
CDR1 defined by/set forth in SEQ ID NO:52, CDR2 defined by/set forth in SEQ ID NO:59, and CDR3 defined by/set forth in SEQ ID NO:24.

In further embodiments, the polypeptidic or polypeptide binding agents, in particular the antibodies and antibody fragments, more particularly the ISVDs, according to the current invention can comprise one or more framework regions (FRs) as comprised in any one of SEQ ID NOs: 1-10, or variants of such FRs, More in particular, such binding agents, antibodies or antibody fragments, or ISVDs may comprise at least one, such as one, two, three or all of an FR1, FR2, FR3, and FR4 region, each independently as comprised in any one of SEQ ID NOs: 1-10, or variants of such FRs. For example, such binding agents, antibodies or antibody fragment, or ISVDs, may comprise an FR1 and FR2 region, an FR1 and FR3 region, an FR1 and FR4 regions, an FR2 and FR3 region, an FR2 and FR4 region, an FR3 and FR4 region, an FR1, FR2 and FR3 region, an FR1, FR2 and FR4 region, an FR2, FR3 and FR4, or an FR1, FR3 and FR4 region as comprised in any one of SEQ ID NOs: 1-10, or variants of such FRs. In certain preferred embodiments, such binding agents, antibodies or antibody fragments, or ISVDs, comprise an FR1 region or an FR4 region or an FR2 and FR3 region as comprised in any one of SEQ ID NOs: 1-10 or variants of such FRs. For delineation of FRs in these protein sequences, any one of the systems or methods for numbering amino acids in immunoglobulin protein sequences as described elsewhere herein and illustrated in FIG. 20 for VHH R3_DC23, and known to a skilled artisan can be applied, By means of an example, sequences of the FRs in certain specific VHHs as described herein by employing the Martin or Kabat methodology are shown in Table 4.

TABLE 4
Example sequences of the FRs in the VHHs of certain embodiments as described herein by
employing the Kabat or Martin methodology.

| Anno- | | | | |
|---|---|---|---|---|
| tation | | | | |
| method | FR1 | FR2 | FR3 | FR4 |
| Kabat | QVQLQESGGGLVQAGD | WFRQAPG | RFTISRDNDKNTVNLEMNSLKP | WGQGT |
| | SLRLSCAVSGRPFS (SEQ | KEREFVA | EDTAVYYCAA (SEQ ID NO: 35), | QVTVSS |
| | ID NO: 28), | (SEQ ID | RFTISRDNAQNTVNLQMNSLKP | (SEQ ID |
| | QVQLQESGGGL VQTGD | NO: 33), | EDTAVYYCAA (SEQ ID NO: 36), | NO: 45) |
| | SLRLSCAVSGRPFS (SEQ | WFRQAPG | RFTISRDNAQNTVNLQMNSLEP | |
| | ID NO: 29), | MEREFVA | EDTAVYYCAA (SEQ ID NO: 37), | |
| | QVQLQESGGGLVQAGD | (SEQ ID | RFTISRDNDKNTVNLQMNSLKP | |
| | SLRLSCAASGRIFS (SEQ | NO: 34) | EDTAVYYCAA (SEQ ID NO: 38), | |
| | ID NO: 30), | | RFSISRDNDQNTVNLQMNSLKP | |
| | QVQLQESGGGLVQAGD | | EDTAVYYCAA (SEQ ID NO: 39), | |
| | SLRLSCAASGRILS (SEQ | | RFTISRDNDQNTVNLQMNSLKP | |
| | ID NO: 31), | | EDTAVYYCAA (SEQ ID NO: 40), | |
| | QVQLQESGGGLVQAGD | | RFTISRDNDKNTVNLQMNNLKP | |
| | SLTLSCAVSGRIFS (SEQ | | EDTAVYYCAA (SEQ ID NO: 41), | |
| | ID NO:3 2) | | RFTISRDNAKNTVYLQMNSLKP | |
| | | | EDTAVYYCAA (SEQ ID NO: 42), | |
| | | | RFTISRDNANSVVYLQMNSTKP | |
| | | | EDTAVYYCAA (SEQ ID NO: 43), | |
| | | | RFTISRDSAKNTVDLQMNSTKPE | |
| | | | DTAVYYCGA (SEQ ID NO: 44) | |
| Martin | QVQLQESGGGLVQAGD | WFRQAPG | YADSVKGRFTISRDNDKNTVNL | WGQGT |
| | SLRLSCAVS (SEQ ID | KEREFVA | EMNSLKPEDTAVYYCAA (SEQ | QVTVSS |
| | NO: 97), | (SEQ ID | ID NO: 101), | (SEQ ID |
| | QVQLQESGGGLVQTGD | NO: 33), | YADSVKGRFTISRDNAQNTVNL | NO: 45) |
| | SLRLSCAVS (SEQ ID | WFRQAPG | QMNSLKPEDTAVYYCAA (SEQ | |
| | NO: 98), | MEREFVA | ID NO: 102), | |
| | QVQLQESGGGLVQAGD | (SEQ ID | YADSVKGRFTISRDNAQNTVNL | |
| | SLRLSCAAS (SEQ ID | NO: 34) | QMNSLEPEDTAVYYCAA (SEQ | |
| | NO: 99), | | ID NO: 103), | |
| | QVQLQESGGGLVQAGD | | YSDSVKGRFTISRDNDKNTVNL | |
| | SLTLSCAVS (SEQ ID | | QMNSLKPEDTAVYYCAA (SEQ | |
| | NO: 100) | | ID NO: 104), | |
| | | | YSDSVKGRFSISRDNDQNTVNL | |
| | | | QMNSLKPEDTAVYYCAA (SEQ | |
| | | | ID NO: 105), | |
| | | | YADSVKGRFTISRDNDQNTVNL | |
| | | | QMNSLKPEDTAVYYCAA (SEQ | |
| | | | ID NO: 106), | |
| | | | YSDSVKGRFTISRDNDKNTVNL | |
| | | | QMNNLKPEDTAVYYCAA (SEQ | |
| | | | ID NO: 107), | |
| | | | YADSVKGRFTISRDNAKNTVYL | |
| | | | QMNSLKPEDTAVYYCAA (SEQ | |
| | | | ID NO: 108), | |
| | | | YADSVKGRFTISRDNANSVVYL | |
| | | | QMNSTKPEDTAVYYCAA (SEQ | |
| | | | ID NO: 109), | |
| | | | YADSMKGRFTISRDSAKNTVDL | |
| | | | QMNSTKPEDTAVYYCGA (SEQ | |
| | | | ID NO: 110) | |

A polypeptidic or polypeptide binding agent, in particular an antibody or antibody fragment, more particularly an ISVD, as described herein may be characterized in that it comprises a framework region 1 (FR1) present in any one of SEQ ID NOs: 1-10, wherein the FR1 is defined according to any one of AbM, Chothia, Martin, Kabat, IMGT or MacCallum, or in that it comprises a variant FR1 which is at least 90% or 95% identical to, or which has at most 3, such as 1, 2 or 3, amino acid substitutions, deletions or additions, such as preferably conservative and/or humanizing substitutions, compared to, a FR1 present in any one of SEQ ID NOs: 1-10, wherein the FR1 is defined according to any one of AbM, Chothia, Martin, Kabat, IMGT or MacCallum

A polypeptidic or polypeptide binding agent, in particular an antibody or antibody fragment, more particularly an ISVD, as described herein may be characterized in that it comprises a framework region 2 (FR2) present in any one of SEQ ID NOs: 1-10, wherein the FR2 is defined according to any one of AbM, Chothia, Martin, Kabat, IMGT or MacCallum, or in that it comprises a variant FR2 which is at least 85% or 90% identical to, or which has at most 2, such as 1 or 2, amino acid substitutions, deletions or additions, such as preferably conservative and/or humanizing substitutions, compared to, a FR2 present in any one of SEQ ID NOs: 1-10, wherein the FR2 is defined according to any one of AbM, Chothia, Martin, Kabat, IMGT or MacCallum.

A polypeptidic or polypeptide binding agent, in particular an antibody or antibody fragment, more particularly an ISVD, as described herein may be characterized in that it comprises a framework region 3 (FR3) present in any one of SEQ ID NOs: 1-10, wherein the FR3 is defined according to any one of AbM, Chothia, Martin, Kabat, IMGT or MacCallum, or in that it comprises a variant FR3 which is at least 80%, 85%, 90% or 95% identical to, or which has at most 9, such as 1, 2, 3, 4, 5, 6, 7, 8 or 9, amino acid substitutions, deletions or additions, such as preferably conservative and/or humanizing substitutions, compared to, a FR3 present in any one of SEQ ID NOs: 1-10, wherein the FR3 is defined according to any one of AbM, Chothia, Martin, Kabat, IMGT or MacCallum.

A polypeptidic or polypeptide binding agent, in particular an antibody or antibody fragment, more particularly an ISVD, as described herein may be characterized in that it comprises a framework region 4 (FR4) present in any one of SEQ ID NOs: 1-10, wherein the FR4 is defined according to any one of AbM, Chothia, Martin, Kabat, IMGT or MacCallum, or in that it comprises a variant FR4 which is at least 90% identical to, or which has at most 1 amino acid substitution, deletion or addition, such as preferably a conservative and/or humanizing substitution, compared to, a FR4 present in any one of SEQ ID NOs: 1-10, wherein the FR4 is defined according to any one of AbM, Chothia, Martin, Kabat, IMGT or MacCallum.

In further embodiments, a polypeptidic or polypeptide binding agent, in particular an antibody or antibody fragment, more particularly an ISVD, as described herein may be characterized in that it comprises, each independently, a FR1 present in any one of SEQ ID Nos: 1-10 or a variant FR1 as defined hereinabove; a FR2 present in any one of SEQ ID Nos: 1-10 or a variant FR2 as defined hereinabove; a FR3 present in any one of SEQ ID Nos: 1-10 or a variant FR3 as defined hereinabove; and a FR4 present in any one of SEQ ID Nos: 1-10 or a variant FR4 as defined hereinabove, wherein the FR1, FR2, FR3 and FR4 are defined according to any one of AbM, Chothia, Martin, Kabat, IMGT or MacCallum.

In particular embodiments, a polypeptidic or polypeptide binding agent, in particular an antibody or antibody fragment, more particularly an ISVD, as described herein may be characterized in that it comprises at least one, or the particular combination of two, three or all of the framework regions (FRs) as present in any one of SEQ ID NOs: 1 to 10, or any variant of said FR or FRs as defined herein above, wherein the FRs are annotated according to any one of Kabat, MacCallum, IMGT, AbM, Martin or Chothia.

In particular embodiments, a polypeptidic or polypeptide binding agent, in particular an antibody or antibody fragment, more particularly an ISVD, as described herein may be characterized in that it comprises at least one, or the particular combination of two, three or all of the framework regions (FRs) present in any one of SEQ ID NOs: 1 to 10, wherein the FRs are annotated according to any one of Kabat, MacCallum, IMGT, AbM, Martin or Chothia.

In further particular embodiments, polypeptidic or polypeptide binding agents, in particular antibodies or antibody fragments, more particularly ISVDs, as described herein can be defined as comprising, each independently, a FR1 present in any one of SEQ ID NOs: 1-10; a FR2 present in any one of SEQ ID NOs: 1-10; a FR3 present in any one of SEQ ID NOs: 1-10, and a FR4 present in any one of SEQ ID NOs: 1-10, wherein the FR1, FR2, FR3 and FR4 are defined according to any one of AbM, Chothia, Martin, Kabat, IMGT or MacCallum.

In further particular embodiments, polypeptidic or polypeptide binding agents, in particular antibodies or antibody fragments, more particularly ISVDs, as described herein can be defined as comprising a FR1, FR2, FR3 and FR4 as present in the same sequence of any of the sequences shown in SEQ ID NOs: 1-10, wherein the FR1, FR2, FR3 and FR4 are defined according to any one of AbM, Chothia, Martin, Kabat, IMGT or MacCallum.

In yet further particular embodiments, polypeptidic or polypeptide binding agents, in particular antibodies or antibody fragments, more particularly ISVDs, as described herein can be defined as comprising all four framework regions (FRs) present in any one of SEQ ID NOs: 1-10, wherein the FRs are defined according to any one of AbM, Chothia, Martin, Kabat, IMGT or MacCallum.

For example, polypeptidic or polypeptide binding agents, in particular antibodies or antibody fragments, more particularly ISVDs, as described herein can be defined as comprising the framework regions (FRs) present in any one of SEQ ID NOs: 1-10, wherein the FRs are defined according to Martin. In certain embodiments, the binding agents, in particular antibodies or antibody fragments, more particularly ISVDs, comprise one of following sets of framework regions (FRs):
FR1 defined by/set forth in SEQ ID NO:97, FR2 defined by/set forth in SEQ ID NO:33, FR3 defined by/set forth in SEQ ID NO: 101, and FR4 defined by/set forth in SEQ ID NO:45; or
FR1 defined by/set forth in SEQ ID NO:97, FR2 defined by/set forth in SEQ ID NO:33, FR3 defined by/set forth in SEQ ID NO: 102, and FR4 defined by/set forth in SEQ ID NO:45; or
FR1 defined by/set forth in SEQ ID NO:97, FR2 defined by/set forth in SEQ ID NO:33, FR3 defined by/set forth in SEQ ID NO: 103, and FR4 defined by/set forth in SEQ ID NO:45; or
FR1 defined by/set forth in SEQ ID NO:98, FR2 defined by/set forth in SEQ ID NO:33, FR3 defined by/set forth in SEQ ID NO: 104, and FR4 defined by/set forth in SEQ ID NO:45; or
FR1 defined by/set forth in SEQ ID NO:98, FR2 defined by/set forth in SEQ ID NO:33, FR3 defined by/set forth in SEQ ID NO: 105, and FR4 defined by/set forth in SEQ ID NO:45; or
FR1 defined by/set forth in SEQ ID NO:98, FR2 defined by/set forth in SEQ ID NO:33, FR3 defined by/set forth in SEQ ID NO: 106, and FR4 defined by/set forth in SEQ ID NO:45; or
FR1 defined by/set forth in SEQ ID NO:98, FR2 defined by/set forth in SEQ ID NO:33, FR3 defined by/set forth in SEQ ID NO: 107, and FR4 defined by/set forth in SEQ ID NO:45; or
FR1 defined by/set forth in SEQ ID NO:99, FR2 defined by/set forth in SEQ ID NO:34, FR3 defined by/set forth in SEQ ID NO: 108, and FR4 defined by/set forth in SEQ ID NO:45; or
FR1 defined by/set forth in SEQ ID NO:99, FR2 defined by/set forth in SEQ ID NO:33, FR3 defined by/set forth in SEQ ID NO: 109, and FR4 defined by/set forth in SEQ ID NO:45; or
FR1 defined by/set forth in SEQ ID NO: 100, FR2 defined by/set forth in SEQ ID NO:33, FR3 defined by/set forth in SEQ ID NO: 110, and FR4 defined by/set forth in SEQ ID NO:29.

For example, polypeptidic or polypeptide binding agents, in particular antibodies or antibody fragments, more particularly ISVDs, as described herein can be defined as comprising the framework regions (FRs) present in any one of SEQ ID NOs: 1-10, wherein the FRs are defined according to Kabat. In certain embodiments, the binding agents, in particular antibodies or antibody fragments, more particularly ISVDs, comprise one of following sets of framework regions (FRs):
FR1 defined by/set forth in SEQ ID NO:28, FR2 defined by/set forth in SEQ ID NO:33, FR3 defined by/set forth in SEQ ID NO:35, and FR4 defined by/set forth in SEQ ID NO:45; or
FR1 defined by/set forth in SEQ ID NO:28, FR2 defined by/set forth in SEQ ID NO:33, FR3 defined by/set forth in SEQ ID NO:36, and FR4 defined by/set forth in SEQ ID NO:45; or
FR1 defined by/set forth in SEQ ID NO:28, FR2 defined by/set forth in SEQ ID NO:33, FR3 defined by/set forth in SEQ ID NO:37, and FR4 defined by/set forth in SEQ ID NO:45; or
FR1 defined by/set forth in SEQ ID NO:29, FR2 defined by/set forth in SEQ ID NO:33, FR3 defined by/set forth in SEQ ID NO:38, and FR4 defined by/set forth in SEQ ID NO:45; or
FR1 defined by/set forth in SEQ ID NO:29, FR2 defined by/set forth in SEQ ID NO:33, FR3 defined by/set forth in SEQ ID NO:39, and FR4 defined by/set forth in SEQ ID NO:45; or
FR1 defined by/set forth in SEQ ID NO:29, FR2 defined by/set forth in SEQ ID NO:33, FR3 defined by/set forth in SEQ ID NO:40, and FR4 defined by/set forth in SEQ ID NO:45; or
FR1 defined by/set forth in SEQ ID NO:29, FR2 defined by/set forth in SEQ ID NO:33, FR3 defined by/set forth in SEQ ID NO:41, and FR4 defined by/set forth in SEQ ID NO:45; or
FR1 defined by/set forth in SEQ ID NO:30, FR2 defined by/set forth in SEQ ID NO:34, FR3 defined by/set forth in SEQ ID NO:42, and FR4 defined by/set forth in SEQ ID NO:45; or
FR1 defined by/set forth in SEQ ID NO:31, FR2 defined by/set forth in SEQ ID NO:33, FR3 defined by/set forth in SEQ ID NO:43, and FR4 defined by/set forth in SEQ ID NO:45; or
FR1 defined by/set forth in SEQ ID NO:32, FR2 defined by/set forth in SEQ ID NO:33, FR3 defined by/set forth in SEQ ID NO:44, and FR4 defined by/set forth in SEQ ID NO:29.

In particular embodiments, the polypeptidic or polypeptide binding agents, in particular antibodies or antibody fragments, comprise one or more ISVDs individually defined by or set forth in any one of SEQ ID NOs: 1 to 10, or comprise one or more ISVDs comprising or consisting of an amino acid sequence selected from the group of SEQ ID NO: 1 to 10.

In further embodiments, said polypeptidic or polypeptide binding agents, in particular antibodies or antibody fragments, more particularly ISVDs, comprise or consist of an amino acid sequence with at least 90% identity to an amino acid sequence selected from the group of SEQ ID NO: 1 to 10, or with at least 95% identity to an amino acid sequence selected from the group of SEQ ID NO: 1 to 10. Such non-identity or variability, is preferably limited to non-identity or variability in FR amino acid residues. In particular, such non-identity or variability may be introduced to obtain a humanized variant of an ISVD defined by or set forth in any of SEQ ID NOs: 1-10. In particular, such humanized variant may be a functional orthologue of the original ISVD, wherein the functional features are one or more of the functional features (1) to (47) outlined extensively hereinabove.

The term “wild-type” or “native” refers to a gene or gene product isolated from a naturally occurring source. A wild-type gene is that which is most frequently observed in a population and is thus arbitrarily designed the “normal” or “wild-type” form of the gene or gene product. In contrast, the term “modified”, “mutant”, “engineered” or “variant” refers to a gene or gene product that displays modifications (such as a substitution, mutation or variation, deletion or addition) in sequence, post-translational modifications and/or modification of biological or functional properties (i.e., altered characteristics) when compared to the wild-type gene or gene product. It is noted that naturally occurring mutants or variants can be isolated; these are identified by the fact that they have altered characteristics when compared to the wild-type gene or gene product. The altered characteristics can solely reside at the sequence level, or can additionally confer altered biological and/or functional properties to the mutants or variants compared to the wild-type gene or gene product. It is understood that conservative amino acid substitutions can be introduced in a protein or polypeptide whereby such substitutions have no essential or substantial effect on the protein's activity, Preferred conservative substitutions are those fulfilling the criteria defined for an accepted point mutation in Dayhoff et al. Atlas of Protein Sequence and Structure, 5, pp, 345-352 (1978 & Supp.), which is incorporated herein by reference, Examples of conservative substitutions are substitutions including but not limited to the following groups: (a) valine, glycine; (b) glycine, alanine; (c) valine, isoleucine, leucine; (d) aspartic acid, glutamic acid; (c) asparagine, glutamine; (f) serine, threonine; (g) lysine, arginine, methionine; and (h) phenylalanine, tyrosine. A “homologue”, or “homologues” of a protein of interest encompass(es) proteins having amino acid substitutions, deletions and/or insertions relative to an unmodified (e.g. native, wild-type) protein of interest and having essentially or substantially similar biological and functional activity as the unmodified protein from which it is/they are derived.

A “percentage (of) sequence identity” is calculated by comparing two optimally aligned (amino acid or nucleic acid) sequences over the window of comparison, determining the number of positions at which the identical amino acid or nucleotide residue occurs in both sequences to yield the number of matched positions, dividing the number of matched positions by the total number of positions in the window of comparison (i.e., the window size), and multiplying the result by 100 to yield the percentage of (amino acid or nucleic acid) sequence identity.

Immunoglobulin single variable domains such as Domain antibodies and Nanobody® (including VHH domains) can be subjected to humanization, i.e. to increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein), Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for case and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody R (including VHH domains) may be partially humanized or fully humanized.

Humanized immunoglobulin single variable domains, in particular Nanobody R, may have several advantages, such as a reduced immunogenicity, compared to the corresponding naturally occurring VHH domains, By humanized is meant mutated so that immunogenicity upon administration in human patients is minor or non-existent. The humanizing substitutions should be chosen such that the resulting humanized amino acid sequence and/or ISVD or VHH still retains the favourable properties of the parental (non-humanized) VHH, such as the antigen-binding capacity, Based on the description provided herein, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art, One alternative includes a method wherein the skilled person aligns a number of human germline alleles, such as for instance but not limited to the alignment of IGHV3 alleles, and to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles and used for humanisation construct design. A humanisation technique applied to Camelide VHHs may also be performed by a method comprising the replacement of specific amino acids, either alone or in combination, Said replacements may be selected based on what is known from literature, from known humanization efforts, as well as from human consensus sequences compared to the natural VHH sequences, or from the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others, Generally, although the invention in its broadest sense is not limited thereto, any substitutions, deletions or insertions (or additions) are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation, Indeed, some Camelidae VHH sequences display a high sequence homology to human VH framework regions and therefore said VHH might be administered to patients directly without expectation of an immune response therefrom, and without the additional burden or need of humanization.

Suitable mutations, in particular substitutions, can be introduced during humanization to generate a polypeptide with reduced binding to pre-existing antibodies (reference is made for example to WO 2012/175741 and WO2015/173325), for example at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined below) or at one or more other framework residues (i.e. non-Hallmark residues) or any suitable combination thereof, Depending on the host organism used to express the amino acid sequence, ISVD or VHH or polypeptide as described herein, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation.

In some cases, at least one of the typical Camelidae hallmark residues with hydrophilic characteristics at position 37, 44, 45 and/or 47 is replaced (see Table A-03 of WO2008/020079), Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 10 103, 104, 108 and/or 111 (see Tables A-05-A08 of WO2008/020079; all numbering according to the Kabat-methodology). In particular embodiments, the humanized antibody, in particular the humanized ISVD, comprises a substitution of a residue at position 1, 5. 14, 16, 19, 63, 73, 79, 82c, 83 and/or, preferably and, 108 according to the Kabat numbering. In other particular embodiments, the humanized antibody, in particular the humanized ISVD, comprises a substitution of a residue at position 1, 5, 14, 16, 19, 63, 73, 79, 83 and/or, preferably and, 108 according to the Kabat numbering, Humanization typically only concerns substitutions, deletions or additions, in the FR and not in the CDRs, as this could/would impact binding affinity to the target and/or potency.

Particular, non-limiting, examples of humanized ISVDs described herein include:

humanized R3_DC23:
(SEQ ID NO: 127)
DVQLVESGGGLVQPGGSLRLSCAVSGRIFSTYTMGWERQAPGKEREFV
AAVRWGAGTIYYADSVKGRFTISRDNAKNTVYLQMNSLRPEDTAVYYC
GAAYVSKANYGSLWYQDSRRYDYWGQGTLVTVSS
humanized R3_C4:
(SEQ ID NO: 128)
DVQLVESGGGLVQPGGSLRLSCAVSGRPFSTYTMGWFRQAPGKEREFV
AAIRWSGGTIYYADSVKGRFTISRDNAKNTVYLQMNSLRPEDTAVYYC
AAAYVSKANYGSLWYRASGLYDYWGQGTLVTVSS
humanized R4_DC20:
(SEQ ID NO: 129)
DVQLVESGGGLVQPGGSLRLSCAVSGRPFSTYTMGWFRQAPGKEREFV
ASIRWSGGTTNYADSVKGRFTISRDNAKNTVYLQMNSLRPEDTAVYYC
AAAYVSKANYGSLWYRNSGLYDYWGQGTLVTVSS

In certain embodiments, the antibody comprises one or more ISVDs as described herein (or variants or humanized forms thereof as described herein) wherein the one or more ISVD (or variant or humanized form thereof as described herein) is bound or fused to an Fc domain.

An “Fc domain” as used herein refers to the fragment crystallizable region (Fc region) of a conventional antibody, which is the tail region known to interact with cell surface receptors called Fc receptors and some proteins of the complement system, Said Fc domain is composed of two identical protein fragments, derived from the second and third constant domains of the antibody's two heavy chains. All conventional antibodies comprise an Fc domain, hence, the Fc domain may be an Fc domain derived from or as a variant of the IgG, IgA and IgD antibody Fc regions, even more specifically derived from an IgG1, IgG2 or IgG4 antibody Fc region. For example, the hinge region of IgG2, may be replaced by the hinge of human IgG1 to generate ISVD fusion constructs, and vice versa. In addition, Fc variants with known half-life extension may be used such as the M257Y/S259T/T261E (also known as YTE) or the LS variant (M428L combined with N434S), These mutations increase the binding of the Fc domain of a conventional antibody to the neonatal receptor (FcRn), Preferably, human Fc domains or humanized Fc domains may be used, Humanized forms, include but are not limited to the IgG humanization variants known in the art, such as C-terminal deletion of Lysine, alteration or truncation in the hinge region, LALA (L234A and L235A) or LALAPG (L234A, L235A, and P329G) mutations, among other substitutions in the IgG sequence.

The term “fused to”, as used herein interchangeably with “connected to”, “conjugated to”, “ligated to” refers in one aspect to “genetic fusion”, e.g., by recombinant DNA technology, as well as to “chemical and/or enzymatic conjugation” resulting in a stable covalent link between two nucleic acid molecules. The same applies for the term “inserted in”, wherein a fragment of one nucleic acid may be inserted in a second nucleic acid molecule by fusing or ligating the two sequences genetically, enzymatically or chemically, Peptides or polypeptides can likewise be fused or connected to one another, such as via peptide bonds or via linking one peptide to a side chain of an amino acid in a second peptide.

Linkers may be used to fuse an ISVD, such as a herein identified ISVD (or variant or humanized form thereof as described herein), to an Fc domain such as the human IgG1 Fc domain or the LS variant thereof, or the YTE variant thereof, or an IgG2 Fc domain. A non-limiting example of a linker comprises a Gly-Ser linker such as (G₄S)ₙ, with n=1-6 (SEQ ID NO:120), preferably 2-3 (SEQ ID NO: 121-122).

In certain embodiments, the antibody comprising one or more ISVDs as described herein (or variants or humanized forms thereof as described herein) is in a “multivalent” and/or “multispecific” form formed by binding, e.g. chemically or by recombinant DNA techniques, together two or more identical or variant monovalent ISVDs (or variants or humanized forms thereof as described herein), Non-limiting examples of multivalent constructs include “bivalent” constructs, “trivalent” constructs, “tetravalent” constructs, and so on, respectively, comprising two, three or four ISVDs. The ISVDs comprised within a multivalent construct may be identical or different. The term “multispecific antibody” as used herein specifically refers to a multivalent antibody wherein at least one of the two or more ISVDs has a different specificity, Non-limiting examples of multi-specific constructs include “bi-specific” constructs, “tri-specific” constructs, “tetra-specific” constructs, and so on, To illustrate this further, any multivalent and multi-specific (as defined herein) antibody of the invention may be directed against two or more different antigens, for example against a Sarbecovirus and one as a half-life extension against Serum Albumin or Staphylococcal protein A (SpA) and/or against two or more different parts of a particular antigen, for example against two or more different parts, regions, subunits or domains of a Sarbecovirus spike protein.

In particular embodiments, an antibody, in particular a multivalent and/or multispecific antibody, may comprise one or more binding agent, such as ISVD(s), as described herein (or variants or humanized forms thereof as described herein), and one or more binding agents, such as ISVD(s), capable of binding to a Sarbecovirus spike protein receptor binding domain (RBD), Non-limiting examples of ISVDs capable of binding to a Sarbecovirus spike protein receptor binding domain (RBD) are described in PCT/EP2021/052885, PCT/EP2022/052919 and PCT/EP2022/062980.

Advantageously, the combination of at least two ISVDs capable of binding Sarbecovirus spike protein through interaction at 2 different regions of the spike protein, in particular the S2 subunit, more particularly the HR2 domain, and the RBD, in the multivalent and/or multispecific antibody may result in cross-reactivity and potent prohibition of infection by Sarbecoviruses, and may further allow for reducing the risk to escape mutant virus emergence.

In certain further embodiments, the one or more ISVDs capable of binding to a Sarbecovirus spike protein RBD are capable of binding to or competing for the VHH72 epitope (or the epitope specifically bound by VHH72). The VHH72 epitope has been described in Wrapp et al. (2020, Cell 184:1004-1015; PCT/EP2021/052885 and PCT/EP2022/062980). The VHH72 epitope as defined herein refers to a conformational epitope in the RBD comprising at least one or more of the amino acid residues S371, S375, T376, or C379 as set forth in SEQ ID NO: 86, or even more specifically, at least one or more of L368, Y369, S371, S375, T376, F377, K378, C379 and Y508 as set forth in SEQ ID NO: 86, which is the sequence of the SARS-Cov-2 spike protein. In particular, an ISVD capable of binding to the VHH72 epitope may be capable of specifically binding to the SARS-COV-2 Spike protein (SEQ ID NO:86), to at least 3, at least 4, at least 5, at least 6, at least 7, at least 8, to at least 9, to at least 10, to at least 11, or to all of the amino acids L368, Y369, S371, S375, T376, F377, K378, C379 and Y508 of the SARS-COV-2 spike protein as depicted in SEQ ID NO:86. An ISVD capable of competing for the VHH72 epitope refers to an ISVD that competes with VHH72 for binding to the spike protein as depicted in SEQ ID NO:86, or the RBD, With ‘competing’ is meant that the binding of VHH72 to the spike protein as depicted in SEQ ID NO:86, or the RBD, is reduced with at least 30%, or at least 50%, or preferably at least 80% in strength in the presence of an ISVD capable of competing for the VHH72 epitope. In particular, an ISVD capable of competing for the VHH72 epitope, or competing with VHH72 binding to the RBD epitope, may be capable of specifically binding to an epitope on the spike protein comprising at least three, at least four, at least five, at least 6 or more of the residues L368, Y369, S371, S375, T376, F377, K378, C379 and Y508 of the Spike protein of SARS-Cov-2, as depicted in SEQ ID NO:86, so as to provide an overlapping epitope. In embodiments, an ISVD capable of binding to or competing for the VHH72 epitope may be characterized in that (i) it competes for human receptor (ACE-2 in the case of SARS-COV-1 and -2) binding upon interaction to the RBD, and/or (ii) is not competing with an ISVD capable of binding to or competing with a VHH3.117 epitope as defined herein, Non-limiting examples of ISVDs capable of binding to or competing for the VHH72 epitope include VHH72 family members (including VHH72 (SEQ ID NO:124), VHH2.50, VHH3.17, VHH3.77, VHH3.115, VHH3.144 and VHHBE4), and variants, including VHH72 (S56A), and humanized forms thereof; VHH3.83 family members (including VHH3.83 (also referred to as VHH83 herein) (SEQ ID NO: 125)) and variants and humanized forms thereof; VHH3.38 family members and variants and humanized forms thereof; VHH3.55 family members and variants and humanized forms thereof; VHH3.36 family members and variants and humanized forms thereof; VHH3.149 family members and variants and humanized forms thereof; and VHH3.29 family members and variants and humanized forms thereof, as described in PCT/EP2021/052885 and PCT/EP2022/062980.

In particular embodiments, an antibody, in particular a multivalent and/or multispecific antibody, may comprise one or more ISVDs as described herein (or variants or humanized forms thereof as described herein), and an ISVD comprising the CDRs present in SEQ ID NO: 125 or SEQ ID NO: 124, such as an ISVD comprising or consisting of the sequence set forth in SEQ ID NO: 125 (e.g. VHH83) or SEQ ID NO: 124 (e.g., VHH72), or a variant or a humanized form thereof, wherein the CDRs are annotated according to Kabat, Martin, MacCallum, IMGT, AbM, or Chothia.

In certain further embodiments, the one or more ISVDs capable of binding to a Sarbecovirus spike protein RBD are capable of binding to or competing for the VHH3.117 epitope (or the epitope specifically bound by VHH3.117). The VHH3.117 epitope has been described in PCT/EP2022/052919. In particular, an ISVD capable of binding to the VHH3.117 epitope may be capable of binding or specifically binding to at least one, or in increasing order of preference at least two, at least three, or at least four, of the amino acids Asn394 (or alternatively Ser394 in some Sarbecoviruses), Tyr396, Phe464, Ser514, Glu516, and Arg355 of the SARS-COV-2 spike protein as defined in SEQ ID NO:86 and optionally may be capable of further binding or specifically binding to amino acid Arg357 (or alternatively Lys357 in some Sarbecoviruses) and/or Lys462 (or alternatively Arg462 in some Sarbecoviruses) and/or Glu465 (or alternatively Gly465 in some Sarbecoviruses) and/or Arg466 and/or Leu518, such as may be capable of further binding or specifically binding to at least two, or in increasing order of preference at least three or all four of amino acid Arg357 (or alternatively Lys357 in some Sarbecoviruses) and/or Lys462 (or alternatively Arg462 in some Sarbecoviruses) and/or Glu465 (or alternatively Gly465 in some Sarbecoviruses) and/or Arg466 and/or Leu518, An ISVD capable of competing for the VHH3.117 epitope refers to an ISVD that competes with VHH3.117 for binding to the spike protein as depicted in SEQ ID NO: 86, or the RBD, With ‘competing’ is meant that the binding of VHH3.117 to the spike protein as depicted in SEQ ID NO:86 is reduced with at least 30%, or at least 50%, or preferably at least 80% in strength in the presence of an ISVD capable of competing for the VHH3.117 epitope. In embodiments, an ISVD capable of binding to or competing for the VHH3.117 epitope may be characterized in that (i) it does not inhibit binding of the RBD with the human receptor (ACE-2 in the case of SARS-COV-1 and -2), meaning that it allows binding of the receptor and the Sarbecovirus RBD when the ISVD itself is bound to the Sarbecovirus RBD, or alternatively, that the ISVD itself can bind to a Sarbecovirus RBD to which the receptor is bound, and/or (ii) is not competing with an ISVD capable of binding to or competing for the VHH72 epitope as defined herein, Non-limiting examples of ISVDs capable of binding to or competing for the VHH3.117 epitope include VHH3.117 family members (including VHH3.117, 3.42, 3.92, 3.94, 3.180) and variants and humanized forms thereof (as described in PCT/EP2022/052919); VHH3.89 family members and variants and humanized forms thereof (as described in PCT/EP2021/052885); VHH3_183 family members and variants and humanized forms thereof; and VHH3C_80 family members and variants and humanized forms thereof (as described in PCT/EP2022/062980).

In particular embodiments, an antibody, in particular a multivalent and/or multispecific antibody, may comprise one or more ISVDs as described herein (or variants or humanized forms thereof as described herein), and an ISVD comprising the CDRs present in SEQ ID NO: 126, such as an ISVD comprising or consisting of the sequence set forth in SEQ ID NO:126 (e.g. VHH3.117), or a variant or a humanized form thereof, wherein the CDRs are annotated according to Kabat, Martin, MacCallum, IMGT, AbM, or Chothia.

In yet other further embodiments, the antibody, in particular the multivalent and/or multispecific antibody, comprises more than one ISVD capable of binding to a Sarbecovirus spike protein receptor binding domain (RBD), wherein at least one ISVD is capable of binding to or competing for the VHH72 epitope as defined herein, and wherein at least one ISVD is capable of binding to or competing for the VHH3.117 epitope as defined herein, Advantageously, the combination of at least two non-competing RBD targeting ISVDs (capable of binding the RBD of the spike protein through interaction at 2 non-competing, different regions of the RBD) and at least one S2 targeting ISVD in the antibody results in cross-reactivity and potent prohibition of infection by Sarbecoviruses, which advantageously allows for further reducing the risk to mutational escape.

In particular embodiments, an antibody, in particular a multivalent and/or multispecific antibody, may comprise one or more ISVDs as described herein (or variants or humanized forms thereof as described herein), an ISVD comprising the CDRs present in SEQ ID NO: 125 or in SEQ ID NO: 124, such as an ISVD comprising or consisting of the sequence set forth in SEQ ID NO: 125 (e.g. VHH83) or SEQ ID NO: 124 (e.g., VHH72), or a variant or a humanized form thereof, and an ISVD comprising the CDRs present in SEQ ID NO: 126, such as an ISVD comprising or consisting of the sequence set forth in SEQ ID NO: 126 (e.g. VHH3.117), or a variant or a humanized form thereof, wherein the CDRs are annotated according to Kabat, Martin, MacCallum, IMGT, AbM, or Chothia.

Multivalent antibodies as described herein may be formed e.g. by connecting, such as chemically or by recombinant DNA techniques, the two or more ISVDs directly or via a linker, and/or through fusing (each of) the two or more ISVDs with an Fc domain.

For example, a single ISVD (or variant or humanized form thereof) as described herein may be fused e.g. at its C-terminus to an Fc domain, such as an IgG Fc domain, such as a construct comprising the amino acid sequence as defined in SEQ ID NO:96 or SEQ ID NO: 118, resulting in a Sarbecovirus antibody of bivalent format wherein two of said ISVDs form a heavy chain only antibody-type molecule through disulfide bridges in the hinge region of the Fc part, such as the IgG Fc part.

In particular embodiments, one or more ISVDs as described herein (or variants or humanized forms thereof as described herein) are linked, fused or connected directly or via a linker to one or more ISVDs capable of binding to a Sarbecovirus spike protein as defined herein, Non-limiting examples of suitable linkers for linking the ISVDs include peptide linkers such as a (G₄S)ₙ, wherein n=1, 2, 3, 4, 5 or 6. Such multispecific binding agents may also be referred to herein as “head-to-tail fusions”.

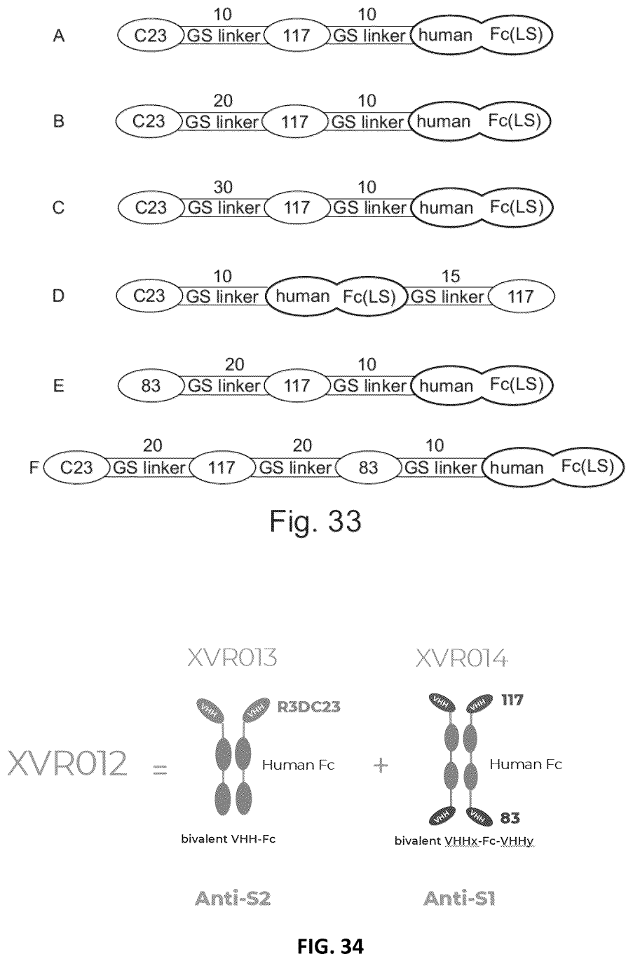

In further embodiments, the C-terminus of a head-to-tail fusion as described herein may be fused, e.g. by a linker, to an Fc domain, which construct upon expression in a host forms a multivalent and/or multispecific antibody through disulfide bridges in the hinge region of the Fc part. Accordingly, in particular embodiments, one or more ISVDs as described herein (or variants or humanized forms thereof as described herein) are linked, fused or connected directly or via a linker to one or more ISVD capable of binding to a Sarbecovirus spike protein RBD to form a multispecific binding agent or construct and said multispecific binding agent or construct is fused to an Fc domain. In preferred embodiments, the antibody comprises a bispecific binding agent or construct fused to an Fc domain, wherein said bispecific binding agent or construct comprises one ISVD as described herein (or a variant or humanized form thereof as described herein) linked, fused or connected directly or via a linker to one ISVD capable of binding to a Sarbecovirus spike protein RBD, such as an ISVD capable of binding to or competing with the VHH3.117 epitope as described herein, A schematic drawing of such multispecific antibody, in particular bispecific antibody, also referred to herein as “VHH-VHH-Fc fusion”, is depicted in FIG. 33A-C, More specific examples of such multispecific antibodies, in particular bispecific antibodies, which are capable of binding the HR2 binding site as described herein and the VHH3.117 epitope, are provided in for instance, but not limited to, SEQ ID NO: 112-114, or any functional variant thereof, or a variant with at least 90% identity thereof, or a humanized form thereof. The sequences defined by SEQ ID NO:112-114 are also shown below.

SEQ ID NO: 112
DVQLVESGGGLVQPGGSLRLSCAVSGRIFSTYTMGWERQAPGKEREFVAAVRWGAGTIYYADSVKG
RFTISRDNAKNTVYLQMNSLRPEDTAVYYCGAAYVSKANYGSLWYQDSRRYDYWGQGTLVTVSSGG
GGSGGGGSDVQLVESGGGLVQPGGSLRLSCAASGKAVSISDMGWYRQPPGKQRELVATITKTGSTN
YADSVKGRFTISRDNTKNTVYLEMNSLRPEDTAVYYCNAWLPYGLGPDYYGLELWGQGTLVTVSSG
GGGSGGGGSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDGSFFLYSK
LTVDKSRWQQGNVFSCSVLHEALHSHYTQKSLSLSPG
SEQ ID NO:113
DVQLVESGGGLVQPGGSLRLSCAVSGRIFSTYTMGWERQAPGKEREFVAAVRWGAGTIYYADSVKG
RFTISRDNAKNTVYLQMNSLRPEDTAVYYCGAAYVSKANYGSLWYQDSRRYDYWGQGTLVTVSSGG
GGSGGGGSGGGGSGGGGSDVQLVESGGGLVQPGGSLRLSCAASGKAVSISDMGWYRQPPGKQRELV
ATITKTGSTNYADSVKGRFTISRDNTKNTVYLEMNSLRPEDTAVYYCNAWLPYGLGPDYYGLELWG
QGTLVTVSSGGGGSGGGGSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPVLD
SDGSFFLYSKLTVDKSRWQQGNVFSCSVLHEALHSHYTQKSLSLSPG
SEQ ID NO: 114
DVQLVESGGGLVQPGGSLRLSCAVSGRIFSTYTMGWFRQAPGKEREFVAAVRWGAGTIYYADSVKG
RFTISRDNAKNTVYLQMNSLRPEDTAVYYCGAAYVSKANYGSLWYQDSRRYDYWGQGTLVTVSSGG
GGSGGGGSGGGGSGGGGSGGGGSGGGGSDVQLVESGGGLVQPGGSLRLSCAASGKAVSISDMGWYR
QPPGKQRELVATITKTGSTNYADSVKGRFTISRDNTKNTVYLEMNSLRPEDTAVYYCNAWLPYGLG
PDYYGLELWGQGTLVTVSSGGGGSGGGGSDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVLHEALHSHYTQKSLSLSPG

In particular embodiments, the antibody comprises a trispecific binding agent or construct fused to an Fc domain, wherein said trispecific binding agent or construct comprises one ISVD as described herein (or a variant or humanized form thereof as described herein), one ISVD capable of binding to or competing with the VHH3.117 epitope as described herein, and one ISVD capable of binding to or competing with the VHH72 epitope as described herein, wherein said ISVDs are linked, fused or connected directly or via a linker to each other, in any order. A schematic drawing of such multispecific antibody, in particular trispecific antibody, also referred to herein as “VHH-VHH-VHH-Fc fusion”, is depicted in FIG. 33F, More specific examples of such multispecific antibodies, in particular trispecific antibodies, which are capable of binding the HR2 binding site as described herein and the VHH3.117 and VHH72 epitopes, are provided in for instance, but not limited to, SEQ ID NO: 117, or any functional variant thereof, or a variant with at least 90% identity thereof, or a humanized form thereof. The sequence defined by SEQ ID NO: 117 is also shown below.

SEQ ID NO: 117
DVQLVESGGGLVQPGGSLRLSCAVSGRIFSTYTMGWERQAPGKEREFVAAVRWGAGTIYYADSVKG
RFTISRDNAKNTVYLQMNSLRPEDTAVYYCGAAYVSKANYGSLWYQDSRRYDYWGQGTLVTVSSGG
GGSGGGGSGGGGSGGGGSDVQLVESGGGLVQPGGSLRLSCAASGKAVSISDMGWYRQPPGKQRELV
ATITKTGSTNYADSVKGRFTISRDNTKNTVYLEMNSLRPEDTAVYYCNAWLPYGLGPDYYGLELWG
QGTLVTVSSGGGGSGGGGSGGGGSGGGGSDVQLVESGGGLVQPGDSLRLSCVLSGGVFTSYAMGWE
RQAPGKEREFLAAITENSDATYYADSVKGRFTISRDNAKNTAYLQMNSLRPEDTAVYSCAAGGNHY
NPQYYHDYDKYDHWGQGTLVTVSSGGGGSGGGGSGGGGSGGGGSDKTHTCPPCPAPELLGGPSVFL
FPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVLHEALHSHYTQKS
LSLSPG

In other particular embodiments, one or more ISVDs as described herein (or variants or humanized forms thereof as described herein) are fused to the N-terminus of an Fc domain, and one or more ISVDs capable of binding to a Sarbecovirus spike protein RBD are fused to the C-terminus of the Fc domain, or one or more ISVDs as described herein (or variants or humanized forms thereof as described herein) are fused to the C-terminus of an Fc domain, and one or more ISVDs capable of binding to a Sarbecovirus spike protein RBD are fused to the N-terminus of the Fc domain. In preferred embodiments, the antibody comprises one ISVD as described herein (or a variant or humanized form thereof as described herein) fused to the N-terminus of an Fc domain and one ISVD capable of binding to a Sarbecovirus spike protein RBD, in particular one ISVD capable of binding to or competing with the VHH3.117 epitope as described herein fused to the C-terminus of the Fc domain, or the one ISVD as described herein (or a variant or humanized form thereof as described herein) is fused to the C-terminus of the Fc domain and the one ISVD capable of binding to a Sarbecovirus spike protein RBD, in particular the one ISVD capable of binding to or competing with the VHH3.117 epitope as described herein is fused to the N-terminus of the Fc domain. A schematic drawing of such multispecific antibody, also referred to herein as “VHH-Fc-VHH fusions” or “moonlander”, is depicted in FIG. 33D, More specific examples of such multispecific antibodies, in particular bispecific antibodies, which are capable of binding the HR2 binding site as described herein and the VHH3.117 epitope, are provided in for instance, but not limited to, SEQ ID NO: 115, or any functional variant thereof, or a variant with at least 90% identity thereof, or a humanized variant thereof. The sequence defined by SEQ ID NO: 115 is also shown below.

SEQ ID NO: 115
DVQLVESGGGLVQPGGSLRLSCAVSGRIFSTYTMGWFRQAPGKEREFVAAVRWGAGTIYYADSVKG
RFTISRDNAKNTVYLQMNSLRPEDTAVYYCGAAYVSKANYGSLWYQDSRRYDYWGQGTLVTVSSGG
GGSGGGGSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKL
TVDKSRWQQGNVFSCSVLHEALHSHYTQKSLSLSPGGGGGSGGGGSGGGGSDVQLVESGGGLVQPG
GSLRLSCAASGKAVSISDMGWYRQPPGKQRELVATITKTGSTNYADSVKGRFTISRDNTKNTVYLE
MNSLRPEDTAVYYCNAWLPYGLGPDYYGLELWGQGTLVTVSS

Multivalent or multi-specific antibodies as described herein may have (or be engineered and/or selected for) increased avidity and/or improved selectivity for the desired Sarbecovirus interaction, and/or for any other desired property or combination of desired properties that may be obtained by the use of such multivalent and/or multi-specific antibodies.

In particular embodiments, the binding agents, in particular the multivalent and/or multi-specific antibodies described herein, more particularly the multivalent and/or multi-specific antibodies comprising an Fc domain described herein, have antibody-dependent cell-mediated cytotoxicity (ADCC) activity, More particularly, the binding agents, in particular the multivalent and/or multi-specific antibodies described herein, more particularly the multivalent and/or multi-specific antibodies comprising an Fc domain described herein, are capable of inducing ADCC on target cells expression a Sarbecovirus spike protein. “Antibody-dependent cell-mediated cytotoxicity” or “ADCC” refers to a form of cytotoxicity in which an antibody binds to certain cytotoxic cells (such as NK cells, neutrophils, and macrophages). The secretion of Ig on the Fcγ receptor enables these cytotoxic effector cells to specifically bind to the target cell carrying the antigen, and then kill the target cell using, for example, a cytotoxin. In order to evaluate the ADCC activity of an antibody of interest, an in vitro ADCC assay can be performed, for example, the method described in the Examples of this application.

Also disclosed herein are other Sarbecovirus binding agents competing with an ISVD defined by an amino acid sequence selected from the group of SEQ ID NO: 1 to 10 for binding to a Sarbecovirus spike protein or part thereof (as described hereinabove).

The term “competes” or “cross-competes” as used herein refers to a compound or binding agent which shares the ability to bind to a specific region of an antigen and inhibits or blocks the binding of another binding agent. In the present disclosure a compound or binding agent that is “competitive” or “cross-competitive” has the ability to interfere with the binding of an antibody or antigen-binding fragment as described herein, in particular an ISVD defined by an amino acid sequence selected from the group consisting of SEQ ID NO: 1 to 10 in a competitive binding assay as known to the skilled person. The term also includes competition between two antibodies or antigen-binding fragments, in both orientations, i.e., a first antibody that binds and blocks binding of a second antibody and vice versa. In certain embodiments, the first antigen-binding agent (e.g., antibody or antigen-binding fragment) and second antigen-binding agent (e.g., antibody or antigen-binding fragment) may bind to the same epitope. Alternatively, the first and second antigen-binding agents (e.g., antibodies or antigen-binding fragments) may bind to different, but, for example, overlapping epitopes, wherein binding of one inhibits or blocks the binding of the second antibody or antigen-binding fragment, e.g., via steric hindrance, Competition between antigen-binding agents (e.g., antibodies or antigen-binding fragments) may be measured by methods known in the art, for example, by ELISA (enzyme-linked immunosorbent assays) or by surface plasmon resonance (SPR), Competition or cross-competition may be present if binding of the ISVD defined by an amino acid sequence selected from the group of SEQ ID NO: 1 to 10 to a Sarbecovirus spike protein such as the SARS-COV-2 spike protein consisting of the amino acid sequence set forth in SEQ ID NO:86 or the SARS-COV-1 spike protein consisting of the amino acid sequence set forth in SEQ ID NO:111, or part thereof, in particular to the SARS-COV-2 S2 subunit or to the SARS-COV-1 S2 subunit, or part thereof, more particularly to the SARS-COV-1/-2 HR2 domain as depicted in SEQ ID NO:87, is reduced with at least 30%, or at least 50%, or preferably at least 80% in strength in the presence of a competing binding agent. In particular, such other binding agents ideally retain one or more of the functional features (1) to (47) outlined extensively hereinabove.

As such, the present disclosure also relates to methods of screening for compounds binding to a Sarbecovirus spike protein, in particular to the S2 subunit of a Sarbecovirus spike protein, more particularly to a Sarbecovirus HR2 domain in a Sarbecovirus spike protein, and competing with an ISVD or functional part thereof (or variant or humanized form thereof) as described herein for binding to a Sarbecovirus spike protein, in particular to a Sarbecovirus S2 subunit, more particularly to a Sarbecovirus HR2 domain. Such methods in general comprise one or more of the following steps:
providing a compound or pool of compounds;
contacting the compound or pool of compounds with a Sarbecovirus spike protein or with a Sarbecovirus S2 subunit or with a Sarbecovirus HR2 domain in the absence of an ISVD or functional part thereof (or variant or humanized form thereof) as described herein;
contacting the compound or pool of compounds with a Sarbecovirus spike protein or with a Sarbecovirus S2 subunit or with a Sarbecovirus HR2 domain in the presence of an ISVD or functional part thereof (or variant or humanized form thereof) as described herein;
measuring, assessing, determining, assaying whether the compound or pool of compounds is capable of reducing the amount of ISVD or functional part thereof bound to the Sarbecovirus spike protein or to the Sarbecovirus S2 subunit or to the Sarbecovirus HR2 domain; or measuring, assessing, determining, assaying whether the ISVD or functional part thereof is capable of reducing the amount of compound or pool of compounds bound to the Sarbecovirus spike protein or to the Sarbecovirus S2 subunit or to the Sarbecovirus HR2 domain;
identifying a compound as competitor of the ISVD or functional part thereof for binding to the Sarbecovirus spike protein or to the Sarbecovirus S2 subunit or to the Sarbecovirus HR2 domain when the amount of ISVD or functional part thereof bound to the Sarbecovirus spike protein or to the Sarbecovirus S2 subunit or to the Sarbecovirus HR2 domain is reduced in the presence of the compound; or identifying a pool of compounds to comprise one or more compounds as competitor of the ISVD or functional part thereof for binding to the Sarbecovirus spike protein or to the Sarbecovirus S2 subunit or to the Sarbecovirus HR2 domain when the amount of ISVD or functional part thereof bound to the Sarbecovirus spike protein or to the Sarbecovirus S2 subunit or to the Sarbecovirus HR2 domain is reduced in the presence of the one or more compounds; or identifying a compound as competitor of the ISVD or functional part thereof for binding to the Sarbecovirus spike protein or to the Sarbecovirus S2 subunit or to the Sarbecovirus HR2 domain when the amount of compound bound to the Sarbecovirus spike protein or to the Sarbecovirus S2 subunit or to the Sarbecovirus HR2 domain is reduced in the presence of the ISVD or functional part thereof; or identifying a pool of compounds to comprise one or more compounds as competitor of the ISVD or functional part thereof for binding to the Sarbecovirus spike protein or to the Sarbecovirus S2 subunit or to the Sarbecovirus HR2 domain when the amount of pool of compounds bound to the Sarbecovirus spike protein or to the Sarbecovirus S2 subunit or to the Sarbecovirus HR2 domain is reduced in the presence of the ISVD or functional part thereof.

The term “compound” or “test compound” or “candidate compound” or “drug candidate compound” as used herein describes any molecule, either naturally occurring or synthetic that is designed, identified, screened for, or generated and may be tested in an assay, such as a screening assay or drug discovery assay, or specifically in the method for identifying a compound competing with an ISVD as described herein (or a variant or humanized form thereof as described herein for binding to a Sarbecovirus spike protein or part thereof (as described hereinabove), As such, these compounds comprise organic and inorganic compounds. For high-throughput purposes, test compound libraries may be used, such as combinatorial or randomized libraries that provide a sufficient range of diversity, Examples include, but are not limited to, natural compound libraries, allosteric compound libraries, peptide libraries, antibody fragment libraries, synthetic compound libraries, fragment-based libraries, phage-display libraries, and the like. Such compounds may also be referred to as binding agents; as referred to herein, these may be “small molecules”, which refers to a low molecular weight (e.g., <900 Da or <500 Da) organic compound. The compounds or binding agents also include chemicals, polynucleotides, lipids or hormone analogs that are characterized by low molecular weights, Other biopolymeric organic test compounds include small peptides or peptide-like molecules (peptidomimetics) comprising from about 2 to about 40 amino acids and larger polypeptides comprising from about 40 to about 500 amino acids, such as antibodies, antibody mimetics, antibody fragments or antibody conjugates.

As used herein, the terms “determining”, “measuring”, “assessing”, “identifying”, “screening”, and “assaying” are used interchangeably and include both quantitative and qualitative determinations.

In yet another aspect, the invention provides nucleic acid molecules such as isolated nucleic acids, (isolated) chimeric gene constructs, expression cassettes, comprising a polynucleotide sequence, such as a coding sequence, that is encoding the polypeptide portion of a polypeptidic or polypeptide Sarbecovirus binding agent, in particular an antibody or antibody fragment, as identified herein, more particularly an ISVD (or a variant or humanized form thereof) as described herein, or a functional part thereof.

“Nucleic acid(s)” or “nucleic acid molecule(s)” as used herein refers to a polymeric form of nucleotides of any length, either ribonucleotides or deoxyribonucleotides; the sequential linear arrangement of the nucleotides together resulting in/forming the “nucleotide sequence”, “DNA sequence”, or “RNA sequence”, This term refers only to the primary structure of the molecule, Thus, this term includes double- and single-stranded DNA, and RNA, It also includes known types of modifications, for example, methylation, “caps”, and substitution of one or more of the naturally occurring nucleotides with an analog, Modifications to nucleic acids can be introduced at one or more levels: phosphate linkage modification (e.g. introduction of one or more of phosphodiester, phosphoramidate or phosphorothioate bonds), sugar modification (e.g. introduction of one or more of LNA (locked nucleic acids), 2′-O-methyl, 2′-O-methoxy-ethyl, 2′-fluoro, S-constrained ethyl or tricyclo-DNA) and/or non-ribose modifications (e.g. introduction of one or more of phosphorodiamidate morpholinos or peptide nucleic acids).

By “nucleic acid construct” it is meant a nucleic acid molecule that has been constructed in order to comprise one or more functional units not found together in nature, thus having a nucleotide sequence not found in nature (non-native nucleotide sequence), Examples include circular, linear, double-stranded, extrachromosomal DNA molecules (plasmids), cosmids (plasmids containing COS sequences from lambda phage), viral genomes comprising non-native nucleic acid sequences, and the like.

A “coding sequence” is a nucleotide sequence that can be transcribed into mRNA and/or translated into a polypeptide when placed under the control of appropriate (gene) regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

With a “chimeric gene” or “chimeric construct” or “chimeric gene construct” is interchangeably meant a recombinant nucleic acid sequence in which a (gene) promoter or regulatory nucleic acid sequence is operably or operatively linked to, or associated with, a nucleic acid sequence of interest that codes for an RNA (e.g. a coding sequence, an shRNA, etc.), such that the regulatory nucleic acid sequence is able to regulate transcription or expression of the nucleic acid of interest. The operable or operative linkage in a chimeric gene between the regulatory nucleic acid sequence and the nucleic acid sequence of interest is not found in nature.

An “expression cassette” comprises any nucleic acid construct capable of directing the expression of a gene/coding sequence of interest, which is operably linked to a (gene) promoter, Expression cassettes are generally DNA constructs preferably including (5′ to 3′ in the direction of transcription); a (gene) promoter region, a polynucleotide sequence of interest with a transcription initiation region, and a termination sequence including a stop signal for RNA polymerase and a polyadenylation signal; all these elements being operably or operatively linked meaning that all of these regions should be capable of operating (being expressed) in a cell, such as prokaryotic (e.g. bacterial) or eukaryotic (e.g. mammalian, yeast, insect, fungal, plant, algal) cells, when transformed into that cell. The promoter region comprising the transcription initiation region, which preferably includes the RNA polymerase binding site, and the polyadenylation signal may be native to the cell to be transformed, may be derived from an alternative source, or may be synthetic, as long as it is functional in the cell. Such expression cassettes can be constructed in e.g. a “vector” or “expression vector” (linear or circular nucleic acids, plasmids, cosmids, viral vectors, phagemids, etc.).

The present invention also provides a vector including the above-mentioned nucleic acid molecule inserted therein.

The term “vector”, “vector construct”, “expression vector”, “recombinant vector” or “gene transfer vector”, as used herein, is intended to refer to a nucleic acid molecule capable of carrying another nucleic acid molecule to which it has been linked.

Said vectors may include a cloning or expression vector, as well as a delivery vehicle such as a viral, lentiviral or adenoviral vector, Expression vectors may comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. In particular, an expression vector as described herein may comprise a nucleic acid molecule as described herein comprising a nucleic acid sequence encoding an antibody or an antigen-binding fragment as described herein operably linked to at least one regulatory sequence, Regulatory sequences are selected to direct the expression of the protein of interest, in particular the antibody or antigen-binding fragment, in a suitable host cell, and include promoters, enhancers, and other expression control elements as known to the skilled person. Hence, in embodiments, the vector includes a promoter for driving expression of the nucleic acid of interest, optionally a nucleic acid sequence encoding a signal peptide that secretes the antibody or antigen-binding fragment, and optionally a nucleic acid sequence encoding a terminator. When the expression vector is manipulated in a production strain or cell line, the vector may or may not be integrated into the genome of the host cell when introduced into the host cell, Cloning vectors are generally used to engineer and amplify a certain desired DNA fragment, Thus, a cloning vectors may contain origin of replication that matches the cell type specified by the cloning vector, and may lack functional sequences needed for expression of the desired DNA fragments, Preferably, the vector contains one or more selection markers. The choice of the selection markers may depend on the host cells of choice, although this is not critical to the present invention as is well known to persons skilled in the art. The construction of expression vectors for use in transfecting cells is also well known in the art, and thus can be accomplished via standard techniques (see, for example, Sambrook, Fritsch, and Maniatis, in: Molecular Cloning. A Laboratory Manual, Cold Spring Harbor Laboratory Press, 1989; Gene Transfer and Expression Protocols, pp, 109-128, ed, E. J, Murray. The Humana Press Inc., Clifton, N.J.), and the Ambion 1998 Catalog (Ambion, Austin, Tex.).

More particular, said vector may include any vector known to the skilled person, including any suitable type, but not limited to, for instance, plasmid vectors, cosmid vectors, phage vectors, such as lambda phage, viral vectors, even more particular a lentiviral, adenoviral, AAV or baculoviral vectors, or artificial chromosome vectors such as bacterial artificial chromosomes (BAC), yeast artificial chromosomes (YAC), or PI artificial chromosomes (PAC). The choice of the vector may bee dependent amongst others on the nature of the host cell of choice.

One further aspect of the invention provides for a host cell comprising an antibody or antigen-binding fragment thereof, such as an ISVD (or variant or humanized form thereof) of an antibody or antigen-binding fragment, or part thereof, as described herein. The host cell may therefore comprise the nucleic acid molecule encoding said antibody or antigen-binding fragment, Host cells can be either prokaryotic or eukaryotic. The host cell may also be a recombinant host cell, which involves a cell which has been genetically modified to contain an isolated nucleic acid molecule encoding the antibody or antigen-binding fragment of the invention, Representative host cells that may be used to produce said antibodies or antigen-binding fragments such as ISVDs, include, but are not limited to, bacterial cells, yeast cells, plant cells and animal cells, Bacterial host cells suitable for production of the antibodies or antigen-binding fragment of the invention include Escherichia spp, cells, Bacillus spp, cells, Streptomyces spp, cells, Erwinia spp, cells, Klebsiella spp, cells, Serratia spp, cells, Pseudomonas spp, cells, and Salmonella spp, cells, Yeast host cells suitable for use with the invention include species within Saccharomyces, Schizosaccharomyces, Kluyveromyces, Pichia (e.g. Pichia pastoris), Hansenula (e.g. Hansenula polymorpha), Yarowia, Schwaniomyces, Schizosaccharomyces, Zygosaccharomyces and the like, Saccharomyces cerevisiae, S carlsbergensis and K. lactis are the most commonly used yeast hosts, and are convenient fungal hosts, Animal host cells suitable for use with the invention include insect cells and mammalian cells (e.g. derived from Chinese hamster (e.g. CHO), and human cell lines, such as HeLa), Exemplary insect cell lines include, but are not limited to, Sf9 cells, baculovirus-insect cell systems (e.g. review Jarvis, Virology Volume 310, Issue 1, 25 May 2003, Pages 1-7), Alternatively, the host cells may also be transgenic animals or plants.

Introduction of a vector in a host cell can be effected by, e.g., calcium phosphate transfection, virus infection, DEAE-dextran-mediated transfection, lipofectamin transfection or electroporation, and any person skilled in the art can select and use an introduction method suitable for the expression vector and host cell used.

A further aspect of the invention relates to a composition comprising a binding agent, such as an antibody or antigen-binding fragment thereof, comprising one or more ISVDs (or variants or humanized forms thereof), or part thereof, as described herein. A ‘composition’, as used herein, refers to a combination of one or more molecules, present in a formulation that retains the binding agents activity, specifically the HR2 (or S2) binding and Sarbecovirus neutralization activity in this case, thus a functional composition. The composition thus comprises one or more molecules which constitute one or more binding agents as described herein which specifically bind the Sarbecovirus Spike protein via interaction with its HR2 domain (S2 targeting binding agents or HR2 domain targeting binding agents). In particular embodiments, the composition may comprise a bivalent antibody comprising an ISVD (or variant or humanized form thereof) as described herein fused to an Fc domain, such as a binding agent, in particular an antibody comprising the amino acid sequence as defined in SEQ ID NO: 118, Said composition may be a soluble or solid composition.

In addition to said S2 targeting, in particular HR2 domain targeting, binding agent molecule(s), the composition may further comprise, for instance but not limited to, buffer components, adjuvants, or additional molecules, which may be functional molecules.

In particular embodiments, the composition may further comprise one or more binding agents capable of binding to a Sarbecovirus spike protein receptor binding domain (RBD) as described elsewhere herein. In particular embodiments, the composition may further comprise one or more binding agents, such as an antibody or antigen-binding fragment thereof, comprising one or more (such as two, three, four, or more) ISVDs (or variants or humanized forms thereof) capable of binding to a Sarbecovirus spike protein receptor binding domain (RBD) as described elsewhere herein, Said composition may thus contain at least two binding agents, characterized in that one binding agent specifically binds the HR2 domain, and the second binding agent specially binds the RBD region, resulting in a composition with at least two binding agents binding in a non-competing manner to the spike protein, possibly simultaneously.

In preferred embodiments, the binding agent capable of binding to a Sarbecovirus spike protein RBD is capable of binding two non-competing binding sites of the RBD, preferably via two different ISVDs present in said binding agent, wherein said binding agent may be a bispecific binding agent, or multispecific binding agent, More specifically, the binding agent may comprise one or more ISVDs capable of binding to or competing for the VHH72 epitope as defined herein, and one or more ISVDs capable of binding to or competing for the VHH3.117 epitope as defined herein, Non-limiting examples of binding agents comprising one or more ISVDs capable of binding to or competing for the VHH72 epitope, and one or more ISVDs capable of binding to or competing for the VHH3.117 epitope are described in PCT/EP2022/062980.

In particular embodiments, the composition may comprise (i) a binding agent, in particular an antibody or an antigen-binding fragment thereof, comprising one or more ISVDs comprising the CDRs present in SEQ ID NO:8, such as one or more ISVDs comprising or consisting of the amino acid sequence set forth in SEQ ID NO:8 (e.g. VHH R3_DC23) or a variant or humanized form thereof; and (ii) a binding agent, in particular an antibody or an antigen-binding fragment thereof, comprising one or more ISVDs comprising the CDRs present in SEQ ID NO: 125 or SEQ ID NO: 124, such as an ISVD comprising or consisting of the sequence set forth in SEQ ID NO: 125 (e.g. VHH83) or SEQ ID NO: 124 (e.g., VHH72), or a variant or a humanized form thereof, and an ISVD comprising the CDRs present in SEQ ID NO: 126, such as an ISVD comprising or consisting of the sequence set forth in SEQ ID NO: 126 (e.g. VHH3.117), or a variant or a humanized form thereof, wherein the CDRs are annotated according to Kabat, Martin, MacCallum, IMGT, AbM, or Chothia. In further particular embodiments, the composition may comprise (i) a binding agent, in particular a (bivalent) antibody, comprising an ISVD comprising the CDRs present in SEQ ID NO:8, such as an ISVD comprising or consisting of the amino acid sequence set forth in SEQ ID NO:8 (e.g. VHH R3_DC23) or a variant or humanized form thereof, fused to an Fc domain, such as an antibody comprising the amino acid sequence set forth in SEQ ID NO: 118; and (ii) a binding agent, in particular a bispecific antibody, comprising an ISVD comprising the CDRs present in SEQ ID NO: 126, such as an ISVD comprising or consisting of the sequence set forth in SEQ ID NO: 126 (e.g. VHH3.117), or a variant or a humanized form thereof, fused to the N-terminus of an Fc domain as defined herein, and an ISVD comprising the CDRs present in SEQ ID NO: 125 or SEQ ID NO: 124, such as an ISVD comprising or consisting of the sequence set forth in SEQ ID NO: 125 (e.g. VHH83) or SEQ ID NO: 124 (e.g., VHH72), or a variant or a humanized form thereof, fused to the N-terminus of the Fc domain; or the ISVD comprising the CDRs present in SEQ ID NO: 126, such as the ISVD comprising or consisting of the sequence set forth in SEQ ID NO: 126 (e.g. VHH3.117), or a variant or a humanized form thereof, may be fused to the N-terminus of the Fc domain, and the ISVD comprising the CDRs present in SEQ ID NO: 125 or SEQ ID NO: 124, such as an ISVD comprising or consisting of the sequence set forth in SEQ ID NO: 125 (e.g. VHH83) or SEQ ID NO: 124 (e.g., VHH72), or a variant or a humanized form thereof, may be fused to the C-terminus of the Fc domain, wherein the CDRs are annotated according to Kabat, Martin, MacCallum, IMGT, AbM, or Chothia.

In yet further particular embodiments, the bispecific antibody (ii) comprises or consists of the amino acid sequence set forth in SEQ ID NO: 119, or any functional variant thereof, or a variant with at least 90% identity thereof, or a humanized variant thereof.

In embodiments, the molecular ratio of the (S2 targeting) binding agent, such as an antibody or antigen-binding fragment thereof, comprising one or more ISVDs (or variants or humanized forms thereof), or part thereof, as described herein and the (S1 targeting) binding agent, such as an antibody or antigen-binding fragment thereof, comprising one or more ISVDs (or variants or humanized forms thereof) capable of binding to a Sarbecovirus spike protein receptor binding domain (RBD) in the composition may range from 3:1 to 1:3, preferably from 2:1 to 1:2, more preferably the molecular ratio is about 1:1.

Moreover said composition may still contain additional binding agent(s) or molecules, which optionally bind further binding regions on the same or different epitopes of the spike protein, or other viral proteins, or may even target totally unrelated target proteins.

A further aspect of the invention relates to medicaments or pharmaceutical compositions comprising a binding agent, in particular an antibody or antigen-binding fragment, a nucleic acid encoding it, and/or a (recombinant) vector comprising the nucleic acid, and/or a composition comprising a binding agent, in particular an antibody or antigen-binding fragment, as described herein. In particular, a pharmaceutical composition is a pharmaceutically acceptable composition; such compositions are preferably further comprising a (pharmaceutically) suitable or acceptable carrier, diluent, adjuvant, excipient, stabilizer, etc.

By “pharmaceutically acceptable” is meant a material that is not biologically or otherwise undesirable, i.e., the material may be administered to an individual along with the compound, in particular the Sarbecovirus binding agent, more particularly the Sarbecovirus antibody or antigen-binding fragment, without causing any undesirable biological effects or interacting in a deleterious manner with any of the other components of the pharmaceutical composition in which it is contained, A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. The term “excipient”, as used herein, is intended to include all substances which may be present in a pharmaceutical composition and which are not active ingredients but may contribute to e.g. long-term stability, or therapeutic enhancement on the active ingredient (such as by facilitating drug absorption, reducing viscosity, or enhancing solubility), Excipients may include, for example, salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, such as in particular a “pharmaceutically acceptable diluent”, includes vehicles such as water, saline, physiological salt solutions, glycerol, ethanol, etc.

Auxiliary substances such as wetting or emulsifying agents, pH buffering substances, preservatives may be included in such vehicles.

A pharmaceutically effective amount of binding agents, in particular antibodies or antigen-binding fragments, of the invention is preferably that amount which produces a result or exerts an influence on the particular condition being treated.

The pharmaceutical composition of this invention can be lyophilized for storage and reconstituted in a suitable carrier prior to use. When prepared as lyophilization or liquid, physiologically acceptable carrier, excipient, stabilizer need to be added into the pharmaceutical composition of the invention (Remington's Pharmaceutical Sciences 22nd edition, Ed, Allen, Loyd V, Jr, (2012). The preparation containing pharmaceutical composition of this invention should be sterilized before injection. This procedure can be done using sterile filtration membranes before or after lyophilization and reconstitution. The pharmaceutical composition can be packaged in a container or vial with sterile access port, such as an i.v. solution bottle with a rubber stopper—the pharmaceutical composition can be present as liquid, or the container or vial is filled with a liquid pharmaceutical composition that is subsequently lyophilized or dried; or can be packaged in a pre-filled syringe.

A further aspect of the invention relates to a binding agent, in particular an antibody or antigen-binding fragment, a nucleic acid encoding it as described herein, a vector comprising such nucleic acid, a composition comprising a binding agent, in particular an antibody or antigen-binding fragment, as described herein or a pharmaceutical composition comprising a binding agent, in particular an antibody or antigen-binding fragment, nucleic acid encoding it, a (recombinant) vector comprising such nucleic acid, and/or a composition comprising a binding agent, in particular an antibody or antigen-binding fragment, as described herein, for use as a medicine or medicament.

Alternatively, use of a binding agent, in particular an antibody or antigen-binding fragment, nucleic acid encoding it, a vector comprising such nucleic acid as described herein, or a composition comprising a binding agent, in particular an antibody or antigen-binding fragment, as described herein, or use of a pharmaceutical composition comprising a binding agent, in particular an antibody or antigen-binding fragment, nucleic acid encoding it, a vector comprising such nucleic acid, and/or a composition comprising a binding agent, in particular an antibody or antigen-binding fragment, as described herein, in the manufacture of a medicine or medicament is envisaged.

In particular, the binding agent, in particular the antibody or antigen-binding fragment, the nucleic acid encoding it, the vector comprising such nucleic acid or the composition comprising the binding agent, in particular the antibody or antigen-binding fragment, as described herein, or the medicament or pharmaceutical composition comprising a binding agent, in particular an antibody or antigen-binding fragment, a nucleic acid encoding it, a (recombinant) vector comprising such nucleic acid, and/or a composition comprising a binding agent, in particular an antibody or antigen-binding fragment, as described herein, is for use in passive immunisation, for use in treating a subject with a Sarbecovirus infection, for use in preventing infection of a subject with a Sarbecovirus, or for use in protecting a subject from infection with a Sarbecovirus.

When for use in passive immunisation, the subject may have an infection with a Sarbecovirus (therapeutic passive immunisation) or may not have an infection with a Sarbecovirus (prophylactic passive immunisation).

A related aspect relates to methods for treating a subject suffering from/having/that has contracted an infection with a Sarbecovirus, the methods comprising administering a binding agent, in particular an antibody or antigen-binding fragment, a nucleic acid encoding it a (recombinant) vector comprising such nucleic acid, or a composition comprising a binding agent, in particular an antibody or antigen-binding fragment, as described herein to the subject, or comprising administering a medicament or pharmaceutical composition comprising a binding agent, in particular an antibody or antigen-binding fragment, a nucleic acid encoding it, a (recombinant) vector comprising such nucleic acid and/or a composition comprising a binding agent, in particular an antibody or antigen-binding fragment, as described herein to the subject.

A further aspect of the invention relates to methods for protecting a subject from infection with a Sarbecovirus or for preventing infection of a subject with a Sarbecovirus, the methods comprising administering a binding agent, in particular an antibody or antigen-binding fragment, a nucleic acid encoding it, a (recombinant) vector comprising such nucleic acid, or a composition comprising a binding agent, in particular an antibody or antigen-binding fragment, as described herein to the subject prior to infection, or comprising administering a medicament or pharmaceutical composition as described herein comprising a binding agent, in particular an antibody or antigen-binding fragment, a nucleic acid encoding it, a (recombinant) vector comprising such nucleic acid, and/or a composition comprising a binding agent, in particular an antibody or antigen-binding fragment, to the subject prior to infection.

In the above medical aspects, a nucleic acid encoding a binding agent, in particular an antibody or antigen-binding fragment or a (recombinant) vector comprising such nucleic acid as described herein can be used in e.g. gene therapy setting, “Gene therapy” as used herein refers to therapy performed by the administration to a subject of an expressed or expressible nucleic acid. For such applications, the nucleic acid molecule or vector as described herein allow for production of the binding agent, antibody or antibody fragment within a cell. A large set of methods for gene therapy are available in the art and include, for instance (adeno-associated) virus-mediated gene silencing, or virus-mediated gene therapy (e.g. US20040023390; Mendell et al 2017, N Eng J Med 377:1713-1722). A plethora of delivery methods are well known to those of skill in the art and include but are not limited to viral delivery systems, microinjection of DNA plasmids, biolistics of naked nucleic acids, use of a liposome or an artificial exosome, administration of the nucleic acid or vector formulated in a nanoparticle or lipid or lipid-comprising particle. In vivo delivery by administration to an individual patient occurs typically by systemic administration (e.g., intravenous, intraperitoneal infusion or brain injection; e.g. Mendell et al 2017, N Eng J Med 377:1713-1722).

A “therapeutically active agent” generally means any molecule that has or may have a therapeutic effect (i.e. curative or prophylactic effect) in the context of treatment of a disease, Preferably, a therapeutically active agent is a disease-modifying agent, which can be a cytotoxic agent, such as a toxin, or a cytotoxic drug, or an enzyme capable of converting a prodrug into a cytotoxic drug, or a radionuclide, or a cytotoxic cell, or which can be a non-cytotoxic agent, Even more preferably, a therapeutically active agent has a curative effect on the disease. The binding agent, in particular the antibody or antibody fragment, or pharmaceutical composition of the invention may act as a therapeutically active agent, when beneficial in treating patients infected with a Sarbecovirus, such SARS-COV-2 or SARS-COV-1 or patients suffering from COVID-19. The binding agent, in particular the antibody or antibody fragment, may comprise a variant of the Sarbecovirus-binding ISVDs as described herein, preferably an improved variant binding to the same binding region of the HR2 domain, and more preferably a humanized variant thereof, and may contain or be coupled to additional functional groups, advantageous when administrated to a subject, Examples of such functional groups and of techniques for introducing them will be clear to the skilled person, and can generally comprise all functional groups and techniques mentioned in the art as well as the functional groups and techniques known per se for the modification of pharmaceutical proteins, and in particular for the modification of antibodies or antibody fragments, for which reference is for example made to Remington's Pharmaceutical Sciences, 16th ed., Mack Publishing Co., Easton, PA (1980). Such functional groups may for example be linked directly (for example covalently) to the ISVD or active antibody fragment, or optionally via a suitable linker or spacer, as will again be clear to the skilled person, One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). For example, for this purpose, PEG may be attached to a cysteine residue that naturally occurs in an immunoglobulin single variable domain described herein (or a variant or a humanized form thereof as described herein), an immunoglobulin single variable domain as described herein (or a variant or a humanized form thereof as described herein) may be modified so as to suitably introduce one or more cysteine residues for attachment of PEG, or an amino acid sequence comprising one or more cysteine residues for attachment of PEG may be fused to the N- and/or C-terminus of an ISVD or active antibody fragment as described herein (or a variant or a humanized form thereof as described herein), all using techniques of protein engineering known per se to the skilled person. Another, usually less preferred modification comprises N-linked or O-linked glycosylation, usually as part of co-translational and/or post-translational modification, depending on the host cell used for expressing the antibody or active antibody fragment. Another technique for increasing the half-life of a binding domain, in particular an antibody or antibody fragment, may comprise the engineering into bifunctional or bispecific domains (for example, one ISVD or active antibody fragment against the target Sarbecovirus HR2 domain and one against a scrum protein such as albumin or Staphylococcal protein A (SpA)—which is a surface protein abundantly present in the lungs aiding in prolonging half-life)) or into fusions of antibody fragments, in particular immunoglobulin single variable domains, with peptides (for example, a peptide against a serum protein such as albumin). In yet another example, an ISVD as described herein (or a variant or humanized form thereof as described herein) can be fused to an immunoglobulin Fc domain as described elsewhere herein, Examples are further shown in the experimental section and are also depicted in the sequence listing. In embodiments, in the above medical aspects, the Sarbecovirus is SARS-COV-2 such as a SARS-COV-2 variant, or SARS-COV-1The SARS-COV-2 variant may be a variant at position N439, K417, S477, L452, T478, E484, P384, N501 and/or D614 (relative to the SARS-COV-2 spike amino acid sequence as defined in SEQ ID NO:86), more particularly a variant at position N501 such as a N501Y variant (e.g. SARS-COV-2 Alpha variant), a variant at position N501 and E484 such as a N501Y and E484K variant (e.g. SARS-COV-2 Alpha+E484K variant), a variant at position K417, E484 and N501 such as a K417N, E484K and N501Y variant (e.g. SARS-CoV-2 beta variant), a variant at position P384, K417, E484 and N501 such as a P384L, K417N, E484K and N501Y variant (e.g. SARS-COV-2 beta+P384L variant), a variant at position L452 and E484 such as a L452R and E484Q variant (e.g. SARS-COV-2 kappa variant), a variant at position L452 and T478 such as a L452R and T478K variant (e.g. SARS-COV-2 delta variant), a variant at position L452 such as a L452R variant (e.g. SARS-COV-2 epsilon variant), a variant at position K417 such as a K417T variant (e.g. SARS-COV-2 gamma variant) or a variant at position D614 such as a D614G variant (e.g. SARS-COV-2 Omicron variant or SARS-COV-2 BA.1 variant). In particular embodiments, the Sarbecovirus is any one or both of SARS-COV-2 and SARS-COV-2. In further particular embodiments, SARS-COV-2 is SARS-COV-2 Wuhan strain or a SARS-COV-2 variant, in particular a SARS-COV-2 variant selected from the group consisting of SARS-COV-2 Alpha variant. SARS-COV-2 Omicron BA.1 variant and SARS-COV-2 Omicron BA.2 variant.

As used herein, the terms “therapy” or “treatment” refer to the alleviation or measurable lessening of one or more symptoms or measurable markers of a pathological condition such as a disease or disorder, in particular a Sarbecovirus infection, Measurable lessening includes any statistically significant decline in a measurable symptom or marker, Generally, the terms encompass both curative treatments and treatments directed to reduce symptoms and/or slow progression of the disease. The terms encompass both the therapeutic treatment of an already developed pathological condition, in particular a Sarbecovirus infection, as well as prophylactic or preventative measures, wherein the aim is to prevent or lessen the chances of incidence of a pathological condition, in particular a Sarbecovirus infection, Beneficial or desired clinical results include, but are not limited to, prevention of a disease, reduction of the incidence of a disease, alleviation of symptoms associated with a disease, diminishment of extent of a disease, stabilisation of the disease, delay or slowing of the progression of a disease, amelioration or palliation of a disease, or combinations thereof. In certain embodiments, the terms may relate to therapeutic treatments. In certain other embodiments, the terms may relate to preventative treatments.

For example, treatment may refer to passive immunisation of a subject having contracted a Sarbecovirus infection (therapeutic treatment), Prevention of infection with a Sarbecovirus may be useful in case of e.g. epidemic or pandemic conditions during which subjects known to be most vulnerable to develop severe disease symptoms can be prophylactically treated (preventive or prophylactic immunisation) with a binding agent, in particular an antibody or antigen-binding fragment or a nucleic acid encoding it or a vector comprising such nucleic acid or a composition comprising a binding agent, in particular an antibody or antigen-binding fragment as described herein such as to prevent infection overall, or such as to prevent development or occurrence of severe disease symptoms.

In embodiments, a therapeutically effective amount of a binding, in particular an antibody or antigen-binding fragment, nucleic acid, vector or pharmaceutical composition is administered to a subject in need thereof. In other embodiments, a prophylactically effective amount of a binding agent, in particular an antibody or antigen-binding fragment, nucleic acid, vector or pharmaceutical composition is administered to a subject in need thereof. A “therapeutically effective amount” or “therapeutically effective dose” indicates an amount of binding agent, in particular antibody or antigen-binding fragment, nucleic acid, vector or pharmaceutical composition that when administered to the subject brings about a clinical positive response with respect to therapeutic treatment of the subject afflicted by a Sarbecovirus infection, such as, e.g. curing infection with a Sarbecovirus. Similarly, a “prophylactically effective amount” or “prophylactically effective dose” refers to an amount of binding agent, in particular antibody or antigen-binding fragment, nucleic acid, vector or pharmaceutical composition that prevents, inhibits or delays the onset of a Sarbecovirus infection and/or prevents or reduces the risk of a clinical manifestation of a Sarbecovirus infection and/or reduces the severity, symptoms and/or duration of a Sarbecovirus infection in the subject. In order to achieve the therapeutic effect or the preventive or prophylactic effect, the binding agent, in particular the antibody or antigen-binding fragment or the nucleic acid encoding it or the vector comprising such nucleic acid or the composition comprising the binding agent, in particular the antibody or antigen-binding fragment as described herein may need to be administered to a subject multiple times, such as with an interval of 1 week or 2 weeks; the interval being dictated by the pharmacokinetic behaviour or characteristics (e.g. half-time or half-life in the subject's circulation) of the binding agent, in particular the antibody or antigen-binding fragment, nucleic acid or vector. Alternatively, therapeutic treatments and prophylactic treatments in which a single dose of a binding agent, in particular an antibody or antigen-binding fragment as described herein is administered to the subject is envisaged. The single dose may be in the range of 0.5 mg/kg to 25 mg/kg.

The term “subject”, “individual” or “patient”, used interchangeably herein, relates to any organism such as a vertebrate, particularly any mammal, including both a human and other mammals, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate, Preferably, the subject is a human. In particular embodiments, a subject is a subject, such as a human subject, with or suspected of having an infection with a Sarbecovirus, also designated “patient” or “subject” herein. However, it will be understood that the aforementioned terms do not imply that symptoms are present. In particular embodiments, the subject is a mammal susceptible to infection with a Sarbecovirus, such as a human subject that is susceptible to infection with SARS-COV-2 such as a SARS-COV-2 variant, or SARS-COV-1.

The pharmaceutical composition of the invention can be administered to any patient in accordance with standard techniques. The administration can be by any appropriate mode, including oral, parenteral, topical, nasal, ophthalmic, intrathecal, intra-cerebroventricular, sublingual, rectal, vaginal, and the like, Still other techniques of formulation such as nanotechnology and aerosol and inhalant are also within the scope of this invention. The dosage and frequency of administration will depend on the age, sex and condition of the patient, concurrent administration of other drugs, counter-indications and other parameters to be taken into account by the clinician.

In particular embodiments of the herein described medical aspects, the binding agent, in particular the antibody or antigen-binding fragment, the nucleic acid, the vector or the pharmaceutical composition may be administered to a subject via intravenous injection, subcutaneous injection, or intranasally, or, alternatively via inhalation or pulmonary delivery.

A further aspect of the invention relates to a binding agent, in particular an antibody or antigen-binding fragment, as described herein for use in diagnosing a Sarbecovirus infection, for use as a diagnostic agent. A nucleic acid encoding a Sarbecovirus binding agent, in particular a Sarbecovirus antibody or antigen-binding fragment as described herein, a (recombinant) vector comprising such nucleic acid, or a composition comprising a Sarbecovirus binding agent, in particular a Sarbecovirus antibody or antigen-binding fragment as described herein, can likewise be for use.

Use of a binding agent, in particular an antibody or antigen-binding fragment, as described herein in the manufacture of a (in vitro) diagnostic agent or diagnostic kit is also envisaged. In particular, the binding agent, in particular the antibody or antigen-binding fragment, as described herein may be for use in detecting the presence (or absence) of a Sarbecovirus or a part thereof (such as a Sarbecovirus spike protein or a part thereof) in a sample, such as a sample obtained from a subject, such as from a subject suspected to be infected with a Sarbecovirus. A nucleic acid encoding a binding agent, in particular an antibody or antigen-binding fragment, as described herein, a (recombinant) vector comprising such nucleic acid or composition comprising a binding agent, in particular an antibody or antigen-binding fragment, as described herein can likewise be used in the manufacture of a diagnostic agent or diagnostic kit, such as an in vitro diagnostic agent or kit.

A further aspect relates to methods for detecting a Sarbecovirus in a sample, such as a sample obtained from a subject, such as from a subject suspected to be infected with a Sarbecovirus. Such methods usually comprise the steps of obtaining a sample, contacting the sample with a binding agent, in particular an antibody or antigen-binding fragment, as described herein, and detecting, determining, assessing, assaying, identifying or measuring binding of the binding agent, in particular the antibody or antigen-binding fragment, with a Sarbecovirus or a part thereof (such as a Sarbecovirus spike protein or a part thereof).

In particular embodiments of the herein described diagnostic aspects, the Sarbecovirus is selected from the group of clade 1a, 1b, 2 and/or clade 3 Sarbecoviruses, such as SARS-Cov-2, GD-Pangolin, RaTG13, WIV1, LYRal1, RsSHC014, Rs7327, SARS-COV-1, Rs4231, Rs4084, Rp3, HKU3-1, or BM48-31 viruses, preferably SARS-COV-2 such as a SARS-COV-2 variant or SARS-COV-1.

In embodiments of the herein described diagnostic aspects, the binding agent, in particular the antibody or antibody fragment, as described herein is comprising a detectable moiety fused to it, bound to it, coupled to it, linked to it, complexed to it, or chelated to it. A “detectable moiety” in general refers to a moiety that emits a signal or is capable of emitting a signal upon adequate stimulation, or to a moiety that is capable of being detected through binding or interaction with a further molecule (e.g. a tag, such as an affinity tag, that is specifically recognized by a labelled antibody), or is detectable by any means (preferably by a non-invasive means, if detection is in vivo/inside the human body), Furthermore, the detectable moiety may allow for computerized composition of an image, as such the detectable moiety may be called an imaging agent, Detectable moieties include, without limitation, fluorescence emitters, phosphorescence emitters, positron emitters, radioemitters, etc., enzymes (capable of measurably converting a substrate) and molecular tags.

Examples of radioemitters/radiolabels include 68Ga, 110mIn, 18F, 45Ti, 44Sc, 47Sc, 61Cu, 60Cu, 62Cu, 66Ga, 64Cu, 55Ca, 72As, 86Y, 90Y, 89Zr, 125I, 74Br, 75Br, 76Br, 77Br, 78Br, 111 In, 114mIn, 114 In, 99mTc, 11C, 32Cl, 33Cl, 34Cl, 123I, 124I, 131I, 186Rc, 188Rc, 177Lu, 99Tc, 212Bi, 213Bi, 212Pb, 225Ac, 153Sm, and 67Ga, Fluorescence emitters include, without limitation, cyanine dyes (e.g. Cy5, Cy5.5, Cy7, Cy7.5), FITC, TRITC, coumarin, indolenine-based dyes, benzoindolenine-based dyes, phenoxazines, BODIPY dyes, rhodamines, Si-rhodamines, Alexa dyes, and derivatives of any thereof, Non-limiting examples of molecular tags include affinity tags, such as chitin binding protein (CBP), maltose binding protein (MBP), glutathione-S-transferase (GST), poly (His) (e.g., 6× His or His6), biotin or streptavidin, such as Strep-tag R, Strep-tag IIR and Twin-Strep-tag®; solubilizing tags, such as thioredoxin (TRX), poly (NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.); luminescent labels or tags, such as luciferase, bioluminescent or chemiluminescent compounds (such as luminal, isoluminol, theromatic acridinium ester, imidazole, acridinium salts, oxalate ester, dioxetane or GFP and its analogs); phosphorescent labels; a metal chelator; and (other) enzymatic labels (e.g., peroxidase, alkaline phosphatase, beta-galactosidase, urease or glucose oxidase).

Binding agents, in particular antibodies and antibody fragments, as described herein and comprising a detectable moiety may for example be used for in vitro, in vivo or in situ assays (including immunoassays known per se such as ELISA, RIA, EIA and other “sandwich assays”, etc.) as well as in vivo imaging purposes, depending on the choice of the specific label.

A further aspect relates to kits comprising a binding agent, in particular an antibody or antigen-binding fragment, a nucleic acid encoding it, a vector comprising such nucleic acid as described herein, a composition comprising a binding agent, in particular an antibody or antigen-binding fragment, or a pharmaceutical composition comprising a binding agent, in particular an antibody or antigen-binding fragment; a nucleic acid encoding it a vector comprising such nucleic acid or a composition comprising a binding agent, in particular an antibody or antigen-binding fragment as described herein.

Such kits may be pharmaceutical kits or medicament kits which are comprising a container or vial (any suitable container or vial, such as a pharmaceutically acceptable container or vial) comprising an amount of binding agent, in particular antibody or antigen-binding fragment, or nucleic acid encoding it or vector comprising such nucleic acid as described herein or composition comprising a binding agent, in particular an antibody or antigen-binding fragment as described herein, and further comprising e.g. a kit insert such as a medical leaflet or package leaflet comprising information on e.g. intended indications (prophylactic or therapeutic treatment of a Sarbecovirus infection) and potential side-effects, Pharmaceutical kits or medicament kits may further comprise e.g. a syringe for administering the binding agent, in particular the antibody or antigen-binding fragment, nucleic acid encoding it vector comprising such nucleic acid or composition comprising the binding agent, in particular the antibody or antigen-binding fragment as described herein to a subject.

Such kits may also be diagnostic kits comprising a container or vial (any suitable container or vial, such as a pharmaceutically acceptable container or vial) comprising an amount of binding agent, in particular antibody or antigen-binding fragment, as described herein, such as a binding agent, in particular an antibody or antigen-binding fragment thereof comprising a detectable moiety. Such diagnostic kits may further comprise e.g. one or more reagents to detect the detectable moiety and/or e.g. instructions on how to use said binding agent, in particular antibody or antigen-binding fragment, for detection of a Sarbecovirus in a sample.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

Certain aspects and embodiments of the present invention are set forth in the below numbered statements:
(1) A binding agent capable of neutralizing a Sarbecovirus, characterized in that said binding agent specifically binds to a region of heptad repeat 2 (HR2) domain of spike protein of the Sarbecovirus proximal to the viral membrane.
(2) The binding agent according to (1), wherein said binding agent specifically binds to or within a region of the HR2 domain located from amino acid 11179 to amino acid E1202, preferably from amino acid D1184 to amino acid E1202, more preferably from amino acid V1189 to amino acid E1202 of the SARS-COV-2 spike protein as defined in SEQ ID NO:86.
(3) The binding agent according to (1) or (2), wherein said binding agent specifically binds to a region of the HR2 domain corresponding to the region from amino acid N1192 to amino acid Q1201 of the SARS-COV-2 spike protein as defined in SEQ ID NO:86.
(4) The binding agent according to any one of (1) to (3), wherein:
   said binding agent is capable of neutralizing the Sarbecovirus with a 50% inhibitory concentration (IC₅₀) of 100 ng/ml or less, preferably 10 ng/ml or less, more preferably 1 ng/ml or less, as determined in a Sarbecovirus spike protein pseudovirus neutralization assay such as a vesicular stomatitis virus (VSV)-Sarbecovirus spike protein pseudovirus neutralization assay;
   said binding agent is capable of neutralizing any one or both of SARS-COV-2 such as one or more of SARS-COV-2 Wuhan strain, SARS-COV-2 Alpha variant, SARS-COV-2 Omicron BA.1 variant, and SARS-COV-2 Omicron BA.2 variant; and SARS-COV-1;
   said binding agent is capable of inhibiting spike-mediated syncytia formation between cells expressing the Sarbecovirus spike protein and cells expressing the angiotensin-converting enzyme 2 (ACE2) receptor; and/or
   said binding agent does not bind a Middle East respiratory syndrome coronavirus (MERS-COV).
(5) The binding agent according to any one of (1) to (4), which comprises or consists of an antibody or an antibody fragment.
(6) The binding agent according to any one of (1) to (5), which comprises an immunoglobulin single variable domain (ISVD), preferably a VHH.
(7) The binding agent according to (6), wherein the ISVD comprises a complementarity determining region 1 (CDR1) defined by any one of SEQ ID NOs: 63, 46, 69 or 77, a complementarity determining region 2 (CDR2) defined by any one of SEQ ID NOs: 64, 47, 70, 73 or 78, and a complementarity determining region 3 (CDR3) defined by any one of SEQ ID NOs: 48, 67, 74 or 79; preferably a CDR1 defined by any one of SEQ ID NOs: 65, 71, 49, or 80, a CDR2 defined by any one of SEQ ID NOs: 66, 72, 50, 75, or 81, and a CDR3 defined by any one of SEQ ID NOs: 51, 68, 76, or 82
(8) The binding agent according to (6) or (7), wherein the ISVD comprises a CDR1, CDR2 and CDR3, each independently as present in any of SEQ ID NOs: 1 to 10, wherein the CDR1, CDR2 and CDR3 are annotated according to any one of Kabat, MacCallum, IMGT, AbM, Martin or Chothia; preferably wherein the ISVD comprises a combination of CDR1, CDR2 and CDR3, wherein the CDR1, CDR2 and CDR3 are as present in a particular one of the sequences set forth in SEQ ID NOs: 1 to 10, wherein the CDR1, CDR2 and CDR3 are annotated according to any one of Kabat, MacCallum, IMGT, AbM, Martin or Chothia.
(9) The binding agent according to any one of (6) to (8), wherein the ISVD comprises a CDR1 defined by SEQ ID NO:63, a CDR2 defined by SEQ ID NO:64, and a CDR3 defined by SEQ ID NO:48; preferably a CDR1 defined by SEQ ID NO:65, a CDR2 defined by SEQ ID NO:66, and a CDR3 defined by SEQ ID NO:51; more preferably a CDR1 defined by any one of SEQ ID NO:52-54, a CDR2 defined by any one of SEQ ID NO:55-62, and a CDR3 defined by any one of SEQ ID NO:21-27
(10) The binding agent according to any one of (7) to (9), wherein the ISVD comprises an amino acid sequence with at least 90% identity to an amino acid sequence selected from the group of SEQ ID NO: 1 to 10.
(11) The binding agent according to any one of (1) to (10), comprising an ISVD comprising an amino acid sequence selected from the group of SEQ ID NO: 1 to 10.
(12) The binding agent according to any one of (1) to (11), which is in a multivalent form, preferably wherein the binding agent comprises an ISVD fused to an Fc domain.
(13) A nucleic acid molecule comprising a polynucleotide sequence encoding the binding agent according to any one of (1) to (12); a vector comprising said nucleic acid molecule; or a cell expressing the binding agent according to any one of (1) to (12) or comprising said nucleic acid molecule or said vector.
(14) A pharmaceutical composition comprising the binding agent according to any one of (1) to (12), the nucleic acid molecule according to (13), or the vector according to (13), and a pharmaceutically acceptable carrier; or a kit such as a diagnostic kit comprising the binding agent according to any one of (1) to (12).
(15) The binding agent according to any one of (1) to (12), the nucleic acid molecule according to (13), the vector according to (13), the pharmaceutical composition according to (14), or the kit according to (14) for use in medicine; preferably for use in the prevention or treatment of a Sarbecovirus infection in a subject, or for use in the diagnosis of a Sarbecovirus infection in a subject.
(16) An in vitro or ex vivo method for detecting a Sarbecovirus in a sample comprising:
   contacting the sample with a binding agent according to any one of (1) to (12), and
   determining binding of the binding agent with a Sarbecovirus or a part thereof.

The herein disclosed aspects and embodiments of the invention are further supported by the following non-limiting examples.

### EXAMPLES

### Material and Methods

### Isolation SARS-COV-2 VHH Phages

To obtain SARS-COV-1 and SARS-COV-2 cross-reactive VHHs, a llama that was previously immunized with recombinant prefusion stabilized SARS-COV-1 and MERS-COV spike protein was additionally immunized 3 times with recombinant SARS-COV-2 spike protein (S-2P) stabilized in its prefusion conformation (Wrapp, D, et al. (2020) Structural Basis for Potent Neutralization of Betacoronaviruses by Single-Domain Camelid Antibodies, Cell 181:1004-1015,e15; Wrapp et al. (2020) Cryo-EM structure of the 2019-nCOV spike in the prefusion conformation, Science 367:1260-1263), After the immunization, peripheral blood lymphocytes were isolated from the llama and an immune VHH-displaying phagemid library was constructed, Phages displaying SARS-COV-2 specific VHHs were enriched from this phage library by 2 rounds of biopanning on 100 ng of of His-tagged SARS-COV-2 spike 6P protein (Hsich et al. (2020) Structure-based design of prefusion-stabilized SARS-COV-2 spikes, Science 369:1501-1505), that was immobilized on a well of a microtiter plate (type II, F96 Maxisorp, Nunc) via coated anti-His antibodies in the presence of 10 μg/ml RBD-SD1-mouse IgG (Sionobiological). In addition, two additional rounds of biopanning were performed using anti-His captured spike proteins (R3_C and R4_C series) or using directly coated spike proteins (R3_DC and R4_DC series), Also in these two series of additional rounds biopanning was performed in the presence of 10 μg/ml RBD-SD1-mouse IgG (Sionobiological). For each panning round an uncoated well was used as a negative control. The wells were then washed 5 times with phosphate-buffered saline (PBS)+0.05% Tween 20 and blocked with 4% milk powder in PBS SEA BLOCK blocking buffer (Thermo Scientific) in the first panning round, Pierce protein free blocking buffer (Thermo Scientific) in the second round, SEA BLOCK blocking buffer (Thermo Scientific) in the third round and 1% BSA in the fourth round, Non-specifically bound phages were removed by extensive washing with PBS+0.05% Tween 20. The retained phages were eluted with TEA-solution (14% trimethylamine (Sigma), pH 10) and subsequently neutralized with 1 M Tris-HCl, pH 8. The collected phages were amplified in exponentially growing E. coli TGI cells, infected with VCS M13 helper phages and subsequently purified using PEG 8,000/NaCl precipitation for the next round of selection, Enrichment after each panning round was determined by infecting TGI cells with 10-fold serial dilutions of the collected phages after which the bacteria were plated on LB agar plates with 100 μg/mL-ampicillin and 1% glucose.

### Preparation of Periplasmic Extracts (PE)

After 3 or 4 panning rounds individual colonies of phage-infected bacteria were randomly selected for further analysis. The individual colonies were inoculated in 2 mL of terrific broth (TB) medium with 100 μg/mL ampicillin in 24-well deep well plates, After growing individual colonies for 5 h at 37° C., isopropyl β-D-1-thiogalactopyranoside (IPTG) (1 mM) was added to induce VHH expression during overnight incubation at 37° C. To prepare periplasmic extract, the bacterial cells were pelleted and resuspended in 250 μL TES buffer (0.2 M Tris-HCl pH 8, 0.5 mM EDTA, 0.5 M sucrose) and incubated at 4° C. for 30 min, Subsequently 350 μL water was added to induce an osmotic shock, After 1 h incubation at 4° C., followed by centrifugation, the periplasmic extract was collected.

### Periplasmic Extract Enzyme-Linked Immunosorbent Assay

Wells of half-well microtiter plates were coated overnight at 4° C. with 50 ng of recombinant SARS-CoV-2 S-2P protein, SARS-COV-2 S2 subunit (AcroBiosystems, S2N-C52H5), SARS-COV-2 RBD (Sinobiologicals), SARS-COV S, MERS-COV S, HKU1 S and BSA. The plates were blocked with 5% milk powder in PBS, Periplasmic extract was diluted 1/10 in PBS and were added to blocked wells, Binding of VHHs was detected with mouse anti-HA antibody (BioLegend 901501, 1/2000), followed by anti-mouse IgG-HRP (GE Healthcare, NA931V, 1/2000), After washing, 50 μl of TMB substrate (tetramethylbenzidine, BD OptEIA) was added to the plaates and the reaction was stopped by addition of 50 μl 1 M H2SO4. The absorbance at 450 nm was measured with an iMark Microplate Absorbance Reader (Bio Rad), Curve fitting was performed using nonlinear regression (Graphpad 8.0)

### Periplasmic Extract-Pseudovirus Neutralization Assay

Pseudoviruses expressing the SARS-COV-2 spike (D614G) were incubated for 30 min at 37° C. with a 1/100 dilution of periplasmic extract in Fluorobrite DMEM medium (Invitrogen), supplemented with 5% heat-inactivated FBS, 1% penicillin, 1% streptomycin, 2 mM L-glutamine, non-essential amino acids (Invitrogen) and 1 mM sodium pyrovate. The incubated pseudoviruses were subsequently added to subconfluent monolayers of Vero E6 from which the original growth medium was removed, Sixteen hours later, the cells were lysed using passive lysis buffer (Promega). The transduction efficiency was quantified by measuring the GFP fluorescence in the prepared cell lysates using a Tecan infinite 200 pro plate reader, GFP fluorescence was normalized using the GFP fluorescence of non-infected cells and infected cells treated with PBS,

### Cell Lines

FreeStyle293F cells (ThermoFisher Scientific) and HEK293-S cells (ThermoFisher Scientific) were cultured in FreeStyle 293 expression media (Life Technologies), at 37° C. with 8% CO₂ while shaking at 130 rpm, HEK293-T cells (ATCC) and Vero E6 cells (ATCC) were cultured at 37° C. in the presence of 5% CO₂ in DMEM supplemented with 10% heat-inactivated fetal bovine serum (FBS), 1% penicillin, 1% streptomycin, 2 mM 1-glutamine, non-essential amino acids (Invitrogen) and 1 mM sodium pyruvate, ExpiCHO-S cells (GIBCO) were cultured at 37° C. with 8% CO₂ while shaking at 130 rpm in ExpiCHO expression media (GIBCO), Vero E6-TMPRSS2 cells that stably express human TMPRSS2 (NIBIOHN, JCRB1819) (Matsuyama et al., PNAS, 2020) were cultured in DMEM containing 10% FBS, penicillin (100 unit/mL), streptomycin (100 μg/mL), Geneticin (G418) (1 mg/ml), When Vero E6-TMPRSS2 cells were seeded for assays medium without Geneticin was used.

Raji cells and Raji cells that stably express the SARS-COV-2 spike protein were cultured at 37° C. with 5% CO₂ in RPMI-1640 medium supplemented with 10% FCS, 0.1 μg/ml puromycin, 1% penicillin and 1% streptomycin.

### Sotrovimab, Cilgavimab, Bebtelovimab and Palivizumab

Bebtelovimab Biosimilar (PX-TA1750), cilgavimab Biosimilar (PX-TA1033) and sotrovimab Biosimilar (PX-TA1637) were commercially purchased from Proteogenix, Clinical grade Palivizumab was obtained from the Ghent University hospital.

Generation of R3_DC23-Fc(YTE) (Also Referred to Herein as huR3DC23-Fc) (SEQ ID NO:96)

A humanized (QID, Q5V, A14P, D16G, T19R, M63V, S73N, D79Y, T82cL, K83R and Q108L, according to Kabat numbering; the T82cL modification in particular serves to inactivate the glycosylation of the N82a position, and can be useful in both humanized and non-humanized version, for expression in mammalian cells) version of R3_DC23 was fused via a (G₄S)ₙ linker to a human IgG1 Fc (EPKSCdel_YTE_K447del) ordered synthetically at IDT as gBlocks, Upon arrival, gBlocks were solubilized in ultraclean water at a concentration of 20 ng/μL, gBlocks were A-tailed using the NEBNext-dA-tailing module (NEB), purified using CleanPCR magnetic beads (CleanNA) and inserted in pcDNA3.4-TOPO vector (ThermoFisher). The ORF of positive clones was fully sequenced, and pDNA of selected clones was prepared using the NucleoBond Xtra Midi kit (Machery-Nagel).

Transient Production of huR3DC23-Fc LS (Also Referred to Herein as R3_DC23Hum-Fc(LS) or XVR013) (SEQ ID NO:118)

The gene encoding huR3DC23-Fc_LS was codon optimized, synthesized, and cloned into the pXLG6 backbone vector at ATUM's laboratories, Upon gene and codon optimization the R3DC23 DNA sequence was inserted into pXLG6 expression vector and transfected in CHOExpress™ cells at a cell density of 4.00E+6 cells/ml, TGE supernatant was harvested by centrifugation and clarified by filtration (0.2 μm) after 10 days when cell viability dropped below 10%. The protein was further purified by Protein A.

### Production VHH73 S56A, GBP, CB6 and S309

Production of VHH73_S56A, GBP, CB6 and S309 was performed as described in Schepens et al. (Schepens et al. (2021) Sci. Transl Med. 13: eabi7826).

### HEK S Transfection and Protein Purification Protocol; Production of YTE Variants of VHH-Fc in Mammalian Cells

HEK293-S cells were transfected with VHH-Fc(S) encoding plasmids using polyethylenimine (PEI).

Briefly, suspension-adapted and serum-free HEK293-S cells were seeded at 3×10⁶ cells/mL in FreeStyle 293 medium (ThermoFisher Scientific), Next, 4.5 μg of pcDNA3,3-VHH-Fc plasmid DNA was added to the cells and incubated on a shaking platform at 37° C. and 8% CO₂, for 5 min, Next, 9 μg of PEI was added to the cultures, and cells were further incubated for 5 h, after which an equal culture volume of Ex-Cell-293 (Sigma) was added to the cells, Transfections were incubated for 4 days, after which cells were pelleted (10′, 300 g) and supernatants were filtered before further use. For purification of the VHH-Fc proteins, supernatants were loaded on a 5 mL MAbSelect SuRe column (GE Healthcare), Unbound proteins were washed away with Mellvaine buffer, pH 7.2, and bound proteins were eluted using Mellvaine buffer pH 3, Immediately after elution, protein-containing fractions were neutralized using 30% (v/v) of a saturated Na₃PO₄ buffer, Next, these fractions were pooled, and loaded on a HiPrep Desalting column for buffer exchange to PBS, pH 7.4, Additionally, huR3DC23-Fc_YTE was expressed in ExpiCHO-S™ cells (ThermoFisher Scientific), according to the manufacturer's protocol, Briefly, a 50 mL culture of 6×10⁶ cells per mL, grown at 37° C. and 8% CO₂, was transfected with 40 μg of pcDNA3,3-VHH72-Fc plasmid DNA using ExpiFectamine™ CHO reagent, One day after transfection, 300 μL ExpiCHO™ enhancer and 8 mL ExpiCHO™ feed was added to the cells, and cultures were further incubated at 32° C. and 5% CO₂, Cells were fed a second time day 5 after transfection, Productions were collected as soon as cell viability dropped below 75%.

For purification of the VHH-Fc proteins, supernatants were loaded on a 5 mL MAbSelect SuRe column (GE Healthcare), Unbound proteins were washed away with Mellvaine buffer pH 7.2, and bound proteins were eluted using Mellvaine buffer pH 3, Immediately after elution, protein-containing fractions were neutralized using 30% (v/v) of a saturated Na3PO4 buffer, Next, these fractions were pooled, and loaded on a HiPrep Desalting column for buffer exchange to PBS pH7.4.

Fed Batch Production of huR3DC23-Fc LS from Stable Pool at 1 L Scale

The gene encoding huR3DC23-Fc_LS was codon optimized, synthesized, and cloned into the pXLG6 backbone vector at ATUM's laboratories, Upon expansion to a density of about 4·10⁶ cells/ml, parental CHOExpress™ cells were co-transfected with the expression vector and the pXLG5 helper vector. The stable pool was generated under 50 mg/L puromycin selective pressure (applied daily) and further expanded. The stable pool research cell bank was banked at day 14 when cell viability reached 95%.

The RCB pool was then expanded for protein production at 1 L scale and cultured until day 12 (cell density 3.5·10⁷ cells/mL, cell viability 96%). The supernatant was harvested by centrifugation and clarified by filtration (0.2 μm). The protein was further purified by Protein A using MabSelect SuRe LX resin, Consecutive washed were performed with 20 mM sodium phosphate and 110 mM NaCl at pH 7.2:100 mM sodium acetate and 500 mM NaCl at pH 5.5; and 20 mM sodium phosphate at pH 7.2. The eluate in 100 mM sodium acetate pH 3.5 was neutralized to pH 7.0 by addition of 1 M Tris pH 11 (10% v/v), After filter sterilization (0.22 μm), the protein was aliquoted at 2 mg/ml,

### Protein Preparation for Biophysical Analysis

Preceding biophysical characterization, MAbSelect SuRe-purified protein samples were further purified via size-exclusion chromatography (SEC) on a 12° C.-cooled Superdex 200 column (GE-Healthcare) equilibrated with either Dulbecco's phosphate-buffered saline (PBS, Sigma-Aldrich) supplemented with 0.02% sodium azide to prevent microbial growth, or a sample buffer comprising 50 mM L-histidine and 150 mM L-arginine (Sigma-Aldrich), 0.02% polysorbate-20 and 0.02% sodium azide, set to pH 7.0 at 25° C. After filter sterilization (0.22 μm), 1 mg/ml aliquots were snap-frozen in polypropylene tubes in liquid nitrogen and stored at 80° C.

### Sample Composition

Purified VHH-Fc samples were characterized by analytical SEC to determine the molecular composition of each sample, After rapid thawing in a warm water bath at 25° C., 10 min centrifugation at 16,000×g and transfer of supernatant to fresh tubes, 5 μg was injected on an AdvanceBio SEC column, 4.6×300 mm (Agilent) with 2.7 μm porous particle size and 300 Å pore size, calibrated with PBS. The separation was monitored by absorbance at 280 nm with a 16 nm bandwidth, no reference subtraction. For additional quality control, proteins were separated on reducing 15% SDS-PAGE with Coomassie staining.

### Generation of Spike Protein Expression Vectors for the Production of VSVdelG Pseudovirus Particles Expressing Spike Proteins Containing RBD Mutations of SARS-COV-2 Variants

The pCG1 expression vector for the SARS-COV-2 spike protein containing the D614G mutation was generated from the pCG1-SARS-2-Sde118 vector by introducing the specific RBD mutations via QuickChange mutagenisis using appropriate primers, according to the manufacturer's instructions (Aligent). For the pCG1-SARS-2-Sde118 expression vector for the Omicron BA.1 variant a codon-optimized spike protein nucleotide sequence containing the BA.1 mutations (A67V, A69-70, T95I, G142D, A143-145, N211I, A212, ins215EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, L981F) and flanking BamHI and SalI restriction sites was ordered at Geneart (Thermo Fischer Scientific) and cloned in the pCG1 vector as an BamHI/SalI fragment. For the pCG1-SARS-2-BA.2 Sde118 expression vector a codon-optimized spike protein nucleotide sequence containing the BA.2 mutations (T19I, AL14-P26, A27S. G142D, V213G, G339D, S371F, S373P, S375F, T376A, D405N, R408S, K417N, N440K, S477N. T478K, E484A, Q493R, Q498R, N501Y, Y505H, D614G, H655Y, N679K, P681H, N764K, D796Y. Q954H, N969K) and flanking BamHI and SalI restriction sites was ordered at Geneart (Thermo Fischer Scientific) and cloned in the pCG1 vector as an BamHI/SalI fragment. For the pCG1-SARS-2-BA.2.75 Sde118 expression vector a codon-optimized spike protein nucleotide sequence containing the BA.2.75 mutations (K147E, W152R, F157L, 1210V, G257S, D339H, G446S, N460K, R493Q) and flanking BamHI and SalI restriction sites was ordered at Geneart (Thermo Fischer Scientific) and cloned in the pCG1 vector as an BamHI/SalI fragment. The pCG1 expression vector for the Omicron BA.2.75.2 variant was generated from the pCG1-SARS-2-BA.2.75 Sde118 by introducing the R346T, F486S, D1199N mutations via the Gibson Assembly cloning technique, using an appropriate gBlock (ordered at IDT), according to the manufacturer's instructions (New England BioLabs).

The pCG1 expression vector for the Omicron BA.4/BA.5 variant was generated from the pCG1-SARS-2-BA.2 Sde118 vector by introducing the H69-, V70-deletions and the L452R, F486V, R493Q mutations via QuickChange mutagenisis using appropriate primers (ordered at IDT), according to the manufacturer's instructions (Agilent).

The pCG1 expression vector for the Omicron BA.4.6 variant was generated from the pCG1-SARS-2-BA.4 Sde118 by introducing the R346T and the N658S mutation via QuickChange mutagenisis using appropriate primers (ordered at IDT), according to the manufacturer's instructions (Agilent), The pCG1 expression vector for the Omicron BF.7 variant was generated from the pCG1-SARS-2-BA.4 Sde118 by introducing the R346T mutation via QuickChange mutagenisis using appropriate primers (ordered at IDT), according to the manufacturer's instructions (Agilent).

The pCG1 expression vector for the Omicron BQ.1.1 variant was generated from the pCG1-SARS-2-BA.5 Sde118 by introducing the R346T, the K444T and the N460K mutation via QuickChange mutagenisis using appropriate primers (ordered at IDT), according to the manufacturer's instructions (Agilent).

The pCG1 expression vector for the Omicron XBB variant was generated from the pCG1-SARS-2-BA.2 Sde118 by introducing the V83A, Y144-, H146Q, Q183E, V213E, D339H, R346T, L368I, V445P, G446S, N460K, F486S, F490S, R493Q mutations via the Gibson Assembly cloning technique, using an appropriate gBlock (ordered at IDT), according to the manufacturer's instructions (New England BioLabs). The pCG1 expression vector for the Omicron XBB.1.5 variant was generated from the pCG1-SARS-2-XBB Sde118 by introducing the F486P mutation via QuickChange mutagenisis using appropriate primers (ordered at IDT), according to the manufacturer's instructions (Agilent).

After sequencing, clones containing the correct spike coding sequence were prepared using the Qiagen plasmide Qiagen kit, Before usage the spike coding sequence of the prepared pCG1 vectors was confirmed by Sanger sequencing.

### Hydrophobic Interaction Chromatography (HIC) Assay

Apparent hydrophobicity was assessed using a hydrophobic interaction chromatography (HIC) assay employing a Dionex ProPac HIC-10 column, 100 mm×4.6 mm (Thermo Fisher 063655), containing a stationary phase consisting of a mixed population of ethyl and amide functional groups bonded to silica. All separations were carried out on an Agilent 1100/1260 HPLC equipped with a UV/VIS detector. The column temperature was maintained at 25° C. throughout the run and the flow rate was 0.8 ml/min. The mobile phases used for HIC were (A) 1.6 M ammonium sulfate and 50 mM phosphate pH 7.0, and (B) 50 mM phosphate pH 7.0, Protein and calibrator samples were diluted 1:1 with buffer A and injected onto the column, Following a 5 min hold at 50% B, bound protein was eluted using a linear gradient from 50 to 100% B in 50 min followed by 5 min hold at 100% B. The column was washed with 100% B, followed by 50 mM ammonium acetate pH 5 and re-equilibration in 50% B for 10 min prior to the next sample. The separation was monitored by absorbance at 280 nm with a 16 nm bandwidth, no reference subtraction.

### Mass Spectrometry Analysis of Proteins.

Intact VHH protein (10 μg) was first reduced with tris(2-carboxyethyl) phosphine (TCEP; 10 mM) for 30 min at 37° C., after which the reduced protein was separated on an Ultimate 3000 HPLC system (Thermo Fisher Scientific, Bremen, Germany) online connected to an LTQ Orbitrap XL mass spectrometer (Thermo Fischer Scientific), Briefly, approximately 8 μg of protein was injected on a Zorbax 300SB-C18 column (5 μm, 300 Å, 1×250 mm IDxL; Agilent Technologies) and separated using a 30 min gradient from 5% to 80% solvent B at a flow rate of 100 l/min (solvent A: 0.1% formic acid and 0.05% trifluoroacetic acid in water; solvent B: 0.1% formic acid and 0.05% trifluoroacetic acid in acetonitrile). The column temperature was maintained at 60° C. Eluting proteins were directly sprayed in the mass spectrometer with an ESI source using the following parameters: spray voltage of 4.2 kV, surface-induced dissociation of 30 V, capillary temperature of 325° C. capillary voltage of 35 V and a sheath gas flow rate of 7 (arbitrary units). The mass spectrometer was operated in MS1 mode using the orbitrap analyzer at a resolution of 100.000 (at m/z 400) and a mass range of 600-4000 m/z, in profile mode. The resulting MS spectra were deconvoluted with the BioPharma Finder™ 3.0 software (Thermo Fischer Scientific) using the Xtract deconvolution algorithm (isotopically resolved spectra). The deconvoluted spectra were manually annotated, Production of VHHs by E. coli.

For the production of VHH in E. coli, a pMECS vector containing the VHH of interest was transformed into WK6 cells (the non-suppressor E coli strain) and plated on an LB plate containing ampicillin. The next day clones were picked and grown overnight in 2 mL LB containing 100 μg/ml ampicillin and 1% glucose at 37° C. while shaking at 200 rpm, One ml of this pre-culture was used to inoculate 25 ml of TB (terrific broth) supplemented with 100 μg/ml Ampicillin, 2 mM MgCl, and 0.1% glucose and incubated at 37° C. with shaking (200-250 rpm) till an OD600 of 0.6-0.9 was reached, VHH production was induced by addition of IPTG to a final concentration of 1 mM. These induced cultures were incubated overnight at 28° C. while shaking at 200 rpm. The produced VHHs were extracted from the periplasm and purified as described in Wrapp et al. (2020, Cell 181:1004-1015,e15). In short, the VHHs were purified from the solution using Ni Sepharose beads (GE Healthcare), After elution using 500 mM imidazole, the VHH containing flow-through fractions were buffer-exchanged with PBS with a Vivaspin column (5 kDa cutoff, GE Healthcare). The purified VHHs were analyzed by SDS-PAGE and Coomassie staining and by intact mass spectrometry.

### Enzyme-Linked Immunosorbent Assay

Wells of microtiter plates (type II, F96 Maxisorp, Nunc) were coated overnight at 4° C. with 100 ng of recombinant SARS-COV S-6P protein (Hsich et al. 2020), SARS-COV-1 S-2P protein (with foldon), His-tagged SARS-COV-2 RBD (Sinobiologicals), the SARS-COV-2 spike S2 subunit (ACRObiosystems), recombinant SARS-Cov-2 S-2P, SARS-COV-2 S-6P protein, recombinant SARS-COV-1 spike protein, recombinant MERS-COV spike protein, recombinant HKU1 spike protein, SARS-COV-2 Omicron BA.1 S protein (ACRObiosystems), mouse Fc-tagged SARS-COV-2 RBD (Sinobiologicals), or BSA. The coated plates were blocked with 5% milk powder in PBS, Dilution series of the VHHs or VHH-Fes or antibodies were added to the wells and incubated for 90 min, After washing, binding was detected by incubating the plates sequentially with HRP-conjugated rabbit anti-camelid VHH antibodies (Genscript) or a mouse anti-HA antibody (BioLegend 901501, 1/2000), followed by anti-mouse IgG-HRP (GE Healthcare, NA931V, 1/2000), Binding of VHH-Fcs or conventional human monoclonal antibodies was detected by rabbit anti-human IgG (Sigma), followed by HRP-conjugated anti-rabbit IgG (Southern Biotech), After washing 50 μL of TMB substrate (Tetramethylbenzidine, BD OptETA) was added to the plates and the reaction was stopped by addition of 50 μL of 1 M H₂SO₄. The absorbance at 450 nM was measured with an iMark Microplate Absorbance Reader (Bio Rad), Curve fitting was performed using nonlinear regression (Graphpad 8.0).

### Flow Cytometric Analysis of Binding to HEK293 Cells Expressing SARS-COV Spike Protein

To investigate the binding of VHHs to spike proteins on the surface of mammalian cells by flow cytometry we used pCG1-expression plasmids containing the coding sequence of the SARS-COV-2 spike protein from which the C-terminal 18 amino acids were deleted and in which the D614G substitution was introduced by QuickChange site-directed mutagenesis (Agilent) according to the manufacturer's instructions (614G), Two days after transfecting HEK293-T cells with a GFP expression plasmid in combination with either a spike expression plasmids or a control expression plasmid, the cells were collected. All further procedures were performed on ice. The cells were washed once with PBS and blocked with 1% BSA. The cells were stained with antibody or VHH dilution series for 90 minutes and subsequently washed 3 times with PBS containing 1% BSA, Binding of VHHs was detected using a mouse anti-His-tag antibody (Biorad) and an AF647 conjugated donkey anti-mouse IgG antibody (Invitrogen), Binding of VHH-Fes or antibodies was detected using a donkey anti-human IgG antibody (Invitrogen) and dead cells were stained using Live/Dead stain (Invitrogen), Following 3 washes with PBS containing 0.5% BSA, the cells were analyzed by flow cytometry using an BD LSRII flow cytometer (BD Biosciences). The binding curves were fitted using nonlinear regression (Graphpad 8.0).

### FcRn Binding Affinity

Surface plasmon resonance (SPR) analysis of antibody binding to purified recombinant human FcRn/FCGRT-B2M protein was performed by FairJourney Biologics (Porto, Portugal) on a Biacore 8 K+instrument, His-tagged recombinant human FcRn/FCGRT-B2M heterodimer protein was purchased from Acro Biosystems, bebtelovimab biosimilar and a human IgG1 isotype control antibody were used as controls in the assay. In brief, R3_DC23hum-Fc(LS) or control antibodies were immobilized at low density on a CM5 sensor chip (Cytiva) by amine coupling to immobilization levels of 130 to 283 RU. At pH 6.0, human FcRn/FCGRT-B2M heterodimer protein was injected in solution at 1.5 μM (anchor point) and in eight step, 2-fold dilution series in the range 1000 nM-7.8 nM for control antibody immobilized channels or nine step, 2 fold dilution series in the range 250 nM-0.97 nM for R3_DC23hum-Fc(LS) immobilized channels. At pH 7.4, human FcRn/FCGRT-B2M heterodimer protein was injected in solution at 1.5 μM (anchor point) and in eight step, 2-fold dilution series in the range 1000 nM-7.8 nM for all immobilized channels, Analyte injections were performed in running buffer during 1 minute at 30 μl/min; assay runs with 0 nM concentration were included as blank reference, SPR running buffer contained 1×PBS with 0.05 Tween 20 at pH 6.0/pH 7.4, A multi-cycle kinetics protocol was applied (off rate measurement 90 seconds); data obtained from antibody or VHH-Fc concentrations above 250 nM was omitted because of bulk effects, After double reference subtraction, data was analyzed either using the steady state affinity predefined evaluation method of the Biacore Insight Evaluation Software, or by fitting a 1:1 binding model in the same software.

### Human Plasma Membrane Protein Cell Array

The Retrogenix Cell Microarray Technology (Charles River Laboratories, UK) was used to screen for specific off-target binding interactions of test antibodies, Firstly, Pre-screens were performed to determine the level of background binding of each test antibody to fixed untransfected HEK293 cells and cells over-laid with SARS-COV-2 FL spike 6-HIS protein. These data were used to assess the suitability and optimal concentrations for onward screening, Secondly, in the Library screen, a pool of the test antibodies was screened for binding against fixed HEK293 cells over-expressing 6101 individual full-length human plasma membrane proteins, secreted and cell surface-tethered human secreted proteins, as well as a further 396 human heterodimers. This identified library interactions, Finally, in the Confirmation screens, all library interactions were re-expressed, and probed with the test antibody individually or control treatments, to determine which interactions, if any, were repeatable and specific to each test antibody. This was performed on both fixed and live cells. For the pre-screen, slides were spotted with expression vectors encoding both ZsGreen1 and human CD20 or EGFR and used to reverse-transfect HEK293 cells, Slides were fixed and subsequently spotted with gelatin+/−SARS-COV-2 spike protein (SARS-COV-2 FL spike 6-HIS protein supplied by Peak Proteins, spotted at 0.2 mg/mL), 1, 2.5 or 10 μg/mL of R3_DC23hum-Fc(LS) and 1 μg/mL of Rituximab biosimilar or PBS only was added to the above cells/slides after fixation, Binding to target-expressing cells and untransfected cells was assessed using an AlexaFluor 647 labelled anti-human IgG Fc (AF647 anti-hIgG Fc) detection antibody, followed by fluorescence imaging. For Library screening, 6101 expression vectors, encoding both ZsGreen 1 and a full-length human plasma membrane protein, secreted or a cell surface-tethered human secreted protein, plus a further 396 human heterodimers were individually arrayed in duplicate across cell microarray slides, An additional slide was subsequently spotted with gelatin+/−SARS-COV-2 spike protein (0.2 mg/mL), An expression vector (pIRES-hEGFR-IRES-ZsGreen1) was spotted in quadruplicate on every slide and was used to ensure that a minimal threshold of transfection efficiency had been achieved or exceeded on every slide, Human HEK293 cells were used for reverse transfection/expression. A pool of test antibodies was added to each slide after cell fixation, Detection of binding was performed using the same fluorescent secondary antibody as used in the Pre-screen (AF647 anti-hIgG Fc). The test antibody pool was screened against 2 replicate slide-sets, Fluorescent images were analyzed and quantitated (for transfection) using ImageQuant software (GE healthcare, Version 8.2). A protein interaction was defined as a duplicate spot showing a raised signal compared to background levels.

This was achieved by visual inspection, Interactions were classified as ‘strong, medium, weak or very weak’, depending on the intensity of the duplicate spots. A significant interaction was defined as signal of weak intensity or greater. For the confirmation screen, vectors encoding all interactions identified in the Library screen, plus control vectors encoding CD20 (positive control) and EGFR (transfection and negative control), were arrayed and expressed in HEK293 cells on new slides. Confirmation screen slides and analyses were carried out as for the Library screen either after cell fixation (n=2) or in the absence of fixation (n=1), Fixed slides only were also spotted with gelatin+/−SARS-COV-2 spike protein (0.2 mg/mL), Slides were treated with 2.5 μg/mL of R3_DC23hum-Fc(LS) or 20 μg/mL of IgG kappa antibody (negative control), 1 μg/mL Rituximab biosimilar (array positive control) or no test molecule (secondary only: negative control), Binding to target-expressing cells and untransfected cells was again assessed by fluorescence imaging.

### Sars-CoV Pseudovirus Neutralization Assay

To generate replication-deficient VSV pseudotyped viruses, HEK293-T cells, transfected with SARS-COV-1 S or SARS-COV-2 S were inoculated with a replication-deficient VSV vector containing eGFP and firefly luciferase expression cassettes (Berger and Zimmer 2011, PloS One 6:e25858 and Hoffmann et al. (2020) Cell 181:271-280,e8), After a 1 h incubation at 37° C. the inoculum was removed, cells were washed with PBS and incubated in media supplemented with an anti-VSV G mAb (ATCC) for 16 hours, Pseudotyped particles were then harvested and clarified by centrifugation.

For the VSV pseudotype neutralization experiments, the pseudoviruses were incubated for 30 min at 37° C. with different dilutions of purified VHH or VHH-Fc fusions or with GFP-binding protein (GBP: a VHH specific for GFP). The incubated pseudoviruses were subsequently added to subconfluent monolayers of Vero E6 or Vero E6-TMPRSS2 cells, Sixteen hours later, the cells were lysed using passive lysis buffer (Promega). The transduction efficiency was quantified by measuring the GFP fluorescence in the prepared cell lysates using a Tecan infinite 200 pro plate reader, GFP fluorescence was normalized using either the GFP fluorescence of non-infected cells and infected cells treated with PBS or the lowest and highest GFP fluorescence value of each dilution series. The IC₅₀ was calculated by non-linear regression curve fitting, log (inhibitor) vs, response (four parameters).

Alternatively, dilution series of VHHs or antibodies were mixed with 100 PFU of GFP expressing replication-competent VSV virus particles pseudotyped with the SARS-COV-2 spike protein derived from an early isolate, Of note during propagation of this viral clone (S1-10a) on Vero E6 cells the furin cleavage site was mutated and as such inactivated (Koenig et al. (2021) Science 371:eabe6230), After 30 minutes incubation at 37° C. the virus-antibody mixtures were added to monolayers of Vero E6 cells and allowed to infect and replicate for three days.

In all neutralization assays using pseudotyped VSV viral particles FluoroBrite DMEM medium (Invitrogen) supplemented with 5% heat-inactivated FBS, 1% penicillin, 1% streptomycin, 2 mM 1-glutamine, non-essential amino acids (Invitrogen) and 1 mM sodium pyruvate was used to prepare the VHH or antibody-virus mixtures. The mixtures were added to cells from which the original growth medium was removed.

### SARS-CoV-2 Plaque Reduction Neutralization Test (PRNT)

The plaque reduction assays using authentic viruses were performed with SARS-COV-2 strain SARS-CoV-2/human/FRA/702/2020 (obtained from the European Virus Archive (EVAG)) and with an SARS-COV-2 BA.1 virus (Planas et al. (2022) Nature 602:671-675) and grown on Vero E6 cells. Further propagation of the virus was performed on Vero E6-TMPRSS2 cells.

Both viruses were titrated using a plaque assay in which monolayers of Vero E6-TMPRSS2 cells were infected with dilutions series prepared in Dulbecco's Modified Eagle Medium (DMEM) supplemented with 2% fetal bovine serum (FBS) in duplicate for 2 vials of each virus, Two hours after infection Avicel was added to a final concentration of 0.3% (w/v).

Dose-dependent neutralization of distinct constructs was assessed by mixing the constructs at different concentrations (5-fold serial dilutions) with 40 PFU of SARS-COV-2, and by incubating the mixture at 37° C. for 1 hour. The VHH-virus mixes were then added to Vero E6-TMPRSS2 cell monolayers in 12-well plates and incubated at 37° C. for 1 hour, Subsequently, Avicel was added to a final concentration of 0.3% (w/v), After 2 days of incubation at 37° C., the overlays were removed, and the cells were fixed with 3.7% paraformaldehyde (PFA) and stained with 0.5% crystal violet.

Half-maximum neutralization titers (PRNT₅₀) were defined as the VHH-Fc concentration that resulted in a plaque reduction of 50% across two independent plates.

### Live Virus Assay

The live virus assay was performed with SARS-COV-2 viruses belonging to different lineages (614G, Delta, Omicron BA.1, Omicron BA.2 and Omicron BA.5) isolated from nasopharyngeal swabs taken from patients/travelers between January 2020 and July 2022.

| | | Date of |
|---|---|---|
| | Name | isolation |
| WT | SARS-CoV-2 Isolate BavPat1/2020; | 1 Jan. 2020 |
| | Germany; 9 Feb. 2020 | |
| Delta | SARS-Related Coronavirus 2, | 27 Apr. 2021 |
| | Isolate hCoV-19/USA/MD-HP05647/2021 | |
| BA.1 | SARS-CoV-2 hCoV-19/Netherlands/ | 29 Nov. 2021 |
| | NH-RIVM-72291/2021 | |
| BA.2 | Clinical isolate hCoV-19/Netherlands/ | 3 Mar. 2022 |
| | VCB-20220303-1/2022 | |
| BA.5 | Clinical isolate hCov19/NL/VCB- | 14 Jul. 2022 |
| | 20220714-2/2022 | |

Dose-dependent neutralization of the test item, the positive controls (bebtelovimab Biosimilar, cilgavimab Biosimilar, sotrovimab Biosimilar and a negative control (isotype control) were assessed in a live virus neutralization assay. For all assays in which live D614G, Delta, BA.1 and BA.2 SARS-CoV-2 were tested, huR3DC23-Fc_LS produced from transiently transfected cells was used. For all assays in which SARS-COV-2 BA.5 was tested, huR3DC23_Fc_LS produced from stable cell pools was used. For each variant, three independent runs were performed, Different system controls were included in the assay: cell only (medium only), virus only, and an internal positive control (human serum), Briefly, 5-fold or 7-fold dilutions of the test items and controls were incubated with a fixed amount of virus for 1 hour at room temperature.

| | Stock titer | |
|---|---|---|
| Virus | (TCID50/mL) | Dilution |
| D614G | 6.77 | 200x |
| Delta | 6.7 | 100x |
| Omicron A.1 | 6.13 | 40x |
| Omicron A.2 | 4.84 | 10x |
| Omicron A.5 | 5.89 | 20x |

Vero-E6 cell monolayers were inoculated with the virus antibody mixtures for 1 hour at 37° C. In a next step, the inoculum was removed and the cells were incubated at 37° C. with infection medium (Minimum Essential Medium (MEM) supplemented with 2 mM L-glutamine, 1× Non-essential amino acids, 25 mM HEPES (N-2-hydroxyethylpiperazine-N-2-ethane sulfonic acid), 1% heat-inactivated fetal bovine serum (FBS) and 1× Antibiotic-Antimycotic (Gibco)) (up to 18-24 hours post-infection), Afterwards, the SARS-COV2 infected cells were fixed and immunostained with a SARS-COV Nucleocapsid Antibody (Sinobiological, Catalogue number: 40143-MM05), followed by HRP-conjugated Goat anti-Mouse IgG (H+L) Secondary Antibody (Invitrogen, catalogue number A16072), Spots (infected cells) were counted using an ImmunoSpot® Analyzer S6 Ultimate (CTL), For each antibody/construct, the concentration showing 50% reduction in infection (IC₅₀) was calculated based on the Zielinska method (Zielinska et al. (2005) Virology Journal 2:84). The geometric mean values were calculated based on three independent runs.S1 shedding assay

Antibody or VHH was added at a final concentration of 10 μg/ml to 1 million Raji cells expressing either no spike, or SARS-COV-2 spike. The antibody/VHH-cell mixture was incubated for 30 min or 1 h at 37° C. and 5% CO₂, After incubation, cells were pelleted by centrifugation, supernatant was transferred to a fresh tube and the cell pellet was lysed with RIPA lysis buffer (50 mM Tris-HCl pH 8.0, 100 mM NaCl, 1 mM EDTA, 1 mM EGTA, 0.1% SDS, 1% NP-40), 20 μl samples of supernatant and lysate were separated on 8% SDS-PAGE gels, and electroblotted onto nitrocellulose membranes, Membranes were blocked with 4% milk, stained with rabbit anti-SARS-S1 antibody (1/1000, Sino biologics, 40591-T62) followed by anti-rabbit IgG-HRP (1/2000, GE Healthcare, NA934V) and developed using Pierce™ ECL Western Blotting Substrate (Thermofisher Scientific).

Fusion Inhibition Assay Using Replication-Competent GFP Reporter VSV Virus Pseudotyped with Wuhan SARS-COV-2 Spikes (Del-18)

Vero E6-TMPRSS2 cells were infected with 40 PFU of SARS-COV-2 spike GFP expressing pseudotyped replication-competent VSV-GFP virus (Koenig et al. 2021), Two hours later, the indicated monoclonal antibodies or VHHs were added, Non-infected cells were used as negative controls, Cells were infected overnight and imaged with a fluorescence microscope, GFP fluorescence was measured with a fluorimeter, Of note, different from the clone of replication-competent pseudotyped VSV particles used in the neutralization assays, the furin cleavage site of the virus used in the fusion assays had an intact furin cleavage site as confirmed by Sanger sequencing.

### Fusion Inhibition Assay Using Spike Expressing Vero E6 Cells

Vero E6 cells were transfected with an GFP expression vector in combination with either a control expression vector (no spike) or an SARS-COV-2 spike expression vector using Fugene, Two hours after transfection PBS, monoclonal antibodies or VHHs were added to a final concentration of 10 μg/ml, Twenty-two hours after transfection the cells were fixed using 3.7% paraformaldehyde and after washing with PBS imaged using a fluorescence microscope.

### Viral Escape Selection

Monolayers of Vero E6-TMPRSS2 cells seeded in 96 well plates were infected with 400 PFU GFP expression replication-competent VSV virus particles pseudotyped with the SARS-COV-2 spike protein containing an intact furin cleavage site (Koenig et al. 2021), Two hours after infection 10 μg/ml of VHH.R3_DC23 was added, To one control well no VHH was added.

The growth medium of wells that displayed syncytia formation or viral replication in the presence of VHH.R3_DC23 was collected and used to isolate single plaques of escape viruses in the presence of 2 μg/ml VHH.R3_DC23 by limiting dilution. This growth medium was used to propagate the virus on monolayers of Vero E6-TMPRSS2 cells seeded in 6 well plates in the presence of VHH.R3_DC23, From these infected cells RNA was prepared using a nucleospin RNA virus kit (Macherey Nagel Bioanalysis) and used to generate cDNA using random hexamer primers. This cDNA was used to amplify the spike S2 coding sequences by PCR. These PCR fragments were purified and sequenced using Sanger sequencing. The obtained nucleotide sequences were analyzed and aligned to spike proteins of WT SARS-COV-2 and clade 1, 2 and 3 sarbecoviruses (Letko et al. (2020) Nature Microbiology 5:5 62-569) using CLC Main Workbench 20.0.4, Mutations were visualized on a model of full length glycosylated spike protein obtained from Charmm-gui.org (PDB: 6VXX_1_1_1 model) or the SARS-COV-2 HR2 coiled coil as determined by NMR (PDB: 3FXP) using pymol.

### Growth Kinetics of Viral Escape Variants

Vero E6 cells seeded in a 96 well plate were infected with 50 PFU of GFP-expressing replication competent VSV virus particles pseudotyped with the SARS-COV-2 spike protein, that were obtained during escape selection, GFP expression was monitored hourly with an Incucyte Zoom device and analyzed with accompanying software.

### HDX-MS Epitope Mapping

3.33 μM SARS-COV-2 S-2P trimer was incubated overnight at 37° C. The protein was then diluted to 1.66 μM trimer in the presence or absence of 6.25 μM R3DC23 in 1×PBS (pH 7.4, Sigma-Aldrich P4417), To initiate exchange, the protein was diluted tenfold into temperature-equilibrated deuterated buffer made by lyophilizing 1×PBS and resuspending in D20 (Sigma-Aldrich 151882), Samples were quenched at each time point (15 s, 3 m, 30 m, 3 h) by mixing 60 μl of the exchange reaction with 60 μl of ice-cold 2× quench buffer (3.6M guanidinium chloride, 500 mM TCEP, 200 mM glycine pH 2.4). The quenched samples were incubated on ice for 1 minute and then flash frozen in liquid nitrogen and stored at −80° C. until LC-MS, LC-MS and data analysis was conducted as previously described (Costello, Shoemaker et, al 2022).

### HR2 Expression and Purification

For structural biology purposes the HR2 protein was expressed in a bacterial expression system, Therefore, the synthetic gene encoding residues H1159-K1211 of the HR2 protein was cloned into a pFloat-SUMO vector, generating a His-tagged SUMO-HR2 fusion protein. The construct also contained a 3C protease cleavage site to remove the His-SUMO-tag. The pFloat-SUMO-HR2 plasmid was transformed in BL21 (DE3) cells and plated on kanamycin (100 μg/ml) containing LB agar plates. A small LB culture, supplemented with 100 μg/ml kanamycin, was inoculated with a single colony of BL21 (DE3) (pFloat-SUMO-HR2) and grown overnight at 37° C. 1 L LB cultures were subsequently inoculated with 20 ml of this preculture and grown at 37° C. until OD₆₀₀ reached 0.8. At this point protein expression was induced using 0.5 mM isopropyl β-D-1-thiogalactopyranoside (IPTG), Cells were incubated further overnight at 20° C. and subsequently harvested by centrifugation (Beckman rotor 8.1000, 5000 rpm, 15 min, 4° C.). The pellet was resuspended in PBS, 500 mM NaCl, 10 mM imidazole, 5 mM β-mercaptocthanol, 0.1 mg/mL 4-(2-aminoethyl)benzenesulfonyl fluoride hydrochloride (AEBSF), 1 μg/mL leupeptine, 50 μg/mL DNasel and 20 mM MgCl₂. The cells were lysed using a French press (Constant Systems) at 20 kpsi and the cell debris was removed by centrifugation. The cell lysate was loaded on a Ni-sepharose FF HiLoad column (GE Healthcare), equilibrated in 20 mM Tris-HCl pH 7.5, 500 mM NaCl, 10 mM imidazole, 5 mM β-mercaptoethanol. The bound proteins were eluted using a linear gradient to 500 mM imidazole, Fractions containing the His-SUMO-HR2 protein were pooled and dialysed overnight to 20 mM Tris-HCl pH 7.5, 150 mM NaCl at 4° C., followed by 2 h incubation with 3C protease at room temperature. The cleaved sample was loaded again on a Ni-sepharose FF HiLoad column, equilibrated in the same buffer. The flow through, containing the HR2 protein, was concentrated and applied to a BioRad Enrich 70 10/30 size exclusion column (SEC), equilibrated in 20 mM Tris-HCl pH 7.5, 150 mM NaCl. The HR2-containing SEC fractions were pooled.

### R3DC23-HR2 Crystallization, X-Ray Data Collection, Processing, and Structure Determination

For crystallization, R3DC23 was added to HR2 in 1.2 times molar excess and concentrated to 19 mg/ml using Amicon Ultra 3 kDa cut off centrifugal filter devices, Crystallization screens were set up using the siting drop vapor diffusion technique, mixing 0.1 ul R3DC23-HR2 and 0.2 μl bottom solution, Crystals were grown from the Molecular Dimensions Proplex crystallization screen, in 0.1 M magnesium chloride hexahydrate, 0.1 M sodium citrate pH 5.0, 15% PEG4000. For X-ray data collection crystals were flash frozen in liquid nitrogen, X-ray data were collected on the i24 beamline at the Diamond Light Source synchrotron facility (Didcot, UK), X-ray data were processed using autoPROC+Staraniso (Vonrhin t al. 2011 Acta Crystallogr D Biol Crystallogr 67:293-302; Vonrhein et al. 2023 Acta Crystallographica Section A: Foundations and Advances). The structure of the R3DC23-HR2 complex was solved using the automatic molecular replacement workflow in the CCP4 cloud (Krissinel et al. 2022 Acta Cryst D 78:1079-1089). The initial model was further build manually in Coot (Emsley and Cowtan 2004 Acta Crystallogr D Biol Crystallogr 60:2126-2132) and refined using phenix, refine (Afonine et al. 2012 Acta Crystallogr D Biol Crystallogr 68:352-367) from the Phenix crystallographic software suite (Adams et al. 2010 Acta Crystallogr D Biol Crystallogr 66:213-221), Data collection parameters, as well as processing and refinement statistics are shown in Table 6.

TABLE 6
Data collection statistics and refinement
parameters for R3_DC23-HR2.

| Dataset | R3_DC23-HR2 |
|---|---|
| Data collection | |
| Synchrotron | Diamond Light Source |
| Beamline | i24 |
| Wavelenght, Å | 0.61990 |
| Data processing | |
| Space group | I222 |
| Cell parameters, Å | 90.413 98.8 150.763 |
| (α = β = γ = 90°) | |
| Resolution, Å (outer shell) ^{(c)} | 82.636-1.937 |
| | (2.052-1.937) |
| Total reflections | 543617 (26260) |
| No. of unique reflections | 39377 (1969) |
| Completeness (spherical) | 78.1 (24.9) |
| Completeness (ellipsoidal) | 95.2 (64.6) |
| Multiplicity | 13.8 (13.3) |
| R_{merge}, % | 0.274 (2.04) |
| Rₚᵢₘ, % | 0.076 (0.586) |
| <I/σ (I)> | 8.3 (1.5) |
| CC(½) | 0.990 (0.611) |
| Mosaicity, ° | |
| Refinement | |
| Resolution range, Å | 82.64-1.94 |
| No. of reflections | 39359 |
| Percentage observed | 78.02 |
| R_{cryst},^{(a)}% | 20.81 |
| R_{free},^{(b)}% | 25.09 |
| RMS | |
| Bonds, Å | 0.024 |
| Angles, ° | 3.119 |
| Ramachandran Plot | |
| Most favored, % | 95.56 |
| Additionally allowed, % | 2.22 |
| Disallowed, % | 2.22 |
| PDB code | XXXX |

^{(a)}R_{cryst} = S||F_{obs}| − |F_{calc}||/S|F_{obs}|, F_{obs} and F_{calc} are observed and calculated structure factor amplitudes.
^{(b)}R_{free} as for R_{cryst} using a random subset of the data excluded from the refinement.
^{(c)} Data in brackets are for the highest resolution shell

### In Vivo Viral Challenge in K18-hACE2 Mice

K18-hACE2 mice: B6.Cg-Tg (K18-ACE2) 2Prlmn/J (7-9 weeks of age) were purchased from The Jackson Laboratory and bred in house under specific-pathogen-free (SPF) conditions, SARS-COV-2 infections were performed under biosafety level 3 (BSL3) conditions, Antibody treatment was performed by intraperitoneal injection using a volume of 100 μl, Animals were anesthetized by isoflurane inhalation and 3*10² PFU of SARS-COV-2 614G variant virus (SARS-COV-2/human/FRA/702/2020, obtained from the European Virus Archive) was administered by intratracheal instillation, Animals were monitored on a daily base by a blind observer who measured weight change and scored for humane endpoints: Hunchback (1 point) Piloerection (1 point), less movement upon opening cage (1 point), motionless upon touching (2 points), neurological symptoms (shaking, balance, 3 points), heavy breathing (3 points), Mice that lost more than 25% of their initial bodyweight or reached a humane endpoint with a score of 5 points were euthanized.

### Titration of SARS-COV-2 Viral Titer in Lung Homogenates of Mice

After dissection, the right lung lobes were stored at −80° C. For the quantification of the lung viral titer the lung samples were homogenized using a Precellys Evolution tissue homogenizer (Bertin-technologies). The lung homogenates were cleared by centrifugation (1,000×g) for 15 min at 4° C. and used to determine the viral titer by plaque assay on VeroE6-TMPRSS2 cells in duplicate using 12-well plates, After addition of the lung homogenate dilution series to the cells, the plates were incubated at 37° C. for 2 hours, Subsequently the cells were washed twice and incubated in medium containing Avicel at a concentration of 0.3% (w/v), After 2 days of incubation at 37° C., the overlays were removed, the cells were fixed with 3.7% paraformaldehyde (PFA) and stained with 0.5% crystal-violet dye to visualize the viral plaques.

To quantify the SARS-COV-2 viral RNA levels, RNA was prepared from the lung homogenates and analyzed by qRT-PCR, cDNA was prepared using the iScript™ cDNA Synthesis Kit and random hexamer primers, QPCR was performed using the SARS-COV-2 Research Use Only (RUO) qPCR Primers & Probes kit (IDT) according to the manufacturer's instructions.

### Syrian Golden Hamster Challenge Model

9- to 10-weeks-old male Syrian golden hamsters (Mesocricetus auratus) weighing 89.8 g to 132.3 g were obtained from Janvier (France), Six hamsters per group were infected intranasally with 10\hat{ }2.0 50% tissue culture infectious dose (TCID50)/dose SARS-COV-2 (Wuhan strain) in a total dose volume of 100 μl, divided equally over both nostrils, XVR012 (a cocktail of XVR014 and XVR013), XVR013, or XVR014 at the indicated doses, palivizumab (10 mg/kg), or bebtelovimab (10 mg/kg) were administered by intraperitoneal injection either 4 hours after the SARS-COV2 challenge (therapeutic setting) or approximately 24 hours prior to infection (prophylactic setting). The irrelevant antibody Palivizumab (Synagis, anti-RSV antibody) was used as a negative control, while bebtelovimab was used as a positive control. The huR3DC23-Fc_LS used in the hamster study was produced from stable cells pools, Hamsters were monitored daily for behavior, appearance and body weight. On day 4 post-infection, animals were euthanized. At the time of necropsy, gross pathology was performed and abnormalities were recorded, Samples from the right lung lobes were collected and frozen for virological analysis, To determine virus titers, quadruplicate 10-fold serial dilutions were used in confluent layers of Vero E6 cells, To this end, serial dilutions of the samples (lung tissue homogenates) were incubated on Vero E6 monolayers for 1 hour at 37° C. Vero E6 monolayers were then washed and incubated for 5 or 6 days at 37° C., after which plates were stained and scored based on cytopathic effect (CPE) by using the vitality marker WST8 (colorimetric readout), Viral titers (Log10 TCID50/g) were calculated using the Spearman-Karber method (Kärber, 1931 Archiv F, Experiment, Pathol, u, Pharmakol, 162:480-483). For the viral titration, the lower limit of detection (LLOD) ranged between 1.1 and 1.3 log 10 TCID/g, To detect viral RNA, lung tissue homogenates were used, RNA was isolated and Taqman PCR was performed. The number of copies (Log 10 CP/g) in the different samples was calculated against a standard included in each run. For the viral RNA in lung tissue, the LLOD was 3.5 Log10 CP/g, Blood samples were collected prior to the start of the study on day-2 (˜200 μl blood was collected for serum under isoflurane anesthesia) and on day 4 post-infection (p.i.) at time of necropsy for pharmacokinetics analysis, Blood samples for serum were immediately transferred to appropriate tubes containing a clot activator, Serum was collected and stored frozen, To inactivate any potential infectious material present and to allow the testing of the serum samples in a BSL-2 environment, serum on day 4 post-infection was heat-treated at 56° C. for 30 minutes.

### ADCC Assay FcyRIIIa Reporter Assay

ADCC assessments were performed by Antibody Analytics using CHO-K1 expressing SARS COV-2 Spike Protein target cell line as target cells and Jurkat FcγRIIIa (CD16) V176-NFAT-RE Luc as reporter cells.

### Biolayer Interferometry (BLI)

Biolayer interferometry was performed on an Octet RED96 system (FortéBio), Streptavidin (SA) biosensors (Sartorius) were soaked in 1× kinetics buffer (10 mM HEPES pH 7.4, 150 mM NaCl, 0.1 mg/ml bovine serum albumin, 0.02% Tween-20 and 0.02% sodium azide) for 20 min before use, trimeric S-2P-foldon-His-Strep was thawed from −20° C. and incubated at 37° C. for 24 h followed by 30° C. for 30 min, before immobilizing on the SA biosensors at 36.5 μg/ml (80 nM) for 450 seconds, to a signal of 1.5 to 1.7 nm, Association (120 s) and dissociation (480 s) of a two-fold dilution series starting from 20 nM monomeric R3DC23 VHH in 1× kinetics buffer were measured at 30° C.

Between analyses, biosensors were regenerated by three times 5 s exposure to regeneration buffer (10 mM glycine pH 3). The response in nm for stoichiometry analysis was determined from the raw data curves, Data for kinetics analysis were double reference-subtracted and aligned to each other in Octet Data Analysis software v9.0 (FortéBio), Association and dissociation of the full dilution series were fit in a global 1:1 model.

### Example 1: Isolation of VHHs that Bind the SARS-COV-2 and SARS-COV-1 Spikes at their S2 Subunit

A llama that was previously immunized with the spike protein of MERS-COV and SARS-COV-1 (Wrapp, D, et al. (2020) Structural Basis for Potent Neutralization of Betacoronaviruses by Single-Domain Camelid Antibodies, Cell 181:1004-1015,e15) was boosted with 3 successive immunizations with SARS-COV-2 spike protein S-2P, in particular recombinant prefusion-stabilized SARS-COV-2-2P spike.

After vaccination a phagemid library containing VHH coding sequences was constructed and used for phage display biopanning, To obtain VHHs that target the spike protein at sites other than the RBD, in each round of biopanning the phages were pre-incubated with 10 μg/ml RBD-SD1, After 2 rounds of biopanning on SARS-COV-2 spike S-2P that was captured on ELISA plates via an anti-His antibody, one or two additional rounds were performed using either anti-His captured SARS-CoV-2-spike protein S-2P (R3C and R4C) or SARS-COV-2-2P spike S-2P that was directly coated (R3DC and R4DC). For each of the obtained libraries, 21 or 24 clones were picked and used for periplasmic VHH protein expression.

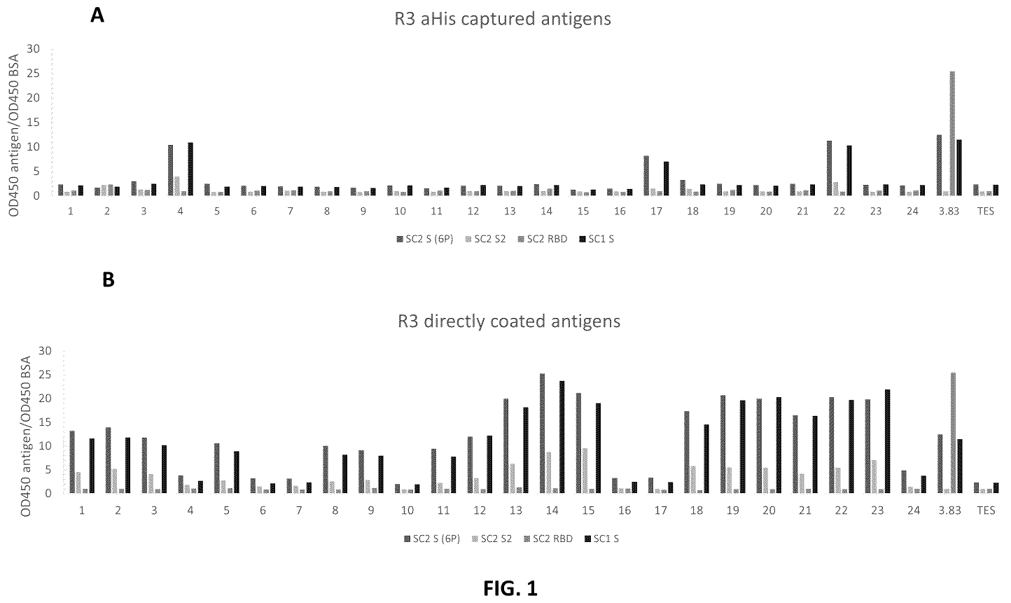

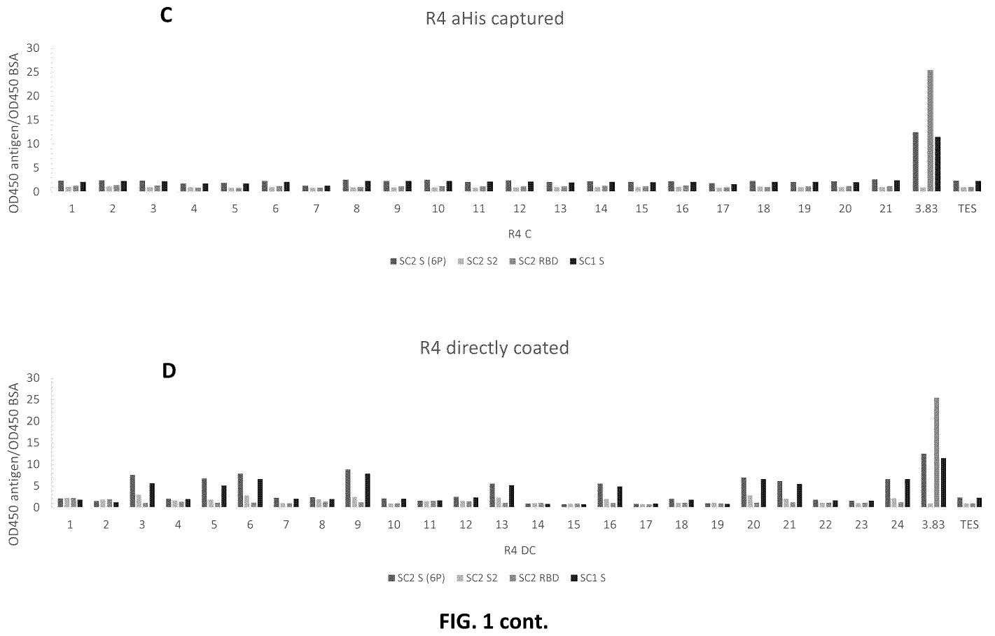

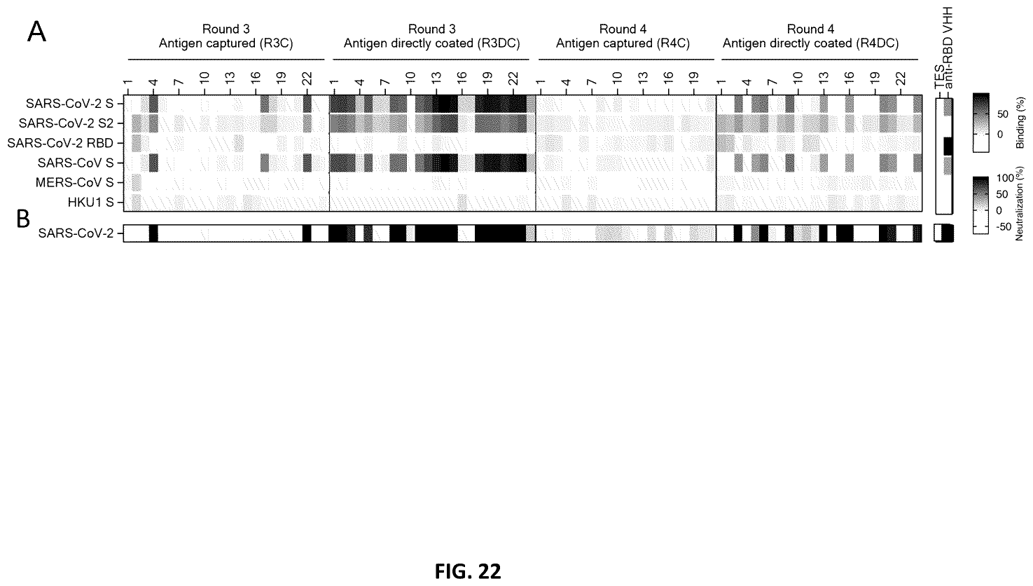

After protein production in E. coli, cleared periplasmic extracts were prepared and tested for binding to recombinant prefusion-stabilized SARS-COV-2 spike protein S-6P that includes 6 proline substitutions at the ectodomain (SC2 S) (Hsich et al. (2020) Structure-based design of prefusion-stabilized SARS-COV-2 spikes, Science 369:1501-1505), SARS-COV-2 RBD (SC2 RBD) and the S2 subunit of S-6P (SC2 S2) by ELISA. In addition, binding to recombinant stabilized SARS-COV-1 Spike protein (SC1 S), MERS-COV spike and HKU1 spike was tested, Out of the 93 tested VHHs 30 were able to bind the spike proteins of both SARS-COV-2 and SARS-COV-1 (FIG. 1). For all these VHHs, binding was directed against the S2 subunit of the spike protein and not the RBD (FIG. 1), These VHHs failed to bind the SARS-COV-2 RBD, MERS-COV or HKU1 spike, but retained binding of SARS-COV-1 spike (FIG. 22A), Interestingly, the majority of the S2-binding VHHs could efficiently neutralize vesicular stomatitis virus (VSV) virus particles pseudotyped with SARS-COV-2 spikes (FIG. 22B).

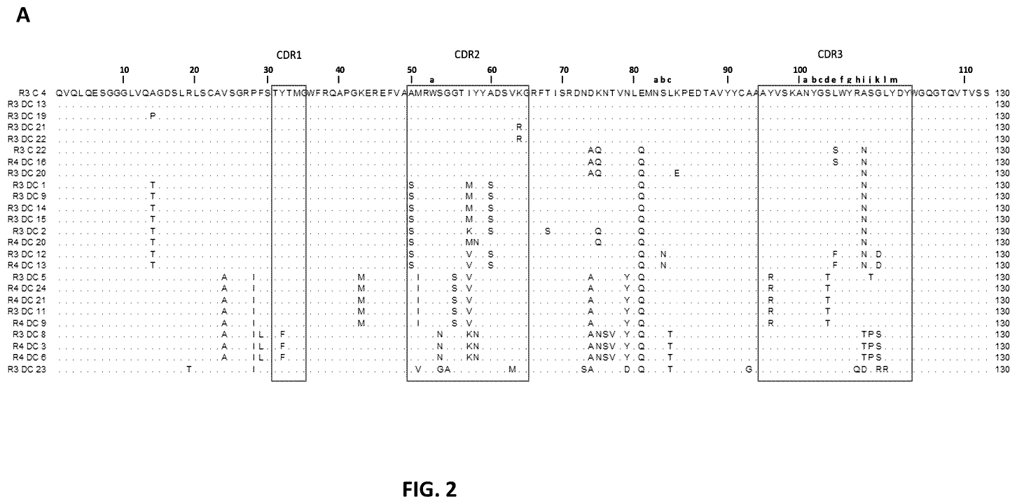

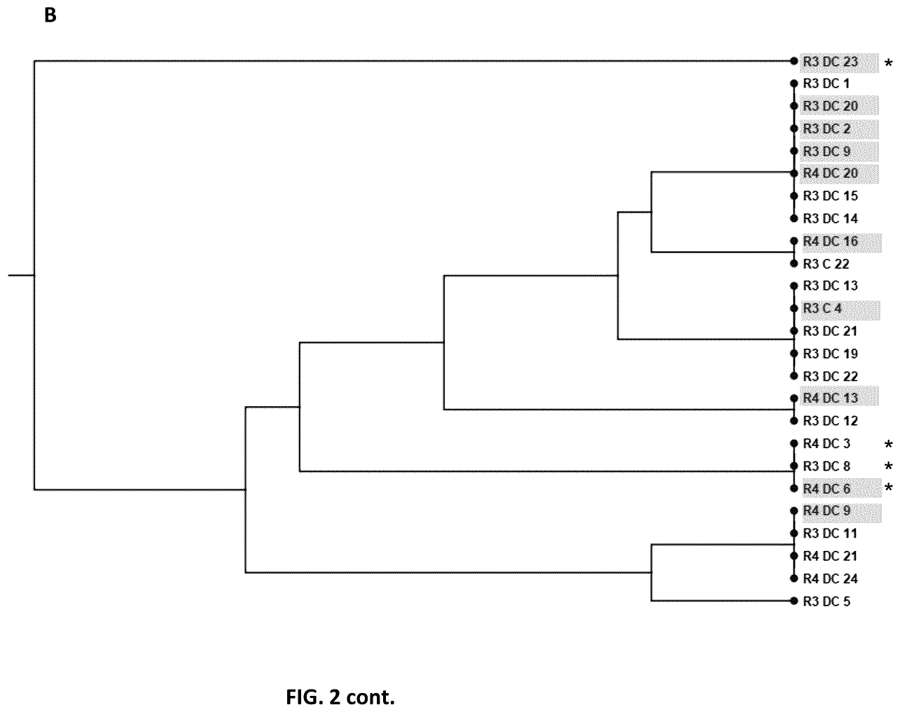

Sequence analysis of the VHH coding sequences of the periplasmic extracts that recognize the spike proteins of SARS-COV-2 and SARS-COV-1 revealed 15 unique VHH sequences belonging to 2 families containing respectively 13 (family 1) and 2 (family 2) VHHs (FIG. 2), Sequence analysis of the neutralizing VHHs revealed that they belong to a VHH family of 13 unique VHHs (family 1). Based on the CDR3 sequences, family 1 can be divided in 7 subfamilies whereas the smallest family contained 1 subfamily (FIG. 2B).

### Example 2: VHHs Targeting the S2 Subunit Bind to the Surface of Spike Expressing Cells

From each subfamily of family 1 at least one VHH was produced in E. coli WK6 cells, purified from the periplasmic extracts by Ni-NTA affinity chromatography and buffer exchanged to PBS. The molecular mass of the produced VHHs was confirmed by intact mass spectrometry. When corrected for the formation of a cysteine bound, the measured mass of each VHH corresponded to the respective theoretical mass (Table 5).

TABLE 5
Theoretical mass (Da) and the mass measured by intact
Mass Spectrometry of the VHHs for the selected VHHs.
All tested VHHs are predicted to contain a single cysteine
bound that accounts for the −2 Da difference between
the measured and theoretical VHH mass.

| | Theoretical | Measured |
|---|---|---|
| VHH | mass (Da) | mass (Da) |
| R3_DC2 | 16658.83 | 16656.89 |
| R3_C4 | 16553.85 | 16551.92 |
| R4_DC6 | 16503.88 | 16501.98 |
| R3_DC23 | 16669.83 | 16557.89 |
| R3_DC9 | 16675.83 | 16673.93 |
| R4_DC9 | 16553.94 | 16551.99 |
| R4_DC13 | 16762.86 | 16760.96 |
| R4_DC16 | 16525.80 | 16523.87 |
| R3_DC20 | 16552.80 | 16550.84 |
| R4_DC20 | 16610.78 | 16608.83 |

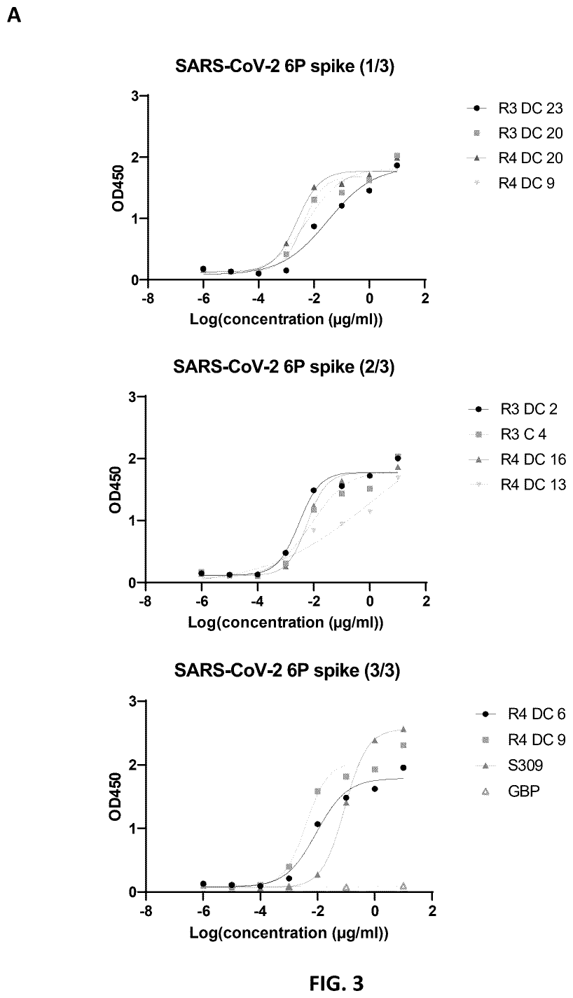

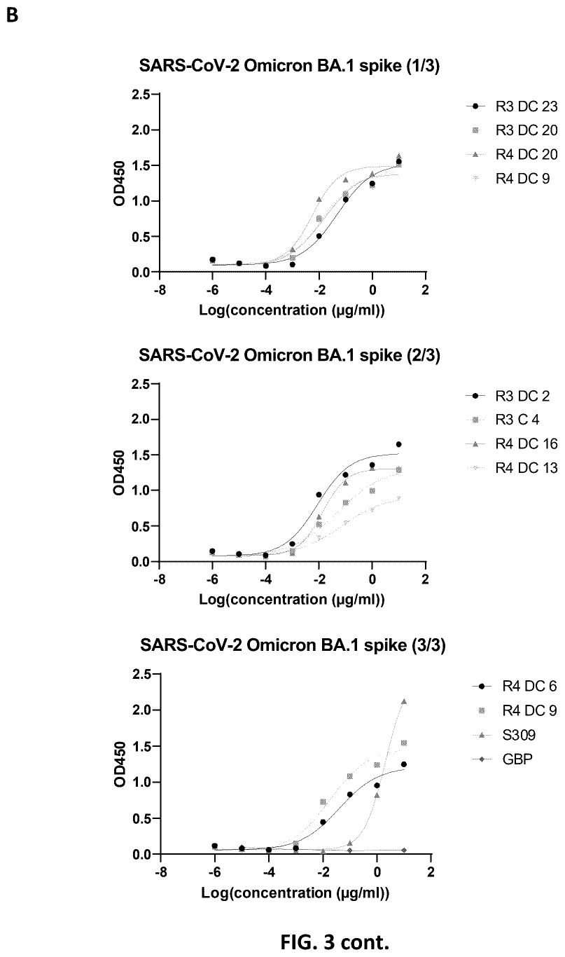

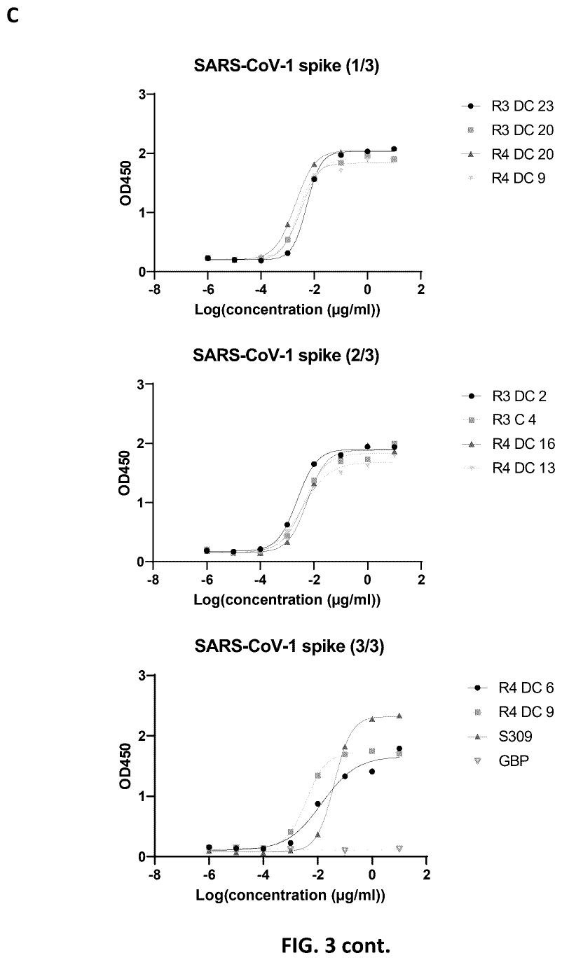

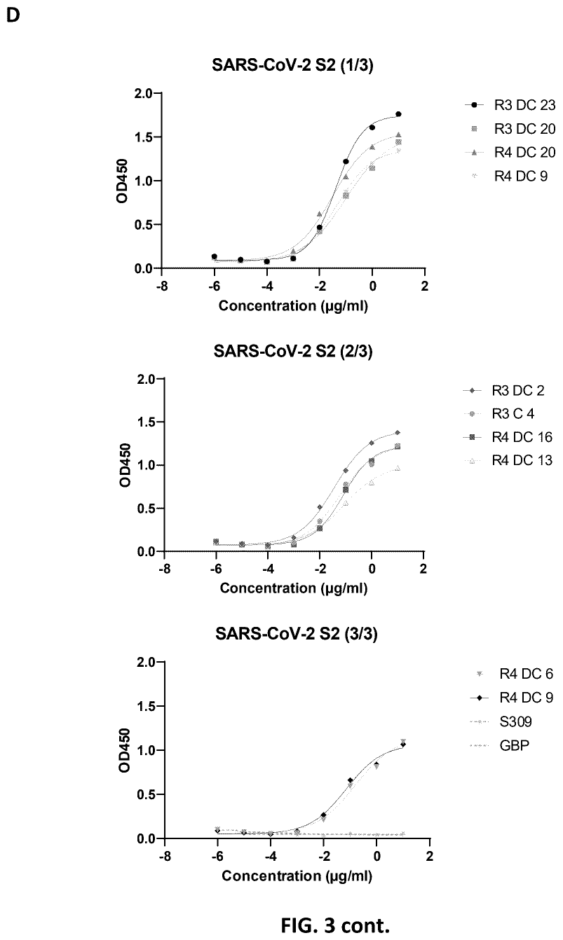

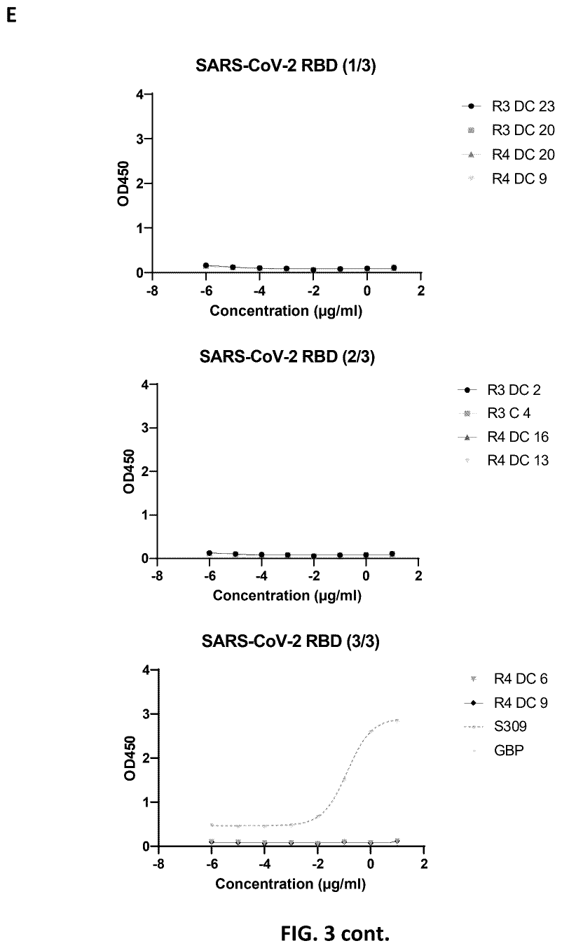

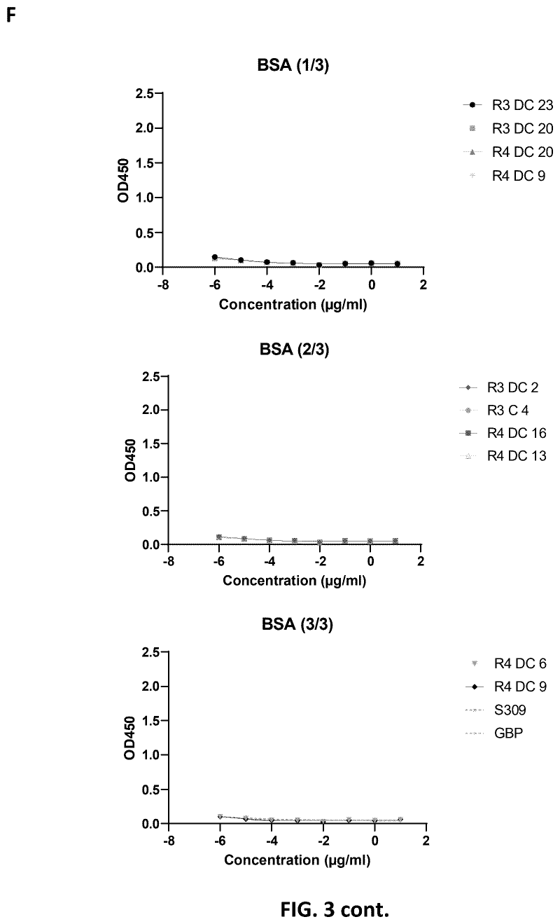

The VHHs of family 1 were tested for binding to S-6P (FIG. 3A), RBD (FIG. 3E) and S2 (FIG. 3D) of Wuhan SARS-COV-2 and the spike protein of Omicron BA.1 SARS-COV-2 (FIG. 3B) and the spike protein of SARS-COV-1 (FIG. 3C) by ELISA. The RBD specific, cross-reactive monoclonal antibody S309 was used as control. All the tested VHHs and S309 were able to bind to the spike proteins of Wuhan SARS-COV-2, Omicron BA.1 SARS-COV-2 and of SARS-COV-1 (FIG. 3), Whereas none of the VHHs were able to bind the SARS-COV-2 RBD, they all recognized the S2 subunit. In contrast, S309 recognized the RBD but failed to bind the S2 subunit.

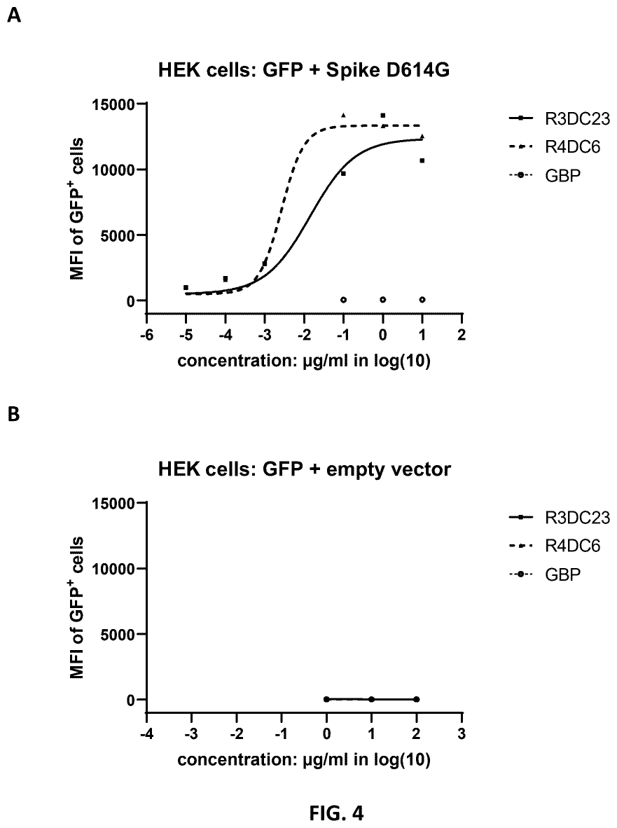

To test if the S2 specific VHHs can also bind to the spike proteins in the context of a cellular membrane, binding of the R3_DC23 and R4_DC6 to the surface of HEK293T cells transfected with either an GFP expression vector alone or in combination with an SARS-COV-2 spike expression vector (614G) was tested by flow cytometry, FIG. 4A illustrates that both R3_DC23 and R4_DC6 potently bound to cells expressing SARS-COV-2 spike proteins at their surface, No binding of these VHHs was observed at the surface of cells not expressing spike proteins (FIG. 4B), illustrating their specificity.

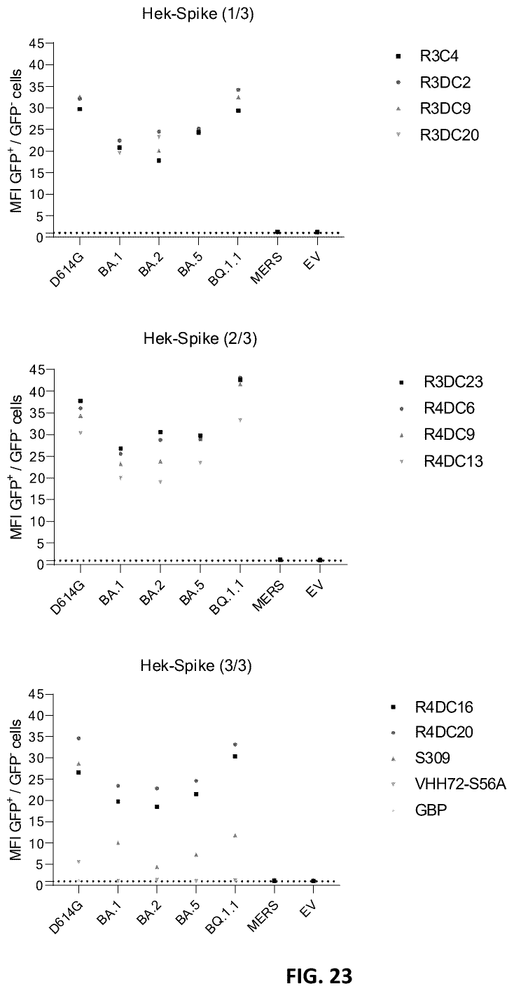

These S2-targeting VHHs also efficiently recognized spike proteins of SARS-COV-2 D64G, BA.1, BA.2, BA.5, and BQ1.1 with an intact furin cleavage site expressed on the surface of transfected mammalian cells, whereas MERS-COV spike was not recognized (FIG. 23).

### Example 3: VHHs Targeting the S2 Subunit Potently Neutralize SARS-COV-1 and -2 S Pseudotyped Viruses

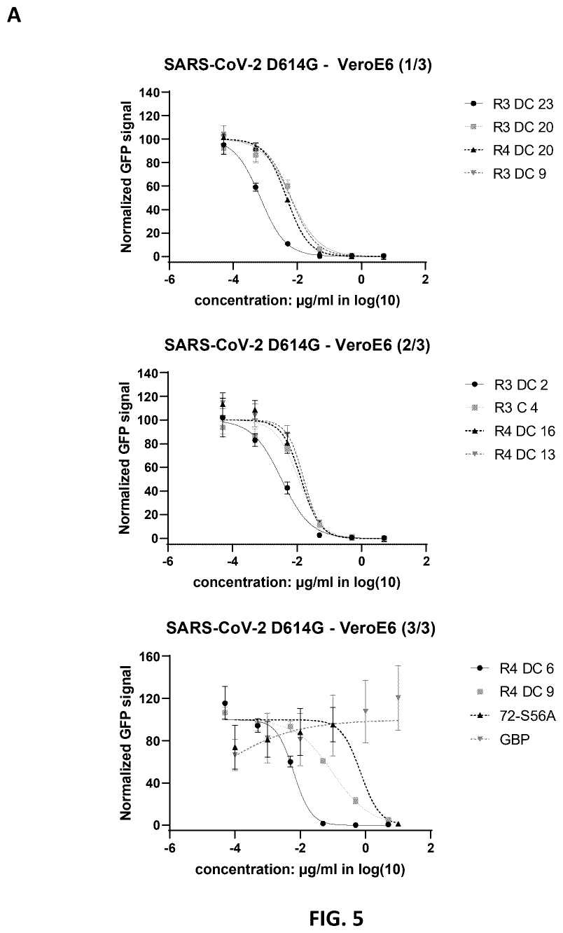

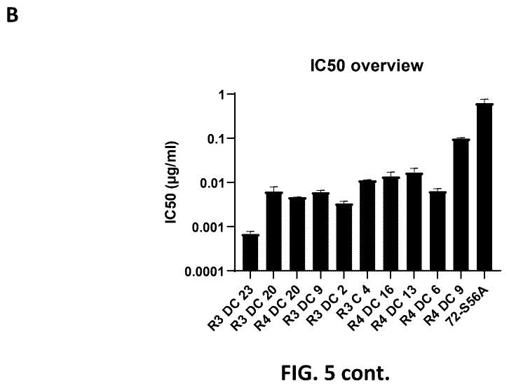

The neutralizing potency of the S2 binding VHHs was tested in a neutralization assay using VSV particles pseudotyped with the spike of SARS-COV-2 (614G). The cross-neutralizing nanobody VHH72-S56A was used as reference (Schepens et al. (2021) Sci. Transl Med, 13:eabi7826). The neutralizing activity (IC₅₀) of the tested VHHs on Vero E6 cells ranged from about 100 ng/ml (R4_DC9) to less than 1 ng/ml (R3_DC23) (FIG. 5), Most VHHs neutralized SARS-COV-2 spike pseudotyped VSV with a potency of about 10 ng/ml (EC₅₀).

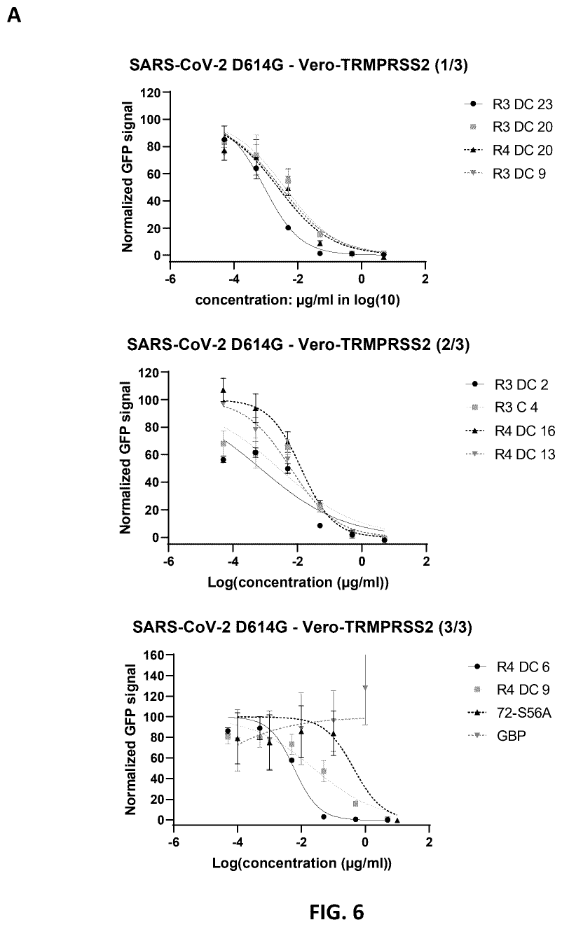

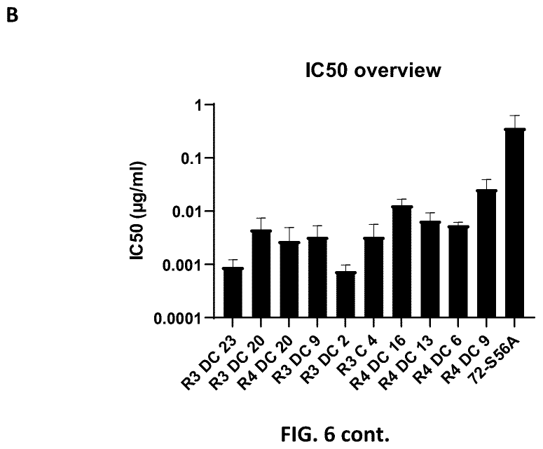

After viral attachment, the spike protein is cleaved at the S2′ site just upfront the fusion peptide. This enables the fusion peptide to insert into the membrane of the target cell to initiate membrane fusion. In cells that express TM protease serine 2 (TMPRSS2) at their surface, fusion can occur at the plasma membrane. In cells such as Vero E6 cells that lack TMPRSS2, fusion occurs in the endosomes after S2′ cleavage by cathepsin L, To test if the S2 binding VHHs can also neutralize when viral fusion occurs at the host cell plasma membrane, their neutralizing activity was also investigated on Vero E6 cells expressing human TMPRSS2 (Vero E6-TMPRSS2), FIG. 6 illustrates that for all VHHs the neutralizing activity on Vero E6-TMPRSS2 cells was either very similar or higher than compared to Vero E6 cells lacking TMPRSS2 (FIG. 5).

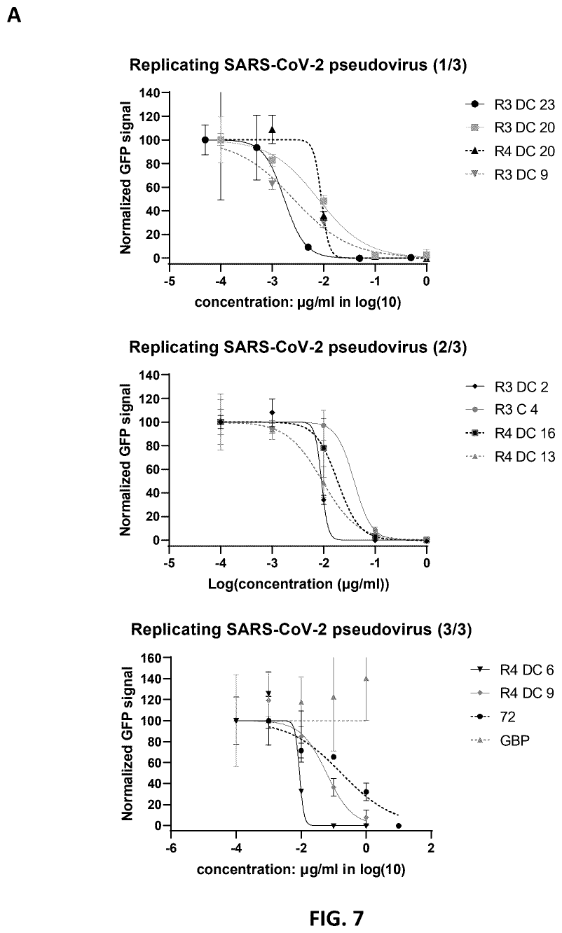

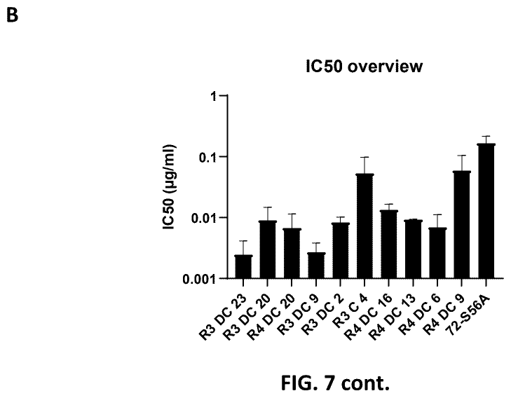

The neutralizing activity of the VHHs was also confirmed using replication-competent VSV pseudotyped with the SARS-COV-2 spike (FIG. 7) (Koenig et al. (2021) Science 371:eabe6230).

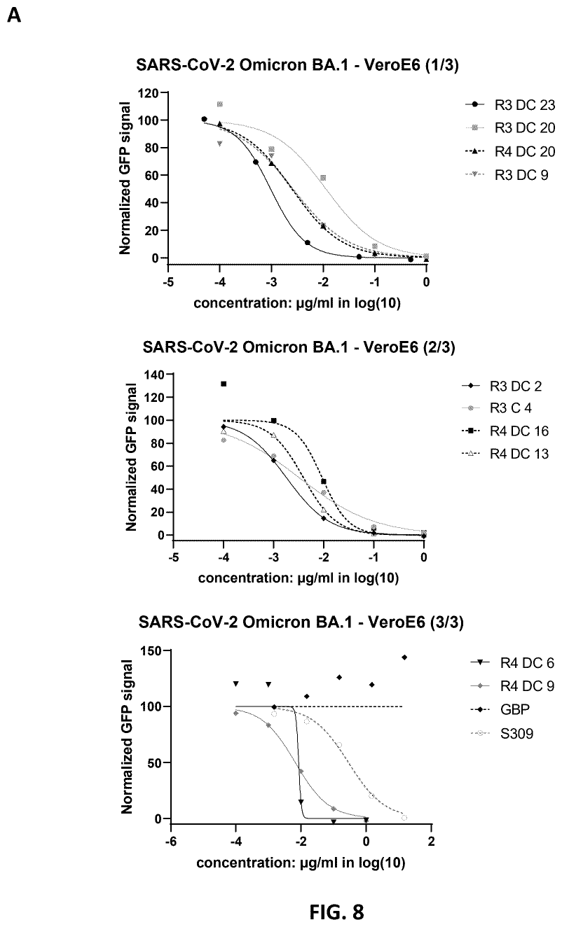

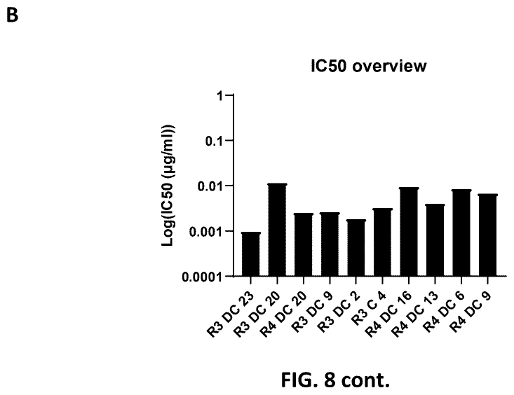

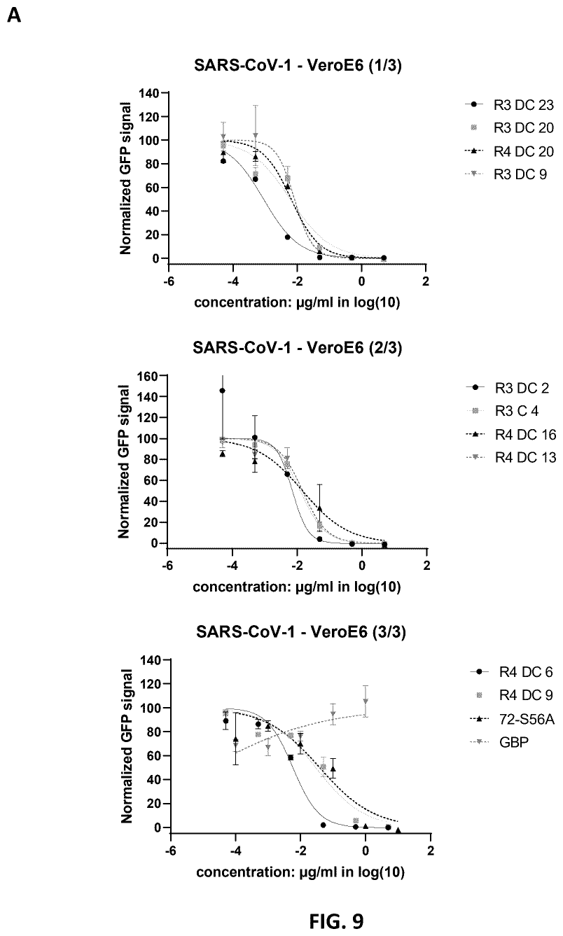

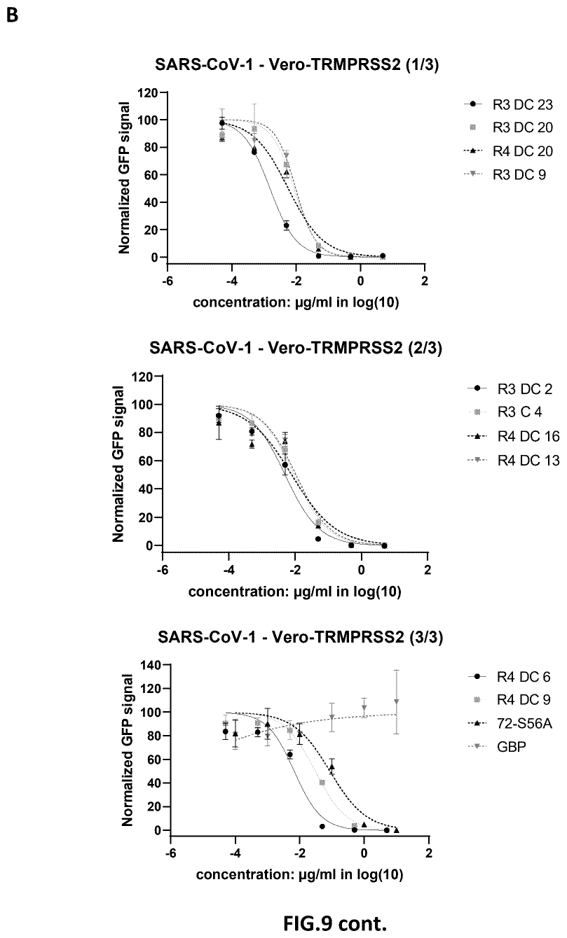

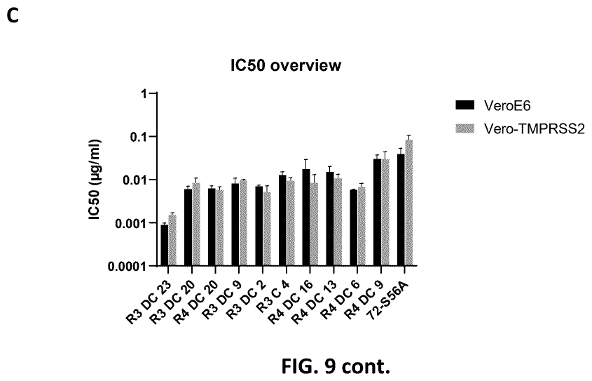

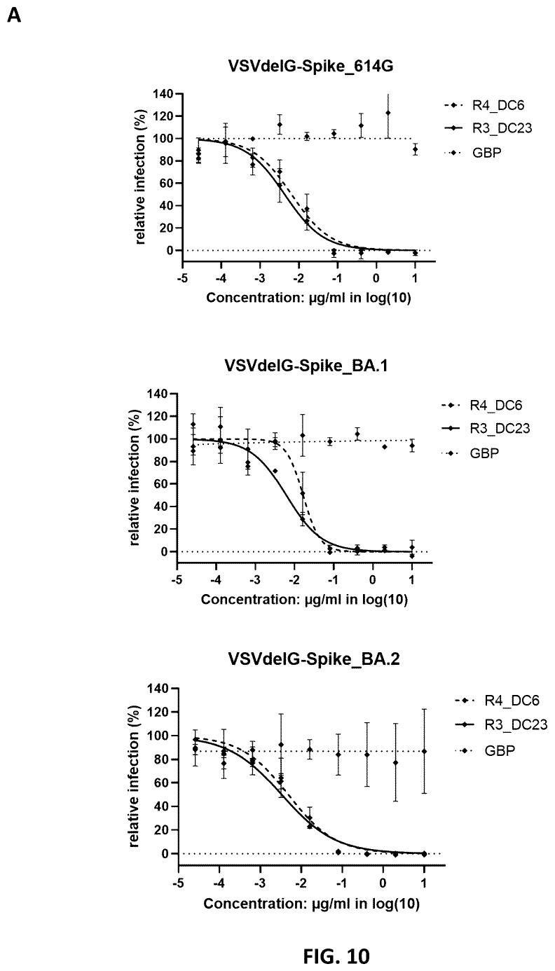

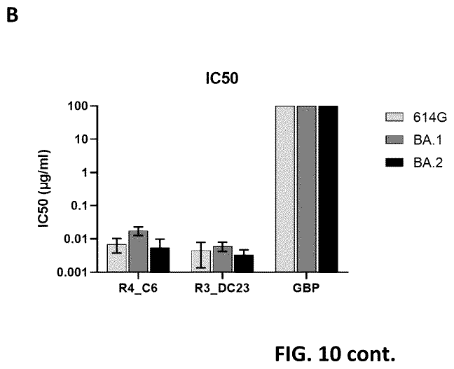

To probe the breath of the neutralizing activity of the family 1 VHHs, neutralization assays using VSV pseudotyped with the spike proteins of the Omicron BA.1 variant or SARS-COV-1 were performed, FIGS. 8 and 9 demonstrate that in agreement with efficient binding to the spike proteins of the Omicron BA.1 variant and of SARS-COV-1 (FIG. 3) family 1 VHHs could also potently neutralize VSV particles pseudotyped with the spikes of the respective viruses, To test if the S2 targeting VHHs can also neutralize the more recent BA.2 Omicron variant, R3_DC23 and R4_DC6 were also tested for their ability to prevent infection of VSV particles pseudotyped with the spikes of the SARS-COV-2 Omicron BA.2 and Omicron BA.1 variants, and 614G spike protein, FIG. 10 demonstrates that both R3_DC23 and R4_DC6 could prevent infection of VSV particles pseudotyped with spike protein of Omicron BA.2 as efficient as viral particles pseudotyped with spike proteins of Omicron BA.1 or with 614G spike of SARS-COV-2.

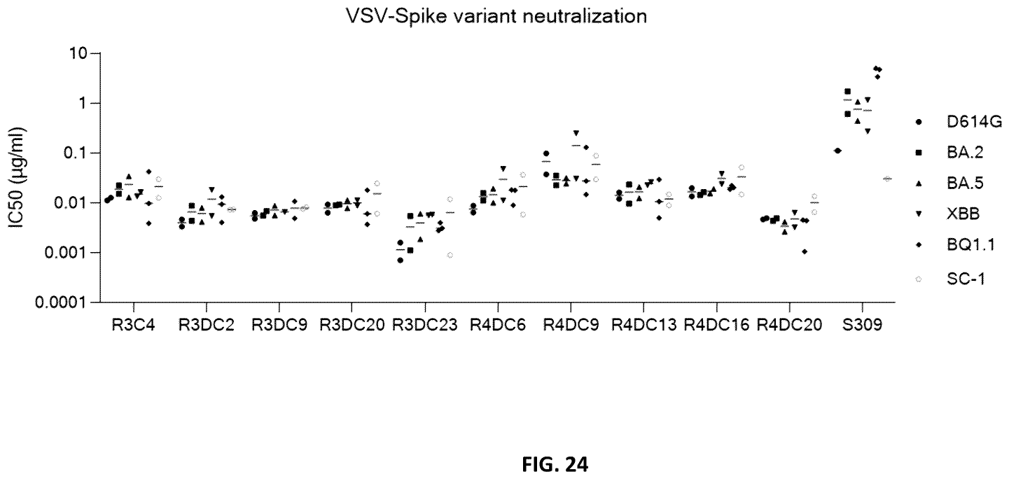

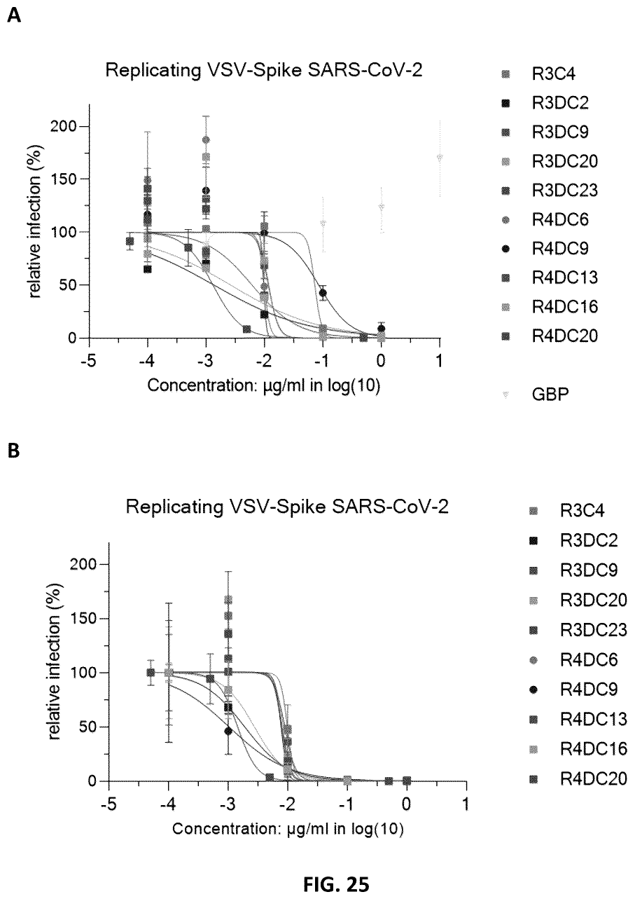

To evaluate the neutralization potency and breadth of the isolated S2 subunit-targeting VHHs neutralization assays were performed with VSV pseudotypes displaying spike proteins of SARS-CoV-2 D614G, -BA.2, -BA.5, -XBB, or -BQ1.1, All VHHs could potently neutralize these SARS-CoV-2 spike pseudotypes with IC₅₀ ranging from 1.2 (R3DC23) to 68.9 ng/ml (R4DC9) (80.0-4680 μM) for SARS-COV-2 both on Vero-E6 and VeroE6/TMPRSS2 cells, which allow viral entry at the cell surface (FIG. 24, FIG. 6), VHH-R3DC23 was one of the most potently neutralizing S2 binders with an IC₅₀ close to 1 ng/ml for SARS-COV-2 D614G and 2.9-5.7 ng/ml for the tested omicron variants (FIG. 24, FIG. 6=TMPRSS2 cells). The S2-binding VHHs also neutralized replicating VSV SARS-COV-2 spike pseudotypes with similar potency as the replication-deficient pseudotypes (FIG. 25). In line with their binding properties, the isolated VHHs also neutralized pseudotypes carrying the spike protein of SARS-COV-1 (FIG. 24).

### Example 4: VHHs Targeting the S2 Subunit Potently Neutralize Authentic SARS-COV-2 614G and Omicron BA.1 Variants In Vitro

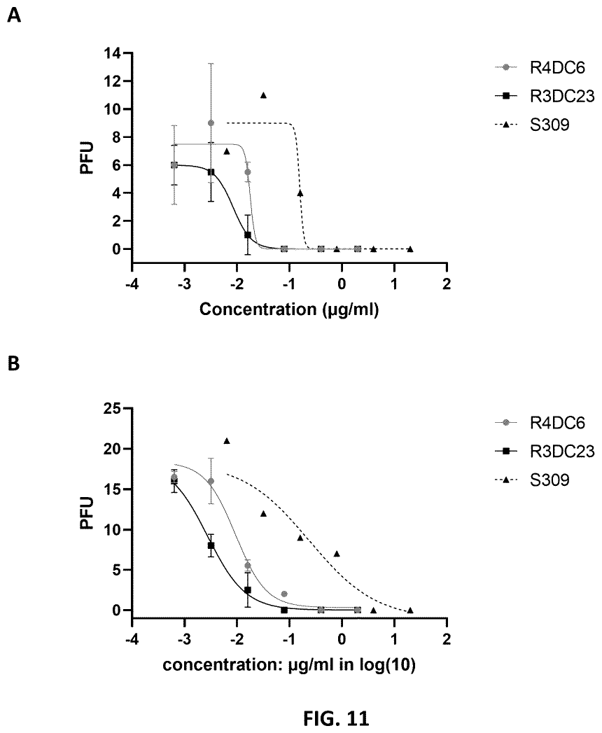

To test if the selected S2 targeting VHHs can also potently neutralize authentic SARS-COV-2 virus, plaque reduction assays were performed using SARS-COV-2 614G and SARS-COV-2 Omicron BA.1 variant viruses, Dilution series of R3_DC23 or R4_DC6, belonging to 2 different subfamilies, were pre-incubated with about 40 PFU of both virus variants for 1 hour at 37° C. and subsequently used to infect Vero E6-TMPRSS2 cells, Antibody S309, one of the few antibodies that is known to potently neutralize Alpha and Omicron BA.1 variants was used as positive control, FIG. 11 demonstrates that R3_DC23 and R4_DC6 could both neutralize 614G and Omicron BA.1 viruses more efficiently than S309, with IC50 values (4.325±3.490 ng/ml for SARS-COV-2 D614G and 4.711 #1.980 ng/ml for SARS-COV-2 BA.1, n=3) closely resembling those obtained for pseudotyped VSV particles (FIG. 5-10).

### Example 5: VHHs Targeting the S2 Subunit do not Evoke Shedding of the S1 Spike Subunit

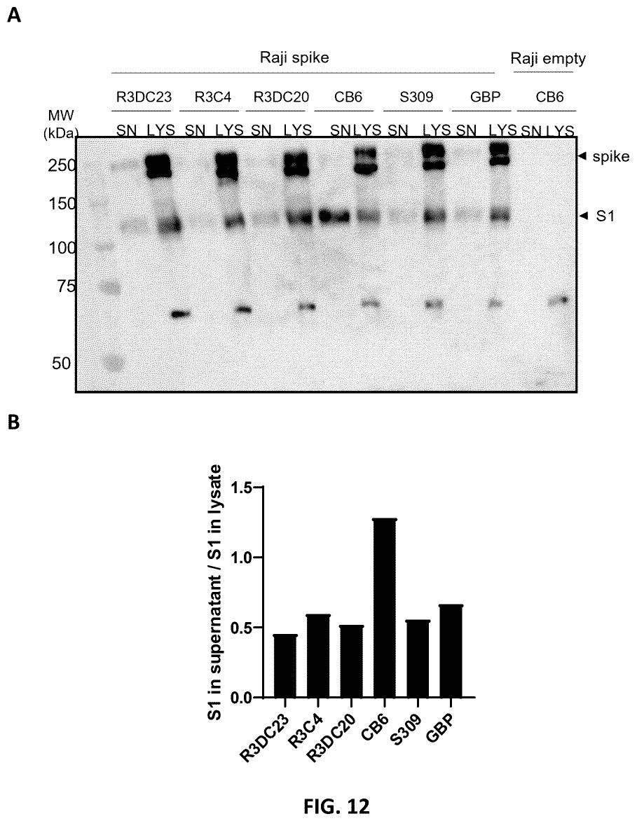

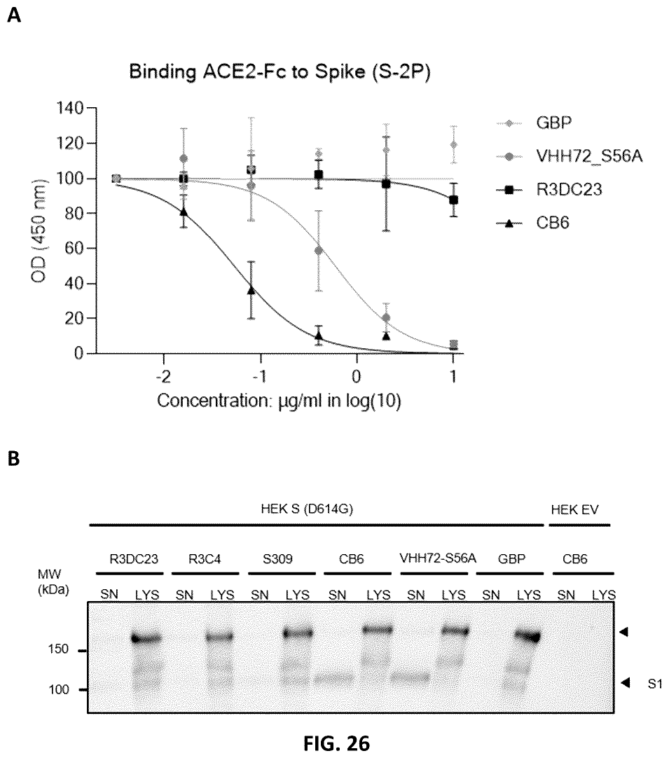

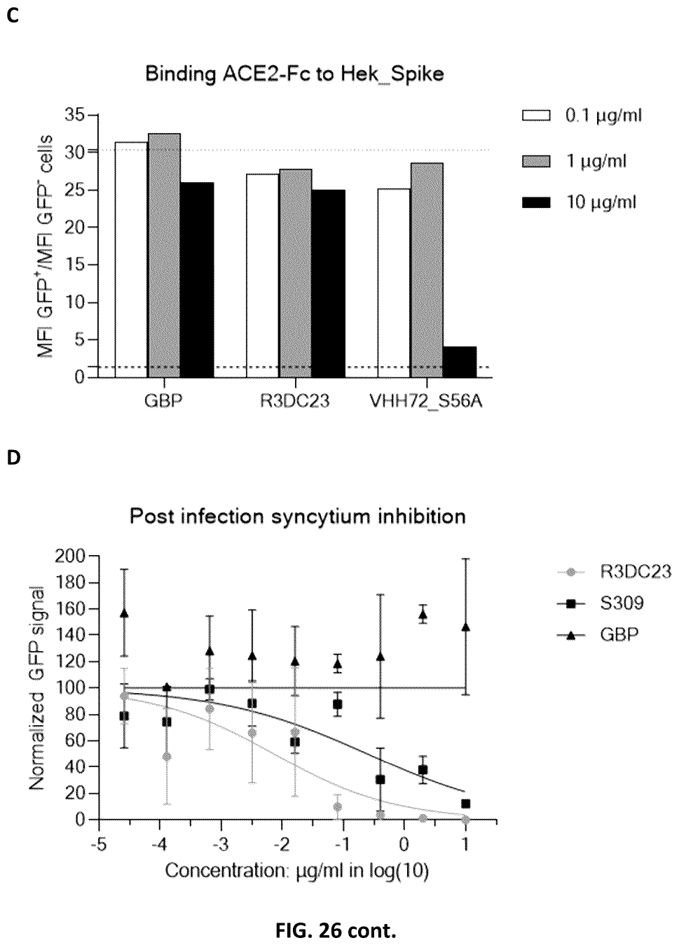

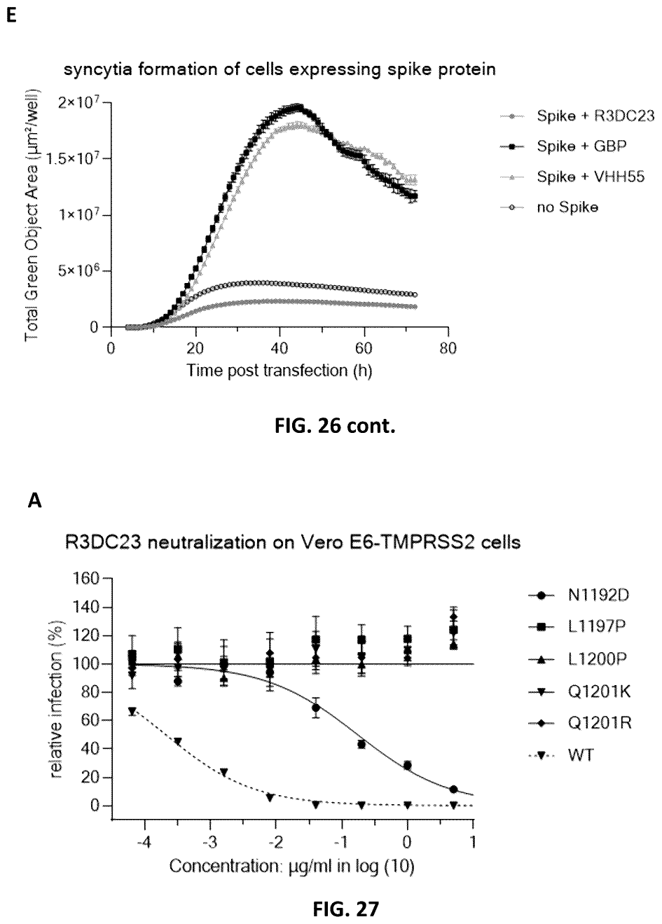

The neutralization assays using non-replicating VSV particles pseudotyped with spike proteins demonstrate that the isolated S2 specific VHH block viral entry, RBD targeting antibodies can prevent entry by blocking viral attachment by direct competition with ACE2 for RBD binding or by prematurely evoking S1 shedding. In line with the notion that the S1 subunit is responsible for receptor engagement, S2-targeting VHH R3DC23 did not prevent the binding of purified ACE2-Fc to recombinant SARS-COV-2 Spike-2P protein (FIG. 26A), To test if the S2 binding VHHs can induce S1 shedding, Raji cells expressing SARS-COV-2 spikes at their surface were treated with the S2 binding VHHs, CB6 and S309 monoclonal antibodies, known to respectively, induce and not induce S1 shedding were used as positive and negative controls. In addition, a GFP binding VHH (GBP) was used as negative control, Western blot analysis of the cell culture medium and cell pellet collected after antibody incubation revealed that similar to S309, none of the VHHs was able to induce S1 shedding (FIG. 12).

Whereas monoclonal antibody CB6 and VHH72_S56A induced S1 shedding, R3C4 and R3DC23 failed to do so (FIG. 26B). In line with this, R3DC23 could not prevent the binding of ACE2-Fc to cells that express SARS-COV-2 spike whereas VHH72_S56A prevented this interaction (FIG. 26C), These results strongly suggest that the S2-specific VHHs prevent virus entry after attachment.

### Example 6: VHHs Targeting the S2 Subunit Block Fusion

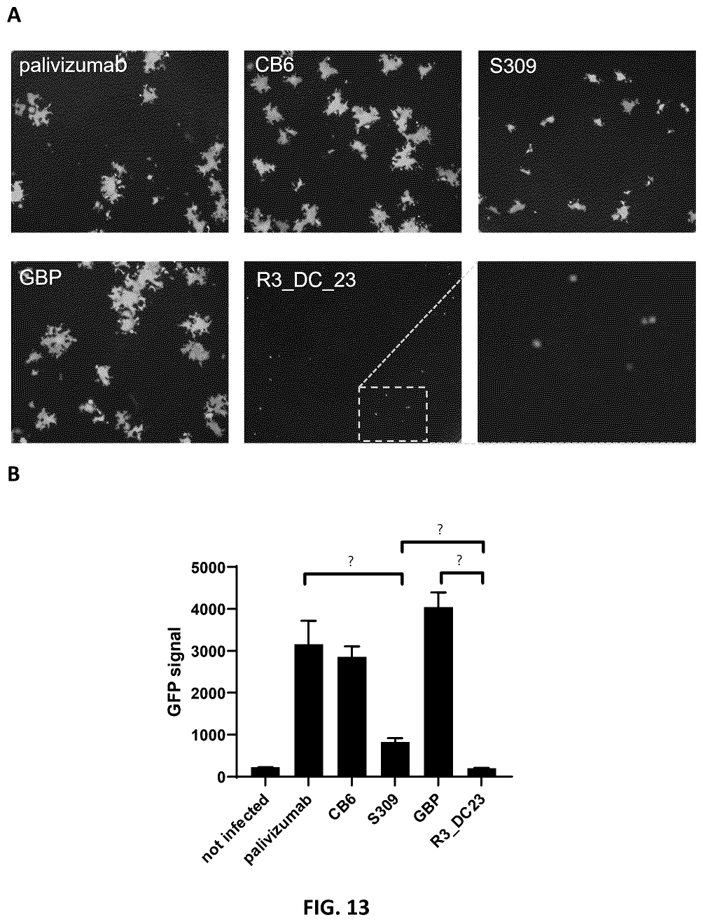

Next to viral attachment, antibodies can also block viral entry by interfering with spike-mediated fusion, To test if the selected VHHs (R3_DC23) can interfere with the fusion process, Vero E6-TMPRSS2 cells were infected with a low multiplicity of infection (MOI) of GFP expressing replication-competent VSV-spike (VSV-delG virus pseudotyped with Wuhan SARS-COV-2 spikes) and 4 hours later treated with 10 μg/ml R3_DC23. The human monoclonal antibody S309 (sotrovimab), known to interfere with viral fusion was used as positive control (Lempp et al. (2021) Nature 598:342-347). The GFP targeting VHH (GBP) and the RSV neutralizing antibody palivizumab were used as negative controls. In addition, the potently neutralizing antibody CB6 (Etesevimab) that competes with ACE2 for RBD binding was also included as reference (Shi et al. (2020) Nature 584:120-124), After overnight infection, the cells were fixed and analyzed for GFP expression by plate reader and microscopic analysis, FIG. 13A illustrates the formation of large GFP expressing syncytia for cells treated with the GBP control VHH and the control antibody palivizumab, Large GFP expression syncytia were also observed for cells treated with the neutralizing CB6 antibody. For samples treated with the S309 antibody smaller syncytia were observed. In sharp contrast, for the samples treated with the S2 targeting VHH R3_DC23 only single GFP expressing cells could be observed (FIG. 13A). The reduction in syncytia formation by R3_DC23 was also reflected by a clearly lower level of GFP expression in these samples (FIG. 13B).

This illustrates the ability of S2 targeting R3_DC23 to prevent syncytia formation and to restrain infection via strong inhibition of viral fusion.

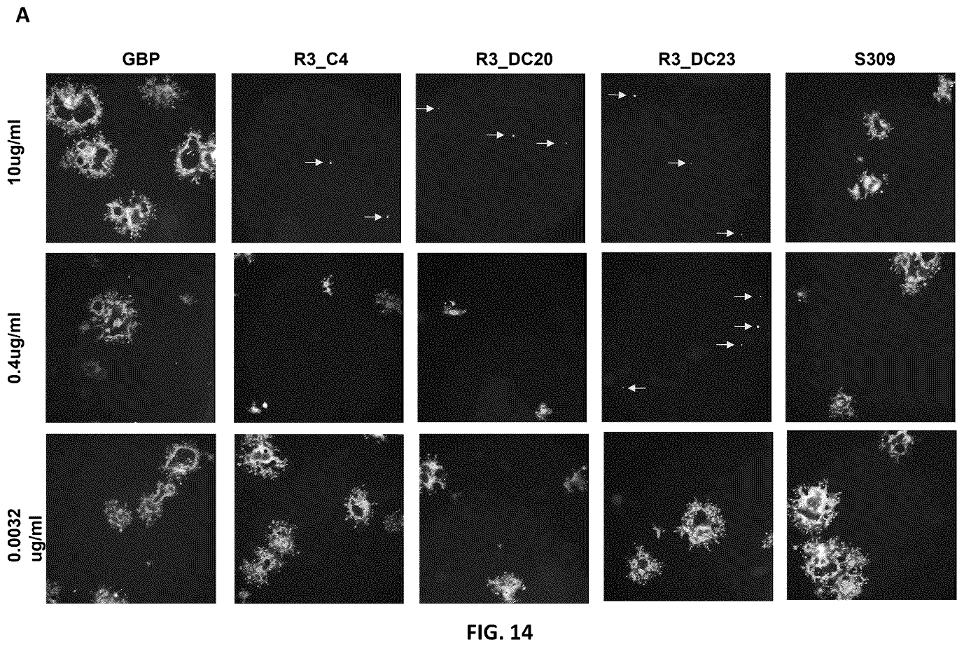

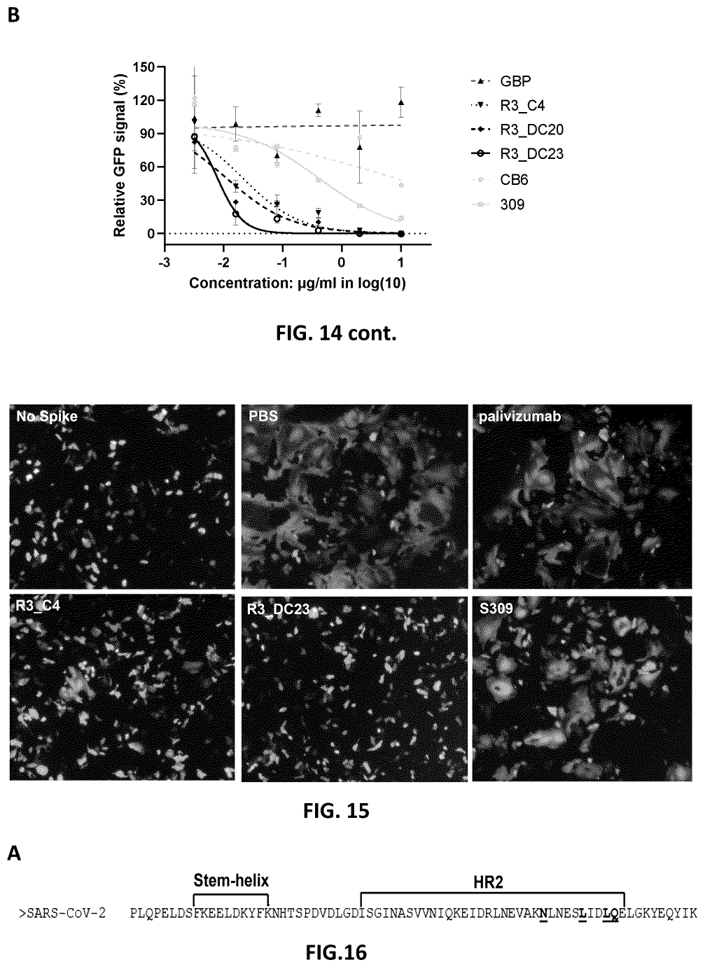

To determine the potency of the isolated S2 targeting VHHs, the ability to prevent syncytia formation after infection has occurred was tested at different concentrations of R3_DC23, R3_DC20 and R3_C4. In wells treated with 10 μg/ml of the S2 targeting VHH no syncytia but only individual infected cells could be observed (FIG. 14A). In contrast, at this concentration S309 treatment could not prevent but rather restrain syncytia formation to some extent (FIG. 14A). At 0.4 μg/ml, R3_DC23 was still able to prevent syncytia formation whereas the syncytia present in the samples treated with R3_DC20 or R3_C4 were smaller than in the GBP or S309 treated samples (FIG. 14A). The observed strong inhibition of syncytia formation by the S2 targeting VHHs was also clearly reflected in a strong concentration-dependent inhibition of GFP expression (FIG. 14B). For each VHH or monoclonal antibody dilution series the IC50 was calculated using a linear regression curve fitting (log (inhibitor) vs, normalized response with variable slope). The average calculated IC50 value (N=2) for CB6 was 8.026 μg/ml; for S309: 0.4064 μg/ml; for R3_C4: 0.02092 μg/ml; for R3_DC20: 0.01104 μg/ml and for R3_DC23: 0.007840 μg/ml. In line with the neutralization data, R3 DC23 was the most potent VHH in this fusion inhibition assay.

To more directly test fusion inhibition, it was investigated if S2 targeting VHHs could block fusion of Vero E6 cells that is induced by SARS-COV-2 spike expression, To this end, Vero E6 cells were transfected with an SARS-COV-2 spike expression vector in combination with an GFP expression vector and 2 hours later treated with VHHs or antibodies, Cells transfected with an GFP expression vector in combination with a control expression vector were used as control, FIG. 15 illustrates that, as expected, expression of the SARS-COV-2 spike protein in Vero E6 resulted in the formation of syncytia expressing GFP. In comparison to treatment with the RSV specific control antibody palivizumab or PBS, treatment with the S309 antibody reduced syncytia formation to some extent.

However, syncytia formation was almost completely prevented in samples treated with the isolated S2 targeting VHHs R3_DC23 and R3_C4 (FIG. 15). These data consistently indicate that the isolated S2 targeting VHHs prevent infection by efficiently interfering with spike-mediated membrane fusion.

It was tested if R3DC23 could block syncytium formation of VeroE6/TMPRSS2 cells following infection with replication-competent GFP-expressing VSV pseudotyped with SARS-COV-2 spike.

Addition of R3DC23 four hours after infection potently (IC50=9.2±2.6 ng/ml) prevented syncytium formation whereas moderate inhibition was observed with S309 (IC50=328.1±110.8 ng/ml) (FIG. 26D). The impact of R3DC23 on the kinetics of fusion of Vero E6 cells co-expressing spike and GFP was monitored via time-lapse imaging. In contrast to wells that contain cells that express only GFP, wells that contain cells expressing both GFP and spike proteins displayed a marked increase in the area of GFP-positive cells after 24 hours of transfection, reflecting syncytia formation. This increase was however strongly impaired by R3DC23 (FIG. 26E), Example 7: The isolated VHHs bind the S2 subunit at the membrane proximal HR2.

Monolayers of Vero E6-TMPRSS2 seeded in a 96 well plate were infected with about 200 PFU of GFP expressing replication-competent VSVdelG-spike virus and treated with 10 μg/ml of R3_DC23, VSV-spike instead of authentic SARS-COV-2 virus was used because replication of VSV is remarkably more error-prone, GFP was used to monitor syncytia formation and viral replication. At 10 μg/ml R3_DC23 GFP completely blocked syncytia formation and second round of infection of WT virus (see also Example 6). Therefore, syncytia formation or replication in the presence of 10 μg/ml R3_DC23 beyond the initially infected cells would likely indicate viral escape. The growth medium of wells that displayed clear syncytia formation or viral replication was collected and used to make dilution series that were mixed with either R3_DC23 (2 μg/ml) or medium, Dilution series that displayed viral replication in both the absence and presence of R3_DC23 were selected, From these dilution series wells that contained single plaque were selected and further propagated on Vero E6-TMPRSS2 cells in a 6 well plate in the presence of 2 μg/ml R3_DC23 for sequence analysis. The S2 coding sequence of 9 viral clones was sequenced and compared to that of the parental virus that was grown in parallel in the absence of R3_DC23.

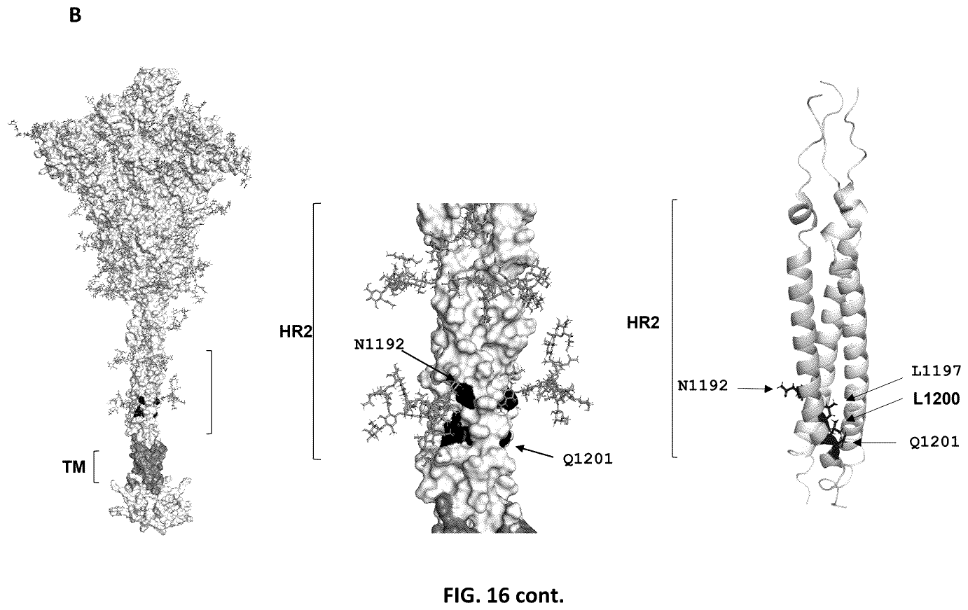

Each viral clone displayed a single non-silent point mutation, which were all located within the Heptad Repeat Region 2 (HR2) of the spike stalk proximal to the viral membrane (FIG. 16). The following 5 substitutions were identified at 4 positions within the HR2: N1192D (1/9), L1197P (2/9). L1200P (1/9), Q1201R (4/9) and Q1201K (1/9). The numbers in parentheses represent the frequencies at which each of the substitutions has been observed among the 9 selected escape variants.

A search on the Global Initiative on Sharing Avian Influenza Data (GISAID) database on April, 13, 2022 revealed that out of the 10,247,044 sequences only 247, 49, 20, 358 and 3865 sequences have respectively the N1192D, L1197P, L1200P, Q1201R and Q1201K substitutions. The positions N1192, L1197, L1200 and Q1201 were respectively, mutated only 1459, 790, 1762 and 5931 times.

Remarkably, a potential N-glycosylation site at position N1194 is located within the confined linear sequence comprising the mutations (AA1192-Q1201R), Site-specific mass spectrometry revealed that this site is almost 100% glycosylated (Watanabe et al. (2020) Science 369:330-333), Without wishing to be bound by any theory, such a narrow epitope might be hard to reach for conventional antibodies.

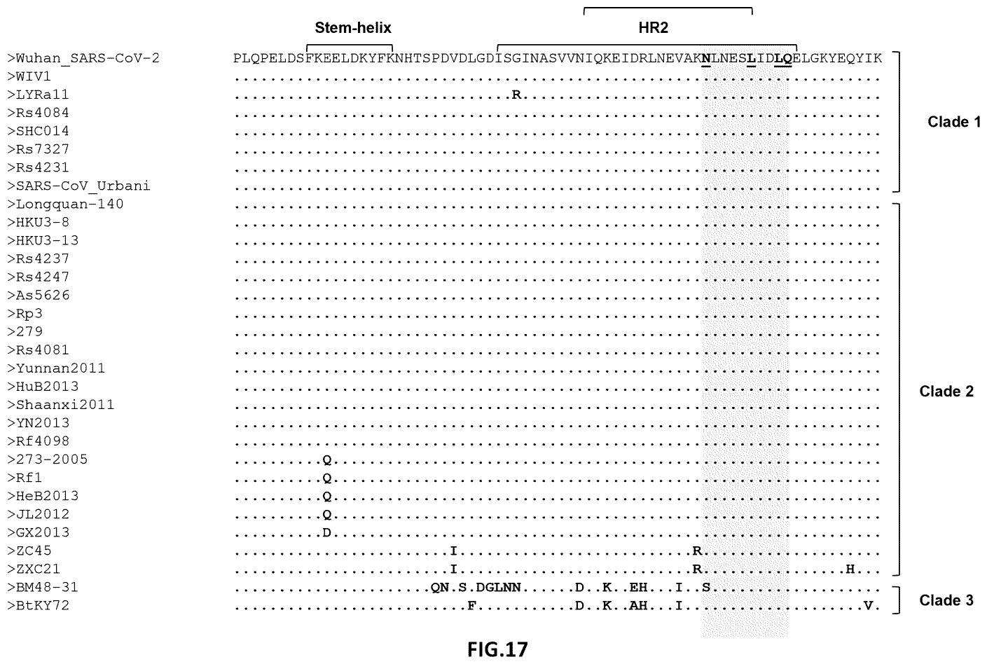

Sequence analysis of clade 1, 2 and 3 sarbecovirus S2 stem regions revealed that this sequence is highly conserved among sarbecoviruses (FIG. 17).

### Example 8: Fc Fusions of the HR2 Targeting VHH.R3_DC23, VHH.R3_C4 and VHH.R4DC20

can efficiently bind to cell surface expressed spike proteins and potently neutralize SARS-COV-2.

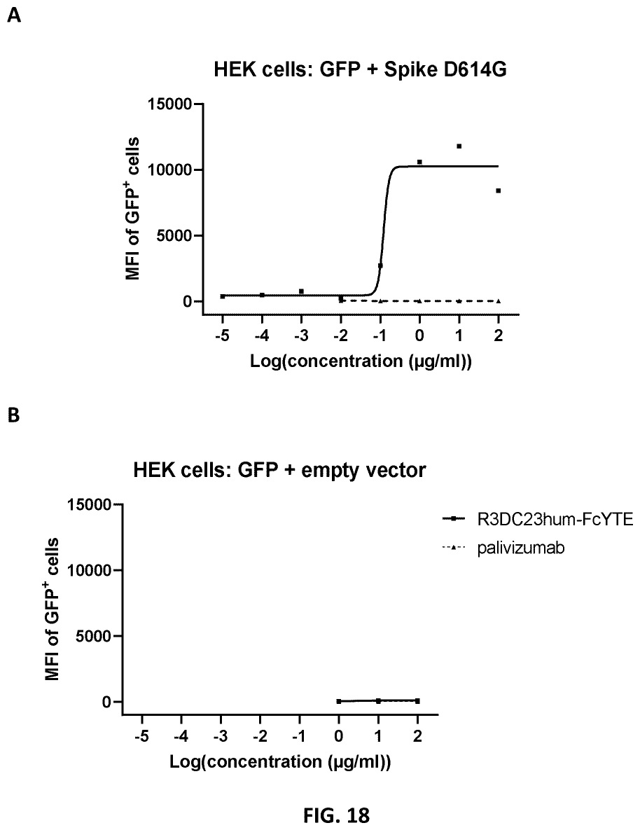

Fc(YTE) fusions of the HR2 targeting VHH R3_DC23 were produced and their binding to cells expressing the SARS-COV-2 spike protein at their surface was tested, HEK293T cells were transfected with either an GFP expression vector alone or in combination with an SARS-COV-2 spike expression vector (614G) and were tested for R3_DC23-Fc(YTE) binding by flow cytometry, FIG. 18 illustrates that R3_DC23-Fc(YTE) could very efficiently bind to cells expressing the SARS-COV-2 spike at their surface. In sharp contrast, no binding of R3_DC23-Fc(YTE), which was added up to 100 μg/ml, was observed to control cells not expressing the SARS-COV-2 spike.

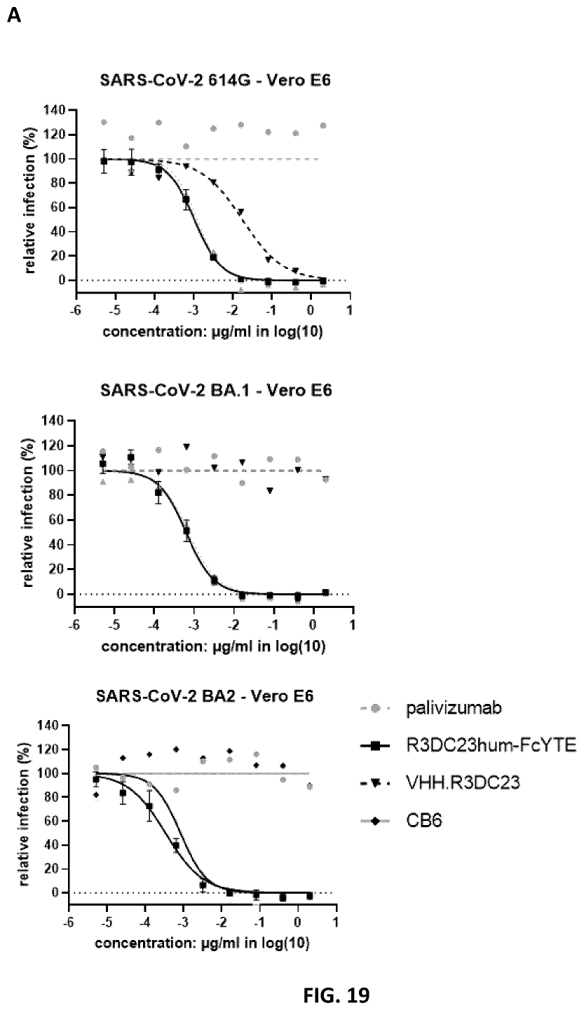

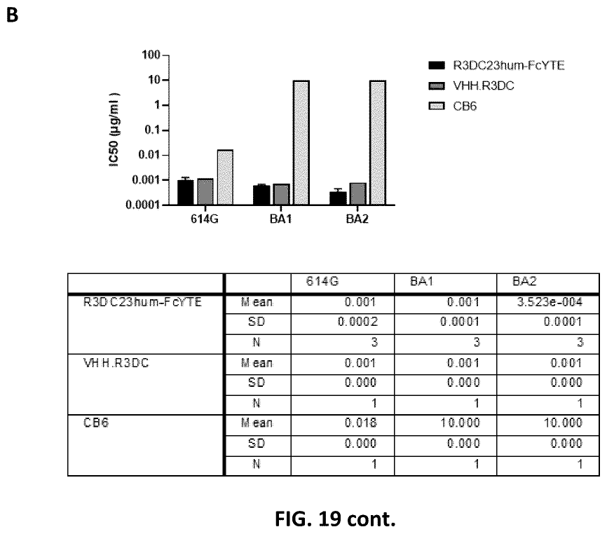

To test the neutralizing activity of R3_DC23-Fc(YTE), neutralization assays using VSV reporter viruses pseudotyped with the spike protein of the SARS-COV-2 (614G), or with the spike protein of the Omicron BA.2 and Omicron BA.1 variants were performed, FIG. 19 illustrates that R3_DC23-Fc(YTE) could potently neutralize VSV particles pseudotyped with the spike proteins of SARS-CoV-2 (614G) and the Omicron BA.1 variant with an EC50 of 1 ng/ml whereas the EC50 for Omicron BA.2 spike pseudotyped VSV particles was only 0.35 ng/ml. This illustrates that VHH-Fc fusion can efficiently recognize the identified membrane proximal epitope (see Example 7) and can neutralize SARS-COV-2 and its variants with exceptional potency.

Similar to R3DC23, the framework regions of two additional related VHHs (R3C4 and R4DC20) were humanized and the N-terminal glutamine replaced by an aspartate residue, and these S2-binding VHHs were genetically fused to a human IgG1-Fc_YTE. The humanized constructs huR3DC23-Fc (SEQ ID NO:96), huR3C4-Fc (SEQ ID NO:130) and huR4DC20-Fc (SEQ ID NO:131) were produced in mammalian cells and compared with their monovalent counterpart for neutralization of pseudotyped VSV displaying the spike protein of SARS-COV-2 614G and BA.5. The sequences defined by SEQ ID NO: 130 and 131 are also shown below.

SEQ ID NO: 130
DVQLVESGGGLVQPGGSLRLSCAVSGRPFSTYTMGWFRQAPGKEREFVAAIRWSGGTIYYADSVKG
RFTISRDNAKNTVYLQMNSLRPEDTAVYYCAAAYVSKANYGSLWYRASGLYDYWGQGTLVTVSSGG
GGSGGGGSDKTHTCPPCPAPELLGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPG
SEQ ID NO: 131
DVQLVESGGGLVQPGGSLRLSCAVSGRPFSTYTMGWFRQAPGKEREFVASIRWSGGTTNYADSVKG
RFTISRDNAKNTVYLQMNSLRPEDTAVYYCAAAYVSKANYGSLWYRNSGLYDYWGQGTLVTVSSGG
GGSGGGGSDKTHTCPPCPAPELLGGPSVFLFPPKPKDTLYITREPEVTCVVVDVSHEDPEVKFNWY
VDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPG

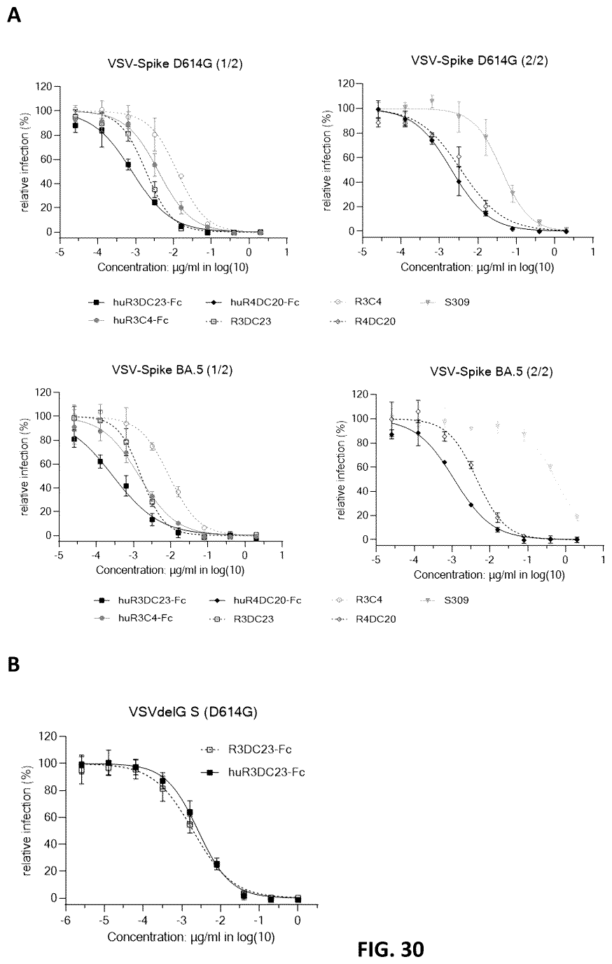

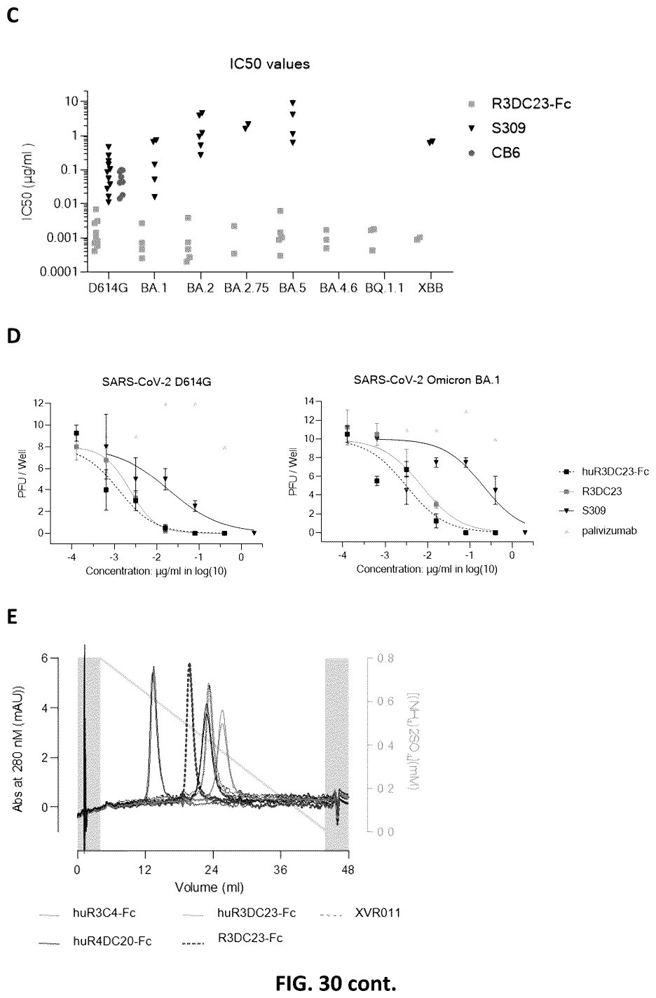

For each of these VHHs the humanized Fc fusions neutralized SARS-COV-2 614G and BA.5 more efficiently than their monovalent formats with huR3DC23-Fc being the most potent. Moreover, humanization of R3DC23 did not affect the neutralizing activity of huR3DC23-Fc (FIG. 30B), An important parameter for the development of biologicals is their solubility. Therefore, the hydrophobicity of the VHH-Fc fusions was investigated by hydrophobic interaction chromatography.

From the three humanized VHH-Fc fusion constructs the retention times on hydrophobic interaction chromatography was shortest for huR3DC23-Fc, below its non-humanized counterpart and well below that of clinically validated VHH-Fc XVR011. These data indicate that based on solubility and neutralizing activity huR3DC23-Fc is most favorable for further development (FIG. 30E), Similar as for SARS-COV-2 614G and BA.5 huR3DC23-Fc could also potently neutralize VSV pseudotypes displaying the spike protein of SARS-COV-2 BA.1, BA.2, BA2.75, BA4.6, BQ1.1, and XBB with IC50 values close to or even below 1 ng/ml (FIG. 30C), Moreover, huR3DC23-Fc could also potently neutralize authentic SARS-COV-2 D614G and BA.1 (FIG. 30D).

### Example 9: Fc Fusions of the HR2 Targeting VHH.R3_DC23 can Protect Mice Against a Lethal Viral Challenge with SARS-COV-2

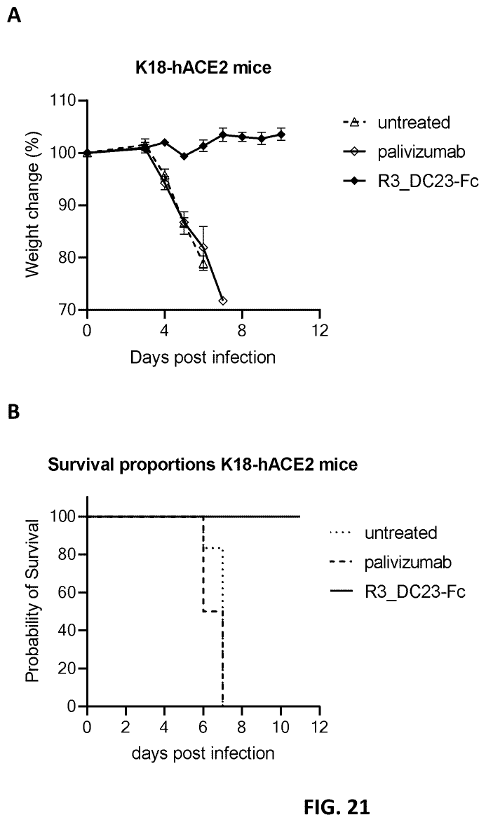

K18-hACE2 mice that express human ACE2 at the surface of their epithelial cells were treated with 100 μg R3_DC23-Fc or 100 μg isotype control IgG (palivizumab) 1 day prior to a lethal infection with 614G SARS-COV-2 virus, Infected mice that were not treated were included as control, FIG. 21 illustrates that in sharp contrast to mice that were either treated with isotype control antibody or untreated mice, all mice that were treated with R3_DC23-Fc survived the challenge and did not display significant bodyweight loss, indicating that R3_DC23-Fc can protect mice from a lethal viral challenge with SARS-COV-2.

### Example 10: S2-Binding VHHs Bind to the Membrane Proximal Region of Heptad Repeat 2 (Escape Virus Selection Experiments)

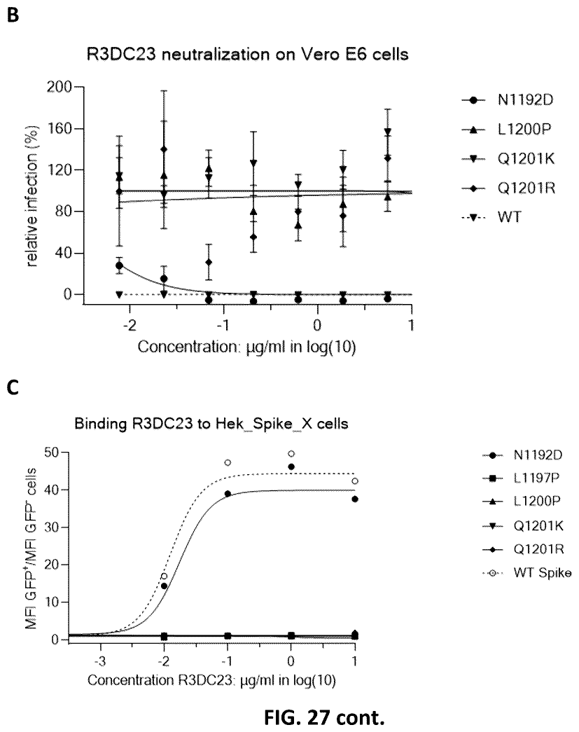

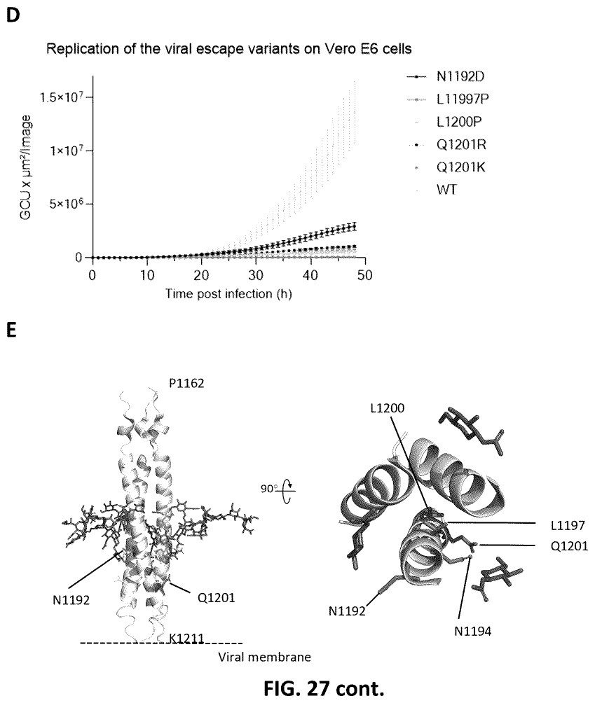

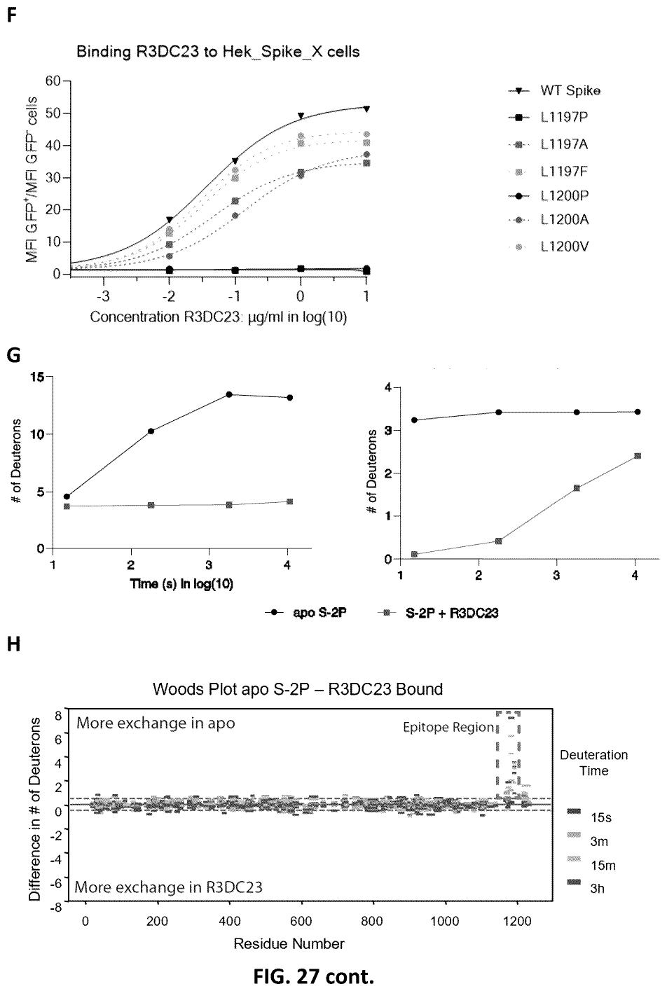

Escape viruses were selected according to Example 6, Escape viruses with mutations L1197P, L1200P, or Q1201R were completely resistant to R3DC23 neutralization whereas the N1192D mutant virus remained sensitive to neutralization by R3DC23, especially on Vero E6 cells (FIGS. 27A and B). In line with this, R3DC23 could still bind to cell surface expressed spike with the N1192D substitution whereas binding to spike mutants with any of the other escape selection mutations was lost (FIG. 27C).

The amino acids in the escape viruses that abolish R3DC23 binding are confined to the part of the spike that sits between the viral membrane and a large tetra-antennary N-glycan at position N1194, The complete HR2 of SARS-COV-2 is identical to that of SARS-COV-1 but differs in MERS and HKU1. In particular, the HR2 of both MERS and HKU-1 contains the Q1201K mutation that was acquired by viral escape mutants and that completely abrogated R3DC23 binding and neutralization, Without wishing to be bound by any theory, this may explain the inability of R3DC23 and the related VHHs to recognize MERS and HKU-1 spikes. In the SARS-COV-2 spike prefusion conformation the membrane proximal part of HR2 assembles into a coiled-coil of 3 parallel alpha helices that, upon transition to the postfusion conformation, initially disassemble and then bring the viral and host cell membrane in proximity by zippering up with the HR1 coiled-coil to form a stable 6-helix bundle (6HB), This crucial function of the membrane proximal HR2 region in the fusion process explains why the VSV pseudotype R3DC23 escape viruses were all highly attenuated (FIG. 27D), According to the available NMR structure of the SARS-COV-1 HR2 Q1201 is oriented outwards but both L1197 and L1200 are oriented towards the center of the coiled-coil (FIG. 27E), This indicates that either R3DC23 binds an open conformation of the HR2 or that the leucines at positions 1197 and 1200 do not make direct contact with R3DC23 but that substitutions at these positions to prolines have a significant impact on the folding of the HR2 coiled-coil, To address this, we evaluated R3DC23 binding to spike muteins with either a L1197F, L1197A, L1200V, or L1200A substitution instead of a proline, which is considered a helix breaker, R3DC23 could bind to spike with L1197F, L1197A, L1200V, or L1200A substitution, indicating that an intact HR2 coiled-coil tertiary structure is essential for its binding (FIG. 27F).

### Example 11: S2-Binding VHHs Bind to the Membrane Proximal Region of Heptad Repeat 2 (HDX-MS)

To further confirm the epitope of R3DC23, hydrogen-deuterium exchange monitored by mass spectrometry (HDX-MS) was conducted on recombinant SARS-COV-2 spike in the presence and absence of R3DC23, Two adjacent peptides from residues 1187 through 1205 (peptide (1187-1199) and peptide (1200-1205)) were found to be highly protected in the presence of R3DC23 (FIG. 27G), Importantly all 4 positions at which viral escape variants acquired mutations locate within those two peptides, Consistent with the binding of the nanobody to the spike protein in this region, this high degree of protection was not seen in any other regions of the protein (FIG. 27H). These data consistently indicate that R3DC23 binds to the membrane proximal region of HR2.

### Example 12: R3DC23 Binds to a Quaternary Epitope in HR2 (Crystal Structure)

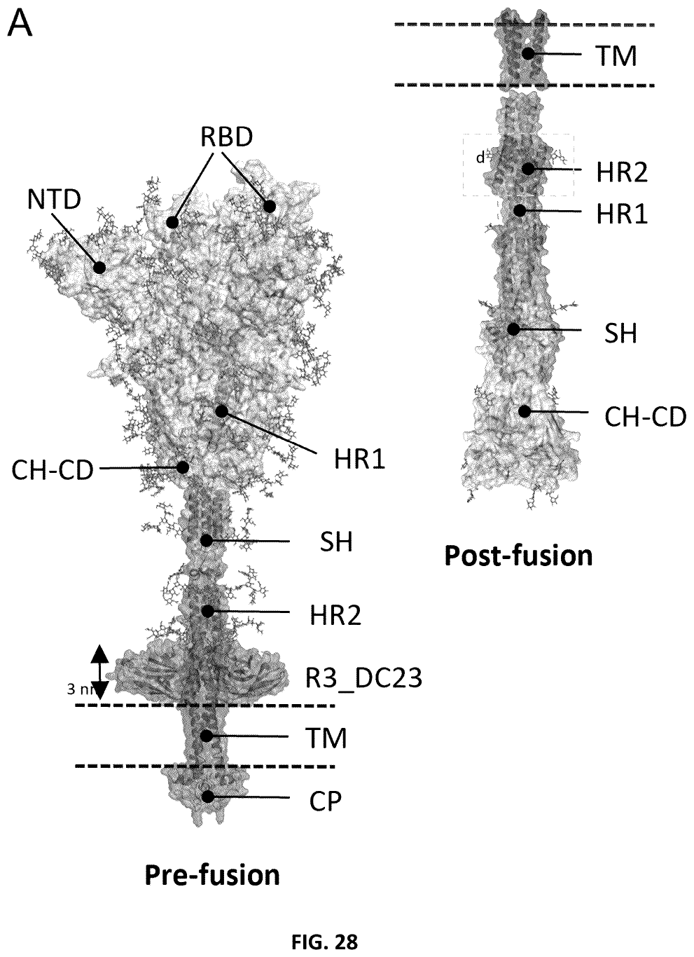

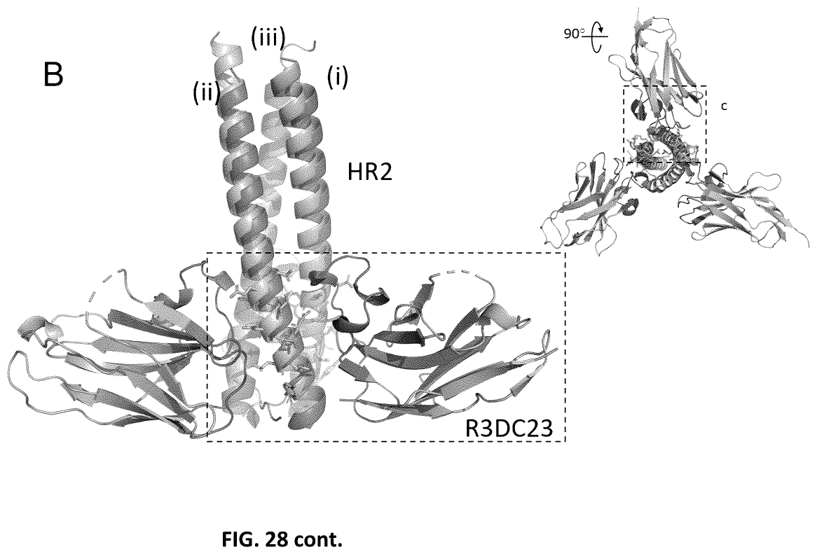

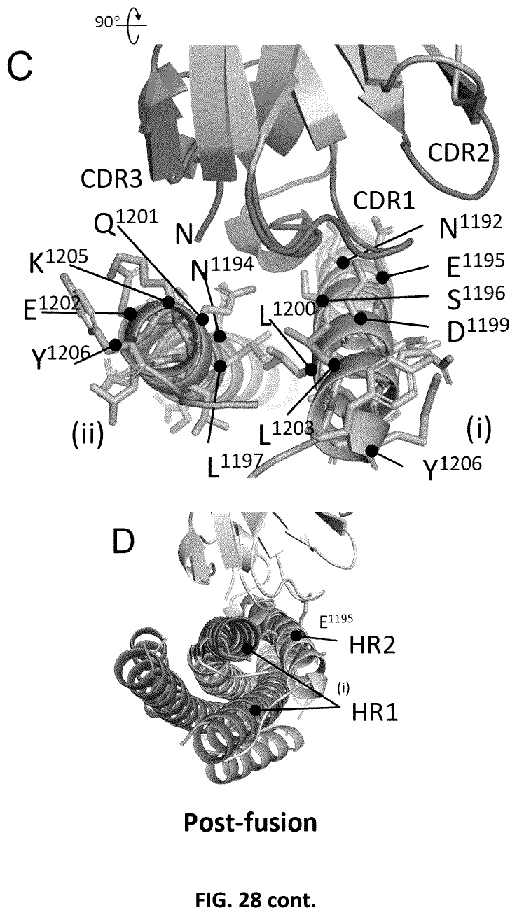

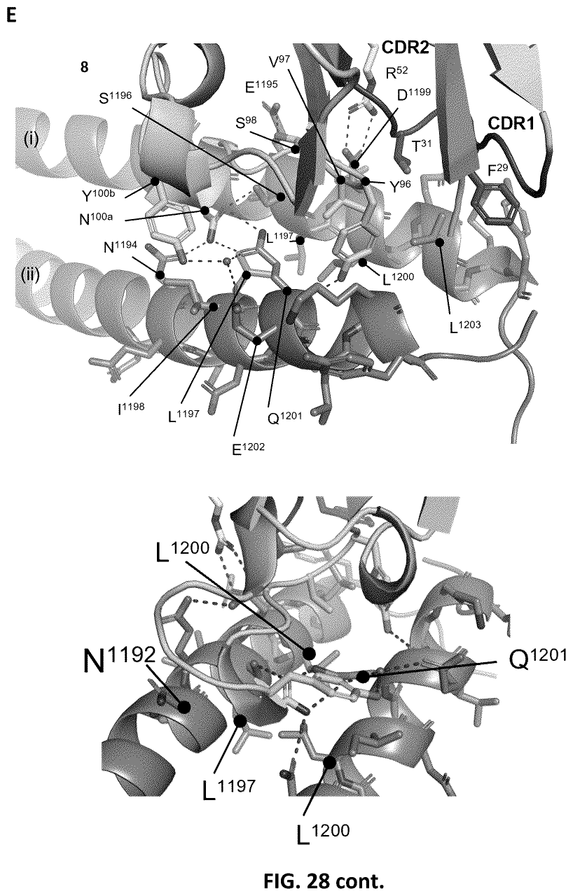

To get detailed insight in the interactions between R3DC23 and its target the crystal structure of R3DC23 in complex with a peptide spanning the complete HR2 (H1159-K1211) was resolved. The crystal asymmetric unit shows an HR2 coiled-coil trimer in complex with three R3DC23 molecules, each binding the interface between two HR2 peptides (FIG. 28A). The R3DC23 binding site spans residues N1192 to Y1206, encompassing the C-terminal region of HR2 (FIG. 28A-C), R3DC23 binds two adjacent HR2 helices, encompassing a 407 Å² buried surface area, 8 H-bonds, 2 salt bridges and a calculated solvation free energy gain ΔⁱG for complex formation (i.e. hydrophobic contribution to binding) of −4.7 kcal/mol for helix (i), and a 461 Å² buried surface area, 6 H-bonds, 1 salt bridge and a calculated ΔⁱG of −2.4 kcal/mol for helix (ii) (FIG. 28C, E). The VHH CDR3 forms the dominant contact surface in the complex, where N100a and Y100b form an extensive H-bond network with N1194, Q1201 and E1202 in helix (ii) and S1196 in helix (i), Y96 goes in H-bond contact with helix (ii) main chain carbonyls, S98 goes in H-bond interaction with E1195 in helix (i), and V97 binds a hydrophobic patch formed by L1200 and L1203 in helix (i). The VHH CDR1 and CDR2 are involved, resp. in hydrophobic interactions and two salt bridges (R52-D1199) with helix (i) (FIG. 28C, E). The identification of Q1201 as R3DC23 escape mutant agrees with its central part in the H-bond network with CDR3, L1197 and L1200, however, are not in direct contact with R3DC23, suggesting their activity as escape position is indirect, likely as a result of a destabilization and/or conformational adjustment of the adjoined binding epitope across the HR2 coiled-coil interface. The conformation of the HR2 trimer in the R3DC23 complex closely matches that seen in the pre-fusion form of the S protein (FIG. 28B), where it corresponds to an approximately 3 nm high region between the membrane and the HR2 and linker regions covered by N-glycosylation (FIG. 28A). In the postfusion form, HR2 rearranges to bind the surface of a HR1 coiled-coil, thereby breaking the pairwise HR2 contacts and resulting in partial unfolding of the HR2 C-terminal region (FIG. 28A,D), This rearrangement breaks the R3DC23 binding site, Conversely, the interfacial binding of R3DC23 to the HR2 coiled-coil is likely to exert a stabilizing activity on the pre-fusion S protein.

### Example 13: R3DC23 Binds to a Quaternary Epitope in HR2 (ELISA)

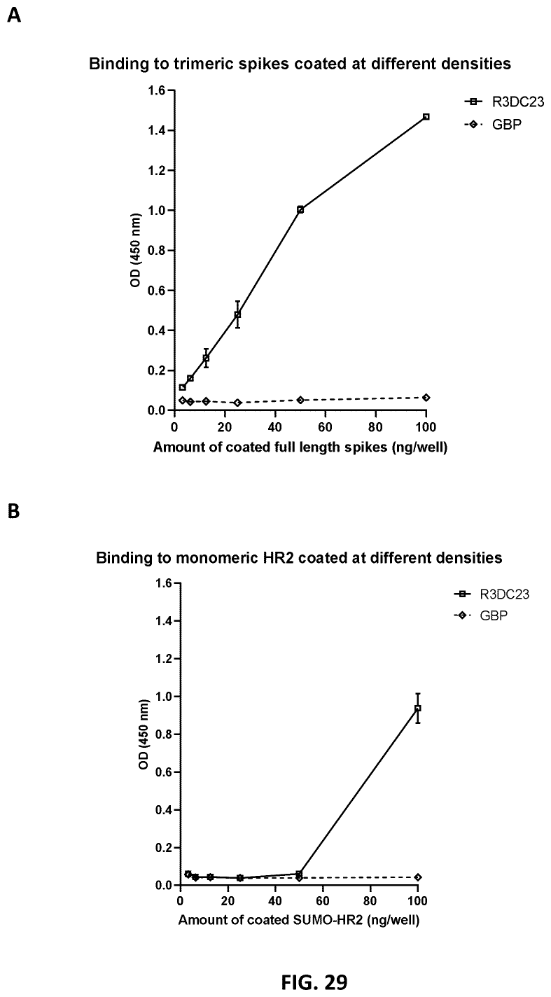

To test whether R3DC23 recognizes either a single HR2 alpha-helix or a quaternary epitope within the HR2 coiled coil, the binding of this VHH to full length spike proteins stabilized in a trimeric conformation by a T4 fibritin (foldon) trimerizing domain and a SUMO-HR2 peptide that was shown by SEC-MALS analysis to be monomeric was tested, Whereas binding of R3DC23 to trimeric spike directly correlated to the amount of full length spike that was coated, R3DC23 only bound to monomeric SUMO-HR2 when coated at high density (FIG. 29), This strongly suggests that R3DC23 binds to a quaternary epitope within the HR2 coiled coil comprising more than 1 HR2 alpha-helix.

### Example 14: LS Mutants of Humanized R3DC23 Fc Fusions Potently Neutralize a Broad Range of SARS-COV-2 Variants

Next to the YTE Fc variant also the LS (M428L/N434S) mutations increase the half-life of engineered antibodies by increasing their affinity of FcRn at low pH. Therefore, a construct was generated wherein a humanized form of R3_DC23 was fused to a human IgG1 Fc containing the LS mutation via a (G₄S)₂ linker (SEQ ID NO:121) at the N-terminus (R3_DC23hum-Fc(LS) or huR3DC23-Fc_LS or XVR013) (SEQ ID NO:118).

R3_DC23hum-Fc(LS) or huR3DC23-Fc_LS or XVR013:
(SEQ ID NO: 118)
DVQLVESGGGLVQPGGSLRLSCAVSGRIFSTYTMGWERQAPGKEREFVAAVRWGAGTIYYADSVKG
RFTISRDNAKNTVYLQMNSLRPEDTAVYYCGAAYVSKANYGSLWYQDSRRYDYWGQGTLVTVSSGG
GGSGGGGSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKL
TVDKSRWQQGNVFSCSVLHEALHSHYTQKSLSLSPG

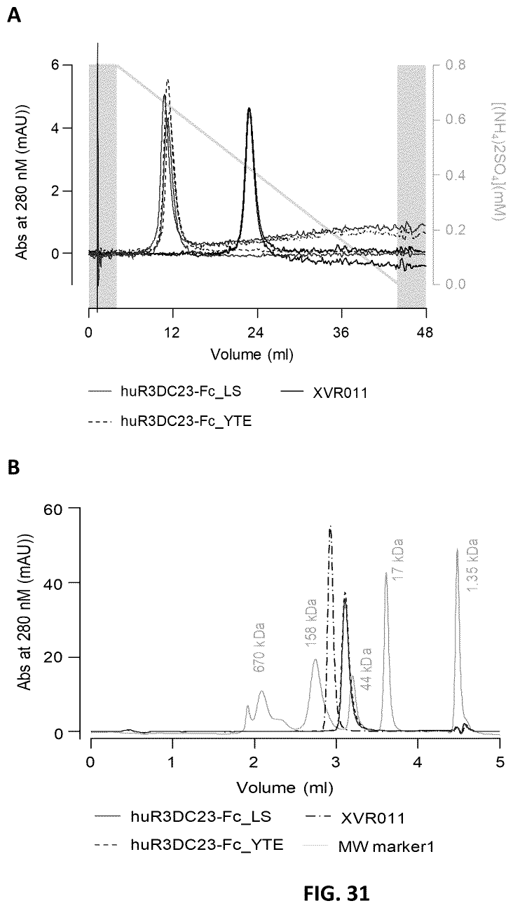

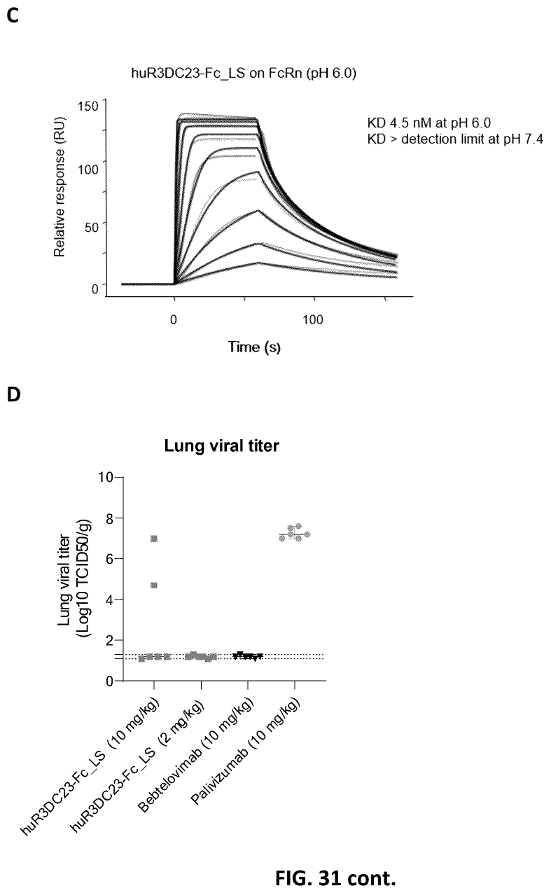

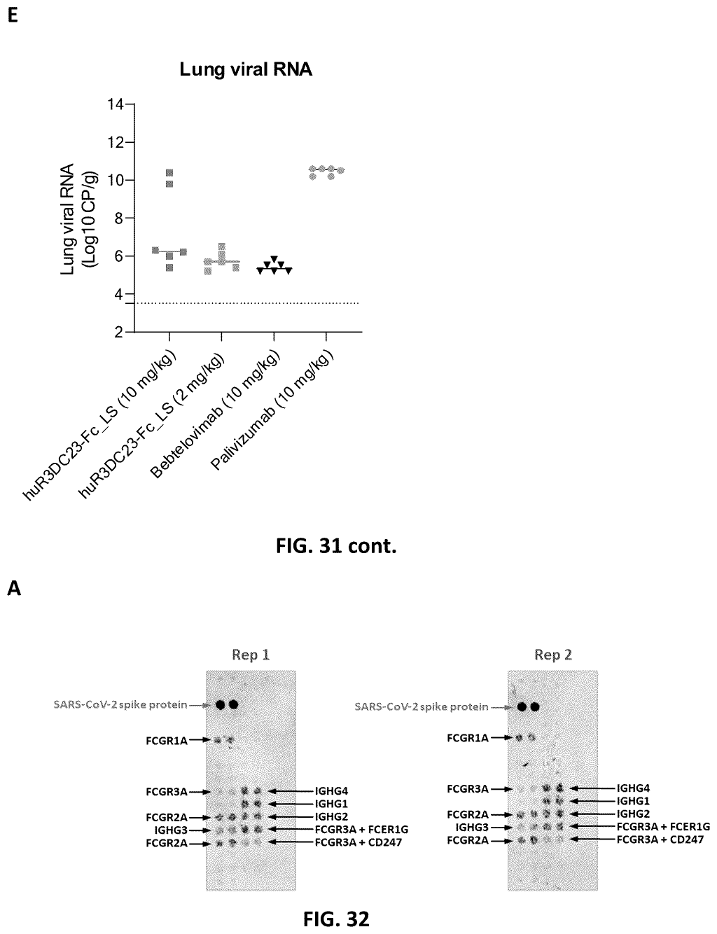

Similarly, to what was observed for the YTE Fc variant (huR3DC23-Fc) (FIG. 30E), huR3DC23-Fc_LS displayed low apparent hydrophobicity (FIG. 31A) and eluted in a single peak from SEC in between 44 and 158 kDa molar weight markers, consistent with a fully assembled VHH-Fc (FIG. 31B).

This construct potently neutralizes pseudotyped VSV displaying spike proteins of SARS-COV-2 D614G, BQ1.1 and XBB with IC₅₀ values similar to what was observed for huR3DC23-Fc containing the YTE mutation (Table 7 and FIG. 30C). In addition, huR3DC23-Fc_LS also potently neutralizes VSV pseudotyped with spikes of SARS-COV-2 BA.4.6, BF.7 and of XBB in which an additional mutation that results in the higher affinity of the XBB1.5 for ACE2 was applied (XBB.1.5-G252V) (Table 7). In contrast, but in line with what was reported previously, sotrovimab and bebletovimab displayed marked reduction in neutralization activity for XBB and BQ1.1, (Imani NEJM 2023).

TABLE 7
Neutralization of VSV pseudotyped with spike proteins
of SARS-CoV-2 variants 614G, BA4.6, BF.7, BQ1.1, XBB and
XBB.1.5(−G252V) by huR3DC23-Fc_LS, sotrovimab and
bebtelovimab. Mean IC50 values (ng/mL) calculated by nonlinear
regression curve fitting are shown, log(inhibitor) versus
normalized response (four parameters).

| | huR3DC23- | | |
|---|---|---|---|
| IC50 ± SD (ng/mL) | Fc_LS | sotrovimab* | bebtelovimab* |
| D614G (n = 5) | 0.8 ± 0.07 | 24.0 ± 1.27 | 1.4 ± 0.15 |
| BA4.6 (n = 3) | 0.6 ± 0.05 | 1076.0 ± 109.27 | 0.8 ± 0.07 |
| BF.7 (n = 3) | 0.6 ± 0.08 | 1145.0 ± 167.59 | 0.8 ± 0.07 |
| BQ1.1 (n = 3) | 0.7 ± 0.07 | 1566.0 ± 232.41 | ND* |
| XBB (n = 2) | 0.9 ± 0.14 | 282.0 ± 40.44 | ND* |
| XBB.1.5(−G252V) | 0.5 ± 0.07 | 247.6 ± 24.30 | ND* |
| (n = 3) | | | |

*Bebtelovimab Biosimilar and sotrovimab Biosimilar were used as positive controls.
ND* No IC50 value could be calculated due to absence of neutralization activity within the tested concentration range (0.0064-20 ng/ml) of bebtelovimab.

Also authentic SARS-COV-2 D614G, Delta, BA.1, BA.2 and BA.5 viruses were potently neutralized by huR3DC23-Fc_LS with an IC₅₀ of 2.1 to 3.7 ng/ml, close to what was observed for monovalent R3DC23 VHH and huR3DC23-Fc carrying the YTE mutation in virus neutralization assays using authentic D614G and BA.1 SARS-COV-2 virus (Table 8 and FIGS. 11 and 30D).

TABLE 8
Neutralization of authentic SARS-CoV-2 virus (614G, Delta, Omicron
BA.1 and Omicron BA.2) by huR3DC23-Fc_LS, sotrovimab, cilgavimab
and bebtelovimab as determined by microneutralization assays. The
table shows the geometric mean IC50 values (ng/mL) and geometric
SD factor (GSD factor) as calculated based on the Zielinska method.

| GM IC₅₀ in ng/mL | huR3DC23- | sotrovimab | cilgavimab | bebtelovimab |
|---|---|---|---|---|
| (GSD factor) | Fc_LS | Biosimilar | Biosimilar | Biosimilar |
| D614G (n = 3) | 2.5 (1.5) | 205.4 (1.2) | 30.9 (1.2) | 3.8 (1.5) |
| Delta (n = 3) | 3.6 (1.2) | 141.1 (1.3) | 28.8 (1.1) | 1.6 (1.5) |
| Omicron BA.1 (n = 3) | 3.8 (1.2) | 1101.3 (2) | 156.5 (2) | 2.5 (1.8) |
| Omicron BA.2 (n = 3) | 2.0 (1.6) | 7147.7 (1.3) | 21.2 (1.3) | 1.7 (1.4) |
| Omicron BA.5 (n = 3) | 3.6 (1.1) | ND* | NT* | 2.4 (1.5) |

ND* Not possible to determine IC50 value for Sotrovimab within the tested concentration range (0.128-10 000 ng/ml).
NT* Cilgavimab was not tested for Omicron BA.5.
*the IC50 of cilgavimab for BA.1 is based on 2 biological replicates in which the highest concentration of cilgavimab (500 ng/ml) resulted in a more than 50% reduction of viral replication but not in a third biological replicate.

### Example 15: LS Mutants of Humanized R3DC23 Fc Fusions Control Viral Replication in Hamsters

The therapeutic potential of huR3DC23-Fc in the Syrian hamster model was evaluated, Hamsters were challenged with an ancestral SARS-COV-2 isolate (BetaCoV/Munich/BavPat1/2020) and, 4 hours later, treated with either 10 mg/kg or 2 mg/kg huR3DC23-Fc_LS, 10 mg/kg Bebtelovivamb (biosimilar) of 10 mg/kg palivizumab (negative control treatment) by intraperitoneal injection. At 4 days post infection high levels of huR3DC23-Fc_LS were detected in all hamsters treated with 2 mg/kg and in 4 hamster treated with 10 mg/kg of this construct (data not shown). In sharp contrast, no or very low levels of huR3DC23-Fc_LS could be detected in in the sera of two animals that were treated with 10 mg/kg huR3DC23-Fc_LS, This most likely results from unsuccessful injection, which has been observed by others (Starr et al. 2021 Nature 597:97-102), Apart from these 2 hamsters the lung virus loads, sampled on day 4 after challenge were below the detection limit in the huR3DC23-Fc treated hamsters whereas control treated animals had significantly higher lung virus loads (FIG. 31D). In accordance in the lungs of hamsters treated with either huR3DC23-Fc_LS or bebtelovimab a strong reduction in viral RNA was observed (FIG. 31E), This experiment shows that S2-binding huR3DC23-Fc can strongly restrict SARS-COV-2 replication in vivo.

### Example 16: Affinity Assessment of R3_DC23-Fc(LS) on the FcRn

To confirm that the introduced LS mutation in the R3DC23 VHH-Fc construct is also associated with a high affinity of the human neonatal receptor FcRn, the kinetic parameters and the equilibrium dissociation constant (K_{D}) of huR3DC23-Fc-LS for FcRn were determined by surface plasmon resonance (SPR) (Table 9), Bebtelovimab biosimilar and a human IgG1 isotype control antibody were used as controls in the assay.

TABLE 9
Kinetic parameters and the equilibrium dissociation constant (K_{D}) of R3_DC23-Fc(LS),
bebtelovimab and a human IgG1 isotype control antibody on FcRn at pH 6.0 and 7.4.

| | FcRn pH 6.0 | | | | | | |
|---|---|---|---|---|---|---|---|
| | | Steady state | | 1:1 binding | | | |
| | | affinity | | model affinity | | FcRn pH 7.4 | |
| | Concentration | RUmax | K_{D} | RUmax | K_{D} | Concentration | RUmax |
| | range | (RU) | (nM) | (RU) | (nM) | range | (RU)* |
| R3_DC23hum- | 250-0.97 nM | 149.8 | 4.5 | 135.2 | 3.5 | 1500-7.8 nM | 22.4 |
| Fc(LS) | | | | | | | |
| bebtelovimab | 1500-7.8 nM | 57.1 | 19.4 | N/D | N/D | 1500-7.8 nM | 8.2 |
| huIgG1 isotype | 1500-7.8 nM | 37.5 | 15.0 | N/D | N/D | 1500-7.8 nM | 16.2 |
| control | | | | | | | |

RU: resonance unti; RUmax: maximum resononace unit.
*RUmax at 1500 nM; N/D: Not determined.

At pH 6.0, huR3DC23-Fc-LS showed improved steady state affinity to FcRn compared to the control bebtelovimab biosimilar. At 3.5-4.5 nM (steady state affinity and 1:1 binding model, respectively), the affinity of huR3DC23-Fc_LS for human FcRn was three- to five-fold lower than that of IgG1 isotype and bebtelovimab biosimilar controls not carrying the LS mutations (FIG. 31C and Table 7), Due to the fast on and off rates, a 1:1 binding model could not be applied to determine the association and dissociation rates for the control antibodies. For R3_DC23-Fc(LS), the affinity values calculated by the steady state and 1:1 binding models were in agreement. At pH 7.4, very low binding to FcRn was observed as expected for all test items.

### Example 17: Specificity of Binding of R3_DC23-Fc(LS) to SARS-COV-2 Spike Protein

To evaluate the lack of specific off-target binding, R3_DC23hum-Fc(LS) was screened using a human plasma membrane protein cell array using fixed human HEK293 cells, individually expressing 6101 full-length human plasma membrane proteins, secreted and cell surface-tethered human secreted proteins plus a further 396 human heterodimers and SARS-COV-2 spike protein. In the pre-screen, investigation of the level of binding of R3_DC23hum-Fc(LS) to fixed untransfected HEK293 cells, and to cells over-laid with SARS-COV-2 spike protein, showed 2.5 μg/mL of R3_DC23hum-Fc(LS) to be a suitable screening concentration. In the library screen a pool of test antibodies including R3_DC23hum-Fc(LS) was screened. In the confirmation screen, each library interaction was re-expressed, along with 2 control receptors, and re-tested with R3_DC23hum-Fc(LS) or control treatments. This was performed on both fixed and live cells.

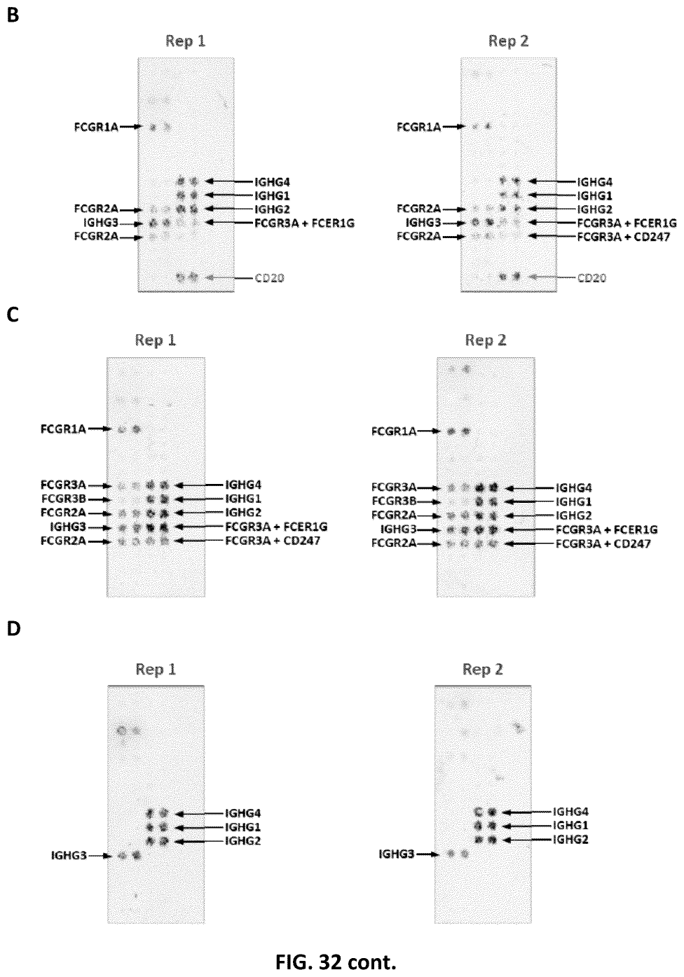

R3_DC23hum-Fc(LS) showed a single significant specific interaction with SARS-COV-2 spike protein, the primary target, on fixed cell microarrays (FIG. 32), No significant specific interactions were observed on the live cell microarray (due to technical limitations it was not possible to spot SARS-COV-2 spike protein on the live cell microarray) (data not shown). These data indicate high specificity of R3_DC23hum-Fc(LS) for its primary target: SARS-COV-2 spike protein.

### Example 18: Generation of Multispecific Constructs Based on S1 and S2 Targeting VHHs

Bispecific tandem (TD) VHHx-VHHy-Fc constructs were generated, wherein an S2 targeting VHH, in particular a humanized form of VHH R3_DC23, and an S1 targeting VHH, in particular a humanized form of VHH3.117, were fused head-to-tail via a 10, 20, or 30 GS linker, in particular (G₄S)₂ (SEQ ID NO:121), (G₄S)₄ (SEQ ID NO:123), or (G₄S)₆ (SEQ ID NO:120), which head-to-tail fusion construct was fused to an Fc domain via a 10 GS linker ((G₄S)₂ (SEQ ID NO:121)), (TD R3DC23-117 (10)-Fc (SEQ ID NO:112), TD R3DC23-117 (20)-Fc (SEQ ID NO:113) and TD R3DC23-117 (30)-Fc (SEQ ID NO:114), corresponding to respectively, FIG. 33 (A), (B), (C)).

Similarly, trispecific tandem VHHx-VHHy-VHHz-Fc constructs were generated, wherein an S2 targeting VHH, in particular a humanized form of VHH R3-DC23, an S1 targeting VHH binding to or competing for the VHH3.117 epitope, in particular a humanized form of VHH3.117, and an S1 targeting VHH binding to or competing for the VHH72 epitope, in particular a humanized form of VHH3.83, were fused head-to-tail via a 20 GS linker ((G₄S)₄ (SEQ ID NO:123)), which head-to-tail fusion construct was fused to an Fc domain via a 10 GS linker ((G₄S)₂ (SEQ ID NO:121)) (TD R3DC23-117-83 (20)-Fc (SEQ ID NO: 117 and FIG. 33 (F)).

Also, a control bispecific construct was generated, wherein an S1 targeting VHH binding to or competing for the VHH72 epitope, in particular a humanized form of VHH3.83 and an S1 targeting VHH binding to or competing for the VHH3.117 epitope, in particular a humanized form of VHH3.117, were fused head-to-tail via a 20 GS linker ((G₄S)₄ (SEQ ID NO:123)), which head-to-tail fusion construct was fused to an Fc domain via a 10 GS linker ((G₄S)₂ (SEQ ID NO:121)) (TD 83-117 (20)-Fc (SEQ ID NO:116) and FIG. 33 (E)). The sequence defined by SEQ ID NO:116 is also shown below.

(SEQ ID NO: 116)
DVQLVESGGGLVQPGDSLRLSCVLSGGVFTSYAMGWFRQAPGKEREFLAAITENSDATYYADSVKG
RFTISRDNAKNTAYLQMNSLRPEDTAVYSCAAGGNHYNPQYYHDYDKYDHWGQGTLVTVSSGGGGS
GGGGSGGGGSGGGGSDVQLVESGGGLVQPGGSLRLSCAASGKAVSISDMGWYRQPPGKQRELVATI
TKTGSTNYADSVKGRFTISRDNTKNTVYLEMNSLRPEDTAVYYCNAWLPYGLGPDYYGLELWGQGT
LVTVSSGGGGSGGGGSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDG
SFFLYSKLTVDKSRWQQGNVFSCSVLHEALHSHYTQKSLSLSPG

A tetravalent bispecific VHHx-Fc-VHHy fusion construct (also referred to herein as a moonlander construct) was generated in which two VHHs respectively targeting S1 and S2, in particular a humanized form of VHH R3_DC23 and a humanized form of VHH3.117, respectively, were respectively fused to the N- and C-terminus of an Fc domain via respectively a 10 GS linker ((G₄S)₂ (SEQ ID NO:121)) and a 15 GS linker ((G4S) 3 (SEQ ID NO:122)) (R3DC23-Fc-117 (SEQ ID NO: 115) and FIG. 33(D)).

The Fc domain used was human IgG1 Fc containing LS mutations (M428L combined with N434S).

FIG. 33 shows a schematic representation of the different constructs.

The constructs were expressed in Pichia pastoris and/or in CHO cells, or any alternative suitable production host, followed by purification and biochemical and biophysical characterization, as described herein and/or as known by the skilled person.

### Example 19: S1 and S2 Targeting Binding Agents Neutralize SARS-COV-2 Variants D614G, Omicron BA.2 and BA.5 (Live Virus Assay)

The neutralization potency of the multispecific constructs targeting S1 and S2 generated in example 18 was tested in a live virus assay (microneutralization method), All multispecific constructs were able to neutralize the tested variants (614G, Omicron BA.2 and Omicron BA.5) (Table 10).

TABLE 10
Neutralization of authentic SARS-CoV-2 variants (614G,
Delta, Omicron BA.2 and Omicron BA.5) by the multispecific
constructs targeting S1 and S2 generated in example 18 as
determined by a microneutralization method. The geometric
mean IC₅₀ values (ng/mL) are shown.

| | | | Omicron | Omicron |
|---|---|---|---|---|
| | | D614G | BA.2 | BA.5 |
| | | (n = 3) | (n = 3) | (n = 3) |
| | | IC50 | IC50 | IC50 |
| | Construct | (ng/mL) | (ng/mL) | (ng/mL) |
| S2 | R3DC23 VHH-Fc | 3.7 | 2.8 | 3.6 |
| S1 | TD 83-117 (20)- Fc | 227.1 | 429.9 | 322.4 |
| S1 + S2 | TD R3DC23-117(10)-Fc | 9.8 | 8.7 | 8.3 |
| S1 + S2 | TD R3DC23-117(20)-Fc | 7.7 | 7.8 | 8.8 |
| S1 + S2 | TD R3DC23-117(30)-Fc | 10.3 | 8.0 | 7.7 |
| S1 + S2 | R3DC23-Fc-117 | 14.4 | 10.7 | 11.1 |
| S1 + S2 | TD R3DC23-117-83 (20)- Fc | 16.5 | 12.6 | 13.2 |

### Example 20: Composition of S1 and S2 Targeting Binding Agents (XVR012) Neutralizes VSV-GFP Reporter Viruses Pseudotyped with SARS-COV-2 Spike Proteins

A VHH-Fc construct was generated wherein a humanized form of R3_DC23 was fused to a human IgG1 Fc containing an LS mutation via a (G₄S)₂ linker (SEQ ID NO:121) at the N-terminus (XVR013) (SEQ ID NO:118). A VHHx-Fc-VHHy construct was generated wherein a humanized form of the S1 targeting VHH3.117 capable of binding to or competing for the VHH3.117 epitope is fused to a human IgG1 Fc containing an LS mutation via a (G₄S)₂ linker (SEQ ID NO: 121) at the N-terminus of the Fc domain, and wherein a humanized form of the S1 targeting VHH3.83 capable of binding to or competing for the VHH72 epitope is fused to the C-terminus via a (G4S) 2 linker (SEQ ID NO: 121) (XVR014) (SEQ ID NO:119).

XVR014:
(SEQ ID NO: 119)
DVQLVESGGGLVQPGGSLRLSCAASGKAVSISDMGWYRQPPGKQRELVATITKTGSTNYADSVKGR
FTISRDNTKNTVYLEMNSLRPEDTAVYYCNAWLPYGLGPDYYGLELWGQGTLVTVSSGGGGSGGGG
SDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRW
QQGNVFSCSVLHEALHSHYTQKSLSLSPGGGGGSGGGGSDVQLVESGGGLVQPGDSLRLSCVLSGG
VFTSYAMGWFRQAPGKEREFLAAITENSDATYYADSVKGRFTISRDNAKNTAYLQMNSLRPEDTAV
YSCAAGGNHYNPQYYHDYDKYDHWGQGTLVTVSS

XVR013 and XVR014 were mixed in a ratio 1:1 to generate a composition or cocktail (XVR012) (FIG. 34).

The neutralization potency of XVR012, XVR013 and XVR014 was tested in a pseudovirus neutralization assay using VSV particles pseudotyped with the spike of either SARS-COV-2 D614G reference strain, or Omicron variants BA.2.75.2, BA.4.6, BF.7, BQ.1.1, XBB, and XBB.1.5, A negative control antibody (isotype control) was included in the assay.

XVR012, XVR013 and XVR014 were able to neutralize all tested SARS-COV-2 variants (D614G, Omicron BA.2.75.2, BA4.6, BF.7, BQ1.1, XBB and XBB.1.5) with mean IC₅₀ values ranging from 1.1 and 23.9 ng/mL for XVR012, from 0.6 ng/mL to 18.5 ng/ml for XVR013, and from 66.8 to 239.8 ng/mL for XVR014 (Table 11), Only a minor increase in IC₅₀ value was observed for XVR012 and XVR013 against the BA.2.75.2 variant.

TABLE 11
Neutralization of SARS-CoV-2 variants (D614G, Omicron BA.2.75.2, BA4.6, BF.7,
BQ1.1, XBB and XBB.1.5) by XVR012, XVR013 and XVR014 as determined in a
pseudovirus neutralization assay. Vero E6 cells were transduced with VSV-GFP
reporter viruses pseudotyped with SARS-CoV-2 D614G spike protein or with the
spike protein of the SARS-CoV-2 variants BA.2.75.2, BA.4.6, BF.7, BQ.1.1, XBB
or XBB.1.5, which viruses had been pre-incubated with different concentrations
of the constructs or composition. Sixteen hours later, the GFP fluorescence was
measured with a fluorimeter. The calculated mean IC₅₀ values (ng/ml;
N = 3 ± SD for all variants except for XBB) are shown. The mean IC50 values
were calculated by nonlinear regression curve fitting, log(inhibitor)
versus normalized response (four parameters).

| Spike | | | | | | | | |
|---|---|---|---|---|---|---|---|---|
| target | Antibody | D614G | BA.2.75.2 | BA.4.6 | BF.7 | BQ.1.1 | XBB | XBB.1.5 |
| S1 + S2 | XVR012 | 1.7 | 23.9 | 1.2 | 1.2 | 1.5 | 1.8 | 1.1 |
| S2 | XVR013 | 0.8 | 18.5 | 0.6 | 0.6 | 0.7 | 0.9 | 0.51 |
| S1 | XVR014 | 47.6 | 180.1 | 57.6 | 66.8 | 186.1 | 239.8 | 152.8 |

### Example 21: Composition of S1 and S2 Targeting Binding Agents (XVR012) Neutralizes SARS-CoV-2 Variants D614G, Omicron BA.2 and BA.5 (Live Virus Assay)

The neutralization potency of the constructs and composition described in Example 20 were tested in a live virus assay (microneutralization method), XVR012, XVR013 and XVR014 were able to neutralize all tested SARS-COV-2 variants with geometric mean IC₅₀ values ranging from 0.6 and 1.9 ng/mL for XVR012, from 2.8 ng/mL to 3.7 ng/mL for XVR013, and from 98 to 124.5 ng/ml for XVR014, (Table 12).

TABLE 12
Neutralization of authentic SARS-CoV-2 variants (D614G, Omicron
BA.2 and Omicron BA.5) by XVR012, XVR013 and XVR014 described
in example 20 as determined by a live virus method. The geometric
mean IC₅₀ values (ng/mL; N = 3) are shown.

| Spike target | Antibody | D614G | Omicron BA.2 | Omicron BA.5 |
|---|---|---|---|---|
| S1 + S2 | XVR012 | 1.9 | 1.4 | 0.6 |
| S2 | XVR013 | 3.7 | 2.8 | 3.6 |
| S1 | XVR014 | 124.5 | 98.0 | 117.1 |

### Example 22: In Vivo Efficacy of XVR012, XVR013 and XVR014 in Syrian Golden Hamster SARS-COV-2 Challenge Model

The in vivo efficacy of XVR012, XVR013 and XVR014 described in Example 20 was evaluated in a Syrian Golden hamster post-infection challenge model (Wuhan strain), Doses of 4 mg/kg and 20 mg/kg of XVR012, and of 2 mg/kg and 10 mg/kg of XVR013 and XVR014, were administered intraperitoneally 4 hours after challenge (therapeutic setting), Control animals received 10 mg/kg of palivizumab, a humanized monoclonal antibody directed against the fusion protein of human respiratory syncytial virus (negative control), and one group of animals received 10 mg/kg of bebtelovimab (positive control).

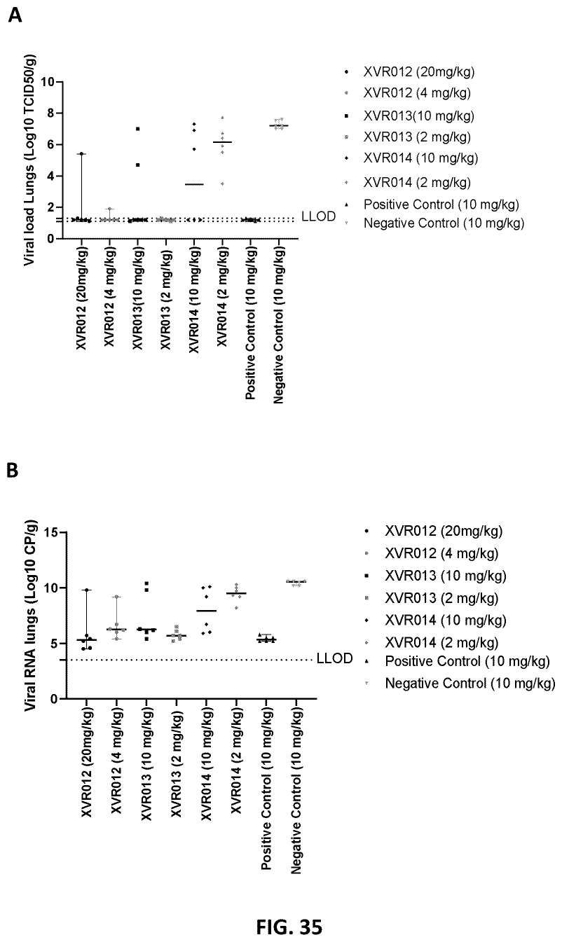

Viral replication in the lungs was completely cleared in animals treated with XVR012 and XVR013 at both the high dose and the low dose, as well as in the positive control (FIG. 35A). A dose dependent effect on viral replication in the lungs was observed with XVR014, Treatment with XVR012, XVR013, XVR014 or the positive control reduced viral RNA load in lung tissue as compared to the negative control group (FIG. 35B).

### Example 23: Antibody-Dependent Cellular Cytotoxicity (ADCC) of XVR012, XVR013 and XVR014

A FcγRIIIa reporter assay was performed to assess the antibody-dependent cellular cytotoxicity of XVR012, XVR013 and XVR014 described in Example 20, CHO-K1 expressing SARS COV-2 Spike Protein target cell line were used as target cells and Jurkat FcγRIIIa (CD16) V176-NFAT-RE Luc as reporter cells, Three independent assay runs were performed. The assay employed an effector to target cell ratio of 40:1, with the samples assessed in an 8-point dilution series starting at 30 μg/mL for XVR013 and XVR014 or at 60 μg/mL for XVR012 as three independent replicates (3 assay plates per run), An isotype control was assessed at a single concentration of 30 μg/mL. The assay plates were incubated overnight (21 hours #1 hour) prior to the addition of SteadyGlo (luminescence endpoint).

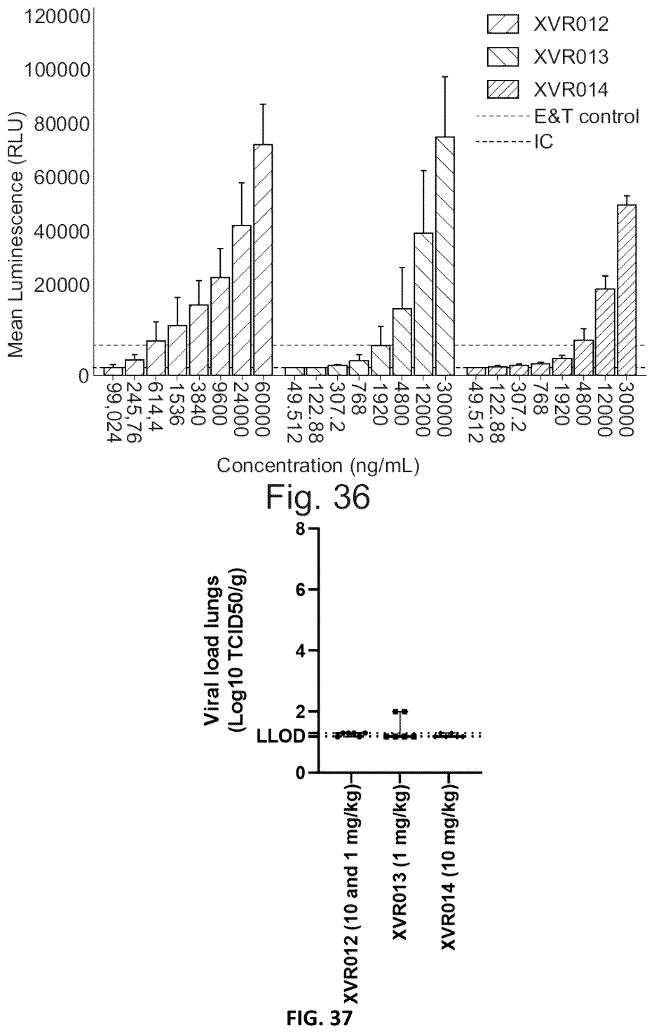

XVR012, XVR013 and XVR014 mediated ADCC responses in a dose-dependent matter and the response was substantially greater compared to the response of the isotype control (IC) or assay control (Effector & Target cells) (FIG. 36).

### Example 24: In Vivo Prophylactic Efficacy of XVR012, XVR013 and XVR014 in Syrian Golden Hamster SARS-COV-2 Challenge Model

The in vivo efficacy of XVR012, XVR013 and XVR014 as described in Example 20 was evaluated in a Syrian Golden hamster challenge model (Wuhan strain). A cocktail of 10 mg/kg of XVR014 and 1 mg/kg of XVR013 (XVR012), 1 mg/kg XVR013 and 10 mg/kg XVR014 were administered by intraperitoneal injection approximately 24 hours prior to infection.

In this prophylactic setting, viral replication in the lungs was cleared in animals treated with XVR012, XVR013 and XVR014 (FIG. 37).

### Example 25: In Vivo Therapeutic Efficacy of XVR012, XVR013 and XVR014 in Syrian Golden Hamster SARS-COV-2 Challenge Model

Cocktails of 5 mg/kg of XVR014 and 0.5 mg/kg of XVR013, of 10 mg/kg of XVR014 and 1 mg/kg of XVR013, and of 20 mg/kg of XVR014 and 2 mg/kg of XVR013 (XVR012): 0.5, 1 and 2 mg/kg XVR013; 5, 10 and 20 mg/kg XVR014 were administered by intraperitoneal injection 4 hours after the SARS-COV2 challenge, Control animals received 10 mg/kg of palivizumab (negative control), and one group of animals received 10 mg/kg of bebtelovimab (used as positive control).

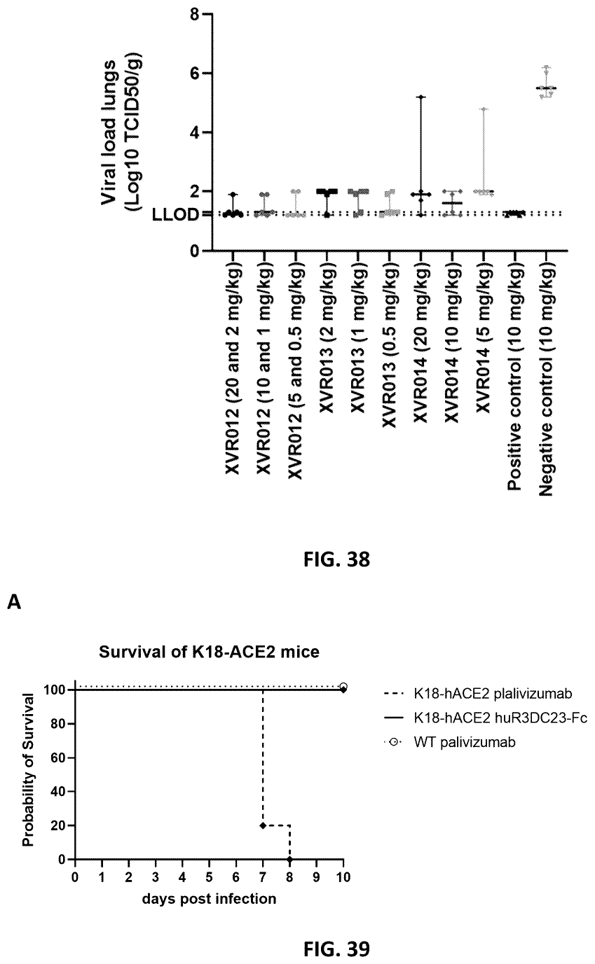

In the therapeutic setting, viral replication in the lungs was inhibited for the animals treated with XVR012, XVR013, XVR014 and the positive control, whereas control treated animals had higher lung virus loads (FIG. 38).

### Example 26: Prophylactic Treatment with R3_DC23-Fc Protects K18-hACE2 Mice Against SARS-COV-2 Infection

K18-hACE2 mice that express human ACE2 at the surface of their epithelial cells were treated with 100 μg R3_DC23-Fc(YTE) (R3_DC23-Fc; SEQ ID NO: 96) or 100 μg isotype control IgG (palivizumab) via intraperitoneal injection 1 day prior to a lethal infection with SARS-COV-2 614G variant virus, Palivizumab treated, infected wild type (WT) mice that are-none permissive for SARS-CoV-2 infection were used as control for protection.

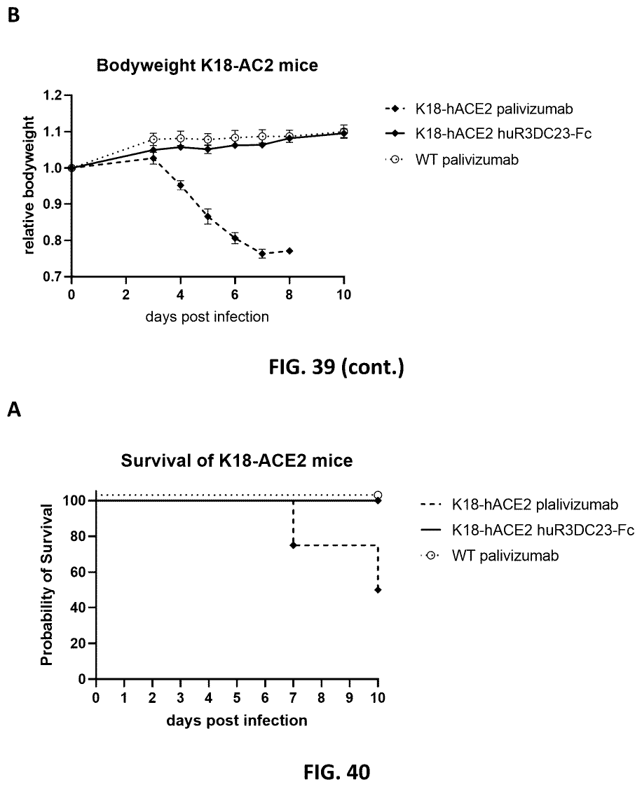

As expected, all palivizumab treated K18-hACE2 displayed marked bodyweight loss and succumbed from the viral challenge. In sharp contrast, R3-DC23-Fc protected infected K18-hACE2 mice from bodyweight loss and death, similar to palivizumab treated WT mice. This illustrates that R3-DC23-Fc can protect mice from lethal SARS-COV-2 infections (FIGS. 39A and B).

To test if R3_DC23-Fc can also control viral replication in the lungs, K18-hACE2 mice were treated with 100 μg R3_DC23-Fc or 100 μg isotype control IgG (palivizumab) via intraperitoneal injection 1 day prior to infection with SARS-COV-2 614G variant virus. At five days post-infection part of the mice were sacrificed to isolate the lungs for quantification of viral replication by plaque assay and qPCR and part of the mice were used to monitor the bodyweight and mortality.

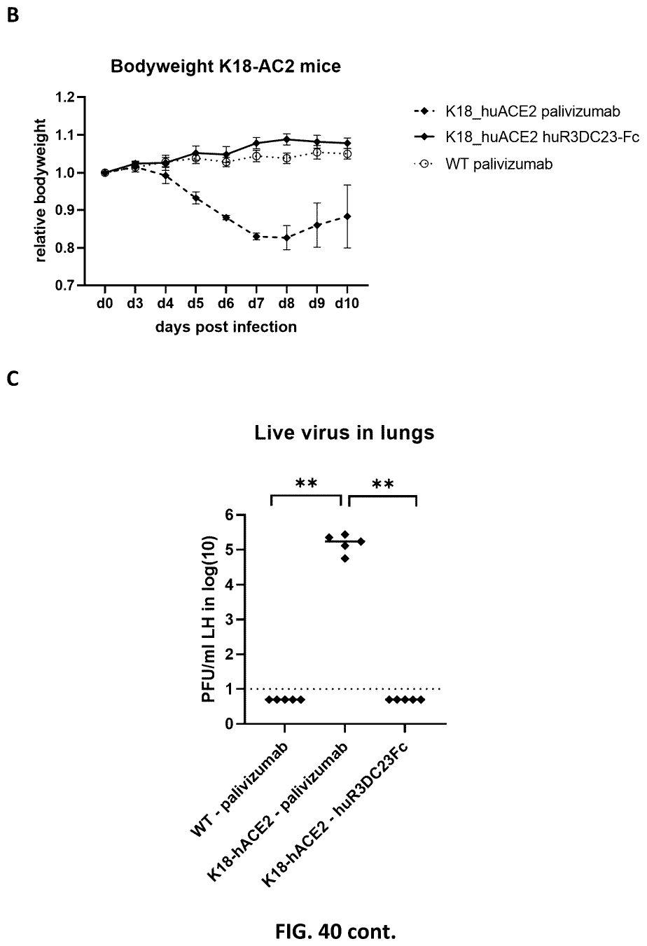

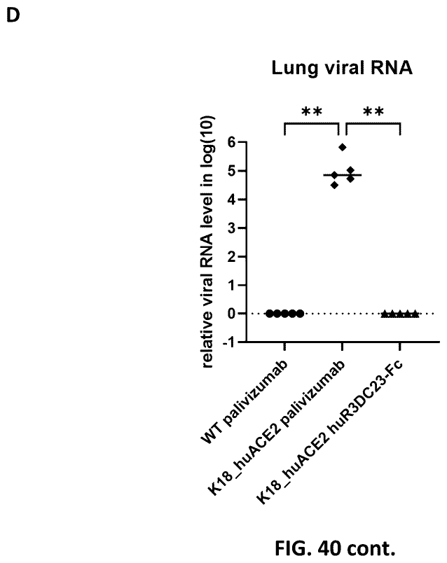

R3_DC23-Fc treatment protected K18-hACE2 mice form bodyweight loss and lethality upon viral challenge (FIGS. 40A and B), Whereas high levels of replicating SARS-COV-2 virus could be isolated from lungs of all K18-hACE2 mice that were treated with palivizumab, no replicating virus could be detected in the lungs of K18-hACE2 mice treated with R3-DC23-Fc or of WT mice (FIG. 40C), Similarly, in contrast to palivizumab-treated K18-hACE2 mice, no viral RNA could be detected in the lungs of K18-hACE2 mice treated with R3-DC23-Fc or of WT mice (FIG. 40D). These data illustrate that prophylactic treatment with R3-DC23-Fc can prevent or strongly reduce viral replication in the highly permissive K18-hACE2 mice.

### Example 27: Stoichiometry and Binding Kinetics of R3_DC23 to SARS-COV-2 Spike 2P (S-2P) Protein

The SARS-COV-2 spike 2P (S2P) binding kinetics of R3_DC23 were assessed via biolayer interferometry.

Upon capturing a ligand to a biosensor in such a way it is fully available for analyte binding, stoichiometry of the interaction between ligand and analyte can be determined upon saturation of the captured ligand by the following formula:

$$stoichiometry = \frac{resp {onse}_{analyte} \cdot {MW}_{ligand}}{resp {onse}_{ligand} \cdot {MW}_{analyte}}$$

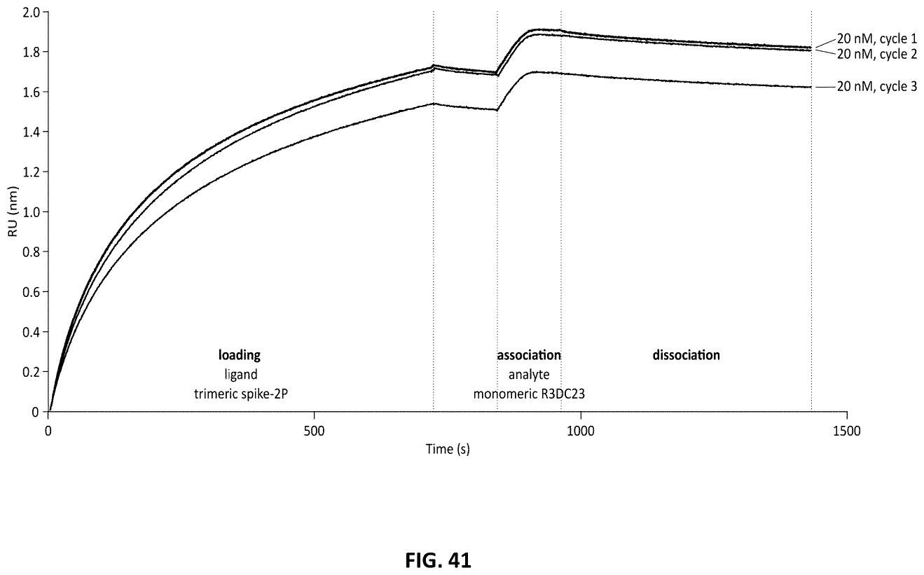

Triplicate experiments showed a stoichiometry of approximately 3.5 molecules of R3_DC23 VHH for each S-2P trimer, suggesting each monomer in the S-2P trimer binds to one R3_DC23 VHH (Table 13, FIG. 41).

TABLE 13
Stoichiometry as determined using the above formula
for the interaction between Strep tag-captured S-2P
trimer (MW: 456.4 kDa; ligand) and saturating (20 nM)
R3_DC13 monomer (MW: 16.6 kDa; analyte).

| Experiment | | S-2P trimer | R3_DC23 | |
|---|---|---|---|---|
| type | Repeat | (RU in nM) | (RU in nm) | Stoichiometry |
| Kinetics | 1 | 1.710 | 0.2283 | 1:3.678 |
| 20 nM*** | 2 | 1.725 | 0.2140 | 1:3.417 |
| | 3 | 1.540 | 0.1956 | 1:3.499 |

The association phase of the interaction between S-2P and a 20 to 1.25 nM dilution series of R3_DC23 could not be described by a global exponential 1:1 binding model. The off-rate was determined at k_{off}=2.3·10⁻⁴±5.2·10⁻⁵ g⁻¹.

### SEQUENCE LISTING FREE TEXT

| SEQ ID NO | Free text (<223>) |
|---|---|
| 1 | VHH R3_C4 |
| 2 | VHH R4_DC16 |
| 3 | VHH R3_DC20 |
| 4 | VHH R3_DC2 |
| 5 | VHH R4_DC20 |
| 6 | VHH R4_DC9 |
| 7 | VHH R4_DC6 |
| 8 | VHH R3_DC23 |
| 9 | VHH R3_DC9 |
| 10 | VHH R4_DC13 |
| 11 | CDR1 of VHH R3_C4, VHH R4_DC16, VHH |
| | R3_DC20, VHH R3_DC2, VHH R4_DC20, VHH |
| | R4_DC9, VHH R3_DC23, VHH R3_DC9, VHH |
| | R4_DC13 Kabat |
| 12 | CDR1 of VHH R4_DC6 Kabat |
| 13 | CDR2 of VHH R3_C4, VHH R4_DC16, VHH |
| | R3_DC20 Kabat |
| 14 | CDR2 of VHH R3_DC9 Kabat |
| 15 | CDR2 of VHH R3_DC2 Kabat |
| 16 | CDR2 of VHH R4_DC20 Kabat |
| 17 | CDR2 of VHH R4_DC13 Kabat |
| 18 | CDR2 of VHH R4_DC9 Kabat |
| 19 | CDR2 of VHH R4_DC6 Kabat |
| 20 | CDR2 of VHH R3_DC23 Kabat |
| 21 | CDR3 of VHH R3_C4 Kabat |
| 22 | CDR3 of VHH R4_DC16 Kabat |
| 23 | CDR3 of VHH R3_DC20, VHH R3_DC9, VHH |
| | R3_DC2, VHH R4_DC20 Kabat |
| 22 | CDR3 of VHH R4_DC16 Kabat |
| 23 | CDR3 of VHH R3_DC20, VHH R3_DC9, VHH |
| | R3_DC2, VHHR4_DC20 Kabat |
| 24 | CDR3 of VHH R4_DC13 Kabat |
| 25 | CDR3 of VHH R4_DC9 Kabat |
| 26 | CDR3 of VHH R4_DC6 Kabat |
| 27 | CDR3 of VHH R3_DC23 Kabat |
| 28 | FR1 of VHH R3_C4, VHH R4_DC16, VHH |
| | R3_DC20 Kabat |
| 29 | FR1 of VHH R3_DC9, VHH R3_DC2, VHH |
| | R4_DC20, VHH R4_DC13 Kabat |
| 30 | FR1 of VHH R4_DC9 Kabat |
| 31 | FR1 of VHH R4_DC6 Kabat |
| 32 | FR1 of VHH R3_DC23 Kabat |
| 33 | FR2 of VHH R3_C4, VHH R4_DC16, VHH |
| | R3_DC20, VHH R3_DC2, VHH R4_DC20, |
| | VHH R4_DC6, VHH R3_DC23, VHH R3_DC9, |
| | VHH R4_DC13 Kabat, Martin |
| 34 | FR2 of VHH R4_DC9 Kabat, Martin |
| 35 | FR3 of VHH R3_C4 Kabat |
| 36 | FR3 of VHH R4_DC16 Kabat |
| 37 | FR3 of VHH R3_DC20 Kabat |
| 38 | FR3 of VHH R3_DC9 Kabat |
| 39 | FR3 of VHH R3_DC2 Kabat |
| 40 | FR3 of VHH R4_DC20 Kabat |
| 41 | FR3 of VHH R4_DC13 Kabat |
| 42 | FR3 of VHH R4_DC9 Kabat |
| 43 | FR3 of VHH R4_DC6 Kabat |
| 44 | FR3 of VHH R3_DC23 Kabat |
| 45 | FR4 of VHH R3_C4, VHH R4_DC16, VHH |
| | R3_DC20, VHH R3_DC9, VHH R3_DC2, VHH |
| | R4_DC20, VHH R4_DC13, VHH R4_DC9, VHH |
| | R4_DC6, VHH R3_DC23 Kabat, Martin |
| 46 | CDR1 Kabat |
| 47 | CDR2 Kabat |
| 48 | CDR3 Kabat, Martin |
| 49 | variation CDR1 Kabat |
| 50 | variation CDR2 Kabat |
| 51 | variation CDR3 Kabat, Martin |
| 52 | CDR1 R3_C4, R4_DC16, R3_DC20, R3_DC9, |
| | R3_DC2, R4_DC20, R4_DC13 Martin |
| 53 | CDR1 R3_DC23, R4_DC9 Martin |
| 54 | CDR1 R4_DC6 Martin |
| 55 | CDR2 of VHH R3_C4, VHH R4_DC16, VHH |
| | R3_DC20 Martin |
| 56 | CDR2 of VHH R3_DC9 Marti |
| 57 | CDR2 of VHH R3_DC2 Martin |
| 58 | CDR2 of VHH R4_DC20 Martin |
| 59 | CDR2 of VHH R4_DC13 Martin |
| 60 | CDR2 of VHH R4_DC9 Martin |
| 61 | CDR2 of VHH R4_DC6 Martin |
| 62 | CDR2 of VHH R3_DC23 Martin |
| 63 | CDR1 Martin, AbM, IMGT |
| 64 | CDR2 Martin, AbM |
| 65 | variation CDR1 Martin, AbM, IMGT |
| 66 | variation CDR2 Martin, AbM |
| 67 | CDR3 AbM, Chothia |
| 68 | variation CDR3 AbM, Chothia |
| 69 | CDR1 Chothia |
| 70 | CDR2 Chothia |
| 71 | variation CDR1 Chothia |
| 72 | variation CDR2 Chothia |
| 73 | CDR2 IMGT |
| 74 | CDR3 IMGT |
| 75 | variation CDR2 IMGT |
| 76 | variation CDR3 IMGT |
| 77 | CDR1 MacCallum |
| 78 | CDR2 MacCallum |
| 79 | CDR3 MacCallum |
| 80 | variation CDR1 MacCallum |
| 81 | variation CDR2 MacCallum |
| 82 | variation CDR3 MacCallum |
| 83 | VHH R3_DC19 |
| 84 | VHH R3_DC21, VHH R3_DC22 |
| 85 | VHH R3_DC5 |
| 96 | R3_DC23(hum)-Fc(YTE) |
| 97 | FR1 of VHH R3_C4, VHH R4_DC16, VHH |
| | R3_DC20 Martin |
| 98 | FR1 of VHH R3_DC9, VHH R3_DC2, VHH |
| | R4_DC20, VHH R4_DC13 Martin |
| 99 | FR1 of VHH R4_DC9, R4_DC6 Martin |
| 100 | FR1 of VHH R3_DC23 Martin |
| 101 | FR3 of VHH R3_C4 Martin |
| 102 | FR3 of VHH R4_DC16 Martin |
| 103 | FR3 of VHH R3_DC20 Martin |
| 104 | FR3 of VHH R3_DC9 Martin |
| 105 | FR3 of VHH R3_DC2 Martin |
| 106 | FR3 of VHH R4_DC20 Martin |
| 107 | FR3 of VHH R4_DC13 Martin |
| 108 | FR3 of VHH R4_DC9 Martin |
| 109 | FR3 of VHH R4_DC6 Martin |
| 110 | FR3 of VHH R3_DC23 Martin |
| 112 | TD R3DC23-117(10)-Fc |
| 113 | TD R3DC23-117(20)-Fc |
| 114 | TD R3DC23-117(30)-Fc |
| 115 | R3DC23-Fc-117 |
| 116 | TD 83-117 (20)- Fc |
| 117 | TD R3DC23-117-83 (20)- Fc |
| 118 | XVR013 |
| 119 | XVR014 |
| 120 | Gly-Ser linker (G₄S)₆ |
| 121 | Gly-Ser linker (G₄S)₂ |
| 122 | Gly-Ser linker (G₄S)₃ |
| 123 | Gly-Ser linker (G₄S)₄ |
| 124 | VHH72 |
| 125 | VHH83 |
| 126 | VHH3.117 |
| 127 | humanized R3_DC23 |
| 128 | humanized R3_C4 |
| 129 | humanized R4_DC20 |
| 130 | huR3C4-Fc |
| 131 | huR4DC20-Fc |

1. A binding agent capable of neutralizing a Sarbecovirus, wherein the binding agent specifically binds to or within a region of spike protein of the Sarbecovirus corresponding to the region from amino acid E1188 to amino acid Y1206 of the SARS-COV-2 spike protein as defined in SEQ ID NO: 86.

2. The binding agent according to claim 1, which specifically binds to or within a region of spike protein corresponding to the region from amino acid E1188 to amino acid L1203 of the SARS-COV-2 spike protein as defined in SEQ ID NO: 86.

3. The binding agent according to claim 1 or 2, which specifically binds to or within a region of spike protein corresponding to the region from amino acid N1194 to amino acid L1203 of the SARS-COV-2 spike protein as defined in SEQ ID NO: 86.

4. The binding agent according to claim 1, wherein said binding agent specifically binds to a region of heptad repeat 2 (HR2) domain of spike protein of the Sarbecovirus proximal to the viral membrane.

5. The binding agent according to claim 1, wherein said binding agent specifically binds to a quaternary epitope in a spike protein trimer.

6. The binding agent according to claim 5, wherein amino acid residues from at least two monomers of the spike protein trimer contribute to said quaternary epitope.

7. The binding agent according to claim 1, wherein said binding agent specifically binds to at least one amino acid residue of spike protein corresponding to the amino acid residues N1194, S1196, D1199 or Q1201 of the SARS-COV-2 spike protein as defined in SEQ ID NO:86; or wherein at least one amino acid residue of spike protein corresponding to the amino acid residues N1194, S1196, D1199 or Q1201 of the SARS-CoV-2 spike protein as defined in SEQ ID NO:86 is indispensable for binding of said binding agent to spike protein.

8. The binding agent according to claim 1, wherein said binding agent specifically binds to at least one amino acid residue of spike protein corresponding to the amino acid residues S1196 or Q1201 of the SARS-COV-2 spike protein as defined in SEQ ID NO:86; or wherein at least one amino acid residue of spike protein corresponding to the amino acid residues S1196 or Q1201 of the SARS-COV-2 spike protein as defined in SEQ ID NO:86 is indispensable for binding of said binding agent to spike protein.

9. The binding agent according to claim 1, wherein said binding agent specifically binds to the amino acid residues of spike protein corresponding to the amino acid residues S1196 and Q1201 of the SARS-COV-2 spike protein as defined in SEQ ID NO:86, optionally to the amino acid residues of spike protein corresponding to the amino acid residues N1194, S1196, D1199 and Q1201 of the SARS-COV-2 spike protein as defined in SEQ ID NO:86; or wherein the amino acid residues S1196 and Q1201 of the SARS-CoV-2 spike protein as defined in SEQ ID NO:86, optionally the amino acid residues of spike protein corresponding to the amino acid residues N1194, S1196, D1199 and Q1201 of the SARS-CoV-2 spike protein, are indispensable for binding of said binding agent to spike protein.

10. The binding agent according to claim 1, wherein said binding agent stabilizes the prefusion conformation of spike protein.

11. The binding agent according to claim 1, wherein:
said binding agent is capable of neutralizing the Sarbecovirus with a 50% inhibitory concentration (IC₅₀) of 100 ng/ml or less, as determined in a Sarbecovirus spike protein pseudovirus neutralization assay such as a vesicular stomatitis virus (VSV)-Sarbecovirus spike protein pseudovirus neutralization assay;
said binding agent is capable of neutralizing any one or both of SARS-COV-2 such as one or more of SARS-COV-2 Wuhan strain, SARS-COV-2 Alpha variant, SARS-COV-2 Omicron BA, 1 variant, and SARS-COV-2 Omicron BA.2 variant, SARS-COV-2 Omicron BA.5 variant, SARS-COV-2 Omicron BA.2.75.2 variant, SARS-COV-2 Omicron BA.4.6 variant, SARS-COV-2 Omicron BF.7 variant, SARS-COV-2 Omicron BQ.1.1 variant, SARS-COV-2 Omicron XBB variant and SARS-COV-2 Omicron XBB.1.5 variant; and SARS-COV-1;
said binding agent is capable of inhibiting spike-mediated syncytia formation between cells expressing the Sarbecovirus spike protein and cells expressing the angiotensin-converting enzyme 2 (ACE2) receptor; and/or
said binding agent does not bind a Middle East respiratory syndrome coronavirus (MERS-COV).

12. The binding agent according to claim 1, which comprises an antibody or an antibody fragment.

13. The binding agent according to claim 1, which comprises an immunoglobulin single variable domain (ISVD), preferably a VHH.

14. The binding agent according to claim 13, wherein the ISVD comprises a complementarity determining region 1 (CDR1) defined by any one of SEQ ID NOs: 63, 46, 69 or 77, a complementarity determining region 2 (CDR2) defined by any one of SEQ ID NOs: 64, 47, 70, 73 or 78, and a complementarity determining region 3 (CDR3) defined by any one of SEQ ID NOs: 48, 67, 74 or 79.

15. The binding agent according to claim 13, wherein the ISVD comprises a CDR1, CDR2 and CDR3, each independently as present in any of SEQ ID NOs: 1 to 10, wherein the CDR1, CDR2 and CDR3 are annotated according to any one of Kabat, MacCallum, IMGT, AbM, Martin or Chothia.

16. The binding agent according to claim 13, wherein the ISVD comprises a CDR1 defined by SEQ ID NO:63, a CDR2 defined by SEQ ID NO: 64, and a CDR3 defined by SEQ ID NO: 48.

17. The binding agent according to claim 14, wherein the ISVD comprises an amino acid sequence with at least 90% identity to an amino acid sequence selected from the group consisting of SEQ ID NO: 1 to 10, and 127 to 129.

18. The binding agent according to claim 1, comprising an ISVD comprising an amino acid sequence selected from the group consisting of SEQ ID NO: 1 to 10, and 127 to 129.

19. The binding agent according to claim 1, which is in a multivalent form.

20. The binding agent according to claim 1, which comprises an amino acid sequence defined by any one of SEQ ID NO: 96, 118, 130 or 131.

21. A multispecific binding agent comprising the binding agent as defined in claim 1 and further comprising a binding agent which specifically binds to a Sarbecovirus spike protein receptor binding domain (RBD).

22. The multispecific binding agent according to claim 21, wherein the binding agent specifically binding to the RBD comprises at least one ISVD capable of binding to or competing for the epitope specifically bound by VHH72 defined by SEQ ID NO: 124.

23. The multispecific binding agent according to claim 21, wherein the binding agent specifically binding to the RBD comprises at least one ISVD capable of binding to or competing for the epitope specifically bound by VHH3.117 defined by SEQ ID NO: 127.

24. The multispecific binding agent according to claim 21, which comprises an amino acid sequence defined by any one of SEQ ID NO: 112-117.

25. A nucleic acid molecule comprising a polynucleotide sequence encoding the binding agent according to claim 1; a vector comprising said nucleic acid molecule; or a cell expressing the binding agent according to claim 1 or comprising said nucleic acid molecule or said vector.

26. A composition comprising the binding agent as defined in claim 1 and further comprising a binding agent which specifically binds to a Sarbecovirus spike protein receptor binding domain (RBD).

27. The composition according to claim 26, wherein the binding agent specifically binding to the RBD comprises at least one ISVD capable of binding to or competing for the epitope specifically bound by VHH72 defined by SEQ ID NO: 124 and at least one ISVD capable of binding to or competing for the epitope specifically bound by VHH3.117 defined by SEQ ID NO: 127, such as the binding agent defined by SEQ ID NO: 119.

28. The composition according to claim 26 comprising the binding agent defined by SEQ ID NO: 118 and the binding agent defined by SEQ ID NO: 119.

29. A pharmaceutical composition comprising the binding agent according to claim 1.

30. A method of treating a Sarbecovirus infection in a subject comprising administering to the subject the binding agent according to claim 1.

31. An in vitro or ex vivo method for detecting a Sarbecovirus in a sample comprising:
contacting the sample with a binding agent according to claim 1, and
determining binding of the binding agent with a Sarbecovirus or a part thereof.

32. The binding agent according to claim 15, wherein the ISVD comprises a combination of CDR1, CDR2 and CDR3, wherein the CDR1, CDR2 and CDR3 are as present in a particular one of the sequences set forth in SEQ ID NOs: 1 to 10, wherein the CDR1, CDR2 and CDR3 are annotated according to any one of Kabat, MacCallum, IMGT, AbM, Martin or Chothia.

33. The binding agent according to claim 16, wherein the ISVD comprises a CDR1 defined by SEQ ID NO:65, a CDR2 defined by SEQ ID NO:66, and a CDR3 defined by SEQ ID NO: 51; or a CDR1 defined by any one of SEQ ID NO:52-54, a CDR2 defined by any one of SEQ ID NO: 55-62, and a CDR3 defined by any one of SEQ ID NO:21-27.
